(19) United States
(12) Patent Application Publication
(10) Pub. No.: US 2023/0053409 A1
(43) Pub. Date: Feb. 23, 2023

(54) ATAC-SEQ DATA NORMALIZATION AND METHOD FOR UTILIZING SAME

(71) Applicant: SEOUL NATIONAL UNIVERSITY R&DB FOUNDATION, Seoul (KR)

(72) Inventors: Hang Rae KIM, Seoul (KR); Hyun Mu SHIN, Seoul (KR); Gwanghun KIM, Seoul (KR)

(73) Assignee: SEOUL NATIONAL UNIVERSITY R&DB FOUNDATION, Seoul (KR)

(21) Appl. No.: 17/791,970

(22) PCT Filed: Oct. 30, 2020
(86) PCT No.: PCT/KR2020/015066

§ 371 Date: Jul. 11, 2022

(30) Foreign Application Priority Data
Jan. 9, 2020 (KR) 10-2020-0002829
Oct. 20, 2020 (KR) 10-2020-0136246

(51) Int. Cl. G16B 30/00; G16B 20/00; G16B 40/10

(52) U.S. Cl. CPC G16B 30/00; G16B 20/00; G16B 40/10

(57) ABSTRACT

The present invention relates to ATAC-seq data normalization for utilizing epigenetic information associated with chromatin openness, and a method for utilizing same. According to the present invention, it is possible to readily normalize and quantitatively compare ATAC-seq in various samples and various cohorts, and selected differential peaks can be used in various epigenetic studies, the diagnosis of diseases, and prediction of the prognoses of diseases.

### TECHNICAL FIELD

The present invention relates to a method of normalizing ATAC-seq data for utilizing epigenetic information related to chromatin openness and a method for utilizing the same.

### BACKGROUND ART

Epigenetics is defined as a field that studies the phenomenon in which specific genes are expressed differently between generations even though the DNA sequence that transmits genetic information between generations does not change. Epigenetic changes may be induced by various factors in cells constituting our body, and these changes may induce different responses in each individual. In particular, epigenetic changes are known to play a very critical role in the development and differentiation of various cells and are found to provide a major cause of most diseases, including cancer. The major mechanisms related to epigenetics that have been revealed until recently include DNA methylation, histone modification, chromatin remodeling, RNA-mediated targeting, and the like.

A large-scale parallel sequencing technique is used for epigenetic research based on the mechanism by which the chromatin structure is altered and access to specific loci by enzymes becomes easier under a specific environment. Formaldehyde-assisted isolation of regulatory elements (FAIRE) and sonication of crosslinked chromatin sequencing (Sono-seq) were developed to observe the nucleosome-depleted, chromatin open state and the nucleosome-occupied, chromatin closed state, respectively. DNase I hypersensitive site sequencing (DNase-seq) can also detect fragments cleaved by DNA degrading enzyme I (DNase I) and thus is used to identify open regions of chromatin. In addition, the Assay for Transposase-Accessible Chromatin using sequencing (ATAC-seq) uses a transposon, known as “jumping gene”, that moves from one location on the genome to another and finds a chromatin open region like the other methods mentioned above. It is used for genome-related research.

It is known that among them, ATAC-seq may analyze the chromatin open region faster and more sensitively compared to the conventionally used micrococcal nuclease (MNase)-seq and DNase-seq. Specifically, ATAC-seq is a technology that identifies ‘accessible DNA regions in chromatin’ with an active-type mutant Tn5 transposase that inserts a sequencing adapter into a chromatin open region. Tn5 transposase cleaves long DNA strands in a process called tagmentation. Tagmentation refers to the action of simultaneously ‘tagging’ and ‘fragmentation’ of DNA with a Tn5 transposase preloaded with a sequencing adapter. Thereafter, the tagged DNA fragment is purified, amplified by polymerase chain reaction (PCR), and sequencing of the amplified product is performed. The sequencing read obtained through this is used to infer an ‘open region’ that is an accessible chromatin region, and regions for transcription factor binding sites and nucleosome locations can be mapped.

However, various studies have not yet been conducted to quantify the “chromatin open region” identified through ATAC-seq and to apply the same to epigenetic studies using the same.

### DISCLOSURE

### Technical Problem

Accordingly, the present inventors quantified the chromatin open region identified by the ATAC-seq method, studied a method for utilizing the same, and selected a normalization control peak to correct the amount of the sample for quantitative analysis to confirm a method for analyzing the ATAC-seq results. In addition, the present inventors have completed the present invention by confirming a method for selecting and verifying biomarkers that can predict the responsiveness of anti-PD-1 therapy in patients with gastric cancer based on this.

Therefore, an object of the present invention is to provide a method for selecting a normalization control peak for quantification of ATAC-seq, a normalization method using the same, and a method of selecting a differential peak for predicting the responsiveness of anti-PD-1 therapy by reflecting the epigenetic characteristics of chromatin based on this.

### Technical Solution

Accordingly, the present invention provides a method for selecting normalization control peak for normalization of ATAC-seq, the method including steps of: a) aligning and peak calling cell-derived ATAC-seq data; b) selecting overlapping peaks between cell samples among the peaks called in step a); c) selecting a peak coincident with the DNase I hypersensitivity consensus peak among the peaks selected in step b); and d) selecting a peak having a coefficient of variation (CV) of less than 0.3 and peak width of less than 500 bp among the peaks selected in step c).

In addition, the present invention provides a normalization control peak selected by the method.

In addition, the present invention provides a method of selecting the ATAC-seq normalization factor.

In addition, the present invention provides a method of normalizing the area value of a peak by using the ATAC-seq normalization factor (F) selected.

In addition, the present invention provides a method of selecting a differential peak for predicting the responsiveness of anti-PD-1 therapy, the method including steps of: a) aligning and peak calling ATAC-seq data from patients who respond to anti-PD-1 therapy and those who do not; b) selecting a peak which is distinct from the responding and non-responding patient samples among the peak calling values of step a); c) normalizing the selected peak area by normalizing the peak area value of the peak selected in step b) by the normalization method; d) selecting a peak having a normalized area value exceeding a mean area value of the normalized peak area values obtained in step c) among the peaks selected in step b); and e) selecting a peak in which the mean difference between normalized peak area values among the anti-PD-1 therapy responding and non-responding patient samples among the peaks selected in step d) satisfies significance p<0.05.

In addition, the present invention provides a differential peak selected by the method.

In addition, the present invention provides a biomarker composition for predicting the responsiveness of anti-PD-1 therapy, the composition including one or more predictive peaks selected from the group consisting of SEQ ID NO: 233 to SEQ ID NO: 299.

In addition, the present invention provides a method of predicting the responsiveness of anti-PD-1 therapy, the method including a step of detecting one or more predictive peaks selected from the group consisting of SEQ ID NO: 233 to SEQ ID NO: 299.

In addition, the present invention provides a use of one or more predictive peaks selected from the group consisting of SEQ ID NO: 233 to SEQ ID NO: 299 for predicting the responsiveness of anti-PD-1 therapy.

### Advantageous Effects

According to the present invention, it is possible to provide easy normalization and quantitative comparison of ATAC-seq data in various samples and various cohorts, and the selected differential peak can be utilized for various epigenetic studies, disease diagnosis, and prognosis prediction.

### DESCRIPTION OF DRAWINGS

FIG. 1 is a view schematically illustrating a protocol of the present invention for deriving a predictive peak (biomarker) using ATAC-seq.

FIG. 2 is a view illustrating a process of normalization control peak selection, differential peak selection, and predictive peak selection for predicting a prognosis for anti-PD-1 therapy from the normalized differential peaks.

FIG. 3 is a view illustrating a method and a used formula for deriving a normalization factor (F).

FIG. 4 is a view illustrating a normalization control peak selection and normalization process.

FIG. 5 shows the result of confirming the change in the normalization factor according to the increase in the number of normalization control peaks in order to select the optimal number of normalization control peaks (a) and the result of confirming the rank change of 121 differential peaks according to the increase of the normalization control peak (b).

FIG. 6A is a view illustrating a process of selecting 67 differential peaks. FIG. 6B is a view showing nine representative differential peaks (M1, 2, 5, 17, 20, 29, 30, 35, 36) among the selected differential peaks visualized in the complete response (CR)+partial response (PR) and progress disease (PD) groups.

FIG. 7A is a view illustrating the sensitivity and specificity of representative differential peaks, which are selected predictive peaks, through receiver operating characteristics (ROC) curve analysis. FIG. 7B is a view illustrating a threshold, sensitivity, and specificity for determining the responsiveness of anti-PD-1 therapy using differential peaks.

FIG. 8 is a view including the left portion showing the difference of the mean normalized area values in the responder (R) and non-responder (NR) groups of representative differential peaks in the discovery cohort, the center portion showing confirmation of the responsiveness of anti-PD-1 therapy according to ‘mean normalized area value threshold,’ and the right portion showing the responsiveness of anti-PD-1 therapy by the differential peak, that is, results of comparing median progression-free survival (mPFS) based on chromatin openness.

FIG. 9 is a view showing the sensitivity and specificity of a representative differential peak in a discovery cohort.

FIG. 10 is a view showing the results of confirming the responsiveness to anti-PD-1 therapy by the combination of weighted score of predictive peaks in the discovery cohort. FIG. 10A includes a left portion showing the difference in the weighted score values in the responder (R) and non-responder (NR) groups and a right portion showing the responsiveness of anti-PD-1 therapy based on the weighted score minus the threshold. FIG. 10B is a view showing the responsiveness of anti-PD-1 therapy by the differential peak, that is, the median progression-free survival (mPFS) based on chromatin openness.

FIG. 11 is a view showing the results of confirming the sensitivity and specificity for the responsiveness of anti-PD-1 therapy using predictive peaks in the validation cohort.

FIG. 12 is a view showing the results of confirming the responsiveness of anti-PD-1 therapy in the metastatic gastric cancer patient group by the combination of weighted score of predictive peaks in the validation cohort. FIG. 12A includes a left portion showing the difference in the weighted score values in the responder (R) and non-responder (NR) groups and a right portion showing the responsiveness of anti-PD-1 therapy based on the weighted score minus the threshold. FIG. 12B is a view of showing the responsiveness of anti-PD-1 therapy by the differential peak, that is, the median progression-free survival (mPFS) based on chromatin openness.

FIG. 13 is a view showing the results of confirming the normalization control peaks in eight hematopoietic cells.

FIG. 14 is a view showing the results of confirming normalization control peaks in three acute myeloid leukemia cells.

FIG. 15 is a view showing the results of confirming normalization control peaks for normal bronchial epithelial cells, small cell lung cancer cells, normal prostate basal epithelial cells, small cell prostate cancer cells, and epidermal growth factor receptor-negative and positive glioblastoma.

### BEST MODE

The present invention provides a method for selecting normalization control peak for normalization of ATAC-seq, the method including steps of: a) aligning and peak calling cell-derived ATAC-seq data; b) selecting overlapping peaks between cell samples among the peaks called in step a); c) selecting a peak coincident with the DNase I hypersensitivity consensus peak among the peaks selected in step b); and d) selecting a peak having a coefficient of variation (CV) of less than 0.3 and peak width of less than 500 bp among the peaks selected in step c).

In the present invention, the term “peak” means a chromatin open region as a chromatin region accessible by Tn5 transposase.

Cell-derived ATAC-seq data of cell samples according to step a) of the present invention can be obtained by directly performing ATAC-seq using a cell sample to be confirmed or obtained from a public database. Alignment of read data may be based on a reference sequence, for example, mouse mm9, mouse mm10, human hg19, or human hg38 genome version of the genomic reference consortium database, and in the present invention, human hg19 version was used in a preferred embodiment.

Alignment of the obtained data and peak calling may be performed through methods known in the art, for example, alignment of the obtained read data may use a BWA, Bowtie, or Bowtie2 program, and peak calling may be performed by Hypergeometric Optimization of Motif EnRichment (HOMER) suite, Model-based Analysis of ChIP-Seq 2 (MACS2) or CisGenome.

In the present invention, step b) is a step of selecting overlapped peaks between cell samples from among the peaks called in step a). In a preferred embodiment of the present invention, common overlapped peaks in various subsets of CD8⁺ T cells were primarily selected.

In the present invention, step c) is a step of selecting a peak coincident with the consensus peak of DNase I hypersensitivity among the peaks selected in step b). This is the step of selecting a peak that 100% matches the DNase I hypersensitivity consensus peak of the ENCODE project, making it suitable for data normalization for analysis of various samples and cohort data. In addition, the peak selected as described above has the characteristic of being located in a transcription start site (TSS) or a 5′ untranslational region (5′ UTR).

In the present invention, step d) is a step of selecting a peak having a coefficient of variation of peak area values less than 0.3 and peak width of less than 500 bp between selected peaks among the peaks selected in step c). Step d) is a step for obtaining more accurate normalization control from the selected consensus ATAC-seq peak. In order to satisfy the ‘consensus’ criterion that can distinguish between samples with small differences in ATAC-seq peaks, peaks are selected based on two criteria: i) the coefficient of variation is less than 0.3 and ii) the peak width is less than 500 bp. That is, this selects with low ‘variability’ between samples. Here, 500 bp means a peak width in a base pair scale including up to two nucleosomes.

In the present invention, the term “normalization control peak” refers to a peak with ‘consensus’ between samples and is used for normalization.

For example, in a preferred embodiment of the present invention, it was obtained by the method including steps of: obtaining peaks from ATAC-seq data of various CD8⁺ T-cell subsets to select overlapped peaks commonly found among all CD8⁺ T-cell subsets; and matching the selected peaks with the consensus peak discovered by the DNase I hypersensitivity assay of the ENCODE project—that is, the ‘DNase I hypersensitivity consensus peak to select the peak located at the transcription start site or 5’ untranslational region while having 100% coincident with these.

More specifically, in the present invention, the above steps were performed in ATAC-seq data of CD8⁺ T cells to identify a total of 32,485 overlapped peaks among all CD8⁺ T-cell subsets, and among them, 4,798 peaks having 100% consistent with the DNase I hypersensitivity consensus peak and located in the transcription start site or the 5′ untranslational region were selected. Thereafter, 533 consensus peaks identically identified in the ATAC-seq data of CD8⁺ T cells used in the study were selected once again, and the peak with a coefficient of variation between the selected peaks of less than 0.3 and peak width between the selected peaks of less than 500 bp was selected, and finally, 232 normalization control peaks for normalization of ATAC-seq were derived.

The normalization control peak obtained by the above method is conserved across various species and can be used universally as a control peak for normalization in various cell populations. Accordingly, the cell may be one selected from the group consisting of cancer cells, stem cells, immune cells, inflammatory cells, epithelial cells, hematopoietic cells, and fibroblasts, and preferably, for example, peripheral blood mononuclear cells (PBMCs), B cells, T cells, CD8⁺ T cells, CD4⁺ T cells, CD8⁺ T cells, CD14⁺ monocytes, acute myeloid leukemia cells, bronchial epithelial cells, small cell lung cancer cells, prostate basal epithelial cells, small cell prostate cancer cells, or epidermal growth factor receptor (EGFR) negative and positive glioblastoma cells can be used for normalization of ATAC-seq data and its analysis. Particularly preferably, the cells may be immune cells.

The normalization control peak obtained through the method of the present invention may consist of sequences represented by SEQ ID NO: 1 to SEQ ID NO: 232. Accordingly, the normalization control peak may be at least one selected from the group consisting of SEQ ID NO: 1 to SEQ ID NO: 232, and the selected peak may constitute a normalization control group. In addition, the normalization control peak may be at least one selected from the group consisting of SEQ ID NOs: 1 to 20, and the selected peak may constitute a normalization control group. More detailed information about the normalization control peak is shown in Table 4.

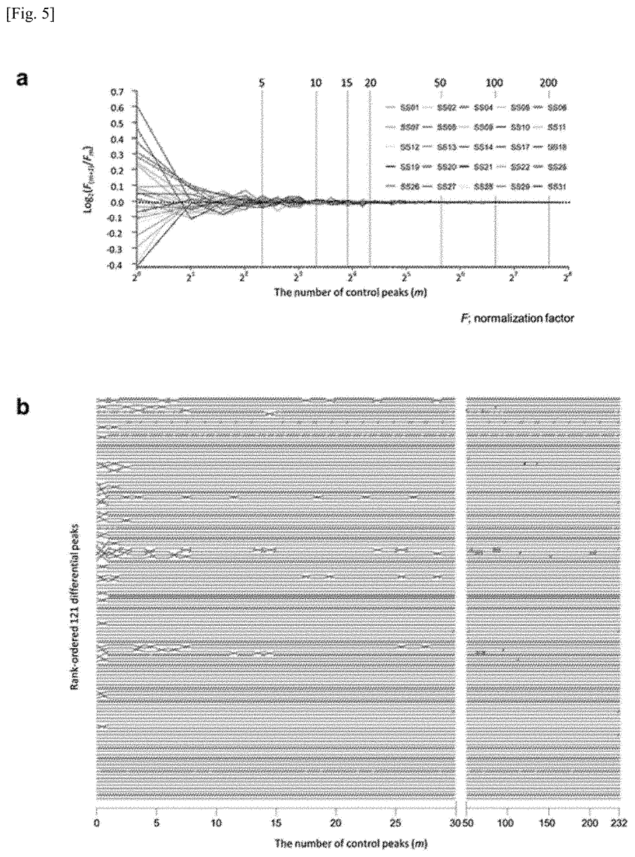

All 232 normalization control peaks of the present invention may be used for subsequent normalization factor calculation and differential peak selection, but after determining the order by arranging the mean of the area values of all peaks from SEQ ID NO: 1 to SEQ ID NO: 232 in descending order, 5 or more normalization control peaks may be utilized according to the rank, for example, 5 to 232 peaks, preferably 5 to 50 peaks, preferably 20 to 50 peaks may be used. As shown in FIG. 5A, in order to reflect the effect of adding the number of normalization control peaks, pairwise variations for all series of Fₘ and F₍ₘ₊₁₎ was calculated so that as the number of normalization control peaks increased, the normalization factor (F) gradually converged to one value. That is, when the number of normalization control peaks is 5 or more, the values of Fₘ and F₍ₘ₊₁₎ gradually converge so that the number of normalization control peaks is preferably 5 or more, and from 50, the normalization factor (F) gradually converges to one value without change according to the increase in the number of normalization control peaks (m). Considering the efficiency of data processing, it is desirable to set the number of control peaks to 50 or less. Therefore, in the present invention, the normalization control peaks are arranged in descending order by arranging the mean of the area values of all control peaks from SEQ ID NO: 1 to SEQ ID NO: 232 to determine the order, and then 5 or more peaks may be selected and utilized according to the rank. For example, 5 to 232 peaks, preferably 5 to 50 peaks, 5 to 20 peaks, 20 to 50 peaks can be used. In this case, 20 preferred normalization control peaks that can be used are shown as SEQ ID NOs: 1 to 20.

In addition, the present invention relates to a method for selecting the normalization factor (F) for quantification of ATAC-seq analysis, the method including steps of:

a) deriving the average height (k) of an individual normalization control peak selected from an individual sample by dividing the area of the individual normalization control peak selected by the above method by the peak width in an individual sample in a cohort as shown in the following formula:

Average height (k) of an Individual peak

$$k = \frac{area}{width} = \frac{the sum of enrichment within the peak}{the base length of peak} ;$$

b) selecting m number of normalization control peaks existing in an individual sample in the cohort, and deriving a mean height (h), which is a mean value of the average heights (k) of the selected m number of normalization control peaks, through the following formula:

Mean Height (h) of Selected Peaks Ach Sample

$$h = \frac{{\sum }_{i = 1}^{m} {k}_{i}}{m} , \\ {k}_{i} = \frac{the area {A}_{i} of the ith peak}{the base length of the ith peak}$$

m=the number of selected peaks in a sample; and

c) deriving an ATAC-seq normalization factor (F) through the following formula:

$${F}_{ab} = \frac{{s}_{a} ( = \frac{{\sum }_{i = 1}^{{n}_{a}} {h}_{ai}}{{n}_{a}} )}{{h}_{bj}} for 1 \leq a \leq b \leq 2$$

hₐᵢ=the mean height of the fifth sample in the cohort Cₐ

h_{bj}=the mean height of the jth sample in the cohort C_{b}

nₐ=the number of samples in the cohort Cₐ

The ATAC-seq normalization factor (F) normalizes the area of the peak region obtained by ATAC-seq, allowing quantitative comparison.

In the present invention, the term “discovery cohort” refers to a population for selecting a differential peak capable of predicting differentiation with respect to a condition of interest. The condition of interest may include various conditions expected or predicted to show a difference between samples according to the degree of chromatin openness, and the differentiation may be used in the same meaning as terms such as responsiveness and suitability.

In the embodiment of the present invention, a population to distinguish response and non-response, that is, responsiveness, to anti-PD-1 therapy as a condition of interest was used as a discovery cohort.

In the present invention, the term “validation cohort” refers to a population for validating the selected differential peak. In the validation cohort, the differential peak selected from the discovery cohort is applied to verify whether the selected differential peak can accurately predict the differentiation for the desired condition of interest.

In the above equation, when a=b, the discovery cohort and the validation cohort are the same, and the same normalization factor (F) is applied for normalization. In the embodiment of the present invention, the normalization factor (F) derived from the discovery cohort was equally applied to the validation cohort by making the discovery cohort and validation cohort the same.

In the present invention, the term “normalization factor (F)” is a factor for adjusting the openness value of ATAC-seq of each sample for quantitative comparison between samples and means a value obtained by dividing the mean height h for the “average height (k) of the normalization control peak” of all samples in the cohort by the mean height h of the normalization control peak selected from the sample to be analyzed. The normalization factor (F) used in the present invention is simpler and less system load for calculation compared to the conventionally used method. When data is processed by multiplying the normalization factor (F) by the peak area value, the area value can converge to the mean value of each group, allowing the quantitative adjustment. Therefore, ATAC-seq data collected from various samples and various cohorts can be adjusted using normalization factors (F), then utilized in the next analysis.

In the present invention, the mean height h means the mean height with respect to the “average height (k) of the normalization control peak” of the samples in the cohort, which means the chromatin openness of the individual samples. That is, the mean height (h) is the mean of k values present in the sample, and this value generally represents the chromatin openness of the normalization control peak of one sample.

In order to derive the normalization factor (F), 5 to 50 normalization control peaks present in individual samples in a cohort may be selected according to rank. As m, the number of normalization control peaks selected from the sample increases, the normalization value appears constant, so that normalization can be performed better. However, if the number is greater than a certain number, a better normalization effect according to the increase in the number of normalization controls is not confirmed, and the maximum effect is exhibited so that the number of optimal efficient normalization control peaks may be selected. The present inventors confirmed that the desired normalization could be sufficiently achieved by setting the number of normalization control peaks to 5 to 50, 5 to 20, and 20 to 50.

In addition, the present invention provides a method for normalizing the peak area of the ATAC-seq normalization factor (F) by multiplying the selected ATAC-seq normalization factor (F) as in the following formula:

Normalized area(A)=F_{ab}×A_{q}

in which F_{ab} is defined as follows:

$${F}_{ab} = \frac{{s}_{a} ( = \frac{{\sum }_{i = 1}^{{n}_{a}} {h}_{ai}}{{n}_{a}} )}{{h}_{bj}} for 1 \leq a \leq b \leq 2$$

hₐᵢ=the mean height of the fth sample in the cohort Cₐ

h_{bj}=the mean height of the fth sample M the cohort C_{b}

nₐ=the number of samples M the cohort Cₐ,

and A_{q} represents the area value of the q-th peak of the j-th sample of cohort C_{b}.

In addition, m, the number of normalization control peaks selected in the sample in the method may be 5 to 50.

In the present invention, the “normalized area (A)” is the same as the “peak area of the normalization factor (F),” and the peak area is used to quantitatively compare “chromatin openness.”

In addition, the present invention provides a method of selecting differential peak for predicting the responsiveness of anti-PD-1 therapy, the method including steps of: a) aligning and peak calling ATAC-seq data from patients who respond to anti-PD-1 therapy and those who do not; b) selecting a peak which is distinct from the responding and non-responding patient samples among the peak calling values of step a); c) normalizing the selected peak area by multiplying the peak area value (A_{d}) of the peak selected in step b) by the ATAC-seq normalization factor (F_{ab}) selected by the method of normalizing as in the following formula; Normalized area (A)=F_{ab}×A_{d}; d) selecting a peak having a normalized area value exceeding a mean area value of the normalized peak area values obtained in step c) from among the peaks selected in step b); and e) selecting a peak in which the mean difference between normalized peak area values from among the anti-PD-1 therapy responding and non-responding patient samples among the peaks selected in step d) satisfies significance p<0.05.

The A_{d} refers to the peak area value of the individual peaks selected in step b), and the normalized area of the individual peaks can be obtained by multiplying this by a normalization factor (F).

By selecting a peak having a normalized area value exceeding the mean of the normalized peak area values obtained in step c) from among the peaks selected in step d), a peak showing a distinct difference among all peaks can be selected and noise can be removed.

In the present invention, the term “differential peak” means a significant peak that appears differently between individuals as a result of ATAC-seq analysis, reflects different degrees of chromatin openness, and can be selected by the following method. Since the differential peak selected in the present invention is clearly different in responding patients and non-responding patients to anti-PD-1 therapy, it can be used as a biomarker for predicting the responsiveness of anti-PD-1 therapy. In particular, it can use differential peaks selected as highly predictable as “predictive peaks.” Here, “predictive peak” is used interchangeably with “predictive marker” and “biomarker.”

The peak calling of step a) is performed using two or more types of peak callers selected from the group consisting of HOMER suite, MACS2, and CisGenome, and the selection of step b) is characterized by selecting a common distinct peak from the two or more types of peak callers, thereby increasing reliability.

In a preferred embodiment of the present invention, among 2,560 differential peaks in the cohort, a peak exceeding a mean area value of the normalized area values by multiplying by a normalization factor (F) was selected. Thereafter, 121 differential peaks were selected by additionally selecting differential peaks in which the mean difference of normalized peak area values between the responder and non-responder groups was statistically significant (p<0.05). The list of normalized peaks in the normalization control sets of C₅, C₂₀, and C₅₀ using the normalization control peak numbers 5, 20, and 50 (C₅, C₂₀, C₅₀) in the differential peaks were compared. In these sets, 67 differential peaks shown in common without changing the order of the differential peaks were selected as predictive peaks finally confirming the responsiveness of anti-PD-1 therapy.

The present invention relates to a differential peak for predicting the responsiveness of anti-PD-1 therapy selected through the above method.

The differential peak is referred to herein as a predictive peak or a biomarker and may be one or more selected from the group consisting of SEQ ID NO: 233 to SEQ ID NO: 299, preferably one or more selected from the group consisting of SEQ ID NO: 233 to SEQ ID NO: 241.

SEQ ID NOs: 233 to 241 are referred to as representative differential peaks in the specification of the present invention, and is the peak selected once again among the 67 differential peaks based on i) a large difference in the relative numerical values between the mean area values of the responder and non-responder groups, ii) a higher mean area value in the responder groups, or iii) the low variance between the area values of the peaks in the responder groups. They can provide information on the responsiveness of anti-PD-1 therapy with high sensitivity and specificity based on a more pronounced difference in chromatin openness.

In addition, the present invention provides a biomarker composition for predicting the responsiveness of anti-PD-1 therapy, including one or more predictive peaks selected from the group consisting of SEQ ID NO: 233 to SEQ ID NO: 299, preferably SEQ ID NO: 233, SEQ ID NO: 234, SEQ ID NO: 237, SEQ ID NO: 249, SEQ ID NO: 252, SEQ ID NO: 261, SEQ ID NO: 262, SEQ ID NO: 267, and SEQ ID NO: 268.

In addition, the present invention provides a method of predicting the responsiveness of anti-PD-1 therapy, the method including a step of detecting one or more predictive peaks selected from the group consisting of SEQ ID NO: 233 to SEQ ID NO: 299.

In addition, the present invention provides a use of one or more predictive peaks selected from the group consisting of SEQ ID NO: 233 to SEQ ID NO: 299 for predicting the responsiveness of anti-PD-1 therapy.

The description of the biomarker composition for predicting responsiveness applies equally to the responsiveness prediction method and responsiveness prediction use, and overlapping descriptions are excluded to avoid the complexity of the description.

The predictive peaks can effectively provide information on responsiveness prediction even with a single one, but when they are used in combination, responsiveness can be predicted with improved sensitivity and specificity. In particular, it was confirmed that only the combination of four predictive peaks achieved the optimal saturation point in sensitivity and specificity in the present invention.

Accordingly, the biomarker composition of the present invention may include a combination of four or more predictive peaks selected from the group consisting of SEQ ID NO: 233 to SEQ ID NO: 299, preferably, a combination of four or more predictive peaks selected from the group consisting of SEQ ID NO: 233, SEQ ID NO: 234, SEQ ID NO: 237, SEQ ID NO: 249, SEQ ID NO: 252, SEQ ID NO: 261, SEQ ID NO: 262, SEQ ID NO: 267, and SEQ ID NO: 268, and most preferably, a combination of SEQ ID NO: 268, SEQ ID NO: 249, SEQ ID NO: 252, and SEQ ID NO: 262 (‘M36+M17+M20+M30’).

In the present invention, the predictive peak can be characterized in that it provides information on the responsiveness of anti-PD-1 therapy by distinguishing the difference in chromatin openness between patients who respond to anti-PD-1 therapy and those who do not.

Hereinafter, the present invention will be described in detail through specific examples, but the scope of the present invention is not limited by the following examples.

[Modes of the Invention]

Experimental Method

ATAC-seq library preparation and ultrahigh-speed sequencing

Nuclei isolated from CD8⁺ T cells underwent a transposition reaction using Illumina transposase in 1× Tagment DNA (TD) buffer. Library fragments were amplified using indexing primers from the Nextera kit (Illumina, San Diego, Calif., USA), and quantified. The quantified library was then verified by quantitative PCR (qPCR) using the KAPA Library Quantification Kit (Roche Applied Science, Basel, Switzerland). The pool of indexed libraries was sequenced using Illumina NextSeq500 for 75 single-read base sequences and analyzed using the CASAVA (v. 1.8.2) base sequencing software (Illumina). Reads were checked using the FASTQC software to assess the quality of single-end or paired-end read sequences. For analysis, sequencing data of each sample required a Phred quality score and a total number of reads equal to or greater than 30 (Q30) and 20 million, respectively.

ATAC-Seq Alignment and Peak Calling

FASTQ data were processed using the Trimmomatic software to increase read quality (per-base sequence quality >30) prior to alignment in the human genome and then aligned to the primary assembly of the GRCh37/hg19 human genome (chr1˜22 and chrX) using the Bowtie2 software with the parameters ‘-k 4 -N 1-R 5-end-to-end’. Finally, aligned reads were converted into browser extensible data (BED) format using the ‘bedtools bamtobed’ parameter. The positive strand was offset by +4 bp and the negative strand was offset by −5 bp. Tn5 transposase occupied 9 bp during transposition.

Three peak callers were used to perform peak calling: HOMER suite, MACS2, and CisGenome. The aligned read information was used for peak calling with the HOMER suite using ‘findPeaks’ and the ‘-style factor-tbp 3-region’ option; MACS2 using ‘callpeaks’ and the ‘-nolambda-nomodel-shift 100-extsize 200’ option; and CisGenome using the ‘Read extension length=150 bp’, ‘Bin size=50 bp’, ‘Max gap=50 bp’, and ‘Min peak length=100 bp’ options. During peak calling, according to the guidelines of Encyclopaedia of DNA Elements (ENCODE), which blacklisted regions with a large number of aligned reads (ultra-high signal artifact, UHS), the regions were excluded from the resulting peaks. Signal tracks were generated using the HOMER suite with the ‘makeUCSCfile’ command and the ‘-fragLength 147-tbp 3-style chipseq-raw’ options and visualized in the UCSC genome browser (https://genome.ucsc.edu/).

Peak Area Calculation

There are two methods for identification of accessible chromatin regions (open chromatin) by transposase marked as peaks: a method using peaks of various ranges or a method using peaks of a fixed range. The method using peaks of the fixed range may be beneficial in the analysis of large peaks or large datasets. When the peak size is small, the area excluded from the peak range may affect data analysis. Therefore, a method using peaks of various ranges (‘variable width peak’) was applied and analyzed using the Homer suite.

While comparing the overall chromatin accessibility (i.e., chromatin openness), limiting the range of the actual peak to a fixed range cannot accurately determine the degree of openness within the actual peak range. Thus, all sample data were concatenated into a single set and peak calling was performed using the HOMER suite for ‘variable-width peak’. Peak regions thus obtained corresponded to each peak in each sample. Instead of tag enrichments, peak area, representing the sum of tag enrichment, was calculated using each genome browser file such as bedGraphs. Briefly, bedGraphs were generated using the HOMER suite with the ‘makeUCSCfile’ command and the ‘-fragLength 147-tbp 3-style chipseq-raw’ options. The area of each peak was then calculated by summing height per base pair in the peak range using a genome browser track file. For each sample, the area of the q-th peak region was computed as follows:

$${A}_{q} = {\sum }_{p = {X}_{q}}^{{Y}_{q}} {d}_{pq} .$$

In the equation, d_{pq} refers to the height per base pair at position p of the coordinate from start (X_{q}) to end (Y_{q}) of the q-th peak. Finally, the peak areas were used to quantitatively compare “chromatin openness.”

Statistical Analyses

Data were expressed as means±standard errors of the mean (SEM), and the Mann-Whitney U test was used to compare difference between groups. The progression-free survival (PFS) and median PFS (mPFS) were determined using the Kaplan-Meier method. Groups were compared using the log-rank (Mantel-Cox) test. All statistical analyses were performed using GraphPad Prism 6.01 (GraphPad Software, La Jolla, Calif., USA) and R statistical software (R Foundation for Statistical Computing, Vienna, Austria). The Cutoff Finder (http://molpath.charite.de/cutoff/) was used to determine an optimal threshold for chromatin openness to classify responding and non-responding patients who were receiving anti-PD-1 therapy. Through receiver operating characteristics (ROC) curve analysis, the predictive accuracy of selected peaks was calculated. In all analyses, p<0.05 was considered to indicate statistical significance.

### Example 1. Patient Group and Experimental Design

In order to predict the responsiveness of gastric cancer patients to anti-PD-1 therapy, characteristics of peripheral blood CD8⁺ T cells of metastatic gastric cancer (mGC) patients being treated with the anti-PD-1 therapy, Keytruda (pembrolizumab; Kenilworth, N.J., USA) were analyzed in this study. CD8⁺ T cells are the main target cells for anti-PD-1 therapy. The response to anti-PD-1 therapy is classified into progressive disease (PD), stable disease (SD), partial response (PR), and complete response (CR). It was confirmed that there was no significant difference in the frequencies of potential CD8⁺ T cells and PD-1+ CD8⁺ T cells between the patients in the group.

Chronic stress caused by factors such as pathogens, microbiota, acidity, hypoxia, glucose deprivation, and the tumor microenvironment can cause epigenetic alterations in cells that make up our body, which can also cause subtle alterations in immune cells that make up the immune system. Accordingly, the present inventors studied the epigenetic characteristics of CD8⁺ T cells in the peripheral blood of metastatic gastric cancer patients who received anti-PD-1 therapy, in particular, determined that the treatment prognosis of patients before anti-PD-1 therapy can be predicted by using the chromatin openness that is distinguished between the responder and non-responder groups to anti-PD-1 therapy and intended to develop a biomarker using the same.

This study was approved by the Institutional Review Boards of Seoul National University Hospital (0905-014-280) and Samsung Medical Center (2015-09-053). Patients participating in the clinical trial (clinicaltrials.gov identifier NCT #02589496) were designated as the discovery cohort. Patients with stage IV metastatic gastric cancer mGC (n=32) who were undergoing anti-PD-1 therapy with pembrolizumab were recruited, and blood was collected before treatment and every three weeks thereafter. In addition, from January to May 2018, a new cohort of patients with stage IV mGC (n=52) who were treated with pembrolizumab was recruited as the validation cohort. Patient information for the discovery and validation cohorts is presented in Tables 1 and 2. Written informed consent was obtained from all patients in accordance with the Declaration of Helsinki. Microsatellite instability (MSI) or Epstein-Barr virus (EBV) positive information for tumor specimens was obtained from a report of a previous study (Kim et al., Nat. Med. 2018).

TABLE 1
Discovery Cohort Patient Characteristics

| | Number of | Age |
|---|---|---|
| Response | patients (n) | (years) |
| Total | 33 | 56.7 ± 1.9 |
| Complete response (CR) | 2 | 62.5 ± 6.5 |
| Partial response (PR) | 8 | 59.3 ± 4.2 |
| Stable disease (SD) | 7 | 64.3 ± 5.2 |
| Progressive disease (PD) | 15 | 51.0 ± 3.7 |

Ages are presented as means ± standard errors of the mean (SEMs).

TABLE 2
Validation Cohort Patient Characteristics

| | Number of | Age |
|---|---|---|
| Response | patients (n) | (years) |
| Total | 52 | 60.9 ± 1.4 |
| Complete response (CR) | 3 | 69.3 ± 6.6 |
| Partial response (PR) | 15 | 63.3 ± 2.6 |
| Stable disease (SD) | 12 | 61.3 ± 3.2 |
| Progressive disease (PD) | 22 | 58.0 ± 3.7 |

Ages are presented as means ± SEMs.

All CD8⁺ T cells were collected from blood samples of patients enrolled in a clinical trial phase II pembrolizumab, anti-PD-1 therapy, and for each sample, the genome-wide assay was performed for Tn5 transposase-accessible chromatin using ATAC-seq.

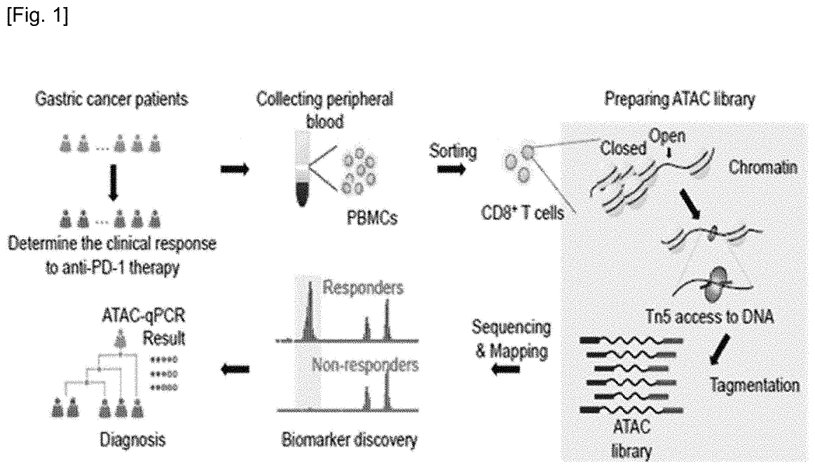

Through this analysis, in the present invention, biomarkers were derived for distinguishing between responder and non-responder patients before anti-PD-1 therapy, and it was confirmed that the prediction method using these biomarkers could have high accuracy and sensitivity. A protocol schematically illustrating the method is shown in FIG. 1.

The published ATAC-seq data were used for quantitative ATAC-seq analysis, and the data used are shown in Table 3.

TABLE 3

| Sample | Total | Aligned | % | % | % | | | | TSS |
|---|---|---|---|---|---|---|---|---|---|
| ID | reads | reads | Aligned | Duplicated | Mitochondria | NRF | PBC | FRIP | enrichment |
| SS01 | 79,369,131 | 72,958,765 | 91.9% | 51.3% | 31.4% | 0.98 | 0.86 | 0.60 | 16.4 |
| SS02 | 87,638,376 | 80,039,100 | 91.3% | 43.0% | 21.4% | 0.98 | 0.83 | 0.47 | 16.0 |
| SS03 | 126,685,260 | 115,837,870 | 91.4% | 45.6% | 21.2% | 0.98 | 0.86 | 0.55 | 26.8 |
| SS04 | 66,576,763 | 60,989,804 | 91.6% | 43.1% | 26.0% | 0.98 | 0.89 | 0.64 | 28.2 |
| SS05 | 69,794,733 | 63,072,918 | 90.4% | 46.4% | 26.2% | 0.98 | 0.85 | 0.54 | 18.1 |
| SS06 | 110,916,650 | 102,146,159 | 92.1% | 46.1% | 26.9% | 0.98 | 0.86 | 0.53 | 11.9 |
| SS07 | 87,534,113 | 80,315,836 | 91.8% | 50.5% | 29.0% | 0.98 | 0.83 | 0.50 | 14.1 |
| SS08 | 79,366,311 | 73,459,580 | 92.6% | 41.6% | 23.9% | 0.98 | 0.88 | 0.53 | 15.7 |
| SS09 | 111,528,381 | 102,440,329 | 91.9% | 47.7% | 21.2% | 0.98 | 0.77 | 0.51 | 16.9 |
| SS10 | 106,022,192 | 97,579,178 | 92.0% | 46.6% | 24.4% | 0.98 | 0.83 | 0.51 | 21.3 |
| SS11 | 71,755,642 | 66,493,486 | 92.7% | 38.3% | 20.8% | 0.98 | 0.88 | 0.46 | 16.3 |
| SS12 | 88,065,358 | 82,845,759 | 94.1% | 47.2% | 24.7% | 0.98 | 0.82 | 0.42 | 14.3 |
| SS13 | 64,060,244 | 60,505,431 | 94.5% | 44.3% | 21.5% | 0.97 | 0.83 | 0.44 | 19.2 |
| SS14 | 103,257,981 | 97,746,391 | 94.7% | 39.3% | 13.3% | 0.98 | 0.76 | 0.51 | 16.8 |
| SS15 | 124,158,216 | 116,490,224 | 93.8% | 53.5% | 16.8% | 0.97 | 0.66 | 0.57 | 16.3 |
| SS16 | 73,535,141 | 69,458,094 | 94.5% | 36.6% | 18.5% | 0.98 | 8.83 | 0.53 | 21.9 |
| SS17 | 84,446,831 | 79,526,537 | 94.2% | 38.0% | 14.7% | 8.98 | 0.80 | 0.38 | 16.3 |
| SS18 | 61,138,757 | 58,835,255 | 96.2% | 36.3% | 18.9% | 0.99 | 0.87 | 0.49 | 26.6 |
| SS19 | 74,499,318 | 71,572,361 | 96.1% | 44.2% | 21.4% | 0.95 | 0.81 | 0.41 | 28.2 |
| SS20 | 80,080,760 | 76,640,704 | 95.7% | 41.4% | 17.6% | 0.98 | 0.80 | 0.48 | 16.1 |
| SS21 | 70,826,527 | 67,917,053 | 95.9% | 40.5% | 18.0% | 0.98 | 0.22 | 0.54 | 17.2 |
| SS22 | 89,838,238 | 86,206,816 | 96.0% | 45.4% | 22.9% | 0.97 | 0.83 | 0.48 | 14.0 |
| SS23 | 25,306,813 | 69,825,407 | 92.7% | 37.3% | 18.0% | 0.98 | 0.86 | 0.35 | 10.8 |
| SS24 | 115,656,817 | 107,321051 | 92.8% | 39.1% | 14.7% | 0.98 | 0.79 | 0.37 | 12.3 |
| SS25 | 89,273,685 | 82,918,784 | 92.9% | 49.1% | 24.5% | 0.98 | 0.79 | 0.42 | 15.3 |
| SS26 | 107,117,518 | 99,277,606 | 92.7% | 32.6% | 11.6% | 0.98 | 0.84 | 0.48 | 13.4 |
| SS27 | 52,636,072 | 48,671,987 | 92.5% | 38.5% | 20.4% | 0.98 | 0.87 | 0.44 | 15.8 |
| SS28 | 64,582,832 | 60,171,173 | 93.2% | 43.7% | 23.8% | 0.98 | 0.86 | 0.42 | 14.8 |
| SS29 | 80,226,651 | 75,580,819 | 94.2% | 57.0% | 33.6% | 0.97 | 0.51 | 0.46 | 23.0 |
| SS30 | 101,999,249 | 95,839,289 | 94.0% | 54.0% | 30.3% | 0.97 | 0.81 | 0.39 | 13.5 |
| SS31 | 49,007,643 | 45,924,143 | 93.7% | 30.1% | 14.7% | 0.98 | 0.89 | 0.61 | 24.0 |
| SS32 | 114,145,932 | 107,358,491 | 94.1% | 53.5% | 24.7% | 0.97 | 0.74 | 0.54 | 20.5 |

* NRF, non-redundant fraction;
PBC, polymerase chain reaction (PCR) bottlenecking coefficient;
FRIP, fraction of reads in peaks;
TSS, Transcription start Site

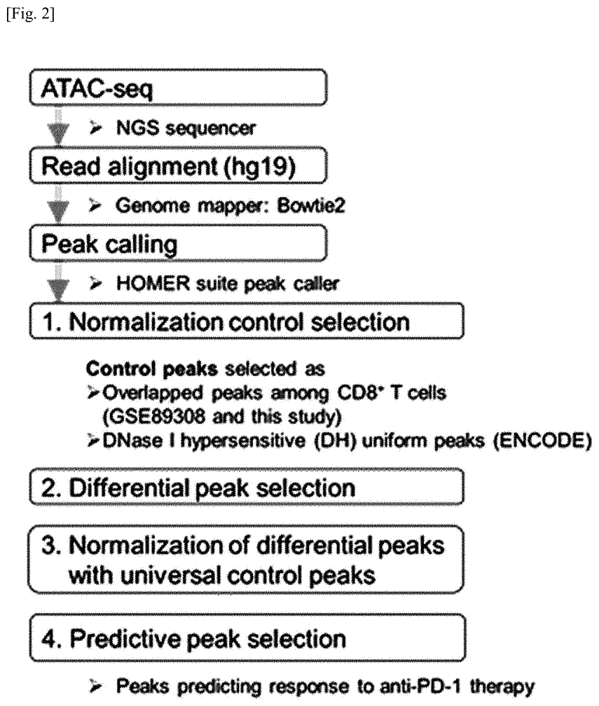

In order to improve the working speed of the computer delayed by the size of the data used for analysis, the Euclidian distance method was used. In the analysis of human genome (hg19)-mapped ATAC-seq data, a two-step selection process was performed to have a normalization control peak selection for normalization as a pre-step to discover a differential peak that is distinguished between responder and non-responder groups and a differential peak selection, and this process and the process of deriving a ‘differential peak’ for finally predicting the responsiveness of anti-PD-1 therapy is shown in FIG. 2.

As shown in FIG. 2, after ATAC-seq library generation, each library pool was quantified using a Bioanalyzer and sequenced on a single lane of the NextSeq 500 system using 75-bp single reads. Sequencing read (fastq) files were mapped using the human genome database (hg19) and Bowtie v. 2.0 software. Further analysis was performed in two stages:

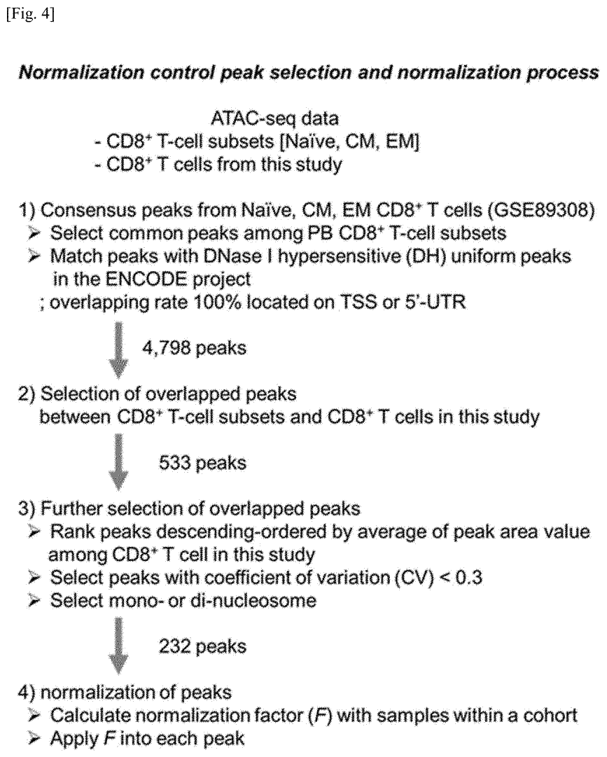

1) Selection of a consensus peak among CD8⁺ T-cell subsets and selection of a control peak by matching it to the consensus peak known as a DNase I hypersensitive site (“normalization control peak” selection step) followed by normalization of peaks from each patient using the normalization control peaks (normalization step); and 2) final selection of predictive peak to distinguish response and non-response to anti-PD-1 therapy after normalization of the selected differential peaks. The normalization control peak and normalization step of step 1) are shown in more detail in FIG. 4.

### Example 2. Selection of Normalization Control Peaks and Derivation of Normalization Factors (F)

The first selection step is to find a consensus peak to be applied as a normalization control and select it as a normalization control peak. That is, the normalization control group was determined for normalization from the results of CD8⁺ T cells ATAC-seq by the selection of peaks with low variability across samples. For this purpose, ATAC-seq data derived from the CD8⁺ T-cell subset including naïve and memory CD8⁺ T cells from PBMCs (GSE89308) obtained from healthy volunteers were used to screen control peaks for normalization. For all sample data, peak calling was performed using the HOMER suite to generate peak regions. Next, the area of selected peaks from each CD8⁺ T-cell subset was calculated using bedGraphs. After comparisons of CD8⁺ T-cell subsets, a total of 32,485 overlapped peaks were obtained among all CD8⁺ T-cell subsets using ‘findOverlaps.’ They were matched to the DNase I hypersensitivity consensus peak discovered by the ENCODE project located at the transcription start site (TSS) or 5′ untranslational region (5′ UTR), a transcriptionally active region of the genome, thereby selecting 4,798 peaks that matched 100% with DNase I hypersensitivity consensus peaks and were located on the TSS or 5′ UTR.

Among the selected peaks, 533 consensus ATAC peaks identified in the ATAC-seq data from CD8⁺ T cells used in this study were selected once again. Among the selected consensus ATAC-seq peaks, in order to satisfy the criterion of low diversity and showed high similarity between samples, two criteria with a coefficient of variation of less than 0.3 and a peak width of less than 500 bp were applied to select peaks. The peak width of 500 bp means a base pair size including up to two nucleosomes.

As a result, 232 ATAC peaks were selected, and they are shown in Table 4 below with information designating them. The numbers of the start and end positions of the peaks shown in Table 4 were designated based on hg19.

The 232 selected normalization control peaks shown in Table 4 are evolutionarily conserved among 17 species, and thus they may be useful for genomic studies of other species as well. In addition, it was confirmed that they could be applied to other cells such as CD4⁺ T cells and CD14+ monocytes, and the results are shown in Example 6 below in detail. Thereafter, the normalization control peak selected was applied for normalization in the cohort.

TABLE 4
Selected normalization control peaks

| | Chromo- | | | | | [symbol] |
|---|---|---|---|---|---|---|
| Control | some | Start | End | Symbol | Annotation | [symbol] |
| C1 | chr2 | 198,317,581 | 198,319,004 | COQ10B | Promoter, TSS | 1 |
| C2 | chr10 | 97,453,104 | 97,454,237 | TCTN3 | 5′ UTR | 2 |
| C3 | chr3 | 93,698,438 | 93,699,698 | ARL138 | Promoter, TSS | 3 |
| C4 | chr7 | 44,121,720 | 44,122,668 | POLM | Promoter, TSS | 4 |
| C5 | chr7 | 124,569,281 | 124,570,400 | POT1 | Promoter, TSS | 5 |
| C6 | chr11 | 124,980,971 | 124,982,078 | TMEM218 | Promoter, TSS | 6 |
| C7 | chr5 | 112,311,713 | 112,313,144 | DCP2 | Promoter, TSS | 7 |
| C8 | chr12 | 49,503,923 | 49,504,972 | LMBR1L | 5′ UTR | 8 |
| C9 | chr11 | 73,881,841 | 73,382,698 | PPME1 | Promoter, TSS | 9 |
| C10 | chr12 | 57,039,601 | 57,040,435 | ATP5F1B | Promoter, TSS | 10 |
| C11 | chr5 | 149,339,838 | 149,340,820 | SLC26A2 | Promoter, TSS | 11 |
| C12 | chr3 | 127,842,341 | 127,843,211 | RUVBL1 | Promoter, TSS | 12 |
| C13 | chr16 | 72,042,308 | 72,043,066 | DHODH | Promoter, TSS | 13 |
| C14 | chr2 | 197,503,899 | 197,504,911 | CCDC150 | Promoter, TSS | 14 |
| C15 | chr10 | 99,446,579 | 99,447,600 | AVPI1 | Promoter, TSS | 15 |
| C16 | chr12 | 54,712,365 | 54,719,307 | COPZ1 | Promoter, TSS | 16 |
| C17 | ch5 | 68,462,420 | 68,463,306 | CCNB1 | Promoter, TSS | 17 |
| C18 | chr20 | 13,619,031 | 13,620,138 | TASP1 | Promoter, TSS | 18 |
| C19 | chr19 | 33,462,571 | 33,463,350 | CEP89 | Promoter, TSS | 19 |
| C20 | chr19 | 104,359,038 | 104,360,317 | TDG | Promoter, TSS | 20 |
| C21 | chr8 | 74,883,696 | 74,885,141 | ELOC | promoter, TSS | 21 |
| C22 | chr3 | 186,287,767 | 186,289,030 | DNAJB11 | promoter, TSS | 22 |
| C23 | chr3 | 99,979,029 | 99,980,513 | TBC1D23 | 5′ UTR | 23 |
| C24 | chr4 | 141,444,608 | 141,446,084 | ELMOD2 | promoter, TSS | 24 |
| C25 | chr14 | 45,603,137 | 45,604,667 | FKBP3 | promoter, TSS | 25 |
| C26 | chr7 | 102,003,917 | 102,004,669 | LOC100630923 | promoter, TSS | 28 |
| C27 | chr9 | 131,842,917 | 131,843,849 | DQPP1 | promoter, TSS | 27 |
| C28 | chr12 | 54,120,913 | 34,121,840 | CALCOCO1 | promoter, TSS | 28 |
| C29 | chr14 | 23,398,267 | 23,399,495 | LOC101926933 | promoter, TSS | 29 |
| C30 | chr16 | 2,205,033 | 2,205,927 | SNHG19 | promoter, TSS | 39 |
| C31 | chr20 | 60,813,046 | 60,813,770 | OSBPL2 | promoter, TSS | 31 |
| C32 | chr2 | 216,176,146 | 216,177,543 | ATIC | 5′ UTR | 32 |
| C33 | chr1 | 202,317,372 | 202,318,359 | PPP1R128 | promoter, TSS | 33 |
| C34 | chr7 | 149,535,044 | 149,536,019 | ZNF862 | promoter, TSS | 34 |
| C35 | chr14 | 23,257,625 | 53,258,794 | GNPNAT1 | 5′ UTR | 35 |
| C36 | chr7 | 23,338,269 | 23,339,795 | MALSU1 | promoter, TSS | 36 |
| C37 | chr17 | 46,124,877 | 46,126,026 | NFE2L1 | promoter, TSS | 37 |
| C38 | chr15 | 100,273,047 | 100,274,120 | LYSMD4 | promoter, TSS | 38 |
| C39 | chr1 | 36,614,610 | 36,615,677 | TRAPPC3 | promoter, TSS | 39 |
| C40 | chr3 | 66,270,564 | 66,271,965 | SLC25A26 | promoter, TSS | 48 |
| C41 | chrX | 119,763,449 | 119,764,519 | C1GALT1C1 | promoter, TSS | 41 |
| C42 | chr14 | 73,924,904 | 73,925,758 | NUMB | promoter, TSS | 42 |
| C43 | chr10 | 104,004,821 | 104,005,489 | GBF1 | promoter, TSS | 43 |
| C44 | chr20 | 23,338,203 | 23,339,458 | LINC01431 | promoter, TSS | 44 |
| C45 | chr7 | 56,173,782 | 56,174,973 | CHCHD2 | promoter, TSS | 45 |
| C48 | chr3 | 127,770,514 | 127,771,610 | SEC61A1 | promoter, TSS | 46 |
| C47 | chr6 | 170,893,029 | 170,894,413 | PDCD2 | promoter, TSS | 47 |
| C48 | chr2 | 27,008,384 | 27,009,106 | CENPA | promoter, TSS | 48 |
| C49 | chr13 | 25,875,227 | 25,876,522 | NUP58 | 5′ UTR | 49 |
| C50 | chr3 | 14,692,665 | 14,693,338 | CCDC174 | promoter, TSS | 50 |
| C51 | chr20 | 5,093,144 | 5,094,245 | TMEM230 | promoter, TSS | 51 |
| C52 | chr6 | 88,299,162 | 88,300,219 | RARS2 | promoter, TSS | 52 |
| C53 | chr12 | 58,145,785 | 88,146,823 | CDK4 | promoter, TSS | 53 |
| C54 | chr6 | 111,279,278 | 111,280,465 | GTF3C6 | 5′ UTR | 54 |
| C55 | chr1 | 155,278,181 | 155,278,987 | FDPS | promoter, TSS | 55 |
| C56 | chr4 | 185,654,644 | 185,655,789 | CENPU | promoter, TSS | 56 |
| C57 | chr10 | 13,203,033 | 13,203,924 | MCM10 | promoter, TSS | 57 |
| C58 | chr2 | 219,575,276 | 219,376,173 | TTLL4 | 5′ UTR | 58 |
| C59 | chr6 | 170,615,298 | 170,616,376 | FAM120B | promoter, TSS | 59 |
| C60 | chr17 | 1,419,528 | 1,420,391 | INPPSK | promoter, TSS | 60 |
| C61 | chr14 | 32,030,613 | 32,031,121 | NUBPL | promoter, TSS | 61 |
| C62 | chr20 | 43,150,146 | 43,151,236 | SERINC3 | promoter, TSS | 62 |
| C63 | chr19 | 36,504,997 | 36,505,620 | LOC101927572 | promoter, TSS | 63 |
| C64 | chr7 | 150,075,855 | 150,076,741 | ZNF775 | promoter, TSS | 64 |
| C65 | chr18 | 39,534,461 | 39,535,915 | PIK3C3 | promoter, TSS | 65 |
| C66 | chr9 | 35,111,101 | 35,111,979 | FAM2148 | promoter, TSS | 66 |
| C67 | chr20 | 49,126,527 | 49,127,249 | PTPN1 | promoter, TSS | 67 |
| C68 | chr20 | 33,264,551 | 33,265,369 | PIGU | 5′ UTR | 68 |
| C69 | chr9 | 127,905,442 | 127,906,255 | SCAI | promoter, TSS | 69 |
| C70 | chr11 | 61,891,126 | 61,891,809 | INCENP | promoter, TSS | 70 |
| C71 | chr12 | 49,463,427 | 49,464,134 | RHEBL1 | promoter, TSS | 71 |
| C72 | chr19 | 11,456,825 | 11,457,389 | CCDC159 | promoter, TSS | 72 |
| C73 | chr12 | 110,939,483 | 110,940,450 | VPS29 | promoter, TSS | 73 |
| C74 | chrX | 77,150,435 | 77,151,643 | MAGT1 | promoter, TSS | 74 |
| C75 | chr6 | 49,430,355 | 49,431,624 | MUT | promoter, TSS | 75 |
| C76 | chr1 | 203,764,424 | 203,765,127 | ZC3H11A | 5′ UTR | 76 |
| C77 | chr3 | 128,902,187 | 126,903,439 | CNBP | promoter, TSS | 77 |
| C78 | chr2 | 85,822,301 | 85,823,256 | RNF181 | promoter, TSS | 73 |
| C79 | chr9 | 95,055,786 | 95,056,422 | IARS | promoter, TSS | 79 |
| C80 | chr7 | 54,826,380 | 54,827,704 | LOC100996654 | promoter, TSS | 80 |
| C81 | chr14 | 24,604,884 | 24,665,759 | PSME1 | promoter, TSS | 81 |
| C82 | chr1 | 156,163433 | 156,164,073 | SLC2544 | promoter, TSS | 82 |
| C83 | chr17 | 7,835,138 | 7,835,885 | CNTROB | promoter, TSS | 33 |
| C84 | chr9 | 115,983,271 | 115,983,998 | FKBP15 | promoter, TSS | 84 |
| C85 | chr18 | 21,082,699 | 21,083,898 | RMC1 | promoter, TSS | 85 |
| C86 | chr2 | 69,663,818 | 69,665,064 | NFU1 | 5′ UTR | 86 |
| C87 | chr3 | 50,396,620 | 50,397,330 | TMEM115 | promoter, TSS | 87 |
| C88 | chr10 | 104,263,341 | 104,264,065 | SUFU | promoter, TSS | 88 |
| C89 | chr10 | 105,991,750 | 105,992,632 | CFAP43 | promoter, TSS | 89 |
| C90 | chr6 | 31,125,807 | 31,126,453 | CCHCR1 | promoter, TSS | 99 |
| C91 | chr16 | 81,348,085 | 81,349,123 | GAN | promoter, TSS | 91 |
| C92 | chr6 | 32,095,827 | 32,096,372 | ATF6B | promoter, TSS | 92 |
| C93 | chr12 | 114,403,635 | 114,404,675 | RBM19 | promoter, TSS | 93 |
| C94 | chr2 | 97,405,416 | 97,406,226 | LMAN2L | promoter, TSS | 94 |
| C95 | chr1 | 203,830,373 | 203,831,113 | SNRPE | promoter, TSS | 95 |
| C96 | chr17 | 47,492,047 | 47,492,791 | PHB | promoter, TSS | 96 |
| C97 | chr17 | 35,715,849 | 35,716,593 | ACACA | promoter, TSS | 97 |
| C98 | chr19 | 36,119,454 | 36,120,170 | RBM42 | promoter, TSS | 98 |
| C99 | chr19 | 39,935,545 | 39,936,590 | SUPTSH | promoter, TSS | 99 |
| C100 | chr7 | 128,116,567 | 123,117,671 | METTL28 | promoter, TSS | 100 |
| C101 | chr12 | 50,419,084 | 50,419,686 | RACGAP1 | promoter, TSS | 101 |
| C102 | chr15 | 64,679,669 | 64,680,238 | TRIP4 | promoter, TSS | M2 |
| C103 | chr3 | 48,481,320 | 48,482,057 | TMA7 | promoter, TSS | 103 |
| C104 | chr19 | 49,990,391 | 49,991,067 | RPLISA | promoter, TSS | 104 |
| C105 | chr9 | 34,178,559 | 34,179,245 | UBAPI | promoter, TSS | 105 |
| C106 | chr19 | 11,266,326 | 11,266,924 | SPC24 | promoter, TSS | 106 |
| C107 | chr1 | 54,665,221 | 54,666,231 | CY35RL | promoter, TSS | M7 |
| C108 | chr22 | 18,586,359 | 18,560,921 | PEX26 | promoter, TSS | 108 |
| C109 | chr12 | 56,727,306 | 56,728,289 | PAN2 | promoter, TSS | 109 |
| C110 | chr17 | 41,277,224 | 41,277,747 | BRCA1 | promoter, TSS | 116 |
| C111 | chr19 | 13,858,361 | 13,858,934 | CCDC130 | promoter, TSS | 111 |
| C112 | chr19 | 5,977,894 | 5,978,520 | RANBP3 | promoter, TSS | 112 |
| C113 | chr20 | 37,100,840 | 37,101,662 | RALGAPB | promoter, TSS | 113 |
| C114 | chr22 | 30,234,080 | 30,234,725 | ASCC2 | promoter, TSS | 114 |
| C115 | chr17 | 27,139,070 | 27,140,040 | FAM222B | promoter, TSS | 115 |
| C116 | chr19 | 36,138,848 | 36,139,368 | COX661 | promoter, TSS | 116 |
| C117 | chr7 | 128,095,471 | 128,096,320 | HILPDA | promoter, TSS | 117 |
| C118 | chr19 | 11,708,005 | 11,708,637 | ZNF627 | promoter, TSS | 118 |
| C119 | chr17 | 26,971,797 | 26,972,686 | KIAA0100 | promoter, TSS | 119 |
| C120 | chr2 | 172,864,231 | 172,865,374 | METAP1D | promoter, TSS | 120 |
| C121 | chr20 | 1,098,917 | 1,099,591 | PSMF1 | promoter, TSS | 121 |
| C122 | chr20 | 37,590,596 | 37,591,507 | DHX35 | promoter, TSS | 122 |
| C123 | chr19 | 51,611,543 | 51,612,188 | CTU1 | promoter, TSS | 123 |
| C124 | chr12 | 51,477,051 | 51,477,666 | CSRNP2 | promoter, TSS | 124 |
| C125 | chr7 | 44,240,271 | 44,240,926 | YKT6 | promoter, TSS | 125 |
| C126 | chr15 | 59,396,962 | 59,397,659 | CCNB2 | promoter, TSS | 126 |
| C127 | chr2 | 73,963,997 | 73,984,995 | TPRKB | promoter, TSS | 137 |
| C128 | chr3 | 48,754,468 | 48,754,973 | IP6K2 | promoter, TSS | 128 |
| C129 | chr10 | 105,156,089 | 105,156,683 | PDCD11 | promoter, TSS | 129 |
| C130 | chrX | 118,986,796 | 118,987,387 | UPF3B | promoter, TSS | 130 |
| C131 | chr7 | 102,937,440 | 102,938,298 | PMPCB | promoter, TSS | 131 |
| C132 | chr19 | 54,605,926 | 54,606,340 | NDUFA3 | promoter, TSS | 132 |
| C133 | chr2 | 55,920,623 | 55,921,389 | PNPT1 | promoter, TSS | 133 |
| C134 | chr20 | 44,044,468 | 44,044,977 | PIGT | promoter, TSS | 134 |
| C135 | chr17 | 57,784,612 | 37,785,199 | PTRH2 | promoter, TSS | 135 |
| C136 | chr1 | 43,311,917 | 43,312,416 | ZNF691 | promoter, TSS | 136 |
| C137 | chr12 | 121,018,994 | 121,019,692 | POP5 | promoter, TSS | 137 |
| C138 | chr2 | 234,762,839 | 234,763,530 | HJURP | promoter, TSS | 138 |
| C139 | chr19 | 18,668,397 | 18,668,838 | KXD1 | promoter, TSS | 139 |
| C140 | chr19 | 53,465,796 | 53,466,658 | ZNF816-ZNF321P | promoter, TSS | 140 |
| C141 | chr12 | 57,881,460 | 57,882,173 | MARS | promoter, TSS | 141 |
| C142 | chr19 | 45,873,666 | 45,874,137 | ERCC2 | promoter, TSS | 142 |
| C143 | chr19 | 58,360,995 | 58,361,445 | ZNF587 | promoter, TSS | 143 |
| C144 | chr22 | 35,795,783 | 35,796,265 | MCM5 | promoter, TSS | 144 |
| C145 | chr12 | 124,118,029 | 124,118,558 | GTF2H3 | promoter, TSS | 145 |
| C145 | chr19 | 1,039,781 | 1,040,256 | ABCA7 | promoter, TSS | 146 |
| C147 | chr1 | 6,259,411 | 6,259,954 | RPL22 | promoter, TSS | 147 |
| C148 | chr18 | 47,900,933 | 47,901,758 | 5KA1 | promoter, TSS | 148 |
| C149 | chr1 | 44,440,063 | 44,440,783 | ATP6V0B | 5' UT | 149 |
| C150 | chr1 | 26,758,482 | 26,759,032 | DHDDS | promoter, TSS | 136 |
| C151 | chr22 | 39,189,835 | 39,190,462 | DNAL4 | promoter, TSS | 131 |
| C152 | chr17 | 5,094,839 | 5,095,522 | ZNF594 | promoter, TSS | 152 |
| C153 | chr22 | 23,483,879 | 23,484,614 | RSPH14 | promoter, TSS | 153 |
| C154 | chr9 | 131,591,658 | 131,592,394 | SPOUT1 | promoter, TSS | 154 |
| C155 | chr8 | 37,707,049 | 37,707,721 | BRF2 | promoter, TSS | 155 |
| C156 | chr9 | 44,079,591 | 44,080,138 | XRCC1 | promoter, TSS | 156 |
| C157 | chr17 | 56,296,205 | 56,296,948 | MKS1 | promoter, TSS | 157 |
| C158 | chr1 | 44,435,330 | 44,435,859 | DPH2 | promoter, TSS | 158 |
| C159 | chr18 | 55,297,133 | 55,297,928 | LOC100505549 | promoter, TSS | 159 |
| C160 | chr22 | 18,111,305 | 18,111,995 | ATP6V1E1 | promoter, TSS | 160 |
| C161 | chr1 | 44,172,843 | 44,173,445 | ST3GAL3 | promoter, TSS | 161 |
| C162 | chrX | 129,299,437 | 129,300,425 | AIFM1 | promoter, TSS | 162 |
| C163 | chr11 | 46,721,735 | 46,722,555 | ARHGAP1 | promoter, TSS | 163 |
| C164 | chr19 | 7,694,281 | 7,694,827 | XAB2 | promoter, TSS | 164 |
| C165 | chr3 | 124,448,809 | 124,449,799 | UMPS | promoter, TSS | 165 |
| C166 | chr1 | 231,376,566 | 231,377,356 | C1orf131 | promoter, TSS | 166 |
| C167 | chr19 | 2,819,568 | 2,820,267 | ZNF554 | promoter, TSS | 167 |
| C168 | chr1 | 222,763,083 | 222,763,717 | TAF1A-AS1 | promoter, TSS | 168 |
| C169 | chrX | 48,554,647 | 48,555,337 | SUV39H1 | promoter, TSS | 169 |
| C170 | chr20 | 62,496,100 | 62,496,814 | TPD52L2 | promoter, TSS | 170 |
| C171 | chr16 | 2,509,755 | 2,510,317 | TEDC2 | promoter, TSS | 171 |
| C172 | chr15 | 65,117,705 | 65,118,167 | PIF1 | promoter, TSS | 172 |
| C173 | chr9 | 130,679,067 | 130,670,693 | ST6GALNAC4 | promoter, TSS | 173 |
| C174 | chr19 | 19,249,083 | 19,249,488 | TMEM161A | promoter, TSS | 174 |
| C175 | chr7 | 127,983,505 | 127,984,235 | RBM28 | promoter, TSS | 175 |
| C176 | chr14 | 100,842,464 | 100,843,974 | WDR25 | promoter, TSS | 176 |
| C177 | chr19 | 17,858,224 | 17,858,712 | FCHO1 | promoter, TSS | 177 |
| C178 | chr11 | 68,816,050 | 68,816,709 | TPCN2 | promoter, TSS | 178 |
| C179 | chr1 | 53,703,996 | 53,704,524 | LOC100507564 | promoter, TSS | 179 |
| C180 | chr9 | 35,102,785 | 35,103,665 | STOML2 | promoter, TSS | 186 |
| C181 | chr8 | 145,980,585 | 145,981,260 | ZNF251 | promoter, TSS | 181 |
| C182 | chr16 | 1,993,129 | 1,993,666 | MSRB1 | promoter, TSS | 182 |
| C183 | chr9 | 131,133,302 | 131,133,896 | URM1 | promoter, TSS | 183 |
| C184 | chr6 | 31,774,452 | 31,774,898 | LSM2 | promoter, TSS | 184 |
| C185 | chr20 | 48,732,309 | 48,732,751 | UBE2V1 | promoter, TSS | 185 |
| C186 | chr1 | 33,502,358 | 33,502,787 | AK2 | promoter, TSS | 186 |
| C187 | chr22 | 47,158,220 | 47,158,853 | TBC1D22A | promoter, TSS | 187 |
| C188 | chr1 | 151,118,980 | 151,119,468 | SEMA6C | promoter, TSS | 188 |
| C189 | chr12 | 57,940,810 | 57,941,242 | DCTN2 | promoter, TSS | 189 |
| C190 | chr17 | 73,452,363 | 73,452,863 | TMEM94 | promoter, TSS | 190 |
| C191 | chr15 | 74,753,150 | 74,753,777 | U8L7 | promoter, TSS | 191 |
| C192 | chr15 | 90,895,226 | 90,095,796 | ZNF774 | promoter, TSS | 192 |
| C193 | chr20 | 62,526,139 | 62,526,683 | DNAICS | promoter, TSS | 193 |
| C194 | chrX | 9,430,908 | 9,431,616 | TBL1X | promoter, TSS | 194 |
| C195 | chr9 | 127,177,528 | 127,178,022 | PSMB7 | promoter, TSS | 195 |
| C196 | chr1 | 39,456,512 | 39,457,243 | AKIRIN1 | promoter, TSS | 196 |
| C197 | chr3 | 113,557,417 | 113,558,074 | GRAMD1C | promoter, TSS | 197 |
| C198 | chr19 | 57,922,343 | 57,922,830 | ZNF17 | promoter, TSS | 198 |
| C199 | chr15 | 44,092,539 | 44,093,175 | HYPK | promoter, TSS | 199 |
| C200 | chr3 | 50,385,450 | 50,365,942 | TUSC2 | promoter, TSS | 200 |
| C201 | chr6 | 32,143,699 | 32,144,130 | AGPAT1 | promoter, TSS | 201 |
| C202 | chr11 | 3,078,509 | 3,078,932 | CARS | promoter, TSS | 202 |
| C203 | chr11 | 67,236,528 | 67,237,017 | TMEM134 | promoter, TSS | 203 |
| C204 | chr7 | 152,372,873 | 152,373,539 | XRCC2 | promoter, TSS | 204 |
| C205 | chr17 | 3,749,353 | 3,749,872 | NCBP3 | promoter, TSS | 205 |
| C206 | chr20 | 34,129,573 | 34,130,131 | EBGIC3 | promoter, TSS | 206 |
| C207 | chr17 | 71,083,638 | 71,089,155 | SLC39A11 | promoter, TSS | 207 |
| C208 | chr20 | 60,982,173 | 60,982,776 | CABLES2 | promoter, TSS | 208 |
| C209 | chr19 | 49,954,957 | 49,955,341 | PIH1D1 | promoter, TSS | 209 |
| C210 | chr3 | 48,646,854 | 48,647,360 | UQCRC1 | promoter, TSS | 210 |
| C211 | chr5 | 141,258,478 | 141,259,116 | PCDH1 | promoter, TSS | 211 |
| C212 | chr15 | 75,315,741 | 75,316,129 | PPCDC | promoter, TSS | 212 |
| C213 | chr22 | 31,607,972 | 31,608,374 | LIMK2 | promoter, TSS | 213 |
| C214 | chr17 | 28,804,115 | 28,804,621 | GOSR1 | promoter, TSS | 214 |
| C215 | chr2 | 239,228,878 | 239,229,541 | TRAF3IP1 | promoter, TSS | 215 |
| C216 | chr10 | 104,192,104 | 104,192,680 | CUEDC2 | promoter, TSS | 216 |
| C217 | chr6 | 43,484,463 | 43,485,031 | POLR1C | promoter, TSS | 217 |
| C218 | chrX | 47,863,242 | 47,863,782 | ZNF182 | promoter, TSS | 218 |
| C219 | chr1 | 226,270,903 | 226,271,686 | UNC01703 | promoter, TSS | 219 |
| C220 | chr6 | 33,547,877 | 33,548,329 | BAK1 | promoter, TSS | 220 |
| C221 | chr1 | 27,718,810 | 27,719,275 | GPR3 | promoter, TSS | 221 |
| C222 | chr9 | 97,021,321 | 97,021,758 | ZNF169 | promoter, TSS | 222 |
| C223 | chr1 | 40,997,054 | 40,997,617 | ZNF684 | 5′ UTR | 223 |
| C224 | chr19 | 55,574,368 | 55,574,744 | RDH13 | promoter, TSS | 224 |
| C225 | chr8 | 23,145,369 | 23,145,952 | R3HCC1 | promoter, TSS | 225 |
| C226 | chr14 | 71,067,168 | 71,067,834 | MED6 | promoter, TSS | 226 |
| C227 | chr2 | 120,739,834 | 120,740,290 | SIRT4 | promoter, TSS | 227 |
| C228 | chr17 | 28,443,500 | 28,443,993 | NSRP1 | promoter, TSS | 228 |
| C229 | chr1 | 38,156,043 | 38,156,578 | C1of109 | promoter, TSS | 229 |
| C230 | chrX | 47,049,953 | 47,050,436 | UBA1 | promoter, TSS | 238 |
| C231 | chr17 | 56,494,850 | 56,495,237 | RNF43 | promoter, TSS | 231 |
| C232 | chr7 | 73,703,473 | 73,704,041 | CLP2 | promoter, TSS | 232 |

Calculation of Normalization Factor (F)

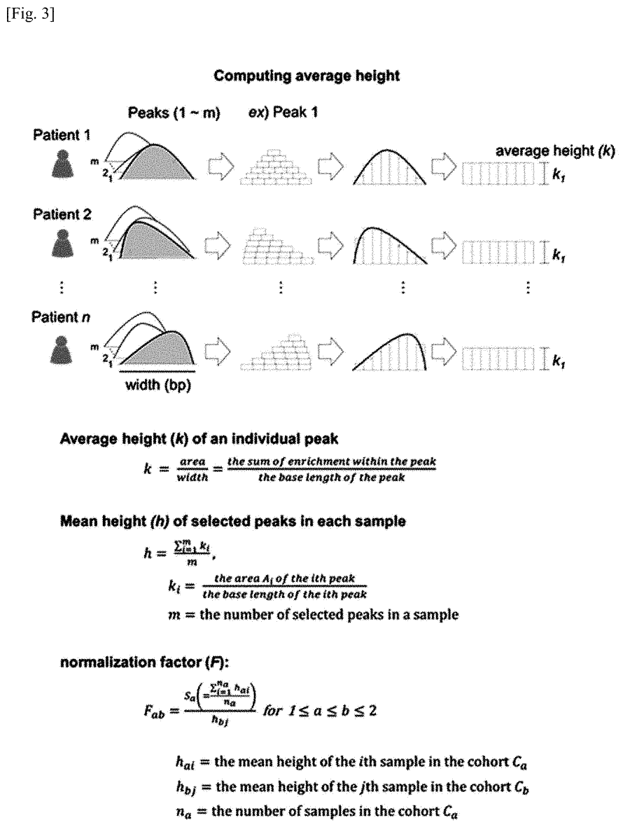

Under the assumption that the shape of the selected control peak is different for each sample, the average height (k) of one peak is calculated by dividing the peak area of the normalization control peak by the peak width. As a result, it was converted into a simple figure as shown in FIG. 3. The mean height (h) of the entire control peak was obtained by dividing the sum of the average peak (k) of each of m number of normalization control peaks selected from a sample of one patient by m. That is, the mean height (h) is the mean of k values in one sample, and this value represents the chromatin openness throughout the normalization control peak of one sample. Finally, a normalization factor (F) was obtained by dividing h of all samples in the cohort by the mean height (h) of the selected peak in the sample to be normalized, that is, each sample in order to adjust the openness value of ATAC-seq peak of each sample for quantitative comparison between samples. A schematic diagram for the formula used and the formula are shown in FIG. 3.

The normalization factor (F) derived in this manner has the advantage that it can be calculated by a simpler and less system load method compared to the conventional method. The normalization factor (F) means a value obtained by dividing the mean height of the normalization control peak of all samples in the discovery cohort by the mean height of the normalization control peak of the sample to be analyzed. If this is multiplied by the area value of the peak region, the value converges to the mean of each group, allowing the amount to be corrected.

This is described in detail through the formula as follows. In order to follow a normal distribution, the discovery cohort C₁ (Table 1) with sample size n₁=32 and validation cohort C₂ (Table 2) with sample size n₂=52 were randomly selected. For each sample, the mean height (h) of the entire normalization control peak was calculated as follows.

$$h = \frac{{\sum }_{i = 1}^{m} {k}_{i}}{m}$$

In the above formula, as the number m of normalization control peaks selected from the sample increases, the normalization value appears uniformly, allowing better normalization. In the present invention, the change in the normalization factor (F) was confirmed by changing the number of selected normalization control peaks from 1 to 232, which is further described with reference to FIG. 5. k is the average height of the i-th peak in the sample and is calculated as follows.

$${k}_{i} = \frac{the area {A}_{i} of the ith peak}{the base length of the ith peak}$$

The peak area was calculated as the sum of the tag enrichment within the peak. Since the widths of the selected peaks in each sample mean the length of the base sequence, they were all considered equivalent, so the mean height of the derived control peaks, was used to represent the mean amount of the control peaks selected for each sample. For each integer pair (a, b) such that 1≤a≤b≤2, the normalization factor (F) was defined as:

$${F}_{ab} = \frac{{s}_{a} ( = \frac{{\sum }_{i = 1}^{{n}_{a}} {h}_{ai}}{{n}_{a}} )}{{h}_{bi}}$$

where hₐᵢ and h_{bj} are the mean heights of the i-th and j-th samples in cohorts Cₐ and C_{b}, respectively. The normalization factor F_{ab} compares the j-th sample in cohort C_{b} with those of cohort Cₐ. For example, f₁₁ compares the j-th sample in discovery cohort C₁ with those in discovery cohort C₁. That is F₁₁ means that the normalization factor of the discovery cohort is equally applied to the validation cohort. F₁₂ compares the j-th sample in discovery cohort C₁ with those in validation cohort C₂, meaning that the discovery cohort and the validation cohort are different, and a normalization factor (F) is derived, respectively.

The peak areas of each sample were normalized by multiplying their normalization factors (F) as follows:

Normalized area (A)=F_{ab}×A_{q}

where F_{ab} is the normalization factor calculated from the j-th sample in cohort C_{b} with those in cohort Cₐ and A_{q} is the area value of the q-th peak in the j-th sample in cohort C_{b}.

Hereinafter, the normalization factor (F) is used to normalize the differential peak, and in this case, the q-th peak means the differential peak of the sample.

FIG. 4 shows the normalization control peak selection and normalization process described above.

### Example 3. Differential Peak Selection

As a second step, a differential peak was selected to identify the chromatin region among ATAC-seq data of CD8⁺ T cells that can reflect the clinical results of anti-PD-1 therapy. The discovery cohort shown in Table 1 was analyzed to select a differential peak that distinguishes response and non-response to anti-PD-1 therapy.

To select differential peaks from ATAC-seq data of CD8⁺ T cells in patients with gastric cancer who were receiving anti-PD-1 therapy, a responder group was chosen that included patients with complete response (CR) and partial response (PR); a non-responder group was chosen that included only patients with progressive disease (PD). That is, in the “selection step” to select the differential peak, the patient group with stable disease (SD) was excluded. Within the discovery cohort, the responder and non-responder groups were classified based on the clinical decision at a certain time point after finishing multiple cycles of anti-PD-1 therapy.

The responder and non-responder group data were pre-processed using the peak calling package, and as a result of peak calling detection using the peak callers Homer, MAC2, and CisGenome, 2,560 peaks commonly found in the three peak callers were selected to increase the reliability.

In order to quantitatively analyze the peaks distinguished between the responder and non-responder groups, the area values of 2,560 differential peaks in each sample were normalized using the normalization factor (F) value determined in the first step. In order to determine the number of normalization control peaks to be used at this time, Table 5 shows the normalization factors (F) calculated from three normalization control peak sets up to 5, 20, and 50 ranks according to rank.

TABLE 5
Normalization factor (F)

| | Sample | Ranked | Ranked | Ranked |
|---|---|---|---|---|
| Response | ID | ~5 | ~20 | ~50 |
| Responders | SS01 | 1.11 | 1.14 | 1.16 |
| (CR + PR) | SS11 | 1.05 | 1.07 | 1.09 |
| | SS04 | 1.07 | 1.11 | 1.14 |
| | SS06 | 0.95 | 0.96 | 0.98 |
| | SS08 | 1.01 | 1.06 | 1.06 |
| | SS10 | 0.75 | 0.75 | 0.75 |
| | SS12 | 0.92 | 0.97 | 0.57 |
| | SS13 | 0.90 | 0.92 | 0.91 |
| | SS28 | 0.97 | 1.02 | 1.04 |
| | SS29 | 0.90 | 0.91 | 0.93 |
| Non- | SS03 | 0.78 | 0.76 | 0.77 |
| responders | SS15 | 0.74 | 0.74 | 0.72 |
| (SD) | SS16 | 1.11 | 1.15 | 1.16 |
| | SS23 | 1.20 | 1.11 | 1.12 |
| | SS24 | 1.24 | 1.18 | 1.17 |
| | SS30 | 0.87 | 0.78 | 0.76 |
| | SS32 | 0.82 | 0.74 | 0.72 |
| Non- | SS02 | 0.90 | 0.94 | 0.95 |
| responders | SS05 | 1.03 | 1.06 | 1.08 |
| (PD) | SS07 | 1.07 | 1.04 | 1.02 |
| | SS09 | 0.82 | 0.82 | 0.82 |
| | SS14 | 1.39 | 1.38 | 1.37 |
| | SS17 | 1.44 | 1.36 | 1.33 |
| | SS18 | 1.29 | 1.36 | 1.41 |
| | SS19 | 1.05 | 1.10 | 1.10 |
| | SS20 | 1.13 | 1.12 | 1.11 |
| | SS21 | 0.93 | 0.97 | 0.96 |
| | SS22 | 0.82 | 0.84 | 0.83 |
| | SS25 | 0.97 | 0.99 | 1.02 |
| | SS26 | 1.15 | 1.04 | 1.02 |
| | SS27 | 1.27 | 1.25 | 1.24 |
| | SS31 | 1.44 | 1.42 | 1.43 |

Specifically, the F value and the corresponding normalized area of each sample converge to a constant value as the number of normalization control peaks (C₅, C₂₀, C₅₀) increased, respectively, or the rank of the normalized area was kept in a stable state. The stabilization process and the selection process of multiple normalization control peaks are shown in FIG. 5.

As shown in FIG. 5, the number of control peaks to be used (1≤m≤232, m is an integer) was selected among the 232 normalization control peaks selected in Example 2 and Table 4, which were ranked in descending order according to the mean area of the sample peaks. Individual normalization factors (F) were calculated by the method confirmed in Example 2.

As shown in FIG. 5A, pairwise variation was calculated for all series Fₘ and F₍ₘ₊₁₎ in order to reflect the effect of adding the normalization control peak. The gray vertical line represents the number of the normalization control peaks for peak normalization between samples. As the number of normalization control peaks increased, the normalization factor (F) gradually converged to one value. After the number of normalization control peaks exceeds 50, the normalization factor (F) gradually converged to one value without any change according to the increase in the number m of control peaks.

In FIG. 5B, for 121 differential peaks, Fₘ was calculated according to the number of normalization control peaks, and they were sorted by normalized area values, respectively, and the rank change of each peak was visualized. In order to obtain normalized area values (A), when ATAC-seq data for 2,560 differential peaks identified in common in the three peak callers was normalized using F calculated with three normalization control sets (C₅, C₂₀, C₅₀), the differential peaks were maintained without reversal of the rank. Therefore, it can be confirmed that normalization can be performed well because the rank of the differential peaks does not change and does not show a large fluctuation even if the number of controls is changed in the normalization control set or more. Therefore, a normalization control set of C₅, C₂₀, and C₅₀ was used in the following.

A process of selecting a desirable predictive peak that can be used for prognosis prediction from the differential peak was additionally performed using the following two-step process.

First, as a first step, the mean of the normalized area values of the differential peaks among 2,560 differential peaks in the cohort was obtained. Distinct differential peaks with normalized area values exceeding the mean area values were selected. 121 differential peaks were selected by additionally selecting differential peaks in which the mean difference in normalized peak area values between the responder and non-responder groups was statistically significant (p<0.05). As a second step, normalized peak lists were compared in the normalization control sets of C₅, C₂₀, and C₅₀ using normalization controls with the number of controls 5, 20, and 50 (C₅, C₂₀, C₅₀) so that there was no reversal of the rank. In these sets, 67 differential peaks that appear in common without reordering the differential peaks were selected. The selected 67 differential peaks were ordered according to differences in the mean values shared by the three sets of controls. They were ranked by the relative distance between responder and non-responder groups, the area of each peak was divided by the largest area among all samples and then calculated means from each group.

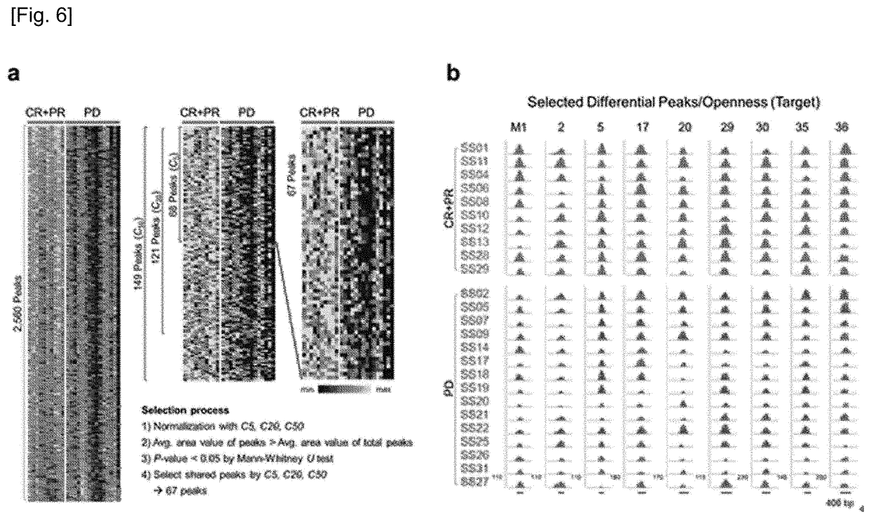

The process of finally selecting 67 differential peaks among the 2,560 peaks described above is shown in FIG. 6.

As shown in FIG. 6A, three peak callers of Homer suite, MACS2, and CisGenome were used to select 2,560 differential peaks that distinguish anti-PD-1 therapy responder groups (complete response+partial response, CR+PR) and non-responder groups (progress disease, PD). 121 peaks were selected according to the criteria and calculation formula described above, and, finally, 67 peaks were selected. The 67 differential peaks were identically selected in the results processed with three sets of normalization control peaks (C₅, C₂₀, and C₅₀).

Chromatin accessibility heatmaps represent normalized peak area values (rows) and patients (columns). Minimum and maximum area values are indicated in navy and yellow, respectively.

FIG. 6B is a view of showing the visual result of nine representative differential peaks (M1, 2, 5, 17, 20, 29, 30, 35, 36) among the selected 67 differential peaks using the genome browser of the University of California (Santa Cruz).

Nine representative differentiation peaks were selected based on three criteria: i) a large difference in the relative numerical values between the mean area values of responder and non-responder groups, ii) higher mean area values in responder groups, or iii) low variance between area values of peaks in responder groups.

The genome browser track in FIG. 6B shows nine differential peaks that serve as targets (predictive markers) predicting anti-PD-1 therapy responder groups (CR+PR, green) and non-responder groups (PD, red), which are used as a chromatin openness. The number above each differential peak denotes the target ID. The y-axis represents the adjusted read count according to the normalized area value calculated using the normalization factor (F). Scale bars indicate base pairs of 400 bp in base sequence length.

### Example 4. Predictability Verification Using Selected Differential Peaks

In order to use the selected differentiation marker determined according to the normalized area value of the peak as a responsiveness prediction marker for anti-PD-1 therapy, the responsiveness prediction effect was verified. For verification, the Cutoff Finder online tool (http://molpath.charite.de/cutoff) was used to find the best cut-off values for use as predictive markers and response guidelines for anti-PD-1 therapy in patients who were receiving anti-PD-1 therapy. The threshold was determined by comparing the responder and non-responder groups to minimize the Euclidean distance between the receiver operating characteristic (ROC) curves. The threshold of each differential peak was determined as follows: first, the predictive performance of the threshold for each differential peak was estimated according to the area under the receiver operating characteristic (AUROC) curve. Then, the discriminative ability of the threshold for both groups was assessed by calculating their sensitivity, specificity, and accuracy (ACC). Finally, the threshold of each differential peak was used in PFS analysis to evaluate the prognostic and diagnostic ability of anti-PD-1 therapy.

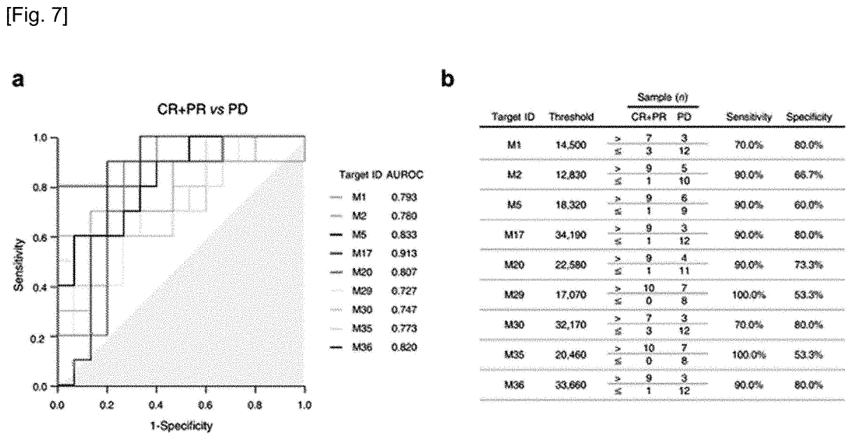

FIG. 7 shows the results of the receiver operating characteristics curve analysis, which verified the predictability of the differential peak selected as the predictive peak for prognostic diagnosis between the responder and the non-responder groups through the above process.

FIG. 7A shows the receiver operating characteristic (ROC) curves for nine representative differential peaks (M1, M2, M5, M17, M20, M29, M30, M35, and M36) selected as predictive peaks in the discovery cohort. FIG. 7B shows the sensitivity and specificity of the predictive peak and the threshold value determined through the Cutoff Finder online tool for the nine predictive peaks. Predictive peaks (target ID) were sorted in descending order with respect to the relative mean difference of normalized area values between responder groups (CR+PR) and non-responder groups (PD). Sensitivity and specificity were determined with samples larger or smaller than the threshold for normalized area values for patients who responded and did not respond to anti-PD-1 therapy, respectively.

The nine representative differential peaks showed statistically fair diagnostic ability with the area under the receiver operating characteristic (AUROC) values 0.7 and specificity and sensitivity of 88.0±3.6% and 70.0±3.9% (means±SEM), respectively. It was thus verified that it has a statistically significant ability to determine the responsiveness of anti-PD-1 therapy prognosis.

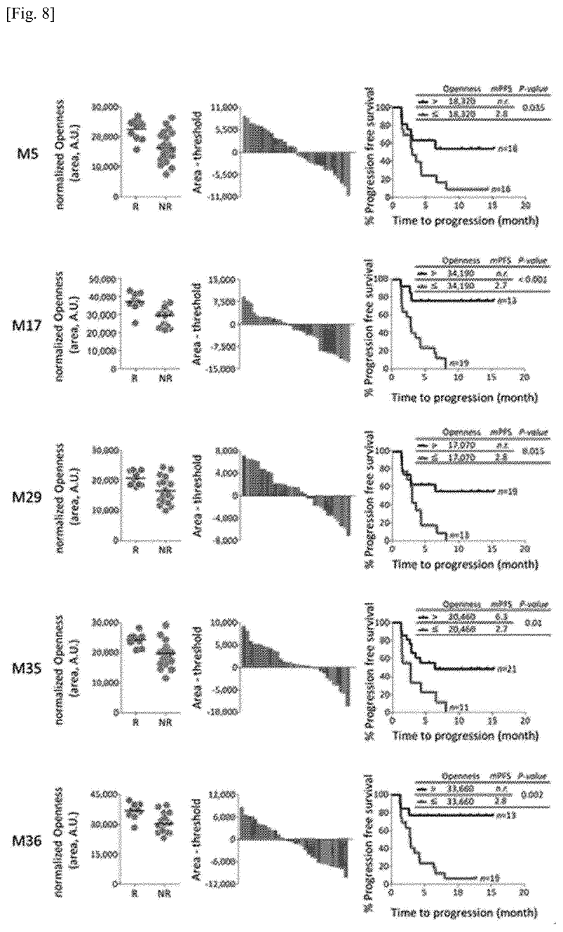

The additional results of predicting the anti-PD-1 therapy results with the predictive peaks discovered in the discovery cohort are shown in FIG. 8. The left plot of FIG. 8 shows the normalized area value results for four representative predictive peaks (M5, M17, M29, M35) in the anti-PD-1 therapy responder group (R, CR+PR) and non-responder group (NR, PD+SD) with normalized area values. The horizontal bar represents the mean value of peak area of each group. Middle plots are waterfall plots of normalized area values for an anti-PD-1 therapy responder group (CR+PR) and a progressive disease group (PD) minus threshold in descending order on the horizontal axis. Right plots show clinical outcomes of anti-PD-1 therapy determined using a threshold on a progression-free survival curve.

As confirmed on the left portion of FIG. 8, normalized area values (i.e., chromatin openness) by ATAC-seq analysis were clearly differentiated into anti-PD-1 therapy responder groups (R) and non-responder groups (NR) in the discovery cohort. Previous other studies have reported that microsatellite instability (MSI) and Epstein-Ban virus (EBV) positivity in tumor specimens can significantly predict responder group to anti-PD-1 therapy. However, according to the present invention, patients belonging to the non-responder group were distinguished despite the tumor MSI and EBV positive. That is, in the discovery cohort, all EBV-positive patients were classified as responder groups by ATAC-seq, and patients with MSI were also classified as responder groups by ATAC-seq. Despite the fact that the tumor was MSI, a patient (SS #20) who did not respond to anti-PD-1 therapy was correctly classified as a non-responder group by ATAC-seq. These results indicate that the predictive marker selected according to the method of the present invention is used to more accurately predict the responsiveness of anti-PD-1 therapy.

The right plot shows the progression-free survival curve, where the area value of the peak is greater than the threshold, for example, when the area value of the M17 predictive marker peak exceeds the threshold of 34,190, the progression-free survival period with no tumor growth was significantly longer compared to the groups below that value. In the M17, M29, and M36 predictive marker peaks, a progression-free survival period exceeding a 20-month measurement period was confirmed, and these were denoted as n.r. (not reached).

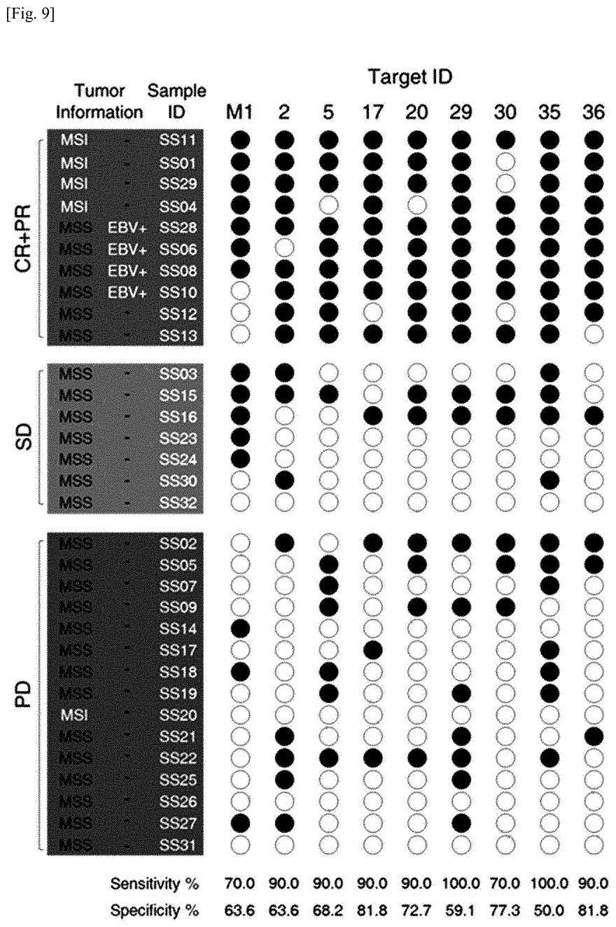

As a result, the differential peak selected according to the criteria of the present invention was classified by open chromatin (openness>threshold) and closed chromatin (openness threshold) with high sensitivity and specificity for the patient's responsiveness to anti-PD-1 therapy. Thus, it was confirmed that they could be used as predictive markers. Their sensitivity and specificity are additionally shown in FIG. 9 and Table 6.

TABLE 6

| | | | Sample | | | |
|---|---|---|---|---|---|---|
| Target | | | (n) | | Sensi- | Speci- |
| ID | AUROC | | R* | NR** | tivity | ficity |
| M1 | 0.745 | > | 7 | 8 | 70.0% | 63.6% |
| | | ≤ | [symbol] | 14 | | |
| M2 | 0.777 | > | 8 | 8 | 90.0% | 63. [symbol] % |
| | | ≤ | 1 | 14 | | |
| M3 | 0.886 | > | 9 | 4 | 90.0% | 81.8% |
| | | ≤ | 1 | 18 | | |
| M4 | 0.718 | > | 7 | 7 | 70.0% | 68.2% |
| | | ≤ | 3 | 16 | | |
| M5 | 0.841 | > | 9 | 7 | 90.0% | 68.2% |
| | | ≤ | 1 | 15 | | |
| M6 | 0.7 [symbol] | > | 7 | 8 | 70.0% | 77.3% |
| | | ≤ | 3 | 17 | | |
| M7 | 0.845 | > | 9 | 7 | 90.0% | 68.2% |
| | | ≤ | 1 | 15 | | |
| M8 | 0.7 [symbol] 9 | > | 5 | 4 | 80.0% | 81.8% |
| | | ≤ | 2 | 17 | | |
| M9 | 0.7 [symbol] 1 | > | 8 | 13 | 80.0% | 40.9% |
| | | ≤ | 2 | 9 | | |
| M10 | 0.718 | > | 9 | 10 | 90.0% | 54.5% |
| | | ≤ | 1 | 12 | | |
| M11 | 0.786 | > | 8 | 11 | 90.0% | 5 [symbol] % |
| | | ≤ | 1 | 11 | | |
| M12 | 0.70 [symbol] | > | 9 | 12 | [symbol] | 45.5% |
| | | ≤ | 1 | 10 | | |
| M13 | 0.809 | > | 5 | 5 | 80.0% | 77.3% |
| | | ≤ | 2 | 17 | | |
| M14 | 0.750 | > | 9 | 8 | 90.0% | 83.8% |
| | | ≤ | 1 | 1 [symbol] | | |
| M15 | 0.723 | > | 7 | [symbol] | 70.0% | [symbol] 9.1% |
| | | ≤ | 3 | 13 | | |
| M16 | 0.750 | > | 9 | 8 | 90.0% | 63.6% |
| | | ≤ | 1 | 14 | | |
| M17 | 0.90 [symbol] | > | 9 | 4 | 80.0% | 61. [symbol] % |
| | | ≤ | 1 | 18 | | |
| M18 | 0.814 | > | 9 | 8 | [symbol] | 88.2% |
| | | ≤ | 1 | 14 | | |
| M19 | 0. [symbol] 34 | > | 10 | 7 | 100.0% | 72.7% |
| | | ≤ | 0 | 15 | | |
| M20 | 0.7 [symbol] | > | 9 | [symbol] | 90.0% | 72.7% |
| | | ≤ | 1 | 16 | | |
| M21 | 0.818 | > | 9 | 6 | 90.0% | 59. [symbol] % |
| | | ≤ | 1 | 19 | | |
| M22 | 0.727 | > | 8 | 9 | 80.0% | 59.1% |
| | | ≤ | 2 | 13 | | |
| M23 | 0.832 | > | 5 | 5 | [symbol] | 77.3% |
| | | ≤ | 2 | 17 | | |
| M24 | 0.736 | > | 7 | 7 | 70.0% | 88.2% |
| | | ≤ | 3 | 15 | | |
| M25 | 0.741 | > | 8 | 8 | 80.0% | 63.5% |
| | | ≤ | 2 | 14 | | |
| M26 | 0.785 | > | 9 | 8 | 90.0% | 59.1% |
| | | ≤ | 1 | 13 | | |
| M27 | 0.818 | > | 7 | 9 | 70.0% | 77.3% |
| | | ≤ | 3 | 17 | | |
| M28 | 0.823 | > | 9 | 6 | 90.0% | 63.5% |
| | | ≤ | 1 | 14 | | |
| M29 | 0.777 | > | 10 | 9 | 100.0% | [symbol] |
| | | ≤ | 0 | 13 | | |
| M30 | 0.766 | > | 7 | 5 | 70.0% | 77. [symbol] % |
| | | ≤ | 3 | 17 | | |
| M31 | 0.73 [symbol] | > | 8 | 8 | 80.0% | 63.6% |
| | | ≤ | 2 | 14 | | |
| M32 | 0.791 | > | 9 | 9 | 90.0% | 59.1% |
| | | ≤ | 1 | 13 | | |
| M33 | 0.81 [symbol] | > | 9 | [symbol] | [symbol] | 59.1% |
| | | ≤ | 1 | 13 | | |
| M34 | 0.7 [symbol] 4 | > | 9 | 8 | 90.0% | 63.8% |
| | | ≤ | 1 | 14 | | |
| M35 | 0.873 | > | 10 | 11 | 100.0% | 50.0% |
| | | ≤ | 0 | 11 | | |
| M36 | 0.68 [symbol] | > | [symbol] | 4 | 90.0% | 81.6% |
| | | ≤ | 1 | 18 | | |
| M37 | 0.750 | > | 8 | [symbol] | 80.0% | 77.3% |
| | | ≤ | 2 | 17 | | |
| M38 | 0.805 | > | 7 | 6 | 70.0% | 72.7% |
| | | ≤ | 3 | 16 | | |
| M39 | 0.795 | > | [symbol] | [symbol] | 90.0% | 852% |
| | | ≤ | 1 | 15 | | |
| M40 | 0.823 | > | 9 | 5 | [symbol] | 77.3% |
| | | ≤ | 1 | 17 | | |
| M41 | 0.745 | > | 6 | 8 | 80.0% | 63.6% |
| | | ≤ | 2 | 14 | | |
| M42 | 0.754 | > | 7 | 5 | 70.0% | 77.3% |
| | | ≤ | 3 | 17 | | |
| M43 | 0.736 | > | 7 | 6 | 70.0% | 72.7% |
| | | ≤ | 9 | 18 | | |
| M44 | 0.727 | > | 7 | 4 | 70.0% | 81.6% |
| | | ≤ | 3 | 15 | | |
| M45 | 0.70 [symbol] | > | 7 | 6 | 70.0% | 72.7% |
| | | ≤ | 3 | 16 | | |
| M46 | 0.682 | > | 7 | [symbol] | 70.0% | 98.2% |
| | | ≤ | 3 | 13 | | |
| M47 | 0.806 | > | 9 | 7 | 80.0% | 88.4% |
| | | ≤ | 1 | 15 | | |
| M48 | 0.814 | > | 7 | 3 | 70.0% | 83.6% |
| | | ≤ | 3 | 19 | | |
| M49 | 0.68 [symbol] | > | 6 | 8 | 80.0% | 59.1% |
| | | ≤ | 2 | 14 | | |
| M50 | 0.791 | > | 9 | 9 | [symbol] | 6 [symbol] % |
| | | ≤ | 1 | 13 | | |
| M51 | 0.786 | > | 9 | 7 | 90.0% | [symbol] |
| | | ≤ | 1 | 15 | | |
| M52 | 0.788 | > | 8 | 9 | 90.0% | [symbol] |
| | | ≤ | 1 | 13 | | |
| M53 | 0.758 | > | 9 | 9 | [symbol] | 59.1% |
| | | ≤ | 1 | 13 | | |
| M54 | 0.700 | > | 7 | 6 | 70.0% | 72.7% |
| | | ≤ | 3 | [symbol] | | |
| M55 | 0.759 | > | 8 | 7 | 80.0% | 68.2% |
| | | ≤ | 2 | [symbol] | | |
| M56 | 0.514 | > | 9 | 7 | [symbol] | 68.2% |
| | | ≤ | 1 | 15 | | |
| M57 | 0.877 | > | 9 | 4 | 90.0% | 81.8% |
| | | ≤ | 1 | 15 | | |
| M58 | 0.764 | > | 7 | 5 | 70.0% | 77.3% |
| | | ≤ | 3 | 17 | | |
| M59 | 0.784 | > | 7 | 4 | 70.0% | 81. [symbol] % |
| | | ≤ | 3 | 18 | | |
| M60 | 0.822 | > | 8 | 4 | [symbol] | 81. [symbol] % |
| | | ≤ | 2 | 18 | | |
| M61 | 0.7 [symbol] 8 | > | 8 | 7 | 80.0% | 69.2% |
| | | ≤ | 2 | 15 | | |
| M62 | 0. [symbol] 7 | > | [symbol] | 7 | 90.0% | 68.2% |
| | | ≤ | 1 | 15 | | |
| M63 | 0.773 | > | 9 | [symbol] | [symbol] | 53.8% |
| | | ≤ | 1 | 14 | | |
| M64 | 0.600 | > | [symbol] | 6 | 90.0% | 72.7% |
| | | ≤ | 1 | [symbol] | | |
| M65 | 0.859 | > | 8 | 3 | [symbol] | 68.4% |
| | | ≤ | 2 | 1 [symbol] | | |
| M66 | 0.777 | > | 7 | 5 | 70.0% | 77.3% |
| | | ≤ | 3 | 17 | | |
| M67 | 0.764 | > | 8 | 8 | 80.0% | 72.7% |
| | | ≤ | 2 | 16 | | |

*R, responder = CR + PR
**NR, non-responder = PD + SD [symbol]
indicates data missing or illegible when filed

As shown in FIG. 9, the mean±SEM of sensitivity and specificity were 82.8±1.1% and 68.6±1.2%, respectively (n=67).

Confirmation of Responsiveness Predictions by Weighting and Combining Predictive Peaks

In order to predict the responsiveness of anti-PD-1 therapy by combining nine predictive peaks selected as representative predictive peaks among several differential peaks, they were sorted as shown in Table 7 below according to the descending order of the accuracy (ACC) values of each predictive peak. The results determined by each predictive peak (i.e., open and closed) were weighted according to the order of accuracy, and the degree of chromatin openness was converted into a “weighted score.”

TABLE 7

| Target ID | ACC** |
|---|---|
| M36 | 84.4% |
| M17 | 84.4% |
| M20 | 78.1% |
| M30 | 75.0% |
| M5 | 75.0% |
| M29 | 71.9% |
| M2 | 71.9% |
| M1 | 65.6% |
| M35 | 65.6% |

**ACC: Accuracy = (TP + TH)/(TP + FP + TN + FN)
TP = True positive;
FP = False positive
TH = True negative;
FN = False negative

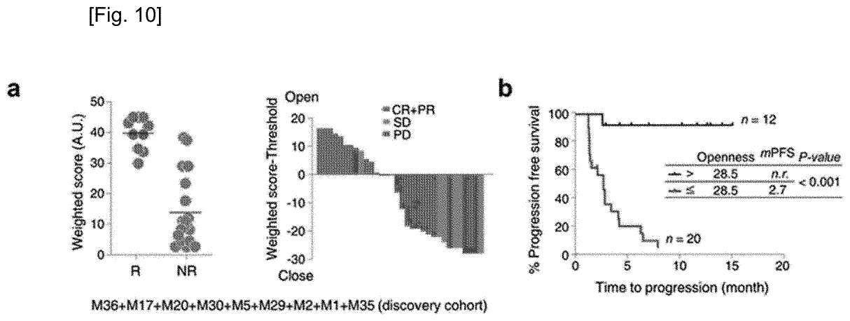

Accordingly, 1 was assigned to the predictive peak M35 having the lowest accuracy among the predictive peaks shown in Table 7, and weights up to 9 were assigned to the predictive peak M36 having the highest accuracy. That is, according to the accuracy, weights, which are integers from 1 to 9, were assigned to chromatin openness samples by each predictive peak. Each combination of predictive peaks was performed by adding the next highest predictive peak one by one from M36 with high accuracy. Responsiveness to anti-PD-1 therapy was predicted by the combination of predictive peaks to which a weighted score was assigned, and the results are shown in Table 8 and FIG. 10.

TABLE 8

| | | Thres- | | Sample (n) | | Sensi- | Speci- |
|---|---|---|---|---|---|---|---|
| Combination of Target (ID) | AUROC | hold | | R* | NR** | tivity | ficity |
| M36 + M17 | 0.927 | 8.5 | > | 9 | 4 | 90.0% | 81.8% |
| | | | ≤ | 1 | 18 | | |
| M36 + M17 + M20 | 0.932 | 15.5 | > | 9 | 3 | 90.0% | 86.4% |
| | | | ≤ | 1 | 19 | | |
| M26 + M17 + M20 + M30 | 0.923 | 15.5 | > | 10 | 3 | 100.0% | 86.4% |
| | | | ≤ | 0 | 19 | | |
| M36 + M17 + M20 + M30 + M5 | 0.941 | 20.5 | > | 10 | 3 | 100.0% | 86.4% |
| | | | ≤ | 0 | 19 | | |
| M36 + M17 + M20 + M30 + M5 + M29 | 0.948 | 24.5 | > | 10 | 3 | 100.0% | 86.4% |
| | | | ≤ | 0 | 19 | | |
| M36 + M17 + M20 + M30 + M5 + M29 + M2 | 0.964 | 27.5 | > | 10 | 2 | 100.0% | 90.9% |
| | | | ≤ | 0 | 20 | | |
| M36 + M17 + M20 + M30 + M5 + M29 + M2 + M1 | 0.973 | 27.5 | > | 10 | 2 | 100.0% | 90.9% |
| | | | ≤ | 0 | 20 | | |
| M36 + M17 + M20 + M30 + M5 + M29 + M2 + M1 + | 0.973 | 28.5 | > | 10 | 2 | 100.0% | 90.9% |
| M35 | | | ≤ | 0 | 20 | | |

*R, responder = CR + PR
**NR, non-responder = PD + SD

As shown in Table 8 and FIG. 10, it was confirmed that compared to the predictive effect on the responsiveness of anti-PD-1 therapy using a single predictive peak, the weighted combination by combining multiple predictive peaks showed that the area under the receiver operating characteristic (AUROC) value exceeded 0.923 and that the ability to predict the responsiveness of anti-PD-1 therapy can be further improved by dividing the open chromatin (>threshold) and closed chromatin (≤threshold) groups corresponding to the clinical results. In particular, the combination consisting of 4 predictive peaks (M36+M17+M20+M30) showed high sensitivity and specificity for the responsiveness of anti-PD-1 therapy determination of 100% and 86.4%, respectively, and the median progression-free survival period (mPFS, 2.7 months versus not reached, p<0.001) was also significantly improved. In addition, the combination of only four predictive peaks achieved the optimal saturation point for sensitivity and specificity. This suggests that the combination of the selected predictive peaks is very effective in predicting the responsiveness of anti-PD-1 therapy.

### Example 5. Independent Validation in Patients with Metastatic Gastric Cancer (mGC)

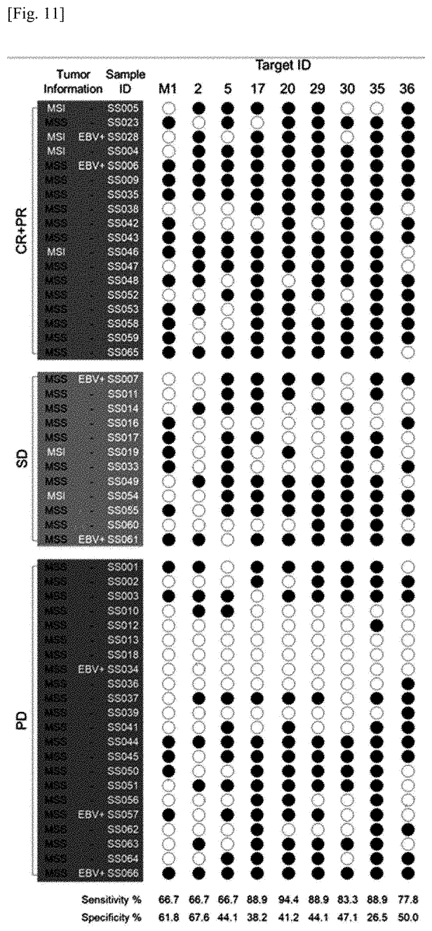

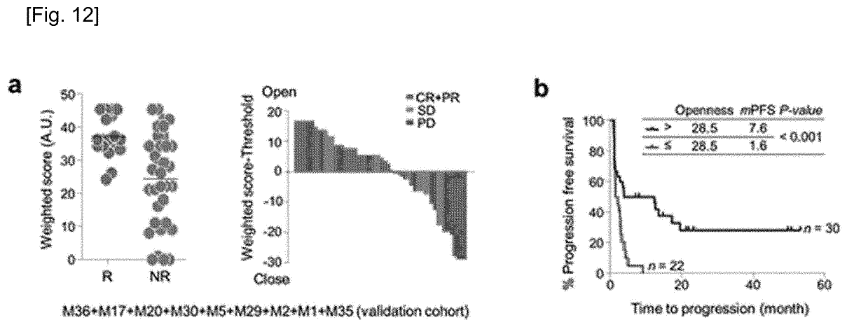

The predictive effect of the selected predictive peak on responsiveness was verified using an independent validation cohort of 52 patients with stage IV mGC who received anti-PD-1 therapy. The patient characteristics of the validation cohort used are summarized in Table 2. All threshold determinations and statistical evaluations used in the previously identified discovery cohort were applied to this new cohort study. As a result of applying nine selected predictive peaks to the validation cohort, responding and non-responding patients were distinguished with high sensitivity and specificity even in a single predictive peak, the same as in the discovery cohort, and the predictive effect on the responsiveness of anti-PD-1 therapy was further improved in the combination of weighted predictive peaks. The predictive effect according to the predicted peak combination is shown in Table 9 and FIGS. 11 and 12.

TABLE 9

| | | Thres- | | Sample (n) | | Sensi- | Speci- |
|---|---|---|---|---|---|---|---|
| Combination of Target (ID) | AUROC | hold | | R* | NR** | tivity | ficity |
| M36 + M17 | 0.626 | 8.5 | > | 14 | 17 | 77.8% | 50.0% |
| | | | ≤ | 4 | 17 | | |
| M36 + M17 + M20 | 0.688 | 15.5 | > | 14 | 13 | 77.8% | 61.8% |
| | | | ≤ | 4 | 21 | | |
| M26 + M17 + M20 + M30 | 0.730 | 15.5 | > | 18 | 18 | 100.0% | 47.1% |
| | | | ≤ | 0 | 16 | | |
| M36 + M17 + M20 + M30 + M5 | 0.720 | 20.5 | > | 18 | 17 | 100.0% | 50.0% |
| | | | ≤ | 0 | 17 | | |
| M36 + M17 + M20 + M30 + M5 + M29 | 0.700 | 24.5 | > | 17 | 16 | 94.4% | 52.9% |
| | | | ≤ | 1 | 18 | | |
| M36 + M17 + M20 + M30 + M5 + M29 + M2 | 0.706 | 27.5 | > | 16 | 14 | 88.9% | 58.8% |
| | | | ≤ | 2 | 20 | | |
| M36 + M17 + M20 + M30 + M5 + M29 + M2 + M1 | 0.718 | 27.5 | > | 16 | 14 | 88.9% | 58.8% |
| | | | ≤ | 2 | 20 | | |
| M36 + M17 + M20 + M30 + M5 + M29 + M2 + M1 + | 0.717 | 28.5 | > | 16 | 14 | 88.9% | 58.8% |
| M35 | | | ≤ | 2 | 20 | | |

*R, responder = CR + PR
**NR, non-responder = PD + SD

As shown in FIG. 11, the single predictive peak showed a sensitivity of up to 94.4% and a specificity of up to 67.6%, so responsiveness could be predicted even with a single marker. In addition, as a result of combining them and applying the weighting method thereto, as shown in Table 9 and FIG. 12, nine predictive peaks selected from the discovery cohort and the corresponding normalization factor (F) were applied to the mGC patient samples in the validation cohort. Thus, it was confirmed that the responsiveness of anti-PD-1 therapy could be predicted equally in the validation cohort. In addition, it was confirmed that regardless of whether the tumors of the validation cohort patients were MSI or EBV-positive, the thresholds of all predictive markers were clearly divided into open chromatin (>threshold) and closed chromatin threshold) groups, which correspond to the responsiveness of anti-PD-1 therapy determinations. The median progression-free survival period was significantly improved in patients (i.e., chromatin openness) whose area values normalized by each predictive peak exceeded the threshold. The possibility that the nine predictive peaks selected through this process could be used as biomarkers (predictive markers) predicting the responsiveness of anti-PD-1 therapy in patients with metastatic gastric cancer was confirmed in both the discovery and validation cohorts.

### Example 6. Confirmation of Normalization Control Peaks in Cells Other than CD8⁺ T Cells

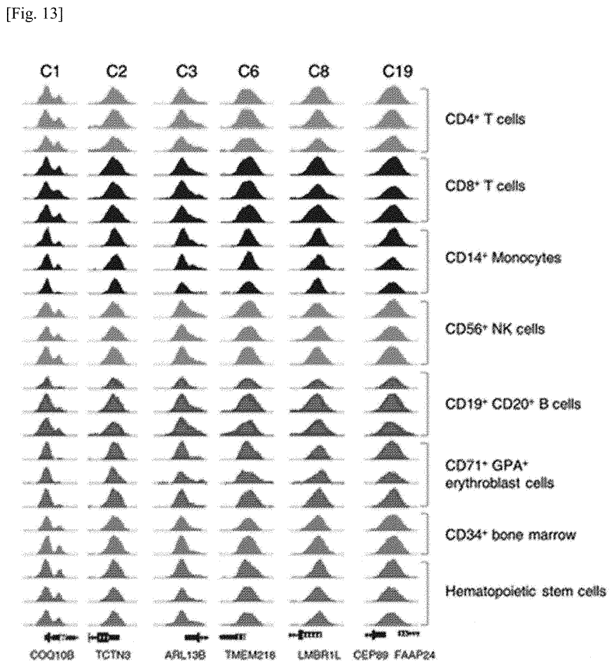

In order to determine the usefulness of the normalization control peak derived from the present invention in cells other than CD8⁺ T cells, normalization control peaks for eight hematopoietic cells, three acute myeloid leukemia cells (genome spatial event database, GSE74912), normal bronchial epithelial cells, small cell lung cancer cells, normal prostate basal epithelial cells, small cell prostate cancer cells (GSE118204), and EGFR negative and positive glioblastoma (GSE117685) were confirmed in the same manner as an Example 2 above. The results are shown in FIGS. 13 to 15.

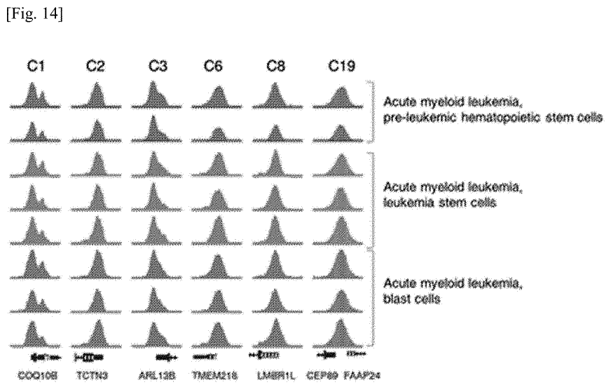

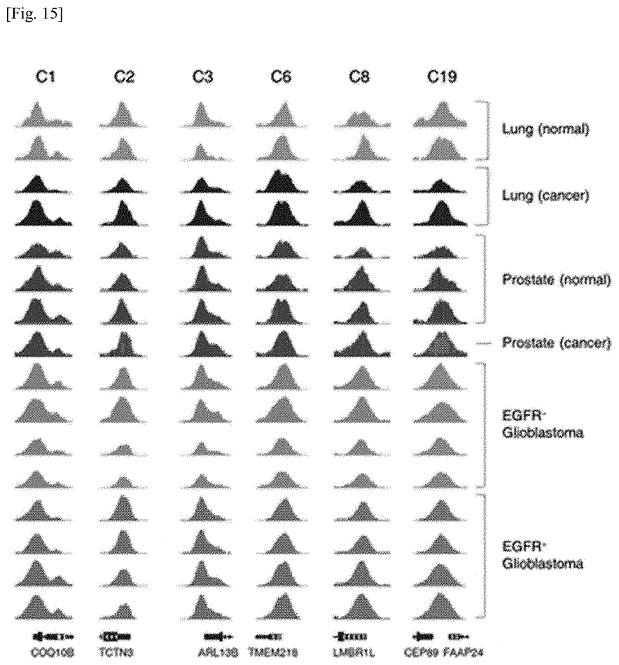

Twenty normalization control peaks were confirmed using the genome browser. The ATAC-seq data before analysis which searched for NCBI GEO depository site was converted to Bigwig files using Homer Suite. Normalization control peaks of eight hematopoietic cells are confirmed in FIG. 13. Normalization control peaks of three acute myeloid leukemia cells are confirmed in FIG. 14. Normalization control peaks of normal bronchial epithelial cells, small cell lung cancer cells, normal prostate basal epithelial cells, small cell prostate cancer cells, and EGFR negative and positive glioblastoma are confirmed in FIG. 15. These results suggest that the peaks to be compared are quantitatively analyzed using a normalization process that converts a differential peak into a normalized area value using the normalization control peak of the present invention in cells other than CD8⁺ T cells, thereby being used as various biomarkers and performing predictions and diagnostics.

1. A method for selecting normalization control peak for normalization of ATAC-seq (Assay for Transposase-Accessible Chromatin using sequencing), the method comprising steps of:
a) aligning and peak calling cell-derived ATAC-seq data;
b) selecting overlapped peaks between cell samples among the peaks called in step a);
c) selecting a peak coincident with DNase I hypersensitivity consensus peak among the peaks selected in step b); and
d) selecting a peak having a coefficient of variation (CV) of less than 0.3 and peak width of less than 500 bp among the peaks selected in step c).

2. The method of claim 1, wherein the peak selected in step c) is located at a transcription start site (TSS) or 5′ untranslational region (5′ UTR).

3. The method of claim 1, wherein the cell is one selected from the group consisting of cancer cells, stem cells, immune cells, inflammatory cells, epithelial cells, hematopoietic cells, and fibroblasts.

4. A normalization control peak selected by the method of claim 1.

5. The normalization control peak of claim 4 comprising at least one selected from the group consisting of SEQ ID NO: 1 to SEQ ID NO: 232.

6. The normalization control peak of claim 5, wherein the normalization control peak includes one or more selected from the group consisting of SEQ ID NO: 1 to SEQ ID NO: 20.

7. A method for selecting ATAC-seq normalization factor (F), the method comprising steps of:
a) deriving an average height (k) of an individual normalization control peak selected from an individual sample by dividing the area of the individual normalization control peak selected by the method of claim 1 by a peak width in an individual sample in a cohort as shown in the following formula:
Average Height (k) of an Individual Peak
$$k = \frac{area}{width} = \frac{the sum of enrichment within the peak}{the base length of peak} ;$$
b) selecting m number of normalization control peaks existing in the individual sample in the cohort, and deriving a mean height (h), which is a mean value of the average heights (k) of the selected m number of normalization control peaks, through the following formula:
Mean Height (h) of Selected Peaks in Each Sample
$$h = \frac{{\sum }_{i = 1}^{m} {k}_{i}}{m} , \\ {k}_{i} = \frac{the area {A}_{i} of the ith peak}{the base length of the ith peak}$$
m=the number of selected peaks in a sample; and
c) deriving an ATAC-seq normalization factor (F) through the following formula:
$${F}_{ab} = \frac{{s}_{a} ( = \frac{{\sum }_{i = 1}^{{n}_{a}} {h}_{ai}}{{n}_{a}} )}{{h}_{bj}} for 1 \leq a \leq b \leq 2$$
hₐᵢ=the mean height of the ith sample in the cohort Cₐ
h_{bj}=the mean height of the jth sample in the cohort C_{b}
nₐ=the number of samples in the cohort Cₐ

8. The method of claim 7, wherein the mean height (h) represents a degree of chromatin openness of the individual sample.

9. The method of claim 8, wherein m, which is the number of the normalization control peaks selected in the sample, is 5 to 50.

10. A method of normalizing the area value of a peak, wherein the method comprising multiplying the ATAC-seq normalization factor (F) selected by the method of claim 7 as in the following formula:
Normalized area(A)=F_{ab}×A_{q}
wherein F_{ab} is defined as follows:
$${F}_{ab} = \frac{{s}_{a} ( = \frac{{\sum }_{i = 1}^{{n}_{a}} {h}_{ai}}{{n}_{a}} )}{{h}_{bj}} for 1 \leq a \leq b \leq 2$$
hₐᵢ=the mean height of the ith simple in the cohort Cₐ
h_{bj}=the mean height of the jth sample in the cohort C_{b}
nₐ=the number of samples in the cohort Cₐ
and A_{q} represents an area value of the q-th peak of the j-th sample of the cohort C_{b}.

11. The method of claim 10, wherein m, which is the number of the normalization control peaks selected in the sample, is 5 to 50.

12. A method of selecting a differential peak for predicting the responsiveness of anti-PD-1 therapy, the method comprising steps of:
a) aligning and peak calling ATAC-seq data from patients who respond to anti-PD-1 therapy and those who do not;
b) selecting a peak which is distinct from the responding and non-responding patient samples from among the peak calling values of step a);
c) normalizing the selected peak area by multiplying a peak area value (A_{d}) of the peak selected in step b) by the ATAC-seq normalization factor (F_{ab}) selected by the method of claim 9 as in the following formula:
Normalized area(A)=F_{ab}×A_{d};
d) selecting a peak having a normalized area value exceeding a mean area value of the normalized peak area values obtained in step c) from among the peaks selected in step b); and
e) selecting a peak in which a mean difference between normalized peak area values from among patient samples responding to and non-responding to anti-PD-1 therapy among the peaks selected in step d) satisfies significance p<0.05.

13. The method of claim 12, wherein the peak calling of step a) is performed using two or more types of peak callers selected from the group consisting of HOMER (Hypergeometric Optimization of Motif EnRichment) suite, Model-based Analysis of ChIP-Seq 2 (MACS2), and CisGenome, and
wherein the selection of step b) is performed by selecting a commonly distinct peak from the two or more types of peak callers.

14. A differential peak for predicting the responsiveness of anti-PD-1 therapy, which is selected by the method of claim 13.

15. The differential peak of claim 14, wherein the differential peak selected by the method includes one or more selected from the group consisting of SEQ ID NO: 233 to SEQ ID NO: 299.

16. The differential peak of claim 15, wherein the differential peak includes one or more selected from the group consisting of SEQ ID NO: 233, SEQ ID NO: 234, SEQ ID NO: 237, SEQ ID NO: 249, SEQ ID NO: 252, SEQ ID NO: 261, SEQ ID NO: 262, SEQ ID NO: 267, and SEQ ID NO: 268.

17. A biomarker composition for predicting the responsiveness of anti-PD-1 therapy, the composition comprising one or more predictive peaks selected from the group consisting of SEQ ID NO: 233 to SEQ ID NO: 299.

18. The biomarker composition of claim 17, wherein the predictive peak includes one or more selected from the group consisting of SEQ ID NO: 233, SEQ ID NO: 234, SEQ ID NO: 237, SEQ ID NO: 249, SEQ ID NO: 252, SEQ ID NO: 261, SEQ ID NO: 262, SEQ ID NO: 267, and SEQ ID NO: 268.

19. The biomarker composition of claim 17, wherein the biomarker composition includes four or more of the predictive peaks.

20. The biomarker composition of claim 17, wherein the predictive peak recognizes differences in chromatin openness between patients responding to and non-responding to anti-PD-1 therapy to provide responsiveness information to anti-PD-1 therapy.


SEQUENCE LISTING (299 sequences)

SEQ ID NO: 1 - DNA, 1424 bp, Artificial
  control peak
ttagtatggaataagggaaacccaaatcatacgagaacaccaattaaaatgtgggacacgcaggaagaaggcagacggaactgctgctggataactgagtctttcctaatgacaggaccttatgttgccctcacaattaccccgtgagataggcaaggcaggcattgtattagacaatcattaacaagcaggacgctgacagccaggcttcgcagccaggagtggtggcctgtctggctccgcactcacgggttcggacagttacagaattaacactcaagccctggggcttcccgcagcccccgggggggtggggctcactgactagcctcccggaagcggaccccaggagaaccaggcagttggttaatccaggaacagctcctgcgcggggcgtggactgagcagtcactgagtattacgcaaattgcgcgagcgcacgggaaaagcgattggtcggtcaggagagagaggtgtgtcctggcgggcccgcagctccgattggccgacaggctgacgggaacgtttacggtcagcgtgtgtcagcgacgtgcaacccggaagggaagaaggggcgtgtcaggctgcgcaggcggccagtccattggctggaagagaccggagccgggctccgggcccgaccagaggagggcggtgctgcagggctggtccgggaggtgacgaccggcttcggagagtctatcatggcagctcggactggtcatacggccttgagaagggtagtctcgggatgccgtccgaagtcggcgacagcggccggggcgcaggcgcccgtgcggaatggcaggtaatcaacagcgggggcgctgagacgagagtagttttcgattttggctgccgagttgggggaccggaaggatttgggggtgaggcagagccggactcggtgcatttccgtgtctttagaggagaaggcttttttttgcgtttggcatctgagggactcaagagcggcgcgcatcaccttgggcccaaggctgtgttcgtggctcgtttgcctcgtgagaggcggtgaatcatcggcgaagcgactttggaagcaatttggccattggtgcctttgggctcttccggtctcctcccctatggcttgttgctactgctgttggaaagttttaactttgcgcagaagggttgccggctgctggctgtatgggagtttgtgtttggcggtgagcccccttctccggttccttctacctgccagatggtcagatgcgttttcccaatttctgaaagagtgctttgccctcgccatttagtgtttacaaaactaaatacagccagcataagggacttggattttttcactctgctgtaacttacaggtcctgtaaccttgaagcatagtcagctttcgattttcctggttggttcattttttggtaggtgggggtggagat

SEQ ID NO: 2 - DNA, 1134 bp, Artificial
  control peak
gctcaccttacgctgcctggaaggcagaaggagaaaactgtcctcggatggagaagatagcagtccctgtcgcagcagcaatttatatcgcaggctccaggagtcaagtcacagacacagatcggtaagactatgaaagtacgacacagagtgagtgggatgaagatccccaaactctcccaggtgctcaccctccttcttccgacagccccactcctgctttgtggcagctcaactttgctcacagaattttagatctcagaagtaagtttccacccccacaacgttttccctcacctgggaagaggtccacagtcctattcccaggggccgagggagtcacgagagtagggaccactgtagggagtccaggcacggccgggcgagttgcagtcgcctctgaaggggactggagggttccgccatccgtccctcgctgcagctccaaagacgtgggcactgcccctgatggggaggaagagggctgaggccggacgccatcggggaacaccagaaagaacacttgcaggagcgcgagctgtggggtgcgcatggggcattcagggcctccgggtccgacgtaggcctccgcggtctccaatcgcattgccagagcgtgggtgggaggagtcgcatagcgtctgacgccatccgcgtgcctcttctcattggctcatgagaaggctgagagctcttcaaccaatcgaaagtggggttgttacagctctttcagtgtccttcggtctgcgccctcctccttaacgcgggccgggccgtgcctccgttgcctggcaaccgtcagtgacgaagaagcccttgcgccttccttccgctaggtggcggtaggagttcggcggaccgagaggttccgcgcccacttcgcggcccagaggactgagcccggcctccggtgcaaggaaaccccagaggccggaactagaattgcaaattttttttcaccccggattccaacggtgagaaaccctttctatggaagaataatgttctcaggtgggggaaacacacctaaggtcacataccttaaaaaacagtctcctctaaatatggcttttttaaaaaaaaaaaaaacacattagaaataatgttcattcagctccttacattttaacattg

SEQ ID NO: 3 - DNA, 1261 bp, Artificial
  control peak
aactactgagaaaatttagcaagcgaatttcccccttgtctttaacttacaagaattgccccagtgtattgttcctgtagtcatgctgcctaagtcggccagaattctagagggtccccatccctgagcaaaaaacaaacaaacaaaaaacaagaaaaaagaaagaaagaaaaagaaaaacccatgcacaagacctgagatctctgaatctcagttggggacactggagaaaggaaaggaacagcaccgtggattggcaacacattttatgtaggtataaagataggccttgctggagatttgtcccggatcctccagcttatctccggacgctccggttgcctagttacgagaacgagctacctcgagctccgcctccttcttatagagtactcccctttctcttttcctagtctcgcgcaggggcgggtagccaggactcttcagccactcagaggcctctacctcaggacctcaaccgatgattgggcgagcgcagtacgggcgttgcctggggaacgccttccgcgcctcgaagtaacgtcagcacgtcgacgcggggcttttctttagccgggtcccgctaactcggctacggtgtatctgcgtctttggtcaggttgttccttggctaagagggcagtcgtcgcggacccacgcggttagcaaggcttagtgctcgggccggccgccttcacttccctcccggcttttcctcccgacttatccactttaggggcgtctcggagtgccggaggcccccggggaagagcggggtgccggtgtccgctccgggctcggatgggaagtggtgggaggagcgacccgggatgttcagtctgatggccagttgctgcggctggttcaagcggtggcgggagcctgtcaggtaggctggagccagctgtcctggccgtcctaggggttggagatggctgcgccccaggggcgaggcgccgccttttccccgcgcctggacgagtctatcccaggccgcaagggatgctttcattcccagggtgtcccgggagggagagattggaggatgtagccttgttgtctggtcgactttctgttgcgattgacaatgcttaccgttgttatattttaaataggtttgaatttgttaaaaagtgtgtgtgtgtgtgtgtgtgtgtgtgtgtgtgtggaattgaaaactatccattgtgtgtttttcctctgaagcttttgtaatggaaattcttttaaaaagagggggacatggaatatagttttcata

SEQ ID NO: 4 - DNA, 949 bp, Artificial
  control peak
atagcaccctttacagaagaggagatggaggctcagggaaaggtcacaagaccacgaagtgccactgcaaacttagaatccaaaaccagaagtgccatggaagccctcgccgcgccgcgccgccccgcacctgcaggcgtcaaggacgcggaagcctttggagcgcgccaggcctgtgaggaaggcccggcggctgcgacccatgcgaggctcgaccaggtagatggcgactcccgggaagcgcgtcgagggcggcgtggaggaagcggcatcgccgctaggggacccgacccgcgctcgccgccgtttggggagcattgggacgacagcctccagcagcgcggagcgaacgcagagggaaactccgagcgagacggaaggaagccccagtgaggctgacggaagcgggtgggcgggcgggcggccgccggctggaagaggacgtggccccgccctatgcaaatgaggcctctccgtggggctcgctacttgggccagtcggagcgctggggagggtccgggctcctacaggattgggtgtcccggccgctccggaagacccgcctccatgcaaataacggcagaggacccgccttttatttaaattaatgtctcggaaagcgctgttcggccccattggcctccgggaggagaggctgggctggagcggggcggggagggtgttggagggggtggaggaggcggggtcaagggggcgtccccggctagggagggtgaactgggacctaggcccgggcggagcggggtgggctgtgtccttggcgccggggctgggtctagggttgagccggcggctcggggcggacgcgtgcgggctgtgcccgatccctctgcgctcgggactgtcacacggaagccggcgggcgcaggaccgacacgcgggtttccaatgcgctggtggggcggtcgcttcaaacacgcgggaaga

SEQ ID NO: 5 - DNA, 1120 bp, Artificial
  control peak
agccagttttaagatctgtaacgtcactaacaaacgcttttaacattgtgctttaaaatgtaaaatttattggggaagagggaggctgaaaaatcgtgtttttttttaacccccacgggagctgtaatattgtatagacaaactgtgcaccaaaagcatcgtactccaacatgtttctgtggccccctcccttcacccgctaaataaatgaagaaacaaaaattactgaactgagattaacttctgcatcgtaggctcaaccttatttcacagacctcttgacggggacactcactagtgtcgatatctaattttaatgtttgcgcaccaaccttcccccaagccacctgagctctgtgagggcggggacttggacttaactcctgtctgcttctcccaaaacaccaagaaagatgctgaacacagcaggctcgagataaacactcgtaagaaagcgtttctctgcgataattacatcaaattttgatctaaactaattcaatcccgggcccgttacgtctggcacccactcaaatgaacacaccaaaccttacaaatttcactcacccgtactctagaaagaaccctaggaagagtttaggcgggcgcgcaggtgacgtcacacgcacgctgacggttcagctccacgcatgcgtgcaggatccctgggcatagtcgcttgttctccgcccgcggtggaccagctgcgtaacggttcacacactgatggcgcctggaggtttggcgaaatactgacaggatgcgacgtcaacgggagcgcagggccaaggtacaggccccgcccagtccggtcacatcccccggaattctcgcagctccccttagctccgcccccagtgactttccagctttcgtggcgggaggcaagcttaggggaagcgggcgggtccgctgggcgggatttgagcttccctttggtagattcgaagctcctgtaggcagagcctttcttttaatgtccagttctcttcctccaagaaataggcacctactgcagtctttacaaggaagcatttacaaggataacaatgttatttgggagtttttatagtaatttcgggcattggcataatgccaccattgagtcttgctatttaca

SEQ ID NO: 6 - DNA, 1108 bp, Artificial
  control peak
atccctctcctggacggtgttgtgggttggaagcgctgcagagcctcggtctatccgctctctggatctcacagcactgcagcctgggaacaccccacttgaactctaagcttctccccaaccctgttgcagattcaaactggcgacaggaaaacaatgccccccatgtctcgtcttcagaatgtgcacctggccacctcccaggagctagagagggattaaggtctcggtcataagagatctgaatcaggcactgacacttccttttcttaagagcatgaaacaaaacagtccttccttctctgcctccaattaagtatttattagccgggccttgggtccagatcagcggtaagggtggtctgcgaggtccgtgcaccgccgtccgcccacgccttcttccaaccacctcagcccgaccctccacttttctcccttccccgcagccgccccgctttcctcacctctgcccccttcacctggatccactctcccccaggctctactcacacctcagattccggcgcgttccagcccacggggctctggtttacttagggcgaatcacaagtgggctttcatttaggtgatttaagattcactaatcagcgttgctctttcaacggccaacttcttcctcattagcttacgcctagcgcggctcccggcaacgccgagcgcggtgggttggctcgtcacccttcgttacgccctccgattggtcggtgcctgtcagcctttggaccgacctcagttcggttgggtagcagcggtccgagctgcactccccgcccccccgccactccactgtcaatcacaccatctctccgtaactcattgggccattggtcagtctagcctgagggcgggttgttgggcggaagagagagacttcttccggcctcactcgctgtcaccatagagattgcccatccaggcagcgaagcagcagggccaggcagctcagatctcgaactccagagaactgcacagtcatctagacatcaatgccttttccggggcctgatgaacatctcaatccccaacccactagctcgagtggggcgcaagatgtggtggacaagtcagctcaggcccggtcattaag

SEQ ID NO: 7 - DNA, 1432 bp, Artificial
  control peak
aagaacagatggatctagtttctccaaagatcccaaatggattggtctccattaagcaagacgaggaatgaataaggacgagcaggtggggattgctttgtgcagagggtggaatacagatcagagttcagcttaaatcagtattttctaagattttgctaggtgccagatactgagataaagtccttgtcttcatgtttggaaccaggtgcctaaggaagcacagaggagagccacctccaaaatggataccctctccaagggtttgtagtgaaagaggcacagctcttggcctggagttggtgggggctgcgataagtgcaagatacttggtgacaggaatcgcgagcatactcttgtgttgtacggattctcagggtcggccctgcagaggaaagagctcagtcaccgcgaggtcctgcacaacatgccaaagtcccggatatgtgtccggggtctacacctaagccccgacccctctgaacttcccagaagcgcgcaggaggcatggtgcgcatgcgtgtagccacctcaaaggcggcgggaagttggttttctgagtagcaactgagcagcttgaagacggattggaagtagcagaccaataaacaaacgtctcattggcttagagtgcttagagtcccaattcttcagcttcccattgggcctttcaagcgagcaggggcgtgactccgggggctccgccccgagccttggcccagtcttggggtccaaggctgagggagcgggacggaagtgagcgggtcccgccccttccccttctcgtctccgttggagtcgtctctgccgcggcttcctcggctgccagctctccggcgagccggagtcctagtgccgtaccgtcagtccccggccgcgcggagccgggatgcactgttcctgctgtgggtcctcatcatggagaccaaacgggtggagattcccggcagcgtcctggacgatttctgcaggtaccgcgctacccgaccccctttcgcccccgtcgggttttctcagtttcgcggacccccagaggcctctgggtcttcttccccgcccacgtccatgtcctcgctttccgctcccgccgctgctcgctttccatcgtcgaccctgctctccgacgacaccgcccctctttccgccgcagcctccctgtttctcaaggccctccgtccttccccgcagaacacccggaatttcctcattttttaagttactcaactattttgctttcccgcaggcgtagccgcttcgccggagcctgggtcttagcgttttgccgccgccgccgcctccctctcccattgttttttctgcctctgtgacttttccacgccgtgcaccccctcctccctgtgtctcattccccgattctgaaggaagacccccagggtagtaatttaaggcttaggatcccaggtacgcggac

SEQ ID NO: 8 - DNA, 1050 bp, Artificial
  control peak
ccatccttccccgattatcagactaaggcagactccgcaggctctcagaccccggagacccccaacaggaagacgcgctgggtgaggggtgggagggaggggcaggggaggggcgtgtgggggaggaacggaaacgacggcctttgtttatcgctcagggacccagctgatcccatctaggggatcgctagccgggcacccgcccttctgcccggacggaggcctccgtcgcccgccgctggcccaggcatggtttgacggcactctgagaccaggtgggctcggaccccaacctccccgtctcccgcaacccccgcttctcctcgccggggccccgcactcacaatacactcgcggatcctctcgtggaatagctgttctcgcacggatagcacttcgtagtcaggtgcttccatactctgctcagcatgactgaagccgaggtgcctctgggcccggggaggacgagcggggaggaagccgccgccgccaagcacccagacccagcctaggggcctttcctcgcagcctgcgacagaaactcggggcagtctggggctcagatacagtcgtccggacgccccgcccttaaaggggcaggcaccacccgcctcgttttaaagggctctgtcctccctttgctccccactctttaaggtcgggtcgcgctcacgtttcaatgcaaacacccgccactaggctcgctacgaggaagcgccgctgagggtccatctttttaagagatctagtgacgacacaagggatgggcgggttccttccagggggaggaactcattggctcctggagagctgtcgttcatcgctggaggagcgctgataggctcgccctgtggaggaaggcgggatcttttgggcctaccgctgcccatactaatagagcaactgtctctcttatctgggaaaagtgaaggttcctggattttagtagcagccaacattttagtatgaccttggttaaatgaaccgtttgaagcaggccagccaggcaggatgtgagggcaccctaggggttctgaaatgtaccgctgc

SEQ ID NO: 9 - DNA, 856 bp, Artificial
  control peak
gagcccgagctcttcttcaccagctcaactccgtctccagcacctaagcagtatcctcccgccatccctccccacggcgcctgcgttccccggcaaccggcgccgctgggcagcctgggaggcaggaaaaagcgactcttcctctaacagtctccggaaaacggtgcgaagagaaggcgccaagacgccttccctccaatacactacaatacccaggacgctttgctacaatacctctccggggttggctggggggcgcctccagctgcgcaagcgcatagggatgcgccctttgtggactccattgcgcagggtgcattgccctgaagagcgctcctccttccggtgaaagtcgtcgggttcccagccgggagtctgcggagttctcatactctgttacccagcttcctttgtatgagtgctgttgcgcacttccggtggaagcgctgaagcgagtgggtggagtttacggagccggtgggcggtaggcggtgctacgggtagctgggtgctgtccaaaggcgacagggcgtcgttaggggagcgagtcgtgaccggttgggccacactcaacgtgggacgaagcttcgcctactgtttgactacgtgcgtgcagcctcccctcgatgtcggccctcgaaaagagcatgcacctcggccgccttccctctcgcccacctctacccggcagcgggggcagtcagagcggagccaagatgcgaatggggtacgtgacccataccccttctccctatgggccaggccccagccgttggagaatggtatctctgggcgttcatagaggcacgggaaaggagtctactgataggagaaaatgttggcggcctggagatat

SEQ ID NO: 10 - DNA, 835 bp, Artificial
  control peak
tcctagagaaaagcacttaccaggatggaccgccgtcggagcggcccgcagtaagagctgagctgggggcagcgacgctgaaggggtgagtctccgcaaggccccggaggccggagcagcggccacccgacccacaaaccccaacatggcgtagtccgggtggagactgaaggctgcagcaaccgcagccgcctgctctcctgccgttgaggcgaggcctacgctgcagtaggcaggtccattgggtgaactggctgctcatcattctctgtgctgcttctacaggttaatacacgctgatcttctcatccattggtcaaaactggtgctaatctgaaactcctttttttattggacagtgtgaggttagagggcgtcactctgagcctcaaaacttaattggatcatagcgacgtccgttggacagggtaatcagaggaactacgtttaggaaaggggccgagttcctgttgaaagtgcgtggcctgtctttgcctgtccttgggctttctccttagataggtccttgatctaggtgacagccataggggcggtggttagggaagtggtagtgcggtgaagcaccactgaaggtgagcgtggcgaagtcaggagccctctttagagccaaaataaggtttttctctcgaagtagtgagattgcaagctcccaggcgattgcacagattagcaacctagacaggatctttatcatttaaacctattaaaatgaaataaactaatgtaaccagtactttagcagggcattgaggagactcaataataagataaataagactaagaaccttccttttaaggtccaggtgaacaaaca

SEQ ID NO: 11 - DNA, 983 bp, Artificial
  control peak
taatttacattttagccttccggttactggaatctttgatggggaatgagttatagtgaggcaagagcctctaacattctaggagctcttttcccgttcggggcctcaatttcccgttctacaaaggcgggtgggagggaacggggtcccaagtagtactggataaagaggtggccgatcgcaaacgctgagcacttcaaaactctgggcagagcgcttccgcgcctctcctgctatttcaccctggccacaggctagccgccgaggccggtcatccgctctctccgcggaggagtcaacgccacggaagcagcggcggaacctctctaagaatccttccagcttgtgctcttctgtccagggcagccaatcgcgaggaggagagtagtcagagggcggcaccctccccggtctgggccgagttattggctggtggtagcgtcgctcgcccggcagtaccacgtgacggcctcggccgcgggcgtttacactggctctgcctccggcatctcttcgccggtgcgtcctcgccgcgcccgtaggtcccggcagccgggccccgcctccttcggagtccgagcgatgggcggggaaagggacaggcaggtatagctctgtcggcgccgcggtgtccacctcagtcaggccacggtggaagacgcgtgccgcggcgcctggttgcctgcagcggcccggacccgagaggtgagaagagggaagcggaccagggaagagggagggagcggtgctggccgccaagcggtcagcgggcccctcccagagtcattctgcctgggtccccgcgcggtagaatcccagatcaggggtcgccgccgtggagcggggcgtgcgtcaggttatttcccgggtggtccggggccgggcgagtggcactgcaccctggtgatccgggcctgcccgtctggctggcggctgcccacccgccctccatcccgcgtatctcgccgtatctcgccgggtgc

SEQ ID NO: 12 - DNA, 871 bp, Artificial
  control peak
tcccgccccgagccaccgacttcagcagctctctcgccgcagcagccctgcttgcagcccccaactccctggtccactggccacacctctcgcgcgttctcctggcccacaagccctgaggccgcctgcttggccaagccgctctcgtccagccccagccctttcacgtggctgtgggaggcgatgcgctgcgtcttcgtagtgctcttcacctcctcaatcttcattttgcagacgccgggagctaaaaccagcgtggaaaaccagcagctaggacagtgcgcccggcgcctgagttaccatgcggccgttactagggcaatttgcaaaggcccgcctagaccggctttccattggtggaagcgggaacacctccgctctccataggctcacggctgcgcccatcgcggctgctgctagagctccggtcacccacttccgtctgtgtctccgggttggcttcccccgtagccggcgcttcctgccgaggctggggtcgggagcacagtgggaccacgccccggatttgatctagggctggggcgttgcacgaggcagtggcccccgggtaccactcctaatccttccgtcctatccattagcacatttgaacgtcatactcttacgcaaatctcaagttattatttccgttttggaaacagaagaactgaggttcagagcttaaactgggattggttactaaccaatgttttctgactcccatacctttgcatctccctggaaaggcccagctcaacccaccaccgtttccgtggaattgtcttcccctgcttctatcctccatcctccaccgagagataaggttcctgctgagttcagttgactgtaaataacattttgcagttttcaa

SEQ ID NO: 13 - DNA, 759 bp, Artificial
  control peak
gtacaaatatttctggtgtgaaacacagtgttttgatgctcgcggactcttggattcgaggctgcggtagtacggacaaacaccgtgcagcggcatctcatctccgccaacagccggcgatcaagcgggcaggctgggagagggcgggcggaagaggcgtggccgccgctactgcgcacgtgcacccaaccagaacctgggggtggtgccgggccttccccgctgcgcggaggcgtcctctcaacccttctgcccggcgactggtgggttggcggctaaggggcggagacaagaggggccgccaccatctcctccaatggaagggagacaggggcgggcttaatgacggaaggagcatggcgtggagacacctgaaagtgagtcccgcgagtgagcagtgtggatgggggaccagggaccggggagggcgagaacggagagtcaggccgggcgaggcatggaccgaaggcggctccgggagtgtgggcccccctgggacgtgtgcgtgtttccggggtctcctgcaaatgctcgtgcgtgtgcatttgcacgtggactcggtcaggcgtgtgcagacagcgcctgcaggtctgggtgggtgctgatctgagtgtctgcgcctgggccatgtttttgagcctggcacaggggtgcttagtgaacacatgaccgcctagcgttgtgtgccggatccaacggggaatgaaactagagtaggacatgctcccaacccttttaggtaaagttttgttaggggcg

SEQ ID NO: 14 - DNA, 1013 bp, Artificial
  control peak
cagatttacaaatacctattaacttgtagacagggcaagaaataatttatgctgaagagacgaaagacctgaaaacagatttctgtgttttactctccatgtggccatcatggacgaggacgaagagggacttgatttttcctttaacgagaagaatgtttaagctctgaatttttaaccaatttttatgctcgttactctgctgtagggtatgtatttaccaagattagcagaacaccgcgagttcaccaggcggagacaagtcactgtgaagtgggcagcacaggaccccccggagggcaggcgccggaaactacatatcccggcgtgccccgctcgcgagcacagtggagcctgccgccattcggttcacggcgctacgcattccctgagcgccgccggatgccggcctgggtcccgcccagggctgagggccctcctgggccgggaaaactacagttcccgacatgccctgccacgggtgcgcctgcgtaccggagctactgcgccggggagcgggttggtttaaaatgctgtgttagttccacggaaacccgctcgcctgctgcagtacggagcctcaggcggacagatggactgtaaggtgaggctgccgggccccgggctggtgaggggtggtcggaggctcgcgaggcttcggctcagataaggacagtcaccactccctggcgccctctccctgtcctgacggagtccagtctcactccctggacctcagggccctgatttaaggcgctttggcgttggctgccggtgaaagccatttaggagcgaagtttgcattggcctggctccagctgagggatgggagatagccaccgaagcctccagataagcccaataccacctttatttatgtgttaagttggagtttaataggttcctgggaaggctatttcggcgttaatgtttgatttttgccaccagttaatcctccctggcttgcttaccagaatataggtgtcaggaatttgcacttgttctttacggtt

SEQ ID NO: 15 - DNA, 1022 bp, Artificial
  control peak
gcccactccaacctcttccacagccttcagaaagggcaggcgcagctctgcgaaggtgcttacaagggccagtcggtgatcacgggcagcacaagagcatggcagagggtatcctggccgaggtcctaaggcgacatctgcagcacgaggaggccccgggactcagacgtggccgcttcgcagaacgacggggtcctaagtggatctggcggtcgcgtcccgctggcaccccggctctgactgtcgccctccggctcccaccgcagcgccgagctgggccgccgacctacgtacctggttgcttgaggcaggccgcacgcagccccaaactggtccgggccacttgggtcacagccgcggttcccgggagaaagcgctccctagccccggaacagccaatcctcggccctagccaggtatgagctcccaggcgagccttccaaacccaaggcggagttccgtgcgccaatcatcggtgggagacagggagggggcgtgtccatccccgccccccgcctccgctggcctcgaaagcggagtgcgaagcttcctcaaacctaatcggcggcggagcggcggtgagggggcgtgtctgcccgggccctgcccaccgccctctccgcccccgcgcggcgcccgaggcgcccccttcattgacgtcagtccccagggccagcgcggttttgctggcgcgggagctgggcggggctgcattcctgccccagggtgcggcgaaagctggccctgccgggtacacgtgacggatgccccttctcctgggcccgaggccagcgcgcgcggcactgactgccttgcagcgcgtggccctctgcagatcctcagactctgccgctcaagtcccctcccagcccaccgtttggctccggctttgagggagaggagggggcagtctgctggagtcagaccttgggcttcaaagccagaatgtcgtgggttcaaatattgggtcatttgtgcgactttgggccggttacctaaacaagaccgtattttaaagtttg

SEQ ID NO: 16 - DNA, 943 bp, Artificial
  control peak
gcagccttggtttatgtagttaacatgttgggcctttcttggctctactctatggttgtgcagactttctgtgaggatgctgaagtgacctgagcgagcatgagggaagggctagccacagagatcatggacctaatgcgggcgctgaaaatagggattagggcaggattccggttagaaagcgccagtgttaaaggctaaggagacacagcgccccctaaagagggtgcggtttaaaaatcgctaaatcagtccgacaatatcacgtgtcctcggccctaccaattgaaaattaacaatcgcttcttctgttccccatggaaacaaccccgccccactaaccctcccgccccaatgagtcgtgaggttttggcgttttattgagtattcaagctgacaatcaaacacctgagccttctggttggacactttgcttccggtaagcaagctagtacaataattggccaataaagcaagccttacccaatgatccttatcctaggcggggcctaatgggaacgtatggccaatcagcggcggcgtttcttttgcggctccacgtcggcaccagctgcggggcaagatggaggcgctgattttggtaggagctggaggggcagcagagatgctgtggtgtccacaggggccgggagtcagggttcagcccgagtgggagacggggaaagagagttccgggtaattcttggggactgagcgttctgctgacctttggaataagtgtgtggcctagtgtaggagctaaagccttggtttagagtaggaagccctgccggggagagggcatagagtcggtggaaagttgaaggcagcacatggactccttaggatacacagacctgtaggcgtactggattaggacccttcaggttgccccttatttttagcattgtggatgataccctgattttaatttaccaaacca

SEQ ID NO: 17 - DNA, 887 bp, Artificial
  control peak
cccgttgctaatgtgtgaccctggcaaagtcatctaagtcgctgagcttcagttcctccaacccagagagttgttgcaacgatcaaatgaaagaatgtctattaaagcctttcatgaactatattattgctgctaccgtagaaatggaaagtgtgcaacactagatccaaaactacttttgacacttctgagactgtggccgcgcctctgtcaccttccaaaggccactaggcctttcctgagctggcattggcaacgcacactcttgcccggctaacctttccaggtgggcggcgcactggcttcactgctctccaggtggccgctgcagctgcccgagagcgcaggcgcagaggcagaccacgtgagagcctggccaggccttccggcctagcctcactgtggccccgcccctctcgaacgccttcgcgcgatcgccctggaaacgcattctctgcgaccggcagccgccaatgggaagggagtgagtgccacgaacaggccaataaggagggagcagtgcggggtttaaatctgaggctaggctggctcttctcggcgtgctgcggcggaacggctgttggtttctgctgggtgtaggtccttggctggtcgggcctccggtgttctgcttctccccgctgagctgctgcctggtgaagaggaagccatggcgctccgagtcaccagggtgagccgcttcggactgcgaactaacgcggccttcttagctgctgcctgctctccctgcctcgcctgcgggagcctcccgagcgggagagggccgcaggagcgatttggggaggaaggtgggaggggactcaccaagagagcgccgaggtgggcccaggcctggtgagagagtgtgggggacgatggatggaggg

SEQ ID NO: 18 - DNA, 1106 bp, Artificial
  control peak
tccatcttctcggtcctttcctcctcctcttcttcctcctcctcctccccctgagtagtaagtgctaacgacccgcaggactctcatcaggcctctattactaagtaaggggctgccttacccccaacccgccacccaggacaaggcgcacattctcggctgtctgcgaacctcggactctgagacatctccagcctgagactgcaaagtgggtgaagaactgcgagaactccccacatacagacagtacttccccggtggccacgaggacgccaagccggagatgaggctgggaagggggacgacggggccgcctagagcgaccaagactagggaactagggacatcctggccccctcctctagccccgcccggggcaaggggtgtcctgcgcgccgccccctctcggacccggagaggacgtcccagctagcgtacggggcccacacacagaggcagcagcgcggaggcggcagcggcgcggacagcgtgggaaagagggacactcacccgcttcagccccgagccttcactgcaccggaagtagtcacccgcccgttgccagggtgaggaagcggggctgacatcaagtctccccattggtcattcggggtaccacgtggtcggaagcagcctcccacaagcggctctgactccgccctccgcttcggagcggctcacctggcctcagaggtgcagcgctaactccacctggggcttcgttctgagctgcgcatgcgctattgcttatctctacccaccccgcccctgaataagcccgcaggatgtgattatatctgagtggttgattgactaatactgtgatgagaaatgaaattaaactgaccatcattttatctaggtgtcgcttaagagttactttctttcaatccttagagcactgttagacggtagggaaaactaaggttcagcctgcattgcttgtaggagttggggcacgagccttgcccagggggaaggagtgtaggcggtcttttatcggctaggatgtaagaccgaaacccagtcatgggttttgatgacccatgactgatggattttccttatacttccaccaaaacatcacgtcgaactgaatgtaaagg

SEQ ID NO: 19 - DNA, 780 bp, Artificial
  control peak
aaaaaaaaaagtttagaacacgaaaaatcaaggcttcctcaactgaaaaccactttctcttgtgccgcccccttgactggatctcaggccaaaccccaacccgccccaacactttacccctaattcccggccccacagagccccacgcgcggcgggcgagcatctacttacgaaatgactcctgcggcctctccgaaatcccaggagcatccttgcagcgcgaggagaatggaccggggcctggactcatcagcagatctatccacagccagggaccactccctaaagcccgccgcaggcccagccctcacgctttctcttcgcaatggactttgcagcaaccacgtcggccattggctcagctcggccctcgaccaatcagagctaaggttacttccacaccggtcgttcttggcaaccagtgggtaccgttcaaatccccgccagggcggaaacgtccgcccttatccgttctcttattggtcaggttagacgtccatttctgacgccacagcctatccggcgctgcgggaggcggagggcgggctggatgaaggactgggcgagcaccgagggcgtgaacccggaaggtggcgcggccaccagtaacatgatctctagactgggacggtggggttcctgccggctgtattcgggccttggactggactgagaagctacggtgcggatccagctggggtatcaggggctaggcacgtgatgaaaatgaaggacgcggacaccaggacaggaatctcctggttcttagagaatttattt

SEQ ID NO: 20 - DNA, 1280 bp, Artificial
  control peak
gatttaaaagaggggttagtctgtaaggttatgggaggtgatgctcgaatgggatctagtctcgtagacgtcaaacgttaatattctcatatatccaaagtaatgcggagagtggtttatgtagagctttacattttacatagcagctttacattcaccctcatttcacagataaggaaactgaggcccaagatggcacagtggcctcgacgcccaggttttccactttcaaagaccctccctcacatctcattacctgataatgcaacacagtctttaatcggggcgaatttcggggtggaaaggactagataatgctgataagcgtcatcatcccatatacacagcaccccaaggacagcaccctatcgcccacgcgtccaccagcccccgaaaatactggagacaaaagctgacgtcggcagagcgctcctagtccccgtcttctggtcctccacccttcccgaaaggccaagcggcggagctggggcgccaacgagaagggggtgagcgtgattgggctgtcctacatcacgtgacggccagtacgcgtgggatttggctccgaggaggcggaagtgcagcacagaaagggggtccgtgggggacggtagaagcctggaggaggagcttgagtccagccactgtctgggtactgccagccatcgggcccaggtctctggggttgtcttaccgcagtgagtaccacgcggtactacagagaccggctgcccgtgtgcccggcaggtggagccgcccgcatcagcggcctcggggaatggaagcggagaacgcgggcaggtaataccggggccagcgccgcccctcccttgcgcccctcactgctgggcaggctggctggcgcgcgcgcgcgcacgcaggggtcttttaaatcccggccggaatacaaaggccggggccgcgcgtgcgcactgtggcctggtcggcctttccttccctggctgcggcggtggtggtggaggtcggggctgctctgagggttacctggtgcacctggacccttctttggttccccgcgataaccagttgactataaaatatattagtaagcattcagcagtctttggttaatgtgtctttctggatgccattcttggctagaagggttttccgtcaccctccattaccacgcagagagctgtaaagatgagtgtcgaacatttccagctccaggagttttcattttaaagttggttgatttgttgcggcttccccccaccccaaaggcctactgaaagcaactttaagggtcacgggtt

SEQ ID NO: 21 - DNA, 1446 bp, Artificial
  control peak
ctcacgctaaatggcacaaactccacagttatatgttacttaacagatctcagttccttaagagaaactcaatgggcacagtatttcacactagatacctcctttcagacaagactggacggccagtagatgcacgatacccccaactcatgaagtcgagcatagaatcacccaacaagttacccagaaggtaaacttctcgggtactgcagctagaagaagcgagagacctctccctgggctagggactgacttcaagaaaagaactaatctgagcggacacgaggaacaagcagaaaggaaaagcgacagggctaccatgctcctaactccaccctcccgcgctcagcccggagaaggcccagtctcgctggctgctgcccctccttccacctctttccaaacccaccgagcggcgccaggaaaggtcccgccgccctcttctcccctcccccaaactaggggctggggaggcccaaggaggttaacagcttccgttccttccccgcctagaagggaaggggtgggaagtgggattgcgagctcgtcccgtctcggcctccctcgccctccacccttacccggagctccgtttccgccccagcccgagactcactcgtcagtcccgcagccaccgcagccgggtccccgcgtactgccacagcccctatcccagggccgcccccccacccccagctgcctgctccggtgggttccggggcagtcgaaagaattacttccgctgggtcagagccgctttcggcgacgcgcgggagatccgtacctggtgaggcgaggacgcagagtgtgtcgctcacttccgtctggaggtagggcgtagcgccctactcttttcccctatagtaggccagcctatgaacgcaaccggcaaatttcgaccaatccgagtctagctcttctggattcccagcatgcagcgcagaccctgatccaattgctgtaaagacgcatccttagagaggctctcgggagtccgagagagcactgcattctgggatttgtagtttctctggtggataccgtcggcgttgggaccgaggcagccctgcatcctgggacttgtagtttctcgcactgcgtgttaagattttggtggttacagagactgtccgtacctctcatttagggttacgcctggaggtgggagactgctgaggaaagattgaaacgtgggagggcggtttctttgctaacgagatcgtactctttatgaagcagcatctgtccctggtgaaaagttgctgtgcgtgctgaagagccagcacagcaacgactagcgcaatcggctggcctcatttcctctcttcccggtagaattcaatgttaactgaaaactaaccacgtcccccactccacccgcgagggtcctgcctttgcctctgggttagctcagctgtcctggatccggagctcacgcacagaaaa

SEQ ID NO: 22 - DNA, 1264 bp, Artificial
  control peak
atacgcccgatgatttaccgatttttttccctgttaaaattcacagaactgccaataagtgctttgcctcagttttgttgataaacttaggctcagacttgggaggaggggaggggtggggggtctcaccactaagaagcggtggaagccgaatccgaacttaagtcggtctgtcatcactgtagcctttccgagacagttgtcttatggagttgcttggagaacgtaaaataagaggcctaagcaatgcgtctgatgtacacctgtcgtggaataactgttcccgctcattcggccgctgtacctctgctccctcattcggagcgtagctgggtatgaatgaaattccgggcttgtagcacagagaaaacttctctcccacaattggttggtgctggggaggaagctcctgattggtggaaagtccgaggaagtgggcaagccggagggatttcgtgtacctcaggtgccctcactctgaccggccccctatcattacgtaattttctacgactcgcggtagctcaggcagagcgggttcctgcagcggcgcgaccctctgacgtctccgcttctaattggtcaagagctgcgactgcagttctgaagcctaactggttgcccgtgccgcctcggaggtgaagtgtactgcgctgattggcttccaggcgccggccgcgtgtgatgtcccgggcaacgattggctctcgcctaccgggggcttctctcaccgggactcgggactcccgggaagtggaccggcagaagagggggctagctagctgtctctgcggaccaaggagacccccgcgcccccccggtgtgaggcggcctcacagggccgggtgggctggcgagccgacgcggcggcggaggaggctgtgaggagtgtgtggaacaggacccgggacagaggaaccatggctccgcagaacctgagcaccttttgcctgttgctgctatacctcatcggggcggtgattgccgggtgaggaacagacactcgcagacaacggggcctgtgggtcagcctttgctggggggtgggagggggtgggggaagtggcattgccagactgacggagtggagaggggcatcgctgtgggttggcggggtggcggaggaaggggtagtatgggccaggctagtggggtgagaggagaaccgtgggtgacaccagtgagagggccttaggtccagagagggtcttaggtagagctagatttgcccagaggagtgggagttggatgcattgccaaacgg

SEQ ID NO: 23 - DNA, 1485 bp, Artificial
  control peak
tttaggtccatcctcctgagcgtgtgctcaccccaaggccccggcggtggccaagtgtccgcccatgcggtgtaaaggggaagtggaacgcgtagagaaggattttggcagctctgcttgcctggaattccagtcctagaattctaaattcggacttgatcttccagactgttaggaagatatatttaaggtaggaggctagaacgtatctcctttagtatatagcttcatttacaacttttaaatattcagtcaaaaagtatgtgggccttcatttacacttttgacggggcctcacaaataggggtggtctgcccctttccttacgtcccactctttcttcaaattaaattgctacagcaggaaattactgaactgtggctcttctcctacgtccgccttccctatgtcaattcccatttcccttgctttctccaatagttaggactgtaaattctaactttgttaatttcgacgccaagctggaatgaagacgctaagtggacgctaaaaagacacgctgtttgtcacacaaggcctccaactgggacggcgtccaccgcgccccgccccgcccgcggcaccgtggcatcttgggacatgtagtctttggaggggcagctgcctccaccaagtctcctcccccaagacgctgcctttcggcctcgggacgccgtgacgttgcgttcctccagtcccttgccacctccctctttcttcccctctggggaagctcagtgctggacttccgaagaccttttacgacattgagtctcggagttggtctcagcgccggatccacttttcggcaaagtgacgtggacgtcaacagcaatggcggaaggagaagatgtgccgccgctgccaacgtcgagcggcgacggctggtgagtgaaagccggggctaatttccaatgcccctttattcttccttctcaccatcttgccggtccccgaggagccgtctggcgtcggcatggaacggagaggccggtcctaggcgaagtccggtgccctgccctacccctctgccagctgggtgccccctgcctgcctggttctgcccaagggactggggcgggactcccaaggagaggggtgtcatccaggtgctgggcctcagaacggtcctcctgtgtcgtctgtcagtctccaaagtgtgctggagtgggtggcttcagggctgggtttggccggagtcctggctgtgggagttggagtaggctgaagaaggaagtttgttggatgcagatctgagtcactaaaatacaatgtgaagaaaagacgttttgaaatgaccactgcgaccttcgcctgagggttcagaggttgacccgaggagaatccctcccacttagttggtatacttttttttattctggctgttatctcgttttgcattccaacagttttcatcgttcgtctccttcacgcaagtcatcttttcccctactaccacatttctcacagctcgatttctgagct

SEQ ID NO: 24 - DNA, 1477 bp, Artificial
  control peak
gtagcatctgacatattaaatatttggctgtttgtttcgtgtttgactgccttttggaacactgcctgtgctaacaatcaacaaatacttgttgaattaagaaacttactgcttgaggtttttaaaaagtaatcacgaaattactttttcaacaaaaacaaccagttagggaataaacatttgataaattaacttttagacctatagtattataggataaagtccaaacttcgtttctcaacacatggactttcctcatctgccacgtattggccacagtcaagtagccggttcccttttctcgtgcataaagtggagttattcataattacttcacagaattaagattcaatgcagatgaaagcgcttttgtaactaaggcactataagttaccgtaagcacagggtttcttccgcgcttggagcctaggaatagtacaaggtgtcaacccgcgctaaggacctgaggttcaaggagggaaaagtctccagcgcagcgctaggaggcgctactccggcgcggggcggggctcctggccgccgttgccagggaaaccgtctgggcgtccctagtatctgagccctcgcgcctgggccactcagctcgacctctctccagcgttagcaataggtggagcgcgggccaatcggaagaggccggaggggcggggcgggcagtggccctgggaaggcgggtccgcggcctcgctccctgacttccgggtcgcggtgcttgaagggagtgttccgtcgtttccgttgccggctgtttgcagtggggaaaccgaggcagctcctgctccccctagttcttccgctcctgtgaggtggctgctgtttcttttcctcttgccccggatgcctcggtgcggctgcactgagagcgcgcgggctcgcgagccccgcggggttctgcgggaacccgggggcgcctgggccggcgggtctttgtgcgagccctgccctcccattcgcgggcggcgcgcgaggggggtcggcgcagagacgcccagagccctggggtgctcaggcgccgctacgcgtgcatcccctctgctcggcagttctgcgcttttgtcctaatgagcgcggactagatggcttgattttcaagataatgatcaacccagctgtgctatgcgctctttctgcactgacctgcgggtacttcgaacttcgacagtccctcctcaggccctagtccagaggtgacgtgtctgtgggaatgtttaaggcaggaaggagacgagcaattttgtttttaagtatctccctctcactagtatctccctgactttttcatttagaagctgaagaattttcttatggcataagatatagttgtatcgagtgtgtacttagcaaaacaaaaacttgttagtatttctgcccttgacctattgaatggaaaatcaagaatgagctaaggtgttatatactcagtgttcaacttgtatacttaagtttgtagtgtttt

SEQ ID NO: 25 - DNA, 1531 bp, Artificial
  control peak
caccgttcccacagaaatcttcacatcccttcccatttccctagtagccggcgccccgctcccaggagtctcatccacaccactattcctcggcctctccgccctagcccccaatatcctcgggttcctatttttctggcctccacccccactcaaggatcccaatgaggttctaactctacaattgcagcctgtgtgccccaagaaataaagctgctcccctgccccagtagagaacgcccgagaggcgggagcgaggcaccaaagcacaagctgggcctctccggccttccaggccaccggccgcgggaagacgagggcgggggcacagaggaatacggaatgtatgggcatctccaggggggtgaggcgtccctcagccgcaccagcccggccgcccctacgcctctggtaccgaatctgaaccgtgttcctgcagaaacttgataatgtccttcttgggcagctgctcactgcgcagctgctccacggtccacgcccgctgtggaacggccgccgccatcttcccccgctgcctccgctttactgagccagcccgccgcagttcggcatcgccccgccccctagggcggggcggggccactcacggaggtcgaggggcgggcctctgctctagcagtcgggattggggcctgtctttgagtgacgtcaaggctgtgctatgagggcttctttgttgagtccgggttgaaaaacctaccgcggtcgtctctgcagtcgactgagtagaagaccgctagagatcttcgattctgaattagacaccttgtgaagcgacctctgatggtagcgacatttatcaaccattacccacatttaaggcagctatgcctttaaagtccagtctataacctgttaaaagaaaacagcattcaggctccttatctcgttttatctagttctagcctaaaaagtcacacctgaagtgcttcagtccttgtcgatccgaaaacccatatcccaccatttcccttttaaagatcacacaaccgaaacttaaatacaaactgcccatccccttccccccatacaaatacgagaaagtttgtccaaaacgaagttcagaaactttaacaaataaaacaatttataaagaagccgcactcaggatgccacttgctgatgagaatgacattattaatgctcgtaactctgtgatgtatatttttttctcagtacacgaggaaacaaatttacagaggttactcaaagtcaggtttagagagaagtcaggaattatttgagacagaaatcttcgttagagtggaataattgcacaccgacgtttattataatgctcttacctataataaagatttaatagttgaacatcaggactccttacttaaaaaaatgtaggctgactctgggcatgttgcctatgagttagctctgctttgcaaggagcattaaaaaaaaaaattctgacgaatagctggaccagaccacgttcacttccctttaaacaatgaaaatattaaaataatgggttgccactcccagcgcggtg

SEQ ID NO: 26 - DNA, 753 bp, Artificial
  control peak
tctaacccagaggacttgggctccaggacccagtaacgcagccctggacttgaccctaaaagggagtaaagacaaaggtgaagttcagtccggggttcagggttgccaaagctcatagcctggagcttcttcacctttaggcggggtagagacatttaaaatggcccctaaacttggagggcgcgtaaagctcataggctggagcttcttaacctttaggcagggtagagacatttaagacggcccctaaacttggggagcgcataggctcatgggaaatagagtccgttacttgtcctgggaccggtggtgggtgtcccgggaggcggacttccggtgcactcctgcgcgtgtgcacctctccctgcgtgcgtgttcgcgcgtgcgtgcttgcacatgcgcgccacctccgcactgccctggcttcctgcgcctcttcaggtcatcgcttgctctcgttcccaggctttggcctctagtggacgagaatcaccgagtctgcggggctggacgctgactgcccgggccagcacctaggcgggcgggagctgtgcggcccagggttcacgcgggccgggtagaggctcgagccgggacccccgagcgtgaaccccggagccggcggcgctggggccagaggggccgggcgggaggtggtggcggaggcgaaggggcgacgggacctgggcctggcccgtgtgtgtcctcggcggcctggcgccggccgtcgctgtacggtgagccccagggagg

SEQ ID NO: 27 - DNA, 933 bp, Artificial
  control peak
gttgcctctcctcttcgagcacgcgctatcggacgcgcaggtccccaagaccgcggatcggcagggggtaccagggaagccttcattcagggcgcaggtcccagccgaaccaaagctcccaccaaagttcaacacgcccccaaagccagcgcctgattggcccgccctgcccacaagctgaggcagcctcaccttctgattggctgttatgctccctgttcggtgctggaaccctccccttctctgattggccagatgcctacctgcgctcgccccagattgggcgccagctggggcggctcgttggctcccaggggccccgctctctgattcgccgctcgggccagcccccacggcctgcacgctgaatggctgcgccgctacctatattggcctccgattgggtgcacggcgcctccccggcgtgataggtcaggaccaccgcccccctggctccccattggctgctcgaagaagcccggtctccgggtaagatggcagcggacggacagtgctcgctccccgcttcatggcggccggtgaccctcacccacgtcgaatatcctgcaggtaaaaggcggtcccggctccaaggacgccttcccagggacggcctctcagtcccgggcgctccggcctgctcgagcccgcggagggggcgccgggggaccggggtgggaactgtcaaatagtgaaccacggaggggctcggccggcgcgcgcgccgcgcaggcgcagtggtgcgaggatgagctgggcgaaattgtgaggcgctcgctcgcgcactctgcggctgcgcggccagggcgctcccccgggtcccggggcgccggtgggctgaaatgggttgaggatctgtgtgaggcgagaaaagatccctggcctggaccttggcccttcttccccgccctcccctgtcccagagaggccgca

SEQ ID NO: 28 - DNA, 928 bp, Artificial
  control peak
agatttcagctcccttgaagggatcagctttatagagacgcattgagaagaaggagaggacgacgagggggcgccccccacccccaatcagagtccccttgaaaccaagaactccccaaacagcgggggtggcttctccagtacggaaagggttacaacccaaacgctcctgtattccccttcgacagttcccttacgttatccttccccaaactggaaagtggggtgaccctcccctcccataagatcgcccccaattaggccaagtttcttacccaacagacggatcagctgttcccctcacctttcaccaacaacaaaaacagcactccgacttctggaggggagggagaagagtgggcgcgagggcttctggggtttgtggttcttctgcgcccttttgccgctgcgtgctcggatctaggactatacatcccagaaggctgcgcgcaaggagggcggggaagagggcgtccgtgacggcggcctcaccgcggcatcctgggaggcgtagttctcactcctatgacagaaaacgagggtgtggccagcaaaggacttcaaggcccatgatcccttaggagggggccggctgcattaggggtggctgctggtttgtaacgtgcgttgttgggggctggggaggatttacggctctaaagccttccatataacgctttaacggtgggcagagcggtttctgttgcgtcccgtcctgtacttttcgcacccacctgtgagataggcgttaagcgttatgcacgctttatagatgtagaaagtgaagtacggttaaggaggcggtgtgaagtgtcaaagctatgaaatgtagaactgcattagccttggctaattccacgtcatatattcttgccactacaccacagctacccaccattttggggatgggaaactgatagctggggta

SEQ ID NO: 29 - DNA, 1229 bp, Artificial
  control peak
gtgaccacaggcctcccacaagttattttcctcacgttactgggtccgaggctcctcccctccaaagagtaatagtctctcccctcctcatcctagccgaccccctcacccctgcttctccgggatgactagtctgcccttctccgtccccgagttcggaccccgcattccgctcgtggaggtccggccctcacccctgcttggccacagcccctagtgtgtcagctatttcggggacgcaattcaggtccctcccgctggacacgcggctcccaccagcacccccgaccgccatcgccgccatctttctcctcgcgctgtccacgccgggattccttgatactagtagccaatcacaaagtcaaactagtgccccagaaggcgggacgagtcgccttaacaaccagagcgtctgccacagctcccgaacaggagggatggggagtggcttttcctgccaatccgcgggctgcacagtggcgtacggcatggatccaccaatctcagggtctggttcctgacgaacttcaatctcccagaatgcttagtctatctgaagaacgccgttgagagactacaccgcccatgaaaccctgcagtataaccttgaagtaccatcacggagagaaacagtgccatctcagggccagtggcgcaatggataacgcgtctgactacggatcagaagattccaggttcgactcctggctggctcggtggggttcctcgcagcttcgctgcgtgagcattttgtaattttcctttcctttcgcagcttgatgcttctttattatcttcacgctttcattatcagatcctgagtcggttgggttatgcagccatgtaatgtcagcttttcccgtaactaccctcaaaagtgtttttcttgtgtcattatttacgtccatatgaacactgagaaaactgggttaatggctggtgctattaaaatgagctttgtcaccttactatgttacatattaaaataaactgctaactcagctactacttcctagtgattctaggaagtagtgttttgtgtttcaaacacaaaaggtgtaaataaacgcctaaacacacacacacacctcaagatagagaaaaggaattcattaattacagtgggcagcagtcatgaaattggctggcatggaggccagagagaatctacattttctcatgtcattagctgctagagatggaaaggaaggaagcaaggaaagacatag

SEQ ID NO: 30 - DNA, 895 bp, Artificial
  control peak
tcttgctaaataatcagactgcacgtgcagttttcatacgaggtgtcagaagtcaaagcaattcatcacagactgggagaataagggaacccaagcccggcccagtcttacctgcaggtagctgagggcctgagattcgggaagaaacctttaaacgctaagggacaaagtggtgcgtaggccaggcctggacgcccagggacctggccggcctgcagcgttcggacggccctcaaagccacgctcgtggctcagttcgtcccaagatcgtagcacatggcgaacgtattaacaaacctcttccccctcggactcgaccccgaaaagacaggggcgggcgaggctcaagcttttatagaaactgtcccacctctgtcattgggctatcctggcggaagtgcccgccttcccccgccgctggtacgctccgctccctgccgcgttgtttccaccaatcgtaccgtggcatttcttttggagacggtctctcagacccagacccgaagctcctcccttatgcagagtccgtgaagcctaacggtgagtgacggatgaacaaagccaatcagacttcgatctgcaacatagagtgattctgaagctgccattcccgccccgcgacgacgaggccgccggaagtcgtagtttcggaagtgcggcgcgtgaggcagccgtccgggcccggagaggcggggaactacacgtcccggcgggcgcagcggcctggcgcttccggctcgcgcgtctagtccctttcccggccggcggccgagggggcatcatgaagcgggctggcggcgctgccgctcccgggcggccgcgggcgggtgagtgtccccgcggcgcgcccgacccggggctcgcgcctcgggaccgcgcggtggggcgggatcgg

SEQ ID NO: 31 - DNA, 725 bp, Artificial
  control peak
attttggttggcggtgaatggcatgcaaaggaggtggacgatattcaggatttggtaggacagtggaagcagctacgccccagctatcgcacggtccccagcatttgtaggaaaaagcccccagggctttgggtagcctcgcaggcccgcactgccgccaggacgcagtgcacctgagggcggagcacccacgccccgggccacctgactcactgtttgtcaactctgctctctgcagccacgccctttcctccttgcctccctccccttttggctcaggggcggcataaccccgcctctctcctgctgtgactggctgtccttgccgtcactccgctgcgtgggcggggctctgagggggcacaggtagaggcccttcgcgaagggattagcatagtcccaccccccggcgcgagcctcgggccgggcgctgattgggcaggcagcggccgacgcggcggcgccgggcgcagcgagtggcgggcgcgggctgtcagtcggcgaggcggcgggccgcacttccgcctcggtgtcagtgggtcgcgggcctacggggcgggggcggggcggcagtgagctcggccggcaaccgagggacccgcgtccaggtgagtggcccccgccgccgccacgcgaaggcccgggaccccggcgagctgcggggcctgggccgcgggctggggctggagccggggcggggaggggagggcgggccgggcaggacc

SEQ ID NO: 32 - DNA, 1398 bp, Artificial
  control peak
ggcatcgtggcgcctgcctataattccagctactcgggaggctgaggcaggataaactcttgaacccgggatgtggaggttgcaggttgcacgtgacatagccgagatcgcgccattgcactccagcctgggcaacaaaagcgaaactccatctcaaaaaaaaaaaaaaaaaacaaaaaaaacaaaaaaacccagaaaaacaaaaaaacaacaaacaaaaagggaggatggtagataactgtccagatactttccagctttgccgctatatgaactattccttttgtttagttttcagcatgggagcttctggcacttttacgtactttccagcttctggggaccggctgcctagaataacaggcatttgccccagaggccgtggagtggcctcactttggggtcgtgggcagatcgctggctcccacgcctggacttcgggatcgcaggcaggatcccttccagcccaagcactccgcccaggcgcgccaggcagagccccaccccatcccgccgttcccttcagccccggggcgcggatcttgcatctgaaactgagcagagcagggcgccgggcagggccggcgggccacgtgataagcccggaaacagctccgccccctcgcttcctgagccgccacatcccggcagccctcctacctgcgcacgtggtgccgccgctgctgcctcccgctcgccctgaacccagtgcctgcagccatggctcccggccagctcggtgaggccctagcggagcggcgcggtctgcgtcctcgcctgcggccccccacgctcccgccttggcggcggccggcgggacccggcactgcaggggcggcgctgcgggattgaaagccagcgtccccgctccccggccgggccgcagcctgcgtggggcccgccttagagcagctcgcgggtgtaagacctggggaggcccggaccaggcctgcgaacgcagggtccaggtgctggccttgcgattcgagaatctcctcccccagaccctcccaaggccttgcagaacacagtgcaatgtgctgcgaatcatgagaaaaaatgtcttctctttcagccttatttagtgtctctgacaaaaccggccttgtggaatttgcaagaaacctgaccgctcttggtttgaatctggtcgcttccggagggactgcaaaagctctcagggatgctggtctggcagtcaggtaaggcatagctagttccatcagaaaggagtgtgatcacattaaccaggaagtattgtattccaggcaccagcagcaaaacgccttatttactcctcccagtagcgctgtgaggttggtgtaatacttccccactttatggggaaaaaaagtgaggctcagagagatttagcaacttttaagaccctggacggct

SEQ ID NO: 33 - DNA, 988 bp, Artificial
  control peak
tcacatttaccgtgattaatacacatgttaataaaaccctagacttagaactaggatatagaatccagtcgtttaggtacttaatgtttagttctctcctccagaaaacaggaataattaccgccttttttccactccaaagaattgatgtgataattagggagataagattagttactagtcataacaagaataaactacaactcccaaaatgcccagaggaagaggctgacgccagtccaggagacagttccagctttagggcaaaggtgaccccactgggtcccgtggcgaaagagagagccgccggtcctcgccttttttctgatgcatccagggatttgtagttcccttacggccaccaagtgcacttagggcaggcctcttactacgagtcccgtgatgacccgaggccgggcagcgcctgcgtattgaagccgagggagcgtgcgggcggtactactggcgggaggagtaaagatggcggcgcgagggtctccgccctctgctccgggctgaagcgctctgagagaggcggcagcggcaactcgagccccaacagtaatttagtgttggtagttttggcagcagctgccgaggccggagcaatggcggaactggagcacctaggagggaagcgggcagagtcggcgcgaatgcggcgggcagagcagcttcggcgctggcggggctcgctgacagagcaggagcctgcggagcgacgaggcgcggggcggcagccgctgaccaggcgcgggagccccagggtccgcttcgaggacggtgctgtctttctggccgcctgctctagcggggacaccgacgaggtgagaaagcttctggcaagaggtgctgatatcaacacggtcaacgtggacggcttgacagccctgcaccaggtaactcctttcttggtcttagaggcgtccagtctcctacagatgagcctttgaccctgcgagccaccccatggggagtatcagtgttg

SEQ ID NO: 34 - DNA, 976 bp, Artificial
  control peak
tgtttctggtctgattccctaatagatcgtgagttccttgaaggcaaggcctttgtctcattcaactgtgttctcccaaaccctagaacacggcctgacccatggtaggcaaccaatgatactttttgagtgaatagaacaaaaagtattgccctccttaataaaagaaaacctagaacttcaaggctgacattggccttcgcgtagcccaatcagagcgctttgcccttgctgcagaggactaaaggacgccacgccgccgttgcctagtaactgtcgccggagggaggcggggagggtcgcaagcgcgccgccggcgtccaatcaggacctgcgttccgggagccaatagcagcttcgagagccgctcacctagagcggactacacttcccgtcacgccccgccgcctggggctgggcagtgaccgtaaagctgttcgctcggtgcgacgcaagtctcagctcagcgcgcttatcctgggtccaccggcgctaccgccccccgacgtgagagagcgaagttcttgggccgcgctccctccctacctgggtgccctccccctccgggagcctgggtccccggggcggtcgcggcggctgcatcctcaggccaggccgcggggggagggggcggcacgggcctccgaaagcggggccatggagcccagagagtcggggaaggtaggactggaaggcccgggggccgcccgctccctcccgaggatccccgagccgggctcgggcgaggcggggcggctcgggcggaggccccgcagatgagccaggcttgcagggaaggactcgccggcccgccctctcttcccgcacttcggcgcccgaccccgggagggtggcggagaggagcggacgcttccgttcggtgtccgcgcatcctccccgagctgccgagcgtccgctgggtcgatgctgggttgggtgacccccgagtggagcgatgcgaaggtgcttcctgttt

SEQ ID NO: 35 - DNA, 1170 bp, Artificial
  control peak
aaaaagagacaaagaatgtccctctgtaccctggggaaagcgccgacctactcggtgcggaggtggtcttcggagcagctgcctcaatggctccgaccaccgtgggatgcgcccgggaatgggagaagagaaggggcctgggacgttcgtgcccattcaaaaccgatggctgcagagagcgcgaccaactgtctcatttcccacgccaaagtctggggagcgaagttccgaccccagtcccagttccagccctgccaggccaagaccagcgtatggccggaccgctggccatcatccttaacctctgctccaggcgcgcgtgcgttcaacttcctgggaaccgcgctccacaccatgtgcacccagctggcgaggattaacgcccagctcctccacacaacacccccgggggcctggccggggcaccagacacacctgttagataaacctgagggcaggcccgccccagcccccaggacacacggtcctgctctcgcgccccggccccggtccctcctcacccgtagaggccctgcggtcggcgctcctagccaggcgcgtcggactctcccccaccagtgtgcacagcaccggatccggatccggcttcagcgccagccccacctcctcctccaccgcccgcgccacagttgggaaccgcggcccagacgtggcagcgccaacgcctccacctcgcctctgccccctcacgcagggccgcgcgcaggcgagcggaggcggaggccaaggcgccacccgcccaggcccggagagcgaggggcggggcccggagagcgggggcgggaccaggagggcgaggggcggggctcggagagcgggggcgggaccaggagggcgaggggcggggcctgaggctcgagtcgcggcgcctggggcggtggccgccggaccaaagctggcctctcccggctcctcctcctcgctccccaaggcctttcccaccgcgtcattgtctcggtttatctgcagatacgtctacctagcagtcagcccagcagcgtaagtggtgaccagtgtgggcctctttgatcctgagaccgaaggctcggccactggccctctccatttgttccttgttgccctcccttcatgattcaatccattaattaggacttttcctaccagctggcagttcagagctccgcatcc

SEQ ID NO: 36 - DNA, 1527 bp, Artificial
  control peak
aaccccccacccccccacccgtgccaggaatctatcgcagttctcaagtcctgtgacttaataccgcagctggcacctcagaattccttattcttacagagctggtccccacagtaccgcccctgggcgtaaactcctccatacagggagtgtagatgatgggctgacctaagaacggtttccaaatggtcctttacacctctgcctcccaccccactctagcccaggataacagattttcctcgcgcaatttcaaatttgcaagggaatcttgaatattttctggtaatgagagagggtagattatatgtcctaatcccagcctaatcagattaacagcaacttctggacattcagtaccaggaactgttttaggtgctgtggagagaagagttaaaaatgaaacaaaaattattgccttcatgagcctcacattagtggacagagtccaaaaaaaaagtgacaacgtaatgcgctactgcgacacaaagcaaagcaaggtgggcaagcaacactagttctttattcccgcacgccctaatccccaagtagctcttccgggtcactgcagtcttcacgggctccagactccgcccatggtccgtccttcaggccttgactccgcccacgcatcgacgtagaatttcccggccgggctccatgaagtaccacccacccgcgacgccgacgcaaggctgctgctatggggccgggcggccgtgtggcgcggctgctcgccccactaatgtggcgcagggcggtttcctcggtggcggggtccgcggttggagccgagcccgggcttcggctgctggccgtgcagcggcttcccgtaggagcagcgttctgccgggcttgccagaccccaaactttgtccgcggcctgcacagcgagcctgggctggaggagcgggcggaggggacggtcaacgagggacgcccagaatcggacgcggcaggtacgggcgtggagaagaacgaaggcgaccctctccgggcagtctggagtcaggtcccagccaaggtggctctgggttcccctctatgtgcatttctcttggagtcacaaaactcctgcacatccagagctggaagggacttcaaaggttgttggcgccaaccttcccgtttcacagtgaagaaattgagggcaagagatgaactgcagaatcccagcctcccggacggccatctcccacccttggcatcacagtctcaccgtcctcacttccttttaccctcttcctaggatcctgggaaatgggtttgtggggactgttggggcttgaggattctgaggagccagggtctgggtgcgaaggggtgcgtaggaagcgccagtaggagatgggtccaagttaatatcctctagttattgctgacgtttttagaaacttaagaatggtaaacagatcacatctaagaatgtagagtatgagagggagaggccttagtgggcttagctactaggtcctttggagtcggctttagaacttgggcgagcttttaaatttacagcctgtaata

SEQ ID NO: 37 - DNA, 1150 bp, Artificial
  control peak
aaaaaatgagaaacatccctaaaaaagcaagactaagctgcgtgggaggccaaacgaggggggtcctttgggctgtttctaccatgacaaaaatcgccattaccccacacctctcacaccgccaacgttccgccaacgttccgccaacgtcccaccaccctcatgatatccgtccgcggtactgctccagcgaaggtacccttgcctgggccatgaggcgcaaccgtccctcacagctggggtggcttctccaggttcgcaccgaggggaagttggggtaccggtttccgccgccagcacccaagatggcggcacgagcgtcgcaaccacaagaaccacatgcttcgtgtcccgcctcagcattctcgggtctcactagacttttgcaagtcactgtgggaaccctcaaggcgagggtgttccgcactctggcggcgatacaccttcctattccctgacctaggccttcattcccccgccacagaaagcgctggaagcgcccaacctggatacttcacccaacgcccaagcccactaacatcgcccgccgcgaaggcgcggccgaggggcccagaatgccagacactagggccttcgccctcctcattggtgggtccataccgcggggcgggcctaggtactcattgggccgggaattatgtcagtcttgggaaatcaggtccggggatgaggaggtacactcagccattgggccctctctgctgccaatcagagggacatccgtcgacggccccggcctcggctcgttgattggagaaactgttcctagggtgtttgcggcgggcgggtttccgagtgcggtagcaggagggggcggggaggtaagcggaggctccgagctctaggccggccggcggtggcggcggcgaggccgggactcgggcttagggcctgctgtggaggcagcggcggacgccgagctaagcagtgggtatcgggcacattcctttcccagaagggagggttgcagcgccgcggggccgaggcttatggggcgacgggatggtgctgaggtgcaggaggcgggggccgagcggggttcccgccagagcccggccctgggcctgacagggaggggccccgcgcggggccgagaggaaagctggctccggggtgggatggactgggc

SEQ ID NO: 38 - DNA, 1074 bp, Artificial
  control peak
gtcacaggcccacaccccgctccgcgcgctcctgagcctgcccggaggcgggaggcggccggcggtcctcggggggttgagggtcacctgcaggaccacggcggcagcccggggacggggccagccttcgcgtccgagctcaggggcggcggcagcagctccaggcccgcttccctccaagtcccttctgcgaaaagatggggcacctcaacaagagctgacaggaaaatggggagcggcccggccccagggctccacggctccacgggcggggccctaacattccagagggcagaacctctgacctctgacccccaaccctagccacctggggccagcccacgacagtcgcggtcaccgcccctccagctggagcgtccccctaggagcccggggaggggcggcccgcccgcgaaccctcgagcgaggccaggtcgccgtaccgccatgagcccgcgcggctccctagccagaccgcggccccctcgtccaggccccggtacctcagcgcccaggctccgccgcgaccggcgaccggcgactcgcgacccgcgacccgcgacccgcagctgccaccgcgcctgcggattggctacgaacatcagcagggcccccctgggggcgggactggggaggcagccaatgaagtcagcgctgggcgggcactggagcgagcctggctgcgcggagagctcatcccgggaccacagggcccggggcggggctcggggcggggctcggggcggggctcgacagggttccacaccccggaagtggccctcgccccgcccacccgcctaccaggtctacccgcgcgggccctggcttgttttcgcgtctgttcggagtaataaggcacctcagctcctccctcccaaagtgcttggctttctgctgagccgcagctttctcgcccgtaaagaggcctttgaccccacttgtgggtaatgatctgtagctgtgactcggggtggattccagtagcgtggggtcccctgagccctcctgcggggaagggctgggcgcagatgggcgaggggtctttctgcggcgggatctgcgctcctgggcctt

SEQ ID NO: 39 - DNA, 1068 bp, Artificial
  control peak
aactgagatgctgtaacgtgcgtggcccggtgcccaacacatagtaagagagaagttaagtggcagttattggtatttattaccgtcgttgttgttggcttagcacagagctgcactcaggccttgggggcggggtctcctccggggaacttctccgacggtcccctcacctgcctggaacgcccccggagtgacccagcaagaggcttccccttgccagagctcacaggaaggcccttttccaaacgcacctcgcgctcggcgccgggccccttccctgtacgcctcaatttcgccccgcccgccgaggtcacgcgctccaagttaccattttcttgctctcggtgccacggttcgcctgcctcgacatggtgccggccgccccgccccactcgcctagccacgggttagctcggcgacccctgcagacgccggagcctaagccgctgcccctcagcccacaagaccgaccggcactgactcactgcgcctgcgcggcctcccgcggggcaccacgggactagtggtccggccgacgtgggcaactcccgcccaacaaacgtcagactacaacacccaacagcccctttggcagcaccgcctcttaacacagtcaactacaactcccggagtgctccgcgcgcgcctgcctggtagagcccagcttgcgctgggcagcatcctggccgctgggtggcgtccgcagccttcggtaatcctgggtcacacacacgcttcctccttgtccccctccccccgcgcccggctcagagatgatgctccacaaattcctctctcgccggggttggtcttggaaagtccctctttcgagctagcgtcttccgctgttgcgattcagctccagaagctcccaagatgtcccagaaaactagcaatgactcacacagaattggcttcttcattagaaaaatatttaatgacctactatgtgccaagcatttcgctagtgtctttctcttcagactgccaaaccccaaatgatgccagcacctgccttctaccacctgtggcctcgtgatggaatatccagtcttttaggtaaagttc

SEQ ID NO: 40 - DNA, 1402 bp, Artificial
  control peak
agtgcttgctctgctgtgttctctgtagccacattgagtcggttacttgttggagcatcaggagcctctgtaaaatgaggacactagtgttgctgacatcatagggttggtgaaaggattaaatgaaaaaatgtctgtcaatcacttagcaccacgtctgagatgtagttggcccttaatatatacagaagtttacaaggtatttgtttgtttaataaagacaggtccactccaagaaatgttattaagtacttaataacgtgtgctttgaggtgtacacatgagtacaacagcgaacaacatgagcaggtaaataactgaacaaacaaataattttaaatgcagtaactctgtaaagagaaatggaggatgctaatttttaactccggtcaggaaacttctgatgtatttttctatatctttatttaataatcttctgataatgagttaatttaatataaactcagataaaattttttgacaaaactaagagataaaccaaaatgaaatatccgattaggatcccgaatcgcgcctcccgaggttagtactacccctcacgactcctccctccgccacagccacgcctcttctgggtttacagttcagaatcacgccggcctcattctaccgctgctctctcctggatctcggccacggatcttttgctcgcgaaagttcctccgtctagctgcacacaacgctgctgcaggaaaccgaggtaagggatttgccgagacttagctccaccacactccccgaggccccgcccctcctctctggccctcccctaggcccaggtgtctcgcgttgcacgtgcagttgttgtggttctacgtcacgtggtcccggaagttcaagacagacccgcctcaaacatggcggcgcccagcgcgcgaggacgtgatccgcttctgctccggcttggattgtagccttgacgaggtctgagcgaccatggaccggccggggttcgtggcagcgctggtggtgagtgcggggcggtggggtgggttgctcagagtgccggcgtccggcattggagcccgcgggggttctctgcactggttttcttccggttttgcgggctggactgtcctcgggttactggcagccaggtctgagagggagcacgaggctcccaggccaccggcgggccaggtgttaaggctatgtctgtactgccaggtcataagagccaggtgggggaaggaaggtgactcattgtcttttgcaccctcatttacttaaaaaagagcttctattatacactgttaactactctttcattaaatagtgtaactactctagagatcttgggaattaaagaaatgtatgtaacacaacaacaaaaaccttactcacatcgctcccagcccttctattcaataccacgtttctc

SEQ ID NO: 41 - DNA, 1071 bp, Artificial
  control peak
acaaatgaaacagaaactaccccaatggtttttagatgaacctttttgggtgaaggtagttggggtgaagtacggaaaacgaagacgaaatagggaagagaagtgagagggaggacatggtaacctcgatatccctctgagggaccaagagagcgcccccaggaagaagactctcggtctcctgtgctgacaatcctcccgctgcacctgcccaagcatcccgcgtggagcatgctgggagagggctggggcccaggcctcccccaagagagggaggggagtcagggtgcgcttggggtgggggtcgaggccccgccactcacccgcgtctagaacggcttggggacaggaaagcgcagccgcgcacgggtttcctctcacgttggcgcaccactccgttacgctcctgaccaggctgttctagctgcaggcggtgttctctcgttggtctcgctaaaggtggggagcgccaggaaacgagcgccgcgaaggcgggggtagtggggcggggtaaaggagccggcggctgggcggagccgtccgtttgcccgcccgcgcaggcgtcggaagggccggcgcgcccgctgcgcgcacactgcgtgccctttctccccgccccctgccgagttcagagacttgctataggcctgcgtgacccggacgctctcctttacccaacgggcgtccagagaagaggccgtgcgtacgactcgctgacacctcccatttcaccttcctggcatggaaattgcgagagcgagagattcttgctcccggtgtcgctttttatcagacctcttgatggtttctctgatgtacccctgcgtccttgaaccctcatattgcccacccccccagtactcctgggctgagaccaaaacgcaagccactgaaatagtcacgtaattatgacaccaacggtttcctattattactggaatgagatctgaagccccactgcctggaatccaatcctgtcttcaccacaccttttgatatcccaggaaagttatttcacctcgctgtgcctcagttccctcatttgtaaagtacagaaaatgtcacagtagt

SEQ ID NO: 42 - DNA, 855 bp, Artificial
  control peak
tagaccggagcagctggacgtcccaagggacctcctggcctgacccgcgcgggtccggccgtagcctaccttcctcgggagaactccgcagccagaagcccagcccctctccccctagcccagtcagcgcgaactcgccgccgcccccgccgcctccgccctggccccggccccggcccgggccccgacccgaagtccaggcccccttcctccggcctccgcctccggagtgctgagctggagattccgccccgcgacggcccccagatctctgacccgcgttaccggcactcacctgccgctgcccccgaccaactcaagcgccactgcctctacctcggccgccagtggccaccaagcgccaccacctcccccatgacaacaggcccaccctctttcatacttctaacctatccattggatagtggggcggcagctctcctctctgattggtttagtatacccgcccgtcatcgctctttaccgccccttttcctaaacccggtattcgcccccgcccggacctggagcgtcctagcccaagctttagttgtaattggttgagggttgggcaattcgaagttatgataggctgctaaaagtgtctgttaagctaactggcaagagacgtgacggttaaggttgattcacgcttgtgcacagtttggttgcgcagtagaaagcaaaggaggggcagacgctcacattcgtggttacccgggaagggttaagtctttgttgtagagttttctctgtaaacttttccacttctccagctcacttcctgcaaggagaagcttatctctgctagctttattcataggggtagtacttacaaaacaccaacattagagg

SEQ ID NO: 43 - DNA, 669 bp, Artificial
  control peak
agctgcctcgaaaaggagtgattcctcaaggggcatggtcttggcccaggtaactttgataatacgtggacaggggagtagggatgcccgctcaagttctcaaagcggaccccgcccctttccagccaattgtagcctttttccccataatgcccagcagccactgccaaaccgcgcactgcaaccgaactctacctaactccacctctctagccgcccagcccagttcctcattttttattggttttttaaactgtcactcagagagtactcgtatccgttggttgtcccctaacataccctttctttcccttgtaatcgcgcttctttaggcactacccgcctcctgaagccaatttcctttattcgattggatagtcggaatgctacttttcacggtgcaaagctatgattggctgcgcgcggcagggctcgcgcagttaccaggcagcgggctagactgaggctagttaccacgtcagtgagctgacagggaaggtacccggctctactgcccgtcccggacgactcatcctggcggactgtgcagtcccaccctgacttcggcctcaatttccaggaaacaggctccttctcttctcccatctgctaccagagccgggagagctgctcggagacgcctccggggtgcgggctggacatgagcag

SEQ ID NO: 44 - DNA, 1256 bp, Artificial
  control peak
atcttaaacatcaccaacgagccttgtgagaaatggtttaacgttgtgataccttgcccaattttaaggcaggtaaaggtgtgtgttaaaactcaactcttgaactgaaccccagctgccctttagggcatgcttaatttgatgtggactgtaacctcttttgcagcattgtagagatttggaatctgcacattaggaagagaccccacgaggcggtggcgttctttctcgttcggagctcacacccctcctggggagccctctcccaacccgggctcccggtaagcgcggtcgccgctcctctgagggctgctcttccctcgcttctgagccgaggcccccgcgacacgaacgccacgcccgaagcgctcttgccaggacgtactgaccgagggtaggctgcccagccgggtggtaacgttgaggaaaggacagcagcttcgcaaaggagctgggtaaaaccacgcccagaccccgctggccaccagcccgagaccaagacgcaggcggtggcgccggtttctcactcaaccgcaggttccagcatccccgatacggccgacgtcattaccctgcgaccgctcaggacacttctgaggactcttttgacagctagtaatcgcgcggtggcgcgaccacgtgactccggcgccctagccccgccctccgtctccaaggagccctggaaggagctataccagcggctccagaaggcggggagaggcggggccggactccgggacagccaatgggcgaggagtgccgcccagcctgacccaccggccgctccctcacgagcctgtgggcgtgcgccggctgtgcaggtgaggggcagcgagggcgcctagtgggcagggcgccggcgttgcgcgtgcgtcagtgttcttccttgtgcagtttagaatttgatacatttctttccagtctatttctatagatttgttttcaaatcacatcagaccctatgtaaaatagtgtaccgtctttactcattataacatggtgagcatttccccaagctattacacatatttttaaacattgttttaatgaccggcataataatccagtgcttggatatatcaaattcttcggccatctccttataggtgagattgtttccaaagtttcactaatgtaagtgcactgcagtgaacgcccttacgtaacaattgttcactcccgtgtacttttctcgtgcagagttctacagcatgaaacactatatttaactttgtcaggttgatgtttatcag

SEQ ID NO: 45 - DNA, 1192 bp, Artificial
  control peak
cgaaatcaagctaggtcaaaatttatgccttcctaccacctggccataacgttatgcctttcccgttgtttggataggaagctttttccgtaagtgaattccctccccacactcccctaacataggctttacgttcaatctacccccgcctggcagaggcgagcccacgccgcagccgaccttagttcacccccgcccgcggcctccctctgcgtcattgccccagtagagtccggacggccgcgctttggtctcaaaccctgcgatggtctcacctggccggaggggccatgcgggaggtgcggcttcggcttccacgcggcatcctaggtaagcgacggctaggcctccggacgtgggacaaccaccgaagagctaagcgacttctgaggagaccggaagatgggaggcggggctggcctcaacgcgcaaccaaccagcacacaggcagagaacttccgttctccgtcgtctccgcaggcctgaagttcattggaagagcaggacgtcacggggacgcctcgtccttcgcggtcaacgccctgaacttggcgccaggatctttaaaacccggaagttcctgtggttcatccgcgaaccttgtcctccggtcctcgcatcagagcacttagcattttgcttccctagggcagctggcctgtcgctccgtccttcaaacatttgtattaaatccaaccttaagagtgactcacatctctggctggattgccaggggaccttgggcagatttcttagcctttctgagcattcagttataagggcaaatatttttggaggcacagtgtggacgcgtagtatgaactgaatgaaagaaatcggtttcgtcaaaaataaacagagttgaattagttgtatttggtatttctctttcttgtctgtggatccagtcagcaaaagttaaattcaggcagcaataatgttttacatttatgtattactatatattttcagctccttttcatatgtattatagaatttgtgtccagaattgggacagtaaccatacactggtaaattcaacaaaaatttactgtctaactgggttatggagatactgcaccaaatcagataagattctaccttcacgaagttaagtttcaaggaggggagacacaaacgcctacgtaaatcaggtggtggtgacatgaaaagagaggaaggagactggaaa

SEQ ID NO: 46 - DNA, 1097 bp, Artificial
  control peak
tctcctcaacctgttccctcattccttctcgccagaatctatcaaggctcggcacaaaggtactctcccaggaggccttcctgaattcttctgtgaggcagactacttcccgagctgccccggcccttctccggccaagtctctccagaaactcatcccattcgttctcagatggaaggggacccacacccacgacaagcctccgggtttgcttaatgactcagacagcgagggtgctcggtaagccccaccagcccgcgccctactcagcaggtaatgacggagacctactaggtgacgggcgccgcggtagacgctgggataaggtccctgctccagggagcttacggtctattcactaacgtcaaagagcagtgagtgctgttacgtgtatgggagccaccagggtctccgcctgcctgcagggccacgcgcgcactcgacgcttcgcgcctgcctcagtttctccgtgaggccgcacagccccagtctcgggaggcgtgacacaccctccccgcgcgctctcgcgttaccaagtctgcgcagccgcacacccccagtcccggcgggccccgcgcgccaggccccttcccgacaggccccaggagccccgctgcatgccggggcttgaggtctcggcgaagcggcgcggcgcggcgaagcgcggcggccggcgggcgcgcgtggcaggaagcggaagcgatccgaggcccggccccggccccgccccgcgccgcgccgcgccgcttgccgccgggctagcactgacgtgtctctcggcggagctgctgtgcagtggaacgcgctgggccgcgggcagcgtcgcctcacgcggagcagagctgagctgaagcgggacccggagcccgagcagccgccgccatggcaagtgagtcctgtagggaagccctattgaggccgggacggaacagatcccccttccccacacccgtgcggtcgggcgcccgccggccaaccctgaccggccccctgcagaacgcggcccgtgcccccggcccttctcgagtatccccacgcgtccggggtgcggccggctcccctggcccctgctctcactcgtcgtgggcagaaggcgt

SEQ ID NO: 47 - DNA, 1385 bp, Artificial
  control peak
ttgagttaatcaaattaataagactgatcgttaagaacgactgccaaaaatacgaaaaagctactgggatccatctttccaagacaatttctattatctgaattaacaccatacctggtacccactgattaaaagctgggggttaccaatgcgcgtgggcacagttagaagcttatgtagcaaaaatgagcacatcctggaagggcccgggagaaggtgctcctggggcagcgcggagagggagctctgaggctggggcggcagcggtgcttgccgccgtccccctggtcgctcccggaattaacgccgcgcacgcgtcggaggcatggccccgtcccgaccccgtttggcggctcacctcgcaggccggcacagcacggctgctcgcggcagcagaagaggaagatgcagcggtggaaggcgtccgggcggccaggcagcggcgcatacacctgcagcaggaaggagagcgggcggccgcacagctcgcaggccagggcctggggccccggcagcccggccgcgcccagccatgccggccgcccgcccaccttgctggggaactgctcgctgcgcagtcgccacgccggcgccgactcggcgaagcccagctccacaggcctggccccggcggcagccatgcggggcgcgggctggcgtggggcgcagcccacagctgggtcggaaggcggaaatcgggcgccgggccggaaggcaagaggcgggcacctttccggaggacaggaggcggaaacgcgtctgacgggagcggttgcaggaccaatgcgagggaacggggcagaggaaacctctcggcatcagccccgcccctggcgcctctgcctccgagccgctttcctggtgcctccgggtgctctgggatggttctggtctttgggagagtggcagctggtgacggcgctccgctcacctctgcacatgtcttgctgtgggcctgcgggtggccgccagggaggcagagccctcccgcaaaccttccctgctggtgtccacctcagggtgtgggaaacctgtgcgctggccgagtgctaaccaagagtaggcagtgaaagacaaatgaaggttgaacaggtaaagtgaggaccctacagcggaaaccaagaatcctgtgtgcctgagagtaatgaagaagcctctgcagaagagtcttttctgtcagtcttaaggtctctgttttaatgttagtgctggcttgctgtacctgaattccaagggaggagtgtataatgaggcatggccaacccccacttcccatcattgcctgaactagtttttcaggttaacttcagaatgcccttgggcaagcagagggtccatcagtcggttggagggtttagaattttactgttggtttgcaaaggtct

SEQ ID NO: 48 - DNA, 723 bp, Artificial
  control peak
agcaaagtttatgcagttgaactggattctgagtatatctgtggatagttcctgggagagtcgcagatacgacacggggttgtcacattctctatgggtctgtttcctcatctttaaaagccatctctgcgttgctaaggcaaagcataaaatgtttgaggaaagcgacttttctagaaagcacgttctacaaacacaccatccacgttccaaactgagcttcggtgttttgactggagggcacctgaggacgtcagtacggtcccgcaaagaccagcaaccagattggcaagcagggggcaggagaatttcccgccccatctctttagttgtctattggttgattttaccatcagtctcccctgtcctcctatcatcgccgctatatttgtgtctcggtgcatactctcattggccctacctgcagtcgctctacgtaaggggcgttccagagtcagccactgggagtcgctggattcgggttttaaaaaacgccggccgtgaagtgggcggagcgagcgatttgaacgcgagcggcgcggacttctgccaagcaccggctcatgtgaggctcgcggcacagcgttctctgggctccccagaagccagcctttcgctcccggacccggcagcccgagcaggagccgtgggaccgggcgccagcaccctctgcggcgtgtcatgggcccgcgccgccggagccgaaagcccgaggccccgagg

SEQ ID NO: 49 - DNA, 1296 bp, Artificial
  control peak
actggctccgcgaggcctggggcgcaggggtggggctcacgtcgtgggacggttgcatggattattttcgattgatttccgaaccttcaggaagcagacacgctccccttccttcttagggatggggcggcagccaacgaaagcccgaagccgcctccttttctgagtggggagcttagagatgctcaggagggacctccccgattctgcgattctgagcatcaattttggcaggaggctagatagaagtagcaattgacattcatcaccctaaagtgcggattatgtcagtacagatgctacgatagaagaactaaaaaatgacagaaatggttttactgtttttaaactctgggagcctactcctttaagagattaggcagtacgcctattctcgcctggtcccagcgtaccttgcgcggctcgccgtcccccactcacgtaccagggctcattgcgtgacggttacacttccggcgggctacgtgcgcgcggcgagcgccgggcgtacttttgcgtggttgctccaccccctcagccttgccttcgccgccgttggggctggaagttcccgccaggtccgtgccgggcgagagagatgctgcccggcccgcctcggctttgaggcgagagaagtgtcccagacccatttcgccttgctgacggcgtcgagccctggccagacatgtccacagggttctccttcgggtccgggactctgggctccaccaccgtggccgccggcgggaccagcacaggcggcgttttctccttcggaacgggagcgtctaggtaaccgcactttctcgccttcctgggccggattcacccccacccccacccccgcaaagctcccttggtgtccccatcctgtctcttccctctggcttccttcccagtggggctggagagaagcacttaatctagccgcccggcgtcgtaggcctctcgccgcggtacccgggcccagcgcggcctggtgaagtggagggcgggatgggttctgtgctgtgaggtttccagaatccacgtatagttcaggcattatttggacctagtggggtcttgactgcgtggaagggccttggggtctaccccggagctttagcgatgtgtcagtcttaggcatagaatgaacattgaatgaatgaatgaaattatagcatactgtggagttttatgatagataattccgatttcattgcagcatttgcggttttggatatgacggtgttttaataactttgtggtttttaaccagcttttcaagcttttgtgccacatgggaccttatct

SEQ ID NO: 50 - DNA, 1174 bp, Artificial
  control peak
gcctcccaaagtgctgggattacaggcgtgagccaccgcgcccggcccaggagttcacgttcttaagagctgaaatatgtaaagtggcccataataagcactacattaatgttagctattattcttgttatttctactaagattaatataataatccttatctcgtacgctcttttaagaaccaactatttaacgtgtgaaaagtgccaggcagcaggtcctggaattgcagttaagattcttccggtcctcccaatccacctttcccgagacgctcgagttcacttacttaagcgtcagccttccccaagatggcgttatctttgtcgccaaccccacatccgccccacccaatatggcgtccatcgcaatcaaaagctttattccgcccttctcaagatggcgcccacaccaatccttgttaccttccgccctcaagatgaccttagcagttagcgtcaacctccgcactctatgacctttccaaaatgatggtgaacgtaatacgcaccacactctgaccttctcaagatggcgctgagctgacaaactcgcgtcgcaaactcacgcaaactcagtgtcgctgtcgaagacacttccggttgcgacggaggtaggcttacgaggcctgtgtcgggtagaaagggtccttcctggaccgggaccctctgccacgaccatggaccgtaggaaaaagcctttggacgtcacggcctcctcggtgagtgagggtatgaggtaactccacgggctccgggttcaccgtgtctccatcagacaagaatgtcgacctagcttgtttcttcactcggggacccagagacctgacctctcccctcaaacaggaacccccgaactggattttcttctccggcggggatccggttcagttttcttacaggaccctgaattgggctactctcctctgacatgcgcccgggccagaaccctcggacacggatctgaacctggtgtcccgtgggttaggaaactccgtgcctcacctctgccctgcccacaaggacctgcacctcgtcagccttctcccttacagtcaggaggaaccctagcttctttttgctatgcagacatatcctcttcctggtttctcctccatccagggacccacaaagtcctcccagaactttagagtgtgacatatttcgcaggaccc

SEQ ID NO: 51 - DNA, 1102 bp, Artificial
  control peak
cgcaggggcgtgagggagaggggagacgaatgtttactcttggcaaagaggattctaaaagacgggaaaaataaaaacgagcattttcaaagtatcagtcttcgttgccaacaggctttttaccagctcaccgctacccccaaactccctcagcacctgaatgttacgagagttctaaaagcatcataaaacgtttggcacacagtgcctggcattacgcgcctctacatacgttattctcctacccgtcttcagaccttgccagggagagaaataagaaccgacgcgaatttagacgtaaaacaaacaaaacgggcttctggaaagaggccaactgtgccaggggggacgaatgccccacacggatgactcggagcttgatttcggcggggagcgcggtctcggacacgcccagagcccgagaggacggttctgtccccgccctgccgcacacacgccttaccggagcgccgcgccaggccgcccgcacacccagagctcgcccacggttggcagcgcccaaggttgcatggcatggcccgcttaagtgccactcagccggccccaggcgggatcagtgcgccggaagtggcgtgccggaaacaggcgcgtcggaagttgcgtctccggaagcgtttgtctctttacccagcttcccgggctaacccttaccgagtgtcacctgcaggaggcaccgggactccggggctgcacggcccgaatgcttcttcgtgggttttgtttaagcagccgcgacctgtgcctgccgccggctacctccgctgtggcgggcgagcacctagggtcctcacctacctgggtcacagtcaggtccagggcaagccggctacgtggtttctcccctcctcagtacaggggccaggctagagaccgccgtggcttcccagggaggcagaagccgagtccgtcgagcctcaggaccagcagtcagagcggccagatatccgctgcaaggcgccagccttccatgggcgcgcgccgtcaacaccccactaccacacttcccttgaatagcacacttgtctgtgctgtcagcaccccactatacttcccttgaatagccccacaaacttgtgcttat

SEQ ID NO: 52 - DNA, 1058 bp, Artificial
  control peak
tgagtctcagatagctcttatatctttgggcgttaccaaaaacaattctagggtaggctgcctaagtgactgcaagagaaataagcagctttaaaatgaggtccttctgaagaacaatccagatccatcatcaatgtcactaaagtgtaaagaacactaacttctatggggtctaatcatggtctaatgacttgccgccacccaggtaaagcattcatagctgtggggtgggaaggagagaagaaagcaccaggtacctttcaaagtttacttttcaccgagacaaactgatcccagccgacgctccacaggacttctgaagctccaccagctccagagaagggggaggaggtggtagaaactaacaggattccgtgggacccaccctccagctgactgaggactggcccgcagcggtgaaaggcctttggggtccctagctcctcagggactcctctgcgcgctccgggatccatacctggcaagcaatagcgcggcgaaagccgcacgccatgtccacctctacggaagtgcgccgcagtccgccagttccggcctcgccccacctccttatacgacccgcctccccgcgccggaaacccgccccggagggtgatgccagccaatcaaggagccgtgtggctcgccactaacctctgccctgcagccgcgagggcgcgcgggaaatcccgagtgcatctggaatacgcagagtcagtaagaccatggctacgtcctcgatgtctaaggtatgtggtggccgatgcgcactgtggctctaccgctgcctcattcccctttgaagaagggatgaagccgaggttacgtaggcatcccttccctggctggagaggagggagggagcggcgtactgagctgaggcgtccttgctccagatcagttgaggccaactttgcttggggagcgaggacgcggatctgccttgtggtatggctaggaggatttttaagttctggttggtggaggtcggatgtcctgatgatgtggcccagtctctcatttttccatctcacaaatactgtgcgccaagcactccg

SEQ ID NO: 53 - DNA, 1039 bp, Artificial
  control peak
ttgcatcgaagatcctacacctcagtccctaaaattatactttcccatgaccacaaaggccccacttcccgcccttgagcgacccttccataaccagctcgcgaaacgaacgcgtggaaagctgtgaggggggcgggcactggttctcattcctgggaagggactgcacttacctcacgccagcccggggtgctgtgggggcggcccgttatcggggccccggagccggttcctacggccccatacacccgagctcggtccggagcagctggacgcagagggcccgaccatagacacaggccgcaagctagagaggccccctcacccccaccctcaccatgtgaccagctgccaaagaggcgcgcggaaactgggagggcggggcgaacgccggacgttctgggcacgtgaccgccacccatgcgctgaggggcggacaggaggtgcttcgactgggaggagggcgaagagtgtaagggggcggaggggcgatggcagccacgtgatctgttgctatcacacgtgaccgcttgacattgctctgaggagcagaggtgtggctacgcggcgggcacgtgactggctgccacaacccccgctgcagggggggagcggtgagggggacaagagagggtggagcccgaggcggtgccatggcaacctaggccacccagagcccaactcccggctgaaggattgtctcactaccggtctgaaggctgaactccagtctcactaccggtctttcagcctgtctgcattggggagggggtgggagttggggtaccagaggtgatgctgtcaccttttcgctccaccatgagaaaacatactctcatttgtgtcattgtttcattcattaattctacaacattttactgagtgcttgcttccagtgtgtatgtgggtttttattttgtttttggtgtctgtcactggccctttaaagtagtggtacgactatctataaagatgtagccaacaatggggtcttctacaatagggagtaaacaatgcccatttgagggcaaaactacga

SEQ ID NO: 54 - DNA, 1188 bp, Artificial
  control peak
acttctcttgcccaggtctctgtgttcctataacaatgtgtatctcttacagcacttgcttggatccatttatgtacctctcacagatcttatgctcctgcaggcctgaaacttaagcccactttctatttttcatagctcctggtgcagtgctttttactgtgtaggcgctcattagtattaaactgaatgtgcctttaacttggcttggggtacgttgaattgaggatgagcgctcacacaatattaagaatttctttccaagaaatgtgctcgggtcaaacacaaggcctttctgtccatgatccgcctgaaaccatggtttccttgttttcgcctttatcccttctgccttgccagtcctccccgccagcggggccgagagcccggcgactcttgcgtgaccgagcgggctccgctgcctacggcagcccacccccagcccctgctctgaggcaccgagaaacgaggaggcccgtggcgagtctccacgtgggtaccggcgctctcggcgcccgtagccacccgcccgccggaagccgacatctcgagttctggcagaagcaatttgcgcggcgaggagcggagcggcaggaacccaataagctgcttcgcctcggagctgaagcccgtactcaagatggcggctccgggcgggcgtggccagtgactagaaggcgaggcgccgcgggaccatggcggcggcggcggacgagcggagtccagaggacggagaagacgaggaagaggaggtagtaagcgctacgccaaaagcctgcaccgcagtggcggtgatgcctgtgctggctgtgtgtggggaagggagttggggagctgcgggccggagggaggccccactgctcctcaccggcttcttgctcctgggcgcgaggagccgggcagcttgggaccttagccctaatcgtcaccccgtacttgggatgggaagtaccgggaagttaccttaggtagcgtagtttcctcttgctgaaaacagggtccggttttcacaaatgtgggaaatttggcttcttttgctaattgctccctcttggccgctcgcgtgctagcatgttaacccctctttctttcaccaggagcagttggttctggtggaattatcaggaattattgattcagacttcctctcaaaatgtgaaaataaatgcaaggttttggtgagttttc

SEQ ID NO: 55 - DNA, 807 bp, Artificial
  control peak
ttaaagcactttgcacacattacctcatggtattaccaccctcattcaacagatgaggaaagctagtttcaagcaagttcactgaggatcagagacgttaaatgatttgcctaaagttagagaactaataagtgagaaagctagtcctgctgcgttttactaacgtgccaatcagctgcccaggaagataatgaaaacttgagtaagacaggcagccaaagccacagcggggacgttcccagcctggctcgcttccggcactgacgcctccagccgcgacctctagacttcagcccttccattggtcgtccgtcacagtgccacagtgcgccatgctcacacgacggccagcagccaatcagcgcggctcatcttcgcccagccaggacacgcaggatcgcgttatctgtccctcccgcgggcacattgggagttgtagtctaattatatatttccgcccttagtgtggggagagcgggaactactcgacccacagagccgatcgcggagcggattctgcttttaggagtacccgccaacaagcgggaccgagcaggaatccgtatctgggaacaggtgagagaggatgtgtgctgggccttggaggaagggggccgagaccgggccttacttctgtaacgatactgtgaggcatcggaaggccagcctgttgtgtccgttttgaaggtcggtgggctagactggctggccttctaggggtgtggagacttcccaactctgcccttgtgctttcctggaatccccaatatgcccggaccccggtttactcctttgctgcgagccctt

SEQ ID NO: 56 - DNA, 1146 bp, Artificial
  control peak
ctcgctttctttggtaaacaaaaaccgctcgagccatcctggagaaccgcaagcggcagctcggtcccccagccggatttatgctccagtttacggtctgtggtttgcagtaagaaaacaccagggtcttcaaaataagaaaactgattcccagtcagagaataaaactccactaacaaagtctggcaaaccgcaggcaatacgtatttgctgaatattttgctttcaagctagcactttgagactcactactttggacggcactccccctccccacaaaacgacacgacactggctgacgtctctcccacccgccaggcagtgttaggccccgacaggcccgtgcaggtgggatcactgttatccccactagactgaggaggaagccggggacccgggcgattggccactcgggcgacctccacagtggagcaccaacagcgccgggaggcgcaaaccacggcaggcgaggaagcagttcaagctggtctccggccccgcgctcccgagggtcggcagtacttacccctcagacctgtgaggccgcggccgccgccgcccccgcggggccatggtgccgctctccgctctcgagcgactggaagctcccgccaagcccctcgccgctccgaacagccaatccgggccgagcacgcgcgcgtgcgcaggacttcctgagcggcccccgggctcctccctggcctggagcgtgagggaaaactgggtctggttattacagcgcaacggagctggagtccatgccgcacaaccagttacctacggtccaaaacatgccaaagttttccgggctttcgcgcccacccgcatcgccagttttccggcgccagaaagcccgggagccacgtgggacggccccttgtggggtggggaaaggggaggagctagtgtccctgagtcgtgggcaggtaagagggtgggtggagacccgtgttcgcagctgttgtcgctgaggccgcagtcgtctttccaggcctacaggatttggcttccagcccctgctgcctttatcagctgtctgagcttagcatgttactttaccgctatcccaataccttcatttgtaaatggacagggttccttttgttcttttattttgtttgtgtttaaaaatgtgtaatgcagaag

SEQ ID NO: 57 - DNA, 892 bp, Artificial
  control peak
agttctcttcagccctgacgccccttagaagtcccaaaatctatggaacaggaattactcgggaaatggtggtttcagtgtaaaagatgtcatctaccctttccctcactgcacagatggtgaaggggaaactcagagagataagtcacttccccaaggtcagacagggtaaaagtggaccggatctgtttggtcctgagcccaaacatttcgaatggacataaggctgacaaaggaacagtcggtcccagagcgtcaggagttgaggtgggatgggaccgcagggaaaatactgggtgggatgacttaaacttcccggcaccagggggcgccagacactctattttacctaaaggagactggcttctaccgacaggaaggtcccttgcccccaacaaagagggcgtccaatgacgtaaaccaatccgattgctctgtgccagggccgggattcgctggcgtagccagtcagtgctgccccacagccgcgtgcgtctgacgtcaaggcaggggcgggatccggttggaattttggcgggttcagctgtgaacgaagaaggcgtcccggcatcgggtgaggagcgcgggccccgggcgtgcgtgtgaccctcgggcggcggggccggggccgcctcgcacgtctctgcgcgggttgcaaggggtccgaggctagaggcgcggggtcccgggagctcgggtgggcgcgatctggatttggcggcagcttgggaccgctcggagctggcatggccgggccgggggctgggagttgctggccgtctactaaggaatcgggttcttccttttaaacgcagccttttaagtgcttgccaagtgcctgccgcctgggctttcttttattcattcattcattcattcattcattggtttc

SEQ ID NO: 58 - DNA, 898 bp, Artificial
  control peak
gagctatgatcacgtcgctgcactccagcctgggcgacagagcgagattctgtctctaaaaaagaaaacagtccatagggagtaggtggaattagagtagaaagagagagtcctggatgctccgaatggagcctttcagagctttaaccacgggactacatctcccagaattccgctcgccggatcccaccgcactgaagggactgcgcgtgcgcgagtcaggtgacgacccgcccctacaggccccagaacccccgagattccccgcgcttgcctcccgccctcttcttccagactctcggtctgtccgctgggggcgcgcgcggtgtgtggcaggcggcagcggcgctggcggccgagtgcgcttgtcacgcgtggcggtgcgtggttgctaggggcgcctgaggctgccgggtagcccagcaggccgagggaggaagtagcgtggagccggtgccgagccggggcgaagctggatcccctaggtgagtgcggctgcggagccaggggagccgaccccagacactccgccggtcccacgctcccgacctccctctacctgagtcctttcgcgcgggtccccagccctccggccactctcaccctagccccgcttcctcccgccacctctcatcccccgggaggccagggagccgcggcctcctacccggccgggacttcgcgcctcctcggagcccttctcctctccgccacgccccgcgaacttagtcggcctcgcctccgggtgcccgaacactgcctcatcccctagttatggcctccgagcttcctctgtcctgccgcgctgaggtttaaattccctctgggtgtgagcccaagctcctcgcaggctccagggccgaatttaacatggatatctgctttttcacttctccat

SEQ ID NO: 59 - DNA, 1079 bp, Artificial
  control peak
atacggaggcgagggtcgaacgccaggacgtccccgctccctccccaaacgtggcggggaagcgcgcggcgggtcccctgtgcaggagccccccgctgtccccgtttccggcccaggcgaggctcccggcctcagggccacccgcctgggccgcgctcgcacaacttcggcctgaggccccgggagccgccagtcctcagtgcctccggcaggacaaagaccggcatgcagcgtcaagacaccgtccgggccgcaggacagtgtatgaacaaagaggttcaagggcggcacccgcccgacgccagcgggcagctacctgactgcgcgacccgaggtccgccagcaccagagcagttgctgtggcaacaggccgcggcgtcccacgccgccggcgcctgcgcgccgcgtccgctgtccgcgcctgcgcgcctctctccccgcccaacccacgacgcgggcgctcctcactctcgcaacggtcacgcagaccccgcctctgcgaacccggccaagtcccgagcggaagcgccttgagcggaagcggaagtgaacgaggcggctgtggcggtggctgaggcggctgggcctagggtgcagcgggcgcgtctgcggctggtgttggcgcatctctaggtagggccgggcccgagggcacccgcgggtcttccgggccgctcccggcctgcgtgaccgtccgttacccgcgtccgaccctcgcgcgcgtcagcccctcggcttctgcccggtcccgatgccttcctggccgtgcctctgtccttgtcaccgcggctgcgacatcatgggatcctcgcgtccccgctgccgagcccgtcgtcatccctgtccctgcgcagaaccctcctgccgagccctggtccgcgtcattgcgtgctccccgacctcgcgaccgtcgccttgcctgccccatcttgaggtcacctctgacccctactccctcccttaagatgtgtgtttggctcctatatgtcatttttgggtcagagctgttatccttcgtagctgcacgacttttctggtcgtctctagaagagagaggcatcctctgatctcttcacttt

SEQ ID NO: 60 - DNA, 864 bp, Artificial
  control peak
cctgctgcgccccagtgggaacgagcttggcggcctcggcttagagggggcgcgaggctcgggcccaacacagcccacctgacacaggcgtgggagaaggcgagagcgcctctgaaccaaaggcagtcatggaggtctggtgccttgtccacccccagcccgcagcccaaggggcgccgatccccccgagcaggcctgcctctgccgccagggtgccctcacctgagcctcctgcctttcggcccgctcagcttccgcgagctcatggccgccgtcgtccccgcgcggtggtccggcaggttcgcgtctcccggccagcgggtcccggccagagcagccctgcgggcggccggtctcacgcgcctagctgtcgcggactgtttttcttccggactccgaccgtccgccccggcttccgtcagagacgcgccctccgcgcaccccgggatctgggtggtgcggttcgcagaaggaggcgggactctcccacgagcgcaaagctgcgagaggctgagcgaagacgggttcgcggacgtaagaggggcgtgtcgggcctgtcatcagcgcacgcgcaacactgccccagacgacgcgaggtgtgcggccgccactagacccggcccagcccgcgcccgccgtccgcagctcccagcgtgccccgcgccaggggccgtcttccggcggaagttgtctcttcgccgccgccgcagcctctgccccactcgtgagggatccgagggctccgtccttgtgctgtcttcgccccggctcccaggggtcaaacgagggtcgggggtggggacacgcagcctagccactgtcctgcctgggatccggtctcccccgccccccggctgcaggtaa

SEQ ID NO: 61 - DNA, 1109 bp, Artificial
  control peak
agtctactgtagtactcggtacatagtagttgctcaacgaatatttgttaaataaatgagtcttaagtctggaagaacttgcaaagttctttaccaggttgctggtaaagcaacctgacctttctttcagggaactgtcagcaacacaagccatgaataccaacgcaaaacagactctcactgtagatcacacgggaaaaaataaaataaaaaggtaaaatccttaacggtggtagtatttaaatgaccatatactgcttagtaagaaatcctggggtcattttcaaggtcccccatttcctacttttgttaagctgggtcaagtctctttaaactttctccgttttcttgtccataaaaagagtatactaggtaagtcttctgtctacctcgggacttctgtaagtatcataagagatgttttatatgacaacacttcacaaacgtttagtaaaacgctgcaaaatgtttgtagctggctataaacaagatcatgcagattgggttcctgagccaacaaacccaaagaagaccggttgctaggaagagaaagccgagcgttgctccgtgacgtcatcactccgcgccacccgcgacagtttcccagcagggctcacagcagcgttccgcgtcatggggatttggcagcgtctgctgctttttggtggggtgtcgctccgggctggtggcggggccactgccccgcttgggggaagccgagcgatggtttgtgggcgccaggtccgtggtgctgcagggcagggggaagcgggctgacagatgctgggctgcagggccgcagatgccctggcattgcttcttgccaaagactcgcctcagttcctgagacccccagtgtgctggacgtgcctacttttcaggcaggcccaggctgaggcgtggcgggacttgaaacacagttagaacgtagatgtggtattctacattgctttcttggctttgggttatttatcaaatagattgaagaaaagcaaaacacgtggaagccttatctccgtaaagaggcagcgtttttacatttttaaacgttcagcgtatcctgctagaaaaatttcaatttaagcaatgtgtctttacaattgttatca

SEQ ID NO: 62 - DNA, 1091 bp, Artificial
  control peak
aaatcccgtgctaggggataaaaatattacttgagccacctagaactaaatgatggcaccttaagaggtgcttcgggaggaaccggagcccctcgctgggagctgtcattccttggtccctctccagctccagaggaacctagtcaggaagcctcgcagggcggagccatccgaattgtaaacaatggcggcctagcctcttccgttccccgaggaacccggagggcgagccgcctaagctggacctgagggtatcgcggatttgcgtctggggccccggagaccctctgtagcccagggcctgctgacatcccgagagcctctggagccaaggcccgtgcagctctggtctcgaggcctttccccgcaaaccgcaggtccggcgaggactcgggaccccgaactcacccagctggcgagggagaagacacccagcacagcccccatggtgacgccagtgatggaggtggccggtcctgaggctgctttctaacacggtggtaactgccagctgaggtgactccccagaaacatgacggtttctcaggccggaaacgcagccttccacagacgcacggatgcgtcacggcgtggccccgcccaccgtccgcgaccggagcggcagaggggggcgcctgtcattggctgagggggaaggtggggctaaaccatcctggggttgcaggaactgtgaaggtgtcattccttacatcatttgtattcattgtgcaaagatctggaagttacagaagtgtacagagaggcacagtacgctcagaatttattctctagttaataggagatctgacaacataaatgcaccgtgccccaatgtaatgaatacgatgaagtagtatgggggaataggctttcaataaaggtgtatttagtgcctcctattgtaaggggaaagtaatttagctgggcagtagtcttggaaggcttctctgaaggatttattttttaatctgcgaactaaaggatgagtccaaattaacctagaaacgatggaaatcgtatcctaaaatgtgggatgcaccccatttgggaaaaatgaatggctctaagggtgcatgaaggacattaagcga

SEQ ID NO: 63 - DNA, 624 bp, Artificial
  control peak
tcctactctgggctcccccggatccccactttcagccctattgacatccatctctacccccagcactccccaaaactgaccgtccaccagcgtcccctccaatttccaaattcaacatccccatccccctcccagccatttccgccccacattacagacgcagagtccgcaggccaatgggagctcccagaggcttcaagggcccgccttccacccaatcggcgctctcactcagtattcgcccgcccccttcatgctcgcctaaggataggccgcgcctcctggcctctgagccattggcaggagggcgtgggcggggcatcgtcgtctggcactggccactcagaacttaggaactgaacctggccattggcggagtggaccccgatgggtgggacgggggcggcagccgtgacagccgcgggaagacatgagaggctgctagaaggacttcccgcggccagcgccatcacctgcccacgccgcagggaggtgagactgaaggaagcacttcctttcccgtcttctcccagcaagggaaacccccaatgacccgacacagagaggccatagaaacgtctttattgaagaggacaggagtcatgctgagtttggggacatgca

SEQ ID NO: 64 - DNA, 887 bp, Artificial
  control peak
acctccctgaaacagaaaacgaaggggtcgggtataatgatttctaaggtccctcgtagtgaaccctaaaagttatcacaccccacggtggggctatcgcggcttgagggcagccacagcctcagccggggtttctgtccgggccctgatgggtccccagcagggagaaacagcgtccggcgcgctgtggcgctcggcgaacgtgcgctgagttcaggcgttacaacggggtcgcacgcggagtccaggcctggcaggggtgccaggacgccgccggccatctggggcccgacctcccaaggtctcctccccgcggccggcggctagcgcgctcacggcgcccgggccttgcgcgcccgccgggggcgaggtcagccctcccccctcggctattggctccagagaacaaaggagccgccccgcgggctgctccgatagggccgggcgggtcatctgagcggcgcagccaatggagtggctgggcggccccggggatggccaggcccgccagccaatcaggcacccgtgcccgccccgcctgccccgggaatggccagggccgggagcccggtgcccaagtcgccctcggggtggcagttcccgttaaccttagccacaaagtcaaaggtatttatttccccgtcgcctccctccgagtccctctgcattttgggcggggaggggcggtgcgagggatggaccagcgcccggccccgctcctcccgtgctgcctccgcccggcccgggctggggcggagggatggggcgggggcctggggtgccccgagccccgcgcgccccccgagccccgggtctcaagcgttccctctgccccgacacagccgaggggaaaggggcgcggccgagggtggggagggcgccaggcc

SEQ ID NO: 65 - DNA, 1455 bp, Artificial
  control peak
tccaagagttttcaatctaattggaagggagtattacaattttagattataataattataaaaacaaacattataggagctcagagggtttagagatctgtgttctgtagttattacaggaggcctctagattctaacttgggttagggcttgaaactacattgaatttgaatattcccagtttaaacatgcacgcaagtattatgtccaaagtagcatgaatactgacaacaccaaacatgaaacccagatttatttattctctatttagtcctttccctactctttagagcagaaagagaaaatccttgcttgagatagcaaattagtgctacactttttgggaaggccactggaatttaaacctggcaaaattccaatttttgtgcttgtaacctgtttagccggcagccagtgaaagctgttataattctttttgaattgaattttctttcgtattgttttaggtacagctcaagctcaaatgatagcgtctctttttctttaaaaaaaaaaaaaagtgctgtcaagacaggaaaactggaatgtaaccagattagccgtttcccgtatacatttgcccttaagcaagatggtgggcgccttgtgcacatgcgcggaagggttgtttcggcggggagttccgccgcgtcggaactcgctccgggcgtggattggcggcttgctggcgggggcggaaccggaagttccgtgttgtggggctcagctggttcatttatgttgtttttcctgtacctaagttcccgctgtaggtggtacctttgcagacggtgcgatgggggaagcagagaagtttcactacatctatagttgtgacctggatatcaacgtccagcttaagatgtaagagaacactcgggacagggagtgggattgctggggcgtagggacgtgggggcaggggcctgttgggagtgagaggcagaaagtacgtgactcgtctcaaggcccagattgggggcggctgtgagggctgtagccagggaggcgcgagagagtggatcgtggagaggggctcttttggaaaaggatttaatacgtggtctccgtgagaagcacagcagtaggaatagcccttaagttccgctgtattttacagtctttaaagtttttgctgttcagccttttgagagtttattactatttcattgtaaattaagaaatcaagtctcgtcgtaagtaagaggacgatctttattttggaacggggcttgtagggtggaatgaaggcttcagaaattatggagaggtagacacaggcagatgcgatagttgagaataagatgagcacatggtgtattagcacgggaggtgatagaggcctagagtgaatctttgggtagtatggcagtgtttagtcagggaagttgggagtaagtcgaagatgagggttccagtttcaaaggtaccaagaagataggagaaaagagtctt

SEQ ID NO: 66 - DNA, 879 bp, Artificial
  control peak
gccaggcagactcacctgactcctggactccttcttctggacgatcccttaccccctgcccagcctcccaggtcccttctcactcctctcctccccacagcctgggacaggccccatactatactggagcttcggagatttgctctttccctctagcacactcaccccgctcccaaattggctccgcggaggtggccgctgctccgggcccttccgtcccgtcccgtcccggtccctccccttggttccccggtcctccctgccacgggcccccaacccagacaaagggtggctcaggctgagccctgctctgctaggctcttcgctgttcagcttcgtgtgcaacactagcgggccctggcgcccgcagccccccagccgctgccgcgcccctccctgcgacggtccctccccggggccgccccggctccgcgccgccgactccactgcccctctgactcacaggccatttcctaccagctgatggggccgcccgccccgcccagtgccccaccacccgcgccgcggcggccacggccgccacgcccccctcaggctacgccctccctggaaagcgaccatgctcattggtccataggaagccagcctccccgcgacctcaagtgcgaggcacgccccctccaagaggcacacgcctattggtccataagggggtaggcttacccaaggtcgctccgcccttctccttaacccgacagctgccggagtccaaagcaacaccctgccctgccatttaaccctttcaaaggcgtaggccttccacatcttacccctccgccctactcccccgcccttcctccttccaggacggttaataggaagcccgagggctttaaagatacggtttgacaaccctca

SEQ ID NO: 67 - DNA, 723 bp, Artificial
  control peak
atactccagcttggattactgaggctggggcccaggccctgcaggaaaggaggtgcatgactactttaaggccactcactctgtgactcaacgggccgggtcggggctggaactcaatgccctcccgggcctggagagcccacgcgccgtgggcggggctcccggggtcgcctaggcaacaggcgcgcgccgcgcccgagcccagagccccaaagcggaggagggaacgcgcgctattagatatctcgcggtgctggggccacttcccctagcaccgcccccggctcctccccgcggaagtgcttgtcgaaattctcgatcgctgattggtccttctgcttcaggggcggagcccctggcaggcgtgatgcgtagttccggctgccggttgacatgaagaagcagcagcggctagggcggcggtagctgcaggggtcggggattgcagcgggcctcggggctaagagcgcgacgcggcctagagcggcagacggcgcagtgggccgagaaggaggcgcagcagccgccctggcccgtcatggagatggaaaaggagttcgagcagatcgacaagtccgggagctgggcggccatttaccaggtgcgggagcgccccggagcgtggcgggcccttcgcttaggccgcttgaacatcccctcagacctccaggccccagactccctctgggtcttgccctctgcctcgctcctactgcttgagga

SEQ ID NO: 68 - DNA, 819 bp, Artificial
  control peak
cctcacgacactgtccccacgccccggtccccacgccccctcccctcaccagccccgccctcaggccccgccctctccaagaaccccacgagacagtcagttagttctctaggaaccgcgcgctggcggtcactgcccccgcctcccattcacagaggccgcgggagggcaggtggggcctgacagtctgctcgagccagtggcctcacctctcttccaagagctcagtggggacaccacctccacccgctcggaaatgaactcggccagactggagcggaacaaggccgcccgcactgtcacagccaccaccagcaccaggaccaagggagccgccatgataactggggcgggcgcgcgggcggggagggaaggcgaggcggaggagcctcgcgcgctgtgcgccacgctctgtagttccgcgggtgggcgcgcgctgccgctggcgccggagaggccagacgtggggactacggctcccagaaagccttgcggcgaggcgggccggagcgacggaaagcgggcgcctcaagctgcgtatgcagaaggtggggcgaaaccttctacgatcgcaggtgttttgtgaagccgcgccgctatagcggggctgaaacgctgctgaaagtagaagcggaagtagtcgccttcctgggattgtgagagacttttaaacagatgttaaattcactcttgcgtgtatgttttcaatgccaaaatgtttatttttatttttatttttattttttttgagacggagtctcgctctgtcgtccggctggagtgcagtggcgcgatcttggctcactgcaacttccgcct

SEQ ID NO: 69 - DNA, 814 bp, Artificial
  control peak
ggcgccccttccctcacacgcccctctcacctggcctcgaggaggcgatcgcgcccagcccgcgccgccgcctgcaggcgcccccaaggtccccccagcctgcaccccccgcccctccggtctctgcgccccgaatgctccaccgccccgagccgctgcccctgggcccgctccctggcccccaccccatccccaaccccggcctccacccagcccccgcacctgggggccaggcgactccgcggctgctggggctgccgggctcctctgaccatccggctcctgctccgccgcgggagctgctccggcggccgcagggctcgctcgggaagctgaggcggcggaggctggagtaggcggagaggcgggaggagggcctcgcgcctctgccaggcttgccgcggggcccgcctcctccgcgcctctcctcggctcccattggctagagcagtagtgtggacggaaatctgtcagtcgattggacgctcggcccgtcacatcccgcgctgcccacccagaggtttggttgagactgcaggtggcggcggaggtgccttagagattgctagcagggccggaggagaccctagcgccaccatgcgtctaaaagtaggaactgcacccccggctccagacacctgccccaggttctcctggctctgcgtcctgcataccacacacctgctggcgcccctccacctctgcaccacgccaggaccgaaaacaagccctgaaatctcccaatttcttctagcaggtgagacagtgaggcaactctcacaaacgtgtttccttttaagccacctcgaatgcg

SEQ ID NO: 70 - DNA, 684 bp, Artificial
  control peak
tacatgtttttatgaggaaaaaaaagcagcagagatcagaagactgccaccgtgcttggagaagaaagaaagccgcgcgtggccttcgcagtgctcgccgggactcgtagttccaggtctgcgggctcctcccaggcggggatctacttcctctctcggccgcggccgcagcacccggaagctgagggggcttaaagtctttcgcttcctttccccgcctcttagtcccgcagattgctagtggatatggccaataggagggctgactggtacttcctgttatttgattggcttaaaattttgcaaccgcccggacaaacttgccgaggattggctgagccttcagttttgaaaatcatagttttcgcccgcgctgcgccgcctggttgctcctcccgtgcggtgagtcccgaaggcccggagccaagtgggtctgggcaagcggggccttacctagggataccaccggacttggatcggagcagccttcaggggacgtggcgcagtcgagtgagtgcgcgtgggcgtggggattggggggtggttcgcgattttttccccggtcctctgctccgtccctgtcgactccggaccagggtcgtgactccccaccccagcccctggctacctgggcatatgagggtcacctcttccaagtggcgaggtcgcctgccttggttctcg

SEQ ID NO: 71 - DNA, 708 bp, Artificial
  control peak
cattcctccctgggtcgcgttcccacggcagcgcgcctcttctggctacaaagctgctgcgcgtccgagctctgcagggcggagactcacctacacagcggtatccgaggatgaccaccttcctgtagcggactagcggcatggcaggagcccgccccaggggcttgcggaactgcgggctcagagagcccgaaaacgaggtcagggtgtgagcaggcgcggcagctggtgcaggaaagtcgctcaccccgaagctgccgtgggcaagttagaaggaaaccaaaacaagcgccgcgcccggagctgcccacgtgatcacaacacagcacgtctaacgccagggagccgggcgcccggggaactactgagccgcgccgcgccgggctccgccccccaggccgctcgccattggtccattagaatgggtaagggcggtgccgccagcaggagggaagggtgggcctcacgtggagcgggagaactgggagctgcgcgtgcgcagactggagccggttcattcataaagaaacagaaaggaacccggggtcctaaaggcgtttgcgcatttacagcctagctgaagtggcttcgaagaattatggatggaaaatacccgttttgtggtttccggtcataagattctagccctcccttttgcatggtctctggggtccttagatagggagaaaaccgccaaagttctagtat

SEQ ID NO: 72 - DNA, 565 bp, Artificial
  control peak
ccgcagcacccggattccgagaacagaggcgtcggggccaaatgggctgaatctggtacctcactcccacgcccccgggtggacagcgaccctcctcggccgcgtcccctcgtgggtttcccccaaccaataattcgtcccgcctcccagctgagcccgaagccaatcgggtcgtagatctgcacctttctatcccgccctcctgggtattgactaatcgtagaaacccccctgggatcccgccccctccacgggcttttatccaattacgccgcggctactaagcgcagctgaagcgtgaaggcgccaatgaggccttcggctactccggcaggggcggaactaaaatgccggccacacccctctctgtgactcagtctctgagcgttttaatacgatggtgtccccgcgggatcaaacttcagcgtcacagctgaggactggcttcgtggtccctgatgggagagcatgaacaggtggtatgtggtaggtggggttctggagcctggcgggtgtagatttcacaacccagggggcggagccaggatgatgaccccgccccctc

SEQ ID NO: 73 - DNA, 968 bp, Artificial
  control peak
gtaggaaccgtctggaaacggaggaccgtgcccctatccccggaacattcgaggtataggaccaaaggccttccctggctcgggcgtgcgtgtctcgtgacaggtcgggctgctagaggggcctttttaccccttggatggcctctggccccttggggccgggatacgagacctaggccagccgcacccagaggtggccgctcccttccttccctagacctcttctgggcctttcccaggccagcgcctgggccctgacgccgaggcctccgggatttcccaattcctcgccctcggcctccccacggctcccaacaacgcagcaccccacccgagagccaggccttggtcgccgcaactgcagccctcaccatcctgtcaccgggctccgctcagtcaccaccaccgtcgccgccctcttcctcaggctcctcggcgacccgcccacttaaccgcaaccaatgagagcagagggagacagaaattgccgtacggaggcccgcacggacccttctccgccgtaaagccgtttgggaacttgtggaggcggggtggtagagtgcagagacgagatcgcgaagctttgaaaagcgcgggcaacatccgggcacctgggccgtcgagctgaggcgcgccttccgagcctgctttttagggcggatggcagccatgctgaagtgcgtgatgagcggcagtcaggtgaaaggtggagcggcctttgttgtcttcccatttagcagagagaaaagcagacgttaataggtcgtccctaccattgtctaatttttcctctttgcctttttgcgcaatgactgaggacgcacgccctggccacagccccacccactcaagtccctgttaacttctgaggggaggatgaggacccatctcgtgtgacttagaggcagatgtaatatgggtggtatccgggaaatagagttgtaccatcgggccacaaactcgggatg

SEQ ID NO: 74 - DNA, 1209 bp, Artificial
  control peak
attcagggagttacatcggccagcagtatagagtctcagatatcctactcctgggatctaaagtcgaactccccacccccaacacctgacggggcccacactcaaaagtgtcaaaactcatagctatgcacattcacaagactgtagaagtcgggctgtcacctacgctactccactctcagattctacatccccagaagacaaagagttacagttccttctctcacgaattccacaactccgaaaaccgcgtaattgaagccaccaaaggcatagaagcggcagggaaagggggctgggaagaggcgaacagaccctcttaagccccagtcattgggaaaagcccatgctgctggaaaccgcgttctcacctccttctttctttgggcagaggctgagggaacgtcgcaaacgatgagcagcgccaccaccatggtcacagagacacaccaaaaccgccaacgcgctgccatgttcgctcctctcccttctataagtgaaactttgctccggctaggtctgagggtggggcgtgagaacaggcaaatcggccccttgcctttcctcattggtccagctcagccctgccgggagacccctcacattacgtcacagcgcgctggcgctacacgcccgctaaaagaaggttgatttctaattggccccggtcggccaatagaaaaaagataatctggagcgttttcattggtccgctaaagataatgacccgcccctttctcgatacgtaaacatctagcgcgctgacgctttccaagcacgcatgcgcagttgtgaaatattgcggagttttcggagtccaaaatggcttgaagttgctagaacagaatctttcaaagactcatttagaagtccttgaaattcgccgccttcatgtaatattctagaaaaatgggacctagccgcgtgagggataagagctatttagtccgttatcctgcctccaaagtacttcaaagtgaaaaaataaaagtaggaaagcaaaaatatacctaataccactgagggaaatttttaaaggtaaatatgaacaagagcttgttgaatacctattgtgtcctagaaactgccaggagcgctgaaaaaagaggacatcaaagattattttttaaagtttttgtttatttatggctttttttgagatggaatttcgctcttgtagcccaggctggcgtgcagtggcgccatctc

SEQ ID NO: 75 - DNA, 1270 bp, Artificial
  control peak
cactcttaatcctgtttacacacagaaaatcacttttattcccgaaaactcttagaaaaaccctgacaataacataacccacaacctagaagggaggcagccatgaagcaggcgcatccctttggaaccatacaaaaagtgacctctgccaacgggaacagttcagggtgcttaagtggccaaagaagataccgacatggcccctaaagtattaaaagcaccttctttccagaagtacctccgaattgccagggacttcgcccaaaactagaggttggtcgaagtgaaaatctaaacctaaggagaaggcagagagtgggatcccgtcacagtaggaaggtaaagaagacagaagtaaaggacagagcgggagagaaaggacaaaagttcaaaatacaggggtagaggagggacaggagccagaagaatggagcgatctgaaaggtgtacgtcctactcactggtaggcctgttttggactccgctgtgggagagggcggctgcacttctgcgtcccccgcctcacctgtcagacatccacacagccaggcctccccgccctcaccaagcccacacaaaccgcgccaccgccagcgtcagccggacgactctgggggcgtaggccgacaccgccctccctccagccaatctaatagcgctctcatgggcgcattcctgatgacgtttaggggtagggttagatgcccgtttccgctttcttcccacccacttccgggtctcctctgtttacgaatgtttgggcgcgaattactctaagcgcggctctcagaagggtgcaagaagaatcagtggtgagtcgtgatatccttaaccggctgggtggatctaaagttcagggatgagttttaaagccaacgcgtttgtggcagactccgcagtttgcatttgaggatagcgaggggggattgcttacggggaaatgcccgatgtcctaaaagatctgcccttaggatcctgagattgagatgtttgattaggggggagggagcaaagccatgaatacttcctatttctcccgaatcatcctcatggctcatcctcattgctaaatttctcccgcgggcactagtgaagtcaagtgcagccttggacattagtgtctgtgtttctccaggagtcctactcactcttgtgcccacatagaggtctaaattgaggtaattgagtcccatgagtatcaacaagctggtaagaaaaacaagacaacctagaatgcctccctctctcctaaagcatatagtctggcggc

SEQ ID NO: 76 - DNA, 704 bp, Artificial
  control peak
taaattaaatgtatacatggtttgggagatttactcattaagcaaacactttggtaagaataaattgagcgcttactgaatggcagtctcagttcctgcggccctcccgcttattacctcatttgatttaaaaacaaaaacaaaacgttatgaaagccggcttcgaggcattcctctgaagggaagggactccccgacccagcgcctcccccgtcacgtgaccctgtgacccggagcagccatcttaggcatcgcctcatagaggaactacatcttccagaatcctcgcgccctgcgtcgtgctggcacttttaccggccgggccttagagcggagcctgtagccaggcgccatctttgacgctggcagtcttggttttctgctagtgctgctgctgctgggaggacgacggacggcagcggccaagcaagaagaaagacgtggcagcaagcgggagtcggggatagtgtcatcggttcggtaaagtttctgttctctggtaactagacccgtttttttgattcccgggtggcttggggccggggaggtgggggagagggcgaggggagtggcagcggcctcccctcgcccctgggtcgcggcctatcccgggaagtcagagcggatccttcagcttcgccgtaacgacccctcccccccacctcccggtcgcggccctcgccggggctggctgggagccccag

SEQ ID NO: 77 - DNA, 1253 bp, Artificial
  control peak
cccccttcatccgcagccgggagcctttccccaccacacggactccttccagtgagttcccgccaaatccaccctcggtccctcattccgcgaccctcgaagcgtcagggcctcggccccaagggcgcacgggcgggcctcctctgccctaagcctgcggcctggggtgcggccgtggcgccgaaagacccgcatgctcggcgacccagctttccaggacgaagaaaggacagaagtgtcgtccgacagcggtgcgccagaaacccgggtccgaggcggcggcagcagccgagcttcccgcctgacgcagcagaggcgcaggccaaagtgcggccgccatcgccacctcgccgctccctaccctctctgtccgccacagacccgacccgcactgcggctccgtcagcgccgggcccctcccccagccctcaccttccgcgtcggagcgggcccgcagcggcggagactcggacgcaggcctggggaagacggctcgcaaggtagaggctgtttattttgagggtccttgcctgcgccacacgcgggtctgcacacggccccacatacgccgctgcgcacggctctcctccgcctcgcccacgcggccaatcagcgcgcggggttcggcagtccaatcagagccgcccgcggcagggccgcccactagagcgcggcgcggggcgggtgtttctgcgcgcagcgtcttttcccgccgtgccccgcgcgcggcctttcacgccgggatccgggtcccggtgcttctcggttcattcccagctgggtccgggcccggccagactgggtggaaatctggctgaccttggtggtctgtcttggccagctggagcaaagggatgccaggggacgggtctcggtgcagtgactgccattagcttggggccgcagaaagtttctcccctttctcgggctcctgccaggcccttggctttcagagccaggcccgatttctagataacatttaagtttgcagaaaaggggatgccaagtcacggtggtcacggctgtgggatgcagcctctgagatttgtattaatttttgtgagccggggaaaggccttgctgctttctccgccaggactgatgtgtcttcccaagatgaaagtgcctggtaagggcgaggggcctccctgggaaggtcatgagcatgtaaggtaatggggcctagactgtgacttttggcacgtttatgagtgacatcccccgggagggcaaggataaccatttgacctggt

SEQ ID NO: 78 - DNA, 956 bp, Artificial
  control peak
agatctctctcagttggggaggcttgccccaccctttattattatgcctaacacataagaaatgcgcaggaatcaaacgcaaactggtgaaccaactagagggccgcgggggcagaagtgtccggcggggatccggacagcgcatccggaagcgccatgggccaacgctcgcaggaacctgaacccctgggccagcgccgcctttgctcgcagaggcctctagcatctgtactgtcgaagccctctgagcttcagggcagactgaagagccttacacaccagacacttggacgcgatcgcgccagaaatccccgggaattcagtcttggctccgactccaaaacaaatgattcctcaccagccagatctccatcattggcgttagtctctggtcacctgacttgttcaagccaatgagagggggccgaattagtacttccgctttccgtgctgtttaacgagccactatgcccgtccctcggatactctgattggccagcctctctcagctcgtcagtttattggccagcgccgctaggccgctatcgagcagggtccgagggctgtgtcagaaggctgggcagccatggcgtcctatttcgatgaacacgactgcgagccgtcggaccctgagcaggagacgcgaaccaacatgctgctggagctcgcaaggtgggtgcgcggggcttggggatagggtccaaccagcaggtttctgggctggggctggctggagaaggcggattaccaggggcagcggattctggggaggacagatgagtaatctggagtgttagtacgggcggcgagactgaggggtgtggggatgagcatctcgataatttgggccgtccgttgggccggcctggggcaaggggattgcgggaaagattggagctggagagtggtgggcgtgggggcggaaggacagcaggctctgggaggtctcagagaag

SEQ ID NO: 79 - DNA, 637 bp, Artificial
  control peak
aaacacgctcttttaaaaattaaatcttgtgctactgtttcggggttgtaacgcgtgcgtgaccctcgtctttgagggccggatcctgcagggaagagagggcgtacctgaggggcctcgcgtgcctggacagccccgcgggccagcaagcctaaaagcaactcatccggcgtccacgctgcaaccgggcgcacggaggtgatgcaacgcgctgaaaatgcgggatccagtgaaggagacaggctccgcaccggccaatcagtgcgcgctgcctgacacctgccaacaagccaggcgccgattggtcgcgccggacatgattgcgcccactggctcggatggtgcgacagtggtgcttgctggttttgcgcctccacgtggaaaacgaaccagcgctgagggcggcgctgtgcgtgctgcttgtatccgttgcagtagtgtagaccgaggtctcactcgggttgtgggcacctccgggtaggtgacggcccactgggaaggggcgccgaggtcggggctcatgttcttcatcttgtttgcttttgtcagtatttatctgactggacacttcgtacctaaattgataacgacaaacagcaacttccggagaggtggctttaataattatacttttggg

SEQ ID NO: 80 - DNA, 1325 bp, Artificial
  control peak
aatgtcaggggtagagatccaaaaaccaactatgatctctcgaactagactctagaccaatgctctcactttaaaccacggaagcggcggcaggaccagagctgagtacagctcttcccagacaggactccacaacagcacacagagccaaccccgaagggatcgggcagcaggacatcatctttggctgcagcacagaagacgccggggcaggacgctaagccctggagccccgcgggccggccacccagctcgccgggagtccgcggccctgggtctgccttccgccagaggccgagttggtgggatgtcggcggccagaccccggccctgagccaggggaccacttaccgcccgccccgcgacggccgcccggggagacacgccggctcgcccacgcccgctgcccccaagagtcgcaaaggtgtggccgccccgttcgcccgtaccgaccggtgggaagaaactcacctacccaaccgacacctaaaatgccagggacacgtagcactggagcttgctgatggaggcccaccggaaccggaatccgggtttctgccaacagcccgcccactcctccgtcctattggttccttacgcgaaacctcagtgcgcaagcgcagcgcgacgtgcgcccggccacgcttcctggaagagatgcattgcggagctcgctagttggggcgttggccttccttaggagtgtgagtctagcgttatcccttttttccggctggatcccgggttaccccaccagcccgacctcaacccactcacgtccacagcacgagccccagcggccttgcgcggtgcttgctctctagtagcggctgactaggtaaatcctaagttgtggttttttttcctgcggtccagcttgatgtgtgtccttacggtacatactttttgtatgtatataatgttggtcgaaatgaagcttatgagcaatataatgttatggcctaattcaaaatagtatccccaccccaaactttgactttggctgtcattcacggtaatgaatgccgctgtaattgaggcctttgtcaaacgctaggtttggttacgggagtggtcataatgtacacacagctccccacttacccacaaggatgcattccaggaaccccccgccggatggctgaaaccgtcagtagagacggggtttcaccatattggccaggctggtcttgaactcctgacctcgcgatccacccgcctcggcctcccaaagtgctgggattacagacgtgagccaccgcgcccggccatgtttccttttagcaatggagcataacgggatctgaggaacaatataactcagg

SEQ ID NO: 81 - DNA, 876 bp, Artificial
  control peak
ccggtgttgctacaggtgcgcaggatgtcggcagagccagaggcccacgctagcaaggctaggaaagacaggccaggtcttattccatctctagggaaagacggaagagccctaagtaaagcttatctgtcccaggcggagcaggggaagaagagagcaggtggggagccagttgggaggcgggaggtggaggttgggacgcggagagactggcttgggcttggcgatgggaggacccggacccgccgtcctcaggcggcgggcggcaggtggcgggaggcgggaggcggggaccgggccagcggaggtggggagcctggaagctggccctcgcgaggcggcgcggggagggtgggtgaagagcgcctgtgcgcgctaggcggagcctcacccgccgccctcctctgcggcaccgccccttccatggcagggctgaccccgcctcctccatccgaccccgcctcctccagccctccccgccccactacccaggaaggcggagctgggtgcgagcgccctaccgctttcgctttcccttcgcggtgcccactccactccttgtgcggcgctaggccccccgtcccggtcatggccatgctcagggtccagcccgaggcccaagccaaggtgagcgccgcggggtctagaaagggcccactggggaggcgtggctggagcggccgggggcatccccgacccgccccccaggctcccacgcgagtcgggggcagtcggccgagctggcgcgcccggagcacctgcgccccggggagggcggcgactgctgcctgcggggagaggcgaggcggccgtggttctgcggggtgaggtgaagcggagagctggcgtggaggggaactccgctggcctggggcc

SEQ ID NO: 82 - DNA, 646 bp, Artificial
  control peak
agtggagacggggtttcgccaaggcggccaggctggtctcgaacttcggacttcaggtgatccgtccgactcggcgtcccagaattctgagattacaggcgtgagccaccgtgcccggccttgatttgaatataaaggttccaacactcccaaacacaaggtgaccccatttacaactggatcttcaataagcgtctggtgattcaatttccgccaattagaaagagcccaaccagaacacgcaggcgccttaacgctgagggcataacgaaggtaaagaaggcgtggctactgcccaaatccaagatggctaccaggaggccgtcagttctcggaagtgcggcacatcctcactcctgggaggctcaaaggctcaaacgccattggcagaatccccgacagctcatcctccgccccctgcagggggcggtaatgacagagtcctaatttccggttccggcagtaccggtagcccagtcgagatggaggaagatgcgaccggcagacggcattcgctgggaacgacggatagactgggggctgcggcctagaggtccgggcttggagttcgcctcagacgcggtggagccgccggagtctgagaaggtaagaagccgccggcttgaggtccctgacaactaaggtc

SEQ ID NO: 83 - DNA, 748 bp, Artificial
  control peak
ggacagtcatctgcagggcagggagtgtgagcctcgctccggggccgcccccactccttgggctcgggttcccggacccacagccttccaaccaggtggggaccccacccacggactcaccggaactgctccgagtgcccgtcaagagttacttccggtttccacggagctccgccccttaggggggttctcgctcggccgcggctgggcattaactccgctccagtgctgcttgtcagactccgcctccctccctcagccaatctgtggccaccggagccgcgcacgcgcggccaggcgctcgggatacagaagtgatgtgagtgtctgcgagcgtggtctcgcgggcgggtgacgtcatcgaggcgagagaggcggagccgtggacagtgggcgggaggctgccaacggttttgagcgtagggggaggcgtgagagggggatctcaggggaggaggtcaatcgcttgccccccactttggcaaattggggactgaggactggaagggtggagagtaggcggaaccaggtggtcgtcggggcagaggatctcgggctaggcttgagggcggcgtgcttcttagggacgacttagggcgtgactgagggttcacaaggtttcttttggggtggtcgggagggagagattctagggaacaaggaagctcgctatggctttcttgccaggaggggtcgaagggaaagtacaagggagctggagctgaccctgggtagaacgggtgaaggg

SEQ ID NO: 84 - DNA, 728 bp, Artificial
  control peak
gccgggtcccgaacctcagatcggcagggtcagcacagttgcaagttctgcccagattctgagaggtgtagagcaccggaatgtggcaagggtctccccccggaagttgggaaaaggcctgagaagagatcgacccccctccaacaatccgccggtatctctcagccacgccctccagcgcatacttctcagtcactcacccgccgctcggcgagaggaaatcggtgtcgtcctcgtcccccgcaccgaacattgcgttggctttcaccgggttgcggggaggaagctgggtattcccagagtcccagggcctgtcaccgccgacgtcgtaaaatgccgcggcggcgtgctgctttccggcagggcgaactccagcacggtctctggaccgaaagtagactgggctctggagaaggtggggcaaaggcaaatgagaggctcgcgcgggcacgagcgccgagtgacgtcaggcctctccagcccgcagcggggcggagcttcacagccccttgcagggattggtcggtaatgctagggggcgggagggggcgggaaatcctcggcctcggtggcggtggtggacacgtcgagccgggtagaagtggaggggccgttcgaagagtcgtgagggggtgacgggttaagattcggagagagaggtgctagtggctggacttgacctggaaaggtaaggcttctctcggcccctctttcgcacccctgtgcat

SEQ ID NO: 85 - DNA, 1200 bp, Artificial
  control peak
tgcaatggacctgaataaatgaacttcccaagtaacacttaccttccactaattttacaccctccccaatatttctcctagtgactctcttagaaatgtatcgccccagccagatgccttttgtgctttcttctcaaatgcatcattctttgtctaaaaagtataaaagcatcttgctgtggccacttcttcagacctcactctcttttgaaggtccccatgaaaaactaataaagtgtgtatgcttttcttttgtttatctgcctcttgtcaattcggtttctggctccagcggaagagcccgcagaagcgctgaggggactggagacggtctctggcgcccctacacctgctcagtgagccttcagccccttctccctgacgaagctcccagaggctggaactctggcgcgggaggcacttgggaaatcctcccgaaagcaaggaggagcctttcctagaccgggagcggtcccacgcgggggtacaggttggggcaggggcgaccgagccgggcacaggaactcgcagcagggtagggttggcggctacgccgccctcggtccgcggggaaccacgcgggcttccgggtcacgccgccgcgttccgaccgccctcggggagaggggaggagcctccagcgccgggcggggcgtggttaaggccggtgggccccaccgcagccagacgctgcccgtttaagcgcagagggccgcgggtcacgtggtgggcggggtgggggcgtggcaccaggaagcggcgtccgcgccgggcccagagccgcagccgcagccgccgctacagtccgggccgggctccaccgcgcatcctgctccactctggcgaccgcccccggggcccccgccgcgggcgcggcgcccgccatgggcgaggaggactactatctggagctgtgcgagcggccggtgcagttcgagaaggcgaaccctgtcaactgcgtcttcttcgatgaggccaacaagcaggtccggcgcgcccgcgcttcctcccccgcgcggccctgccggggtcgtgggcgcgccaaggccggtcccgcgcgaccaggcccgagcgtcccctcctgtcctgtcccgtcccgtcccagcgcgggaggcggcgcgcgctccttccttgttgggtgggctcagccgcggccaggtgtcctcgcaggtgacctcggggctggacccccgagccagc

SEQ ID NO: 86 - DNA, 1247 bp, Artificial
  control peak
taggccattttctccagacgtaaaattaaggtacacctacataaatgtcctcttagcaatgttcacagaagtctttgaaaggtgaaactcaaaggtagggctcagggaggcggaaaaaacaagggaacagagtgggcttaatggtggtgggtgcagggaatctaggggtcctggttctcgtcggaattccatcacttagaaactgtgtaaccttggacatgtcacctcctctttcagggccttactgggaaaatgaggaaacgagacaaggtattttcttcccttctgtgtgacattctgattctgcgtttagaagaggagggaatgcacccggtagagggcctgggggcgggagaggcacacgattttaaaacagctcctccgttctggctctacagccagtctctacaagcttccgtaccgcagagactcaagaactgggtctctgagcctgagaggaagctccagagagtccgcccggattaggccacagaagcgccgagctcccgcacagacagcctcagggctcacccccaactacggcgacagggcggacccgccggctccatcagatgcactacccacccgcctcgagagagggtctgcaaggcggcgaccgctccgctggctaagcccagcggcacacctattcggagctccaggctcgtcacctaccgcctgcgcagcccggcggcaacagccgcagctccccagccccgcctggccgtcgccgccatcttagtccggagtgcctaagggtctccctgacagaaccacgaaagatctgcgcagccgcaggctggccggtagctgggcgggcactgggaagagcccaccctaccggctgcgggcggtcctgctgcggataagtgcggtggcctacgcgccgaggtttgcaggctactgcgtcaccctgaccaagagaggccggggaacctttcccgtttgcgccaacgcttggttaattcacgcagcactgtgaacgcatgcgtgtttccgtactttgttttccctgttggctaggtttttagctggtgctggtgctgccttgcgaatgctgtttccagcccctaaacctgacccattccctcctctaggaccctactcagcctccaggcccagctcagtgactgcccccaaagactccctggagcattccttctaaaaggttctcgcatttctcctgcccttttcccatgggtctgatgtggaactgtgtctaacccttcctcatagctgtgcttgtc

SEQ ID NO: 87 - DNA, 711 bp, Artificial
  control peak
aaggatccgcttccgatcgggtggggctctggtcccgaggggccgagtcgggccttgttgccgggccgcagcgtaggcccctcgcccgggacctgagggaaggccctggacggctgcggcacgcccgcctacccaccctgtaggccccgtgccaggctagcctggttggcgttgacctcagggctgggcctcctccatccactgcgaggcccttcggttcgcaaggggactggtgcctcgttctgtacccgagtcaagcctggtcagctcccgctcggcctggctcctggcttcgaccgtacctcttctctcggggggccgacaaacaggggtgctgcgcctgcgcgtccccggcggcagcgggctgtcctctaagcgcctgtcttccggctcctctgcatgctgggatacggagtccttggttgcagccggggaagagttgaacagggttctggctagaactacaagtcccaggaggccatgagcacggaccggtcccgttgtggtgagcgcggggtgtgctgggagttgtagttctcagggaccgtttccgtcggttctcaggaggtgggaagggatgaattagcatctctaacccctgaaagcggcggtgagggttcgggaagcaacacagctctagcccccacacttgctgaattccctaggaagagtagctctatttgtgcgtgtacctctgagcgagtgtgtcct

SEQ ID NO: 88 - DNA, 725 bp, Artificial
  control peak
agaaaacattagatttattatcatctattaatactaacgccccatggatgaatgaatcaatccattgtcagatttatgtttaaatcctagttagcagcgcgtcagcaaaacagtaacaaatcctttgttggcgcacttctcgctgctttcgtgggaccttagcacctcagagggtgctcagcatctctaaacttgggttcctcctaacactagggggagaccaagagccttgggacactcccaacacagggaggggaggcggggcctattgtcaagtcacaccttccctgcctgtgactggcacagacattagccaatgggtgcttggatagggtgcgccggcgccccgccccccttagcgccccgccgccccgaggcaccctctggcagactcggcggcggcgacagcctgggcggacagtgcgccgtgcgcaggcgcggagctagacctcgctgcagcccccatcgcctcggggagtctcacccaccgagtccgcccgctggcccgtcagtgctctccccgtcgtttgccctctccagttcccccagtgcctgccctacgcaccccgatggcggagctgcggcctagcggcgcccccggccccaccgcgcccccggcccctggcccgactgcccccccggccttcgcttcgctctttcccccgggactgcacgccatctacggagagtgccgccgcctttaccctgaccagccgaacccgctc

SEQ ID NO: 89 - DNA, 883 bp, Artificial
  control peak
ggggatggggagcggacaggggaggtcacacggggagggaggctgagggaaggcactgggggcagaggggaagaggtcgaagctgcggggaaagccgccctaaggaaccggagggagaggagggaggaggggacttggggcaggaggttccgcgagacccagatcggtcgcggggccgtgaacaccgcacctcacggacaaggacgcgccgccggcggagtgggggccttcgtcgcgctcccggccttgcgccatgggcagtgttttcctcaggcgggagcaggcagcgcacgcagcaccccagggcgggtcggttacctttccgccgccgcggggctgcgggccgcgacgccgctgctgtgtacacccgtatcccggagaccgtaagccccgcgcccattggctctgcgctttggcccgctcccttctgagccgagcagattggtggaaggtctgccgctctcggccaatgggtgagcggcaagccaagcgaggggcgggccccttgctgttcaaagtgtggtctgtttaccagcaacattggtgctacctgggaagcttgttataaatgcagaaacttggctcccacaccagatctactcaatcagaatctgcattttaacaaatttccccaggtccgggccaataattgcaagtaagactcatgcctacaatctccgcgctttcgaaggtcagagcgggaggatcgcttgaggctaggagttgaagaccagcctggaagacatagcgaaacctgccgcgcccgtgccgcatctctacaaaaaaatttaaaaagcagccgggcatggtggtgcacgcctgtagtcccagctacttgggagactgaagtgggaggatcgcttgagcccaggagttcaaggct

SEQ ID NO: 90 - DNA, 647 bp, Artificial
  control peak
taaggctgttctgatctcttcatctgtcccttcacctggcccctgtacccccttcccctttggaccccttgaaccctcccaggacccccgctcagccccttcccgcccccaaccgactcttcccgaacgtcccttaccaaccgcgagagccccctactgcgctttggccacaccccctacgcctcgctcccggccccgcctctgcccctgaccgcgcctgcgcaaggcgggcgccctaaagtcctatttcactctgttgggaggagggggaaaggtgtacgcaggcgcagtggcgtctaaatttgggcccactaaatgcgtcggagcatctccgcgcccaggcggctcctcctcactgcggcaacccgggaaaacttgtgaactaatcagaaaaagtggaaggcgggagatcttggggcgctgtccaatggcgcggaagagaacacatgagctggccaatcgggaacggcacgggggcgggctcgctcggcgcgaagttcgggcccgggaattccgaaggaggggtaggcgctgcccgcgcgcagaggccgcgcccctcctggccccggcttcttggctgtcaaacagatgcagcaacgtcggctcctgccgaggagcccaaggggtcccgggatccgccgcacagg

SEQ ID NO: 91 - DNA, 1044 bp, Artificial
  control peak
gggtctgttttctcgggtttttgtcctctcacctgggcagctccttttctctcataaataaagaaagctacagcgacttcgcccttgcttccatgcatcatctcatgagtccacaacaacactaagaattcgctttcattttgcagacggagaaactgaggatgggcaccttacccatggccacagagctagtaagtggaacagctgcaaccaggattcaaactcgcggctccagaggccacaggcttaaccacggaatcctagcccgcccgcacatccctccatcgcgggagcgacctatctcatccgtagacaccttccgctgcacccagccgcagctggtcaggcgggcggaggaaggaggcttctgattggccatctccagcagcggaggcgagaaggcggggagcgacggtgcgggggcgtcgcggagaaatgacttcatgacgcggcttcttaaaggcgccggcccagcgcgccgcggatagcacaggcacgtcccgggggctccagcttctgctcagagcgcggagagccgggccgggcgggcgcgcgcgcaggactcgggccgctcgaggggtccggccggacggtgtcgggagccggacccgtcggcagaggagcgggcgccgcgatggctgagggcagtgccgtgtctgaccctcagcacgccgcgcgtctgctgcgagcgctcagctctttccgcgaggagtctcgcttctgcgacgcgcacctggtcctcgacggggaggagatcccggtgcagaagaacatcctggcggcggccagcccgtacatcaggtggggagggggctacggcgggcgggcgcggcggtgctgcccggagccggaggcggggcggccgggccgggcgtggcccccagaccctgtcccctgtccccggcgccggggtccccgcgtcaccgttggcgcggcgtccctcccggcaagcgccggaacccggccccgctgaccgcgtcccccaggcggcctctgtggcttccaggcccgcgctccccactggacccccgcgccccttggagt

SEQ ID NO: 92 - DNA, 546 bp, Artificial
  control peak
ccccgcttctttcccgcgtgcctcagggacagtttgccacagccctctcccctccccggtgcctcactctgcagaccccagtcctccgggctaagcaggttgtcggtgaagaaacgcgtcgggtcagcaatctcgctgagcagcatcagctccgccatctttcccccccaccccccaaccaggagacggttcccaaggcccgcctctccccatcaccaactaatcagttgactctttcaaaaaagggggcgtcccggaagctctaccgaccagtaagagacctgggtgctgagcacgtgaatcaacagatgcggatgactgtgtaggcgggcccaatgggagcacagcagcaggaagtaacttccagacagtgccataccgccaagcgcatgcgccaaacaggctccagtgataaagcgcctgggagatgtagtacccagtattaaggtccaccatctcccccagccactactaagtttagtttagacagcttctatgtgtcctcggagatagataagcccctcaaaaggggcgcactgtgaccac

SEQ ID NO: 93 - DNA, 1041 bp, Artificial
  control peak
gaggaatataaatacagtgtcggaccatgtcccgggtcaacaactaaaaaacagtgatcgaatgaaagaggttgtgggagcagggtattcgccacaaggtgggagttgtcatctccccgagaagaggtgtgttaggagtcaggtaccaaacaaccggccgtgaaaccgtaagcagtcactattcctctttcctcgggatcaaatggggataagcccaggatcccgccccctttgagttcagaattggcccggagtcctgccccagagcaaaaattcttcgatcacctccaggcatctcttccctacctcacagctcccggctggtctcatttcagattcccaagtcctgcctctgcactcaccccattcgggagattcttcacgatcagtcgcgacatggcgcagggtccccgctgttttgattccaacacgactggtcagcgtcttccaccaagtttcacgctaccgccctgggcgccgccatctttaccgagccggaacacagtcacgtggctgagaccttggcctgacctgtccggaagtaggcgtggctaatccaaaactactcagggaggtcacaccccacaaggggtgggcctgggcgcttgcattttgcgcaggcgcagggccttgggcggattctgtgagacgccaatgcagcggagaaggcggcagggggcgccggtccctaacatgcgccggtttagctcggtgttaagggtcgcgccaaccgggcaatcgcgttctctcctttttcttattttatttttattattgagccttaggactcccctgagaacgggggcaagattagggacccttgccttcctcgtagaaatggggaaagtgaagtttagagagattgccagccaacacaaatcaagtattgagccttttgtgtaaatacttggaatgaacatgttggaaataaacgctctcattttgcaggcagataaactgggaatcgtgcgtgtaaagcagcttgctcaaagtcttataactatgaattggaaagtcagattcgagctagggactgcctag

SEQ ID NO: 94 - DNA, 811 bp, Artificial
  control peak
taactcctgcctttcagacctgtggctcccaaacgcggagggagtctccgtgggccccaccaccgccagcagtttcctccgggccacgaagcccttcgccgcccccgcctctacccctgaaatcaagtcccgaagcccgccccactgcacgaccacctcaccctgggcgcctcaccctggtagggcttcgacagcgagtgctcccgtttcaagtactcgaacgtttgacccgccccgacttgctgtggcccctgcccagaccccaacaaaagaagaaggagtaacatcctggacccatcccgagccgacaaacatcgccgccactgctgccacgacccaaggggtcccagagtcgccgccatctttcccaccaacgacccttcatcaaaagcccgccccgtcgcccaacccattggcccgaagagccatctgccctgcgtcctattggctttcgtcaagcccccgcccctgcaggtgactatatcaccgattggctggcttctgtatcagttgccttgccaacccagaaaaggggcgtagccgaatgcccagcccccccccttatttgcatttgccctgagttccactaggcttgctgggatacgtagtttatttgtattttgttttctgcgtacgagagtaaatttaaagagataaaacgtttcttctggtcccaaacattctatcctctctgcaatagtacgctcctgactgcagctctctgaatcaatttggaagatatagcagtgaaaggagaaatagacaaatccgtaaaatagctggaaaactatcaatttataaaagacttgaa

SEQ ID NO: 95 - DNA, 741 bp, Artificial
  control peak
ctgaggcaggagaatcgcttgaaccctggaggctgaggttgcagtgagctgaaatcgcgccactgcactccagcctgggcgacagagcgagacttcgcctcaaaaaaaaaaaaagaaaagaaattggagacaggacggcactcctcctaccacggccactatacaaccgaaggcacctcctcccgcctccctccaggtggcgctgtaggaccggcccaccttccacagcgcagacgcctcagtttggcctccctcggaggccatgcagcgaagaaacgtgacttcatggctagaggggcatttcccaggagccgcccatgccgccgcgtgaccttcacacttccgcttccggttctttattccggaagttgctctcagaggcagcgtgcgggtgtgctctttgtgaaattccaccatggcgtaccgtggccagggtcagaaagtgcagaaggttatggtgcagcccatcgtatcctacgcaggatgtcaggactaggaggttcgggtcagaatacggggtgcgaaggcgcaggctgagggcaggcctgggatagtggagtacggatccacatcccatgagcccccggggctaccaagactggaagaaagcgcgggtagttgaaccgtgatggtggggaggtggtcttggggggacccctgccgtggggaatgcaggaatggacgcggcaaaaggagggaagagaacttcaggtgcggagtgaaaacgggagttaacggttt

SEQ ID NO: 96 - DNA, 745 bp, Artificial
  control peak
ccctggctcgtcccgggctcagccccttctcccctccctcacaagtcggactcacgcacgcactctcctcgcccaaagtgcacatcatggagcagaggaggactgcagagatgcccagaaacgagcctttgccgccccctccattctcacctgcttccactctgacctccacatgaattccccaaccacacatactgcgcatgagctcctctgccccgcccgctttccaagatccacgcttcctattggtgtggaagagcgctgtgagttctgggaagggtggcttagaacctcaccctggttctgcggctcttagcagatgaactacaactcccaaaaggctatgcagcggcgggcccgcggagaaaagcgttttctctttggtgatcaacgcgaggatgttgccgcaaagccttttgggggctgtagttttctggggctaaatgcacgtgcctatcctgtacagaggctaagactgccgcaaggagagccttagggctccgggaaagaggaggtgggcgtccagctggtggcaaaaattagtaggggcaagttcatggctgatgaagaggacaggtggaactctggaggaagtcgtggtgaaatcaccggcaggctgagaccctttgggggaaacgaagggccgtcttccttccaaagccctgtctgcaaacgtcctcagagttgtgagacgttgattaccagacccagagagaaccatctgtgctgaaaaactgagaccaac

SEQ ID NO: 97 - DNA, 747 bp, Artificial
  control peak
ctgggaggcggccccctgcctgccgccgcccctcccttggccccagtttctccaggtccccggtcacaggttctgggccggccctcacctcacctcagggtggcaacgtgtgcggtcgggcgattccggagcccctgcgtggaggaactgctgggcgggaggagacgccggcggctcgggcgatggctgacaggcgtgcagcaccaggccggcccggcacacggagaaggggcggggcttcaggggcctgacccgcccccgccccgcccctgtctcccacctcagccggcgagctccatgaggtgactacgtccggggttactccaggccgttcacctccgaccccgctccgggctgcggcgctgccagggccgccggggggcgaggcgcgggcccgagggctcagcggcagccaatgggagggcggcgcggggcggagcgatgtgaggcgatgccgatggtccgagcgcggatctgccattcggggcccagcggctagagggcgggcgggccgcgggcgggccgcgggcgggccctgggcagtcgtggcgcccctggcccggccttcgcggccccttctgccacagccgggtacggggccaacccctcggctcctcctcctggcggagaggggagtgggggaagcggtcccgcatgcgcctcgcatcagggacagaggcgtcccaagtgaggggccaggactagccggcctgtagaggcggggaaagggaacccagaattaaggcg

SEQ ID NO: 98 - DNA, 717 bp, Artificial
  control peak
tctcatctttaaatgtcttcagctgtcctcctccaggaagccgggtcaggcgcttcctcccgggcgtctccggtcttcctggaaaccacaggtcgggtgtcaacctactccaggataggggccccgaggggccagccaggcctcccagccttgcccaaccctggggttgacacggactttagcgtctttttaaacttccggcgcgggctctgcggtggcacaggagggtcctccaaactttcctgcgatctcaaaggtccagattccacattggggatgggcccaactgactttgcttcgctctcattagccggtggtcctccaggaaagcggggccgcctctccgctgtgctctcataggcccaggttcttgcgttcgtgtgtccttctctcactctgattggcctatattccccctgagggtgtggccaaatctcattggctctcgcgcccgaatgtatgacgtcatgcgccagcgcccgtcgcttttgctggacgtcatcctcgggagcccacccggacgaagggggagagtagacagcagaaccagcggcggcggctaagcagagactgtagtagcggcgacagcgacgacggcagcgatggctggggcggggccagccccgggactcccgggtgcaggaggacccgtggtcccgggtcctggcgctggcatcccgggcaaaagcggcgaggaacgcttgaaggaaatggagg

SEQ ID NO: 99 - DNA, 646 bp, Artificial
  control peak
gcctcctcgcagcttggcctaccgcgctagtgttccggactaacgaacaaacggctcactaaattcaaacatgagtttcagcctggtcgccgagccggcgggtagaaaaaacctttcccctcctaaccctccgcagatgtcggccccgcactccgcccaggccagccctgcctctctcaagatggcgacctggaagtgcgtcgcccgtgccagccccggaaatctcgtgttaggcgaggtcaacgggtaggttctcgcgagaggacccgtcagccccagtcaggcgtcgtgcgaacagcagctggtaccgaaggcggaggtggagcccgagaggtaagtgcgtgtgcagaggtggcagttccgggcgccggggaggtgtagagaacagattcggaaactggggaggtctagcatgtggcgtaggagggggtcctcactccgcttcgcgattgccaaaacgagcctgccggaagcgccctaaggggttttcttctcccagggaaccagcggggaaactgaggctcggggtggagcgcaggattgtgggacgcgccaaggctgctgtctttcccagcagcagcggaagatgtcggacagcgaggacagcaacttttccgaggaggaggacagcgagcgcagcagtgac

SEQ ID NO: 100 - DNA, 505 bp, Artificial
  control peak
tcagcacctcatcaatgagatacgactcctccggggagaatttccccctgcctggtccccttccctagagagcccttcgccaaccctgggaaagccccaaacgacagctcccgagcgccacccggaccagactccaggaccgccctgtcccgccaccctttttcccgcccagactccacgtcttctgaaagagggcgtggcccgcaggaagttctcgcgttacggaaagtgaagtgtttccggctccggtgtcatggccggctcctaccctgaaggtgcacctgcaatcctcgccgataagaggcagcagttcggaagccggttcctgagcgatccggcgcgcgtcttccaccacaatgcctggtaatcaccctgccccctcgcccggcctgtcgctggccgtctgtcccgccgcctcggagcattccgaaaagcccctgaccgccggccacgagtcaagctgccctacccgaggcactctccaaggggagagaaactcctaggc

SEQ ID NO: 101 - DNA, 603 bp, Artificial
  control peak
cggacccccgcggcggccacggctcaccacacgaacactctgcttcctatcacaatccagcggcagacgcacctggtctggcacccccactactcacttcagtcagcctggccccacctgggccacccttcacttcgctccgcgcctcacccaacggcagagcagcgccgcgcccacagctccggtttgaaaaccttcaccaaccaatcacagagaaatttattcctcccgcgccgtccctctacggtgtgacgtacgcgtcagggccacgcggaggtgacgggaacgggaatgagcatgcgcattctgacaaagcgaagccaaaggtggccattttgactaatgtcaacggtttttcggttttttttgtttttttttgcggtgccctttccggaagtcattcaggatgccatattgactaagggcaagtggtacttcctgaatgcgaattattcttggtctcactgtctgcgggactagggcccttggaaacgattaactgaacaatgtcagctctcacagtagaatcagaagaataccaggagttagaacagcagttagtttcaagcacattcactgagctatattatcccttccattgat

SEQ ID NO: 102 - DNA, 570 bp, Artificial
  control peak
gctgttttattttacataaaaccatgacgattaaaaagctggcctctcagctttccgggtattggtttgttttccccctaaactgtcgtttttataggcacttgcactgtcttcttagcagaattgccaatggccttcctcgagtcctacgtttagcgattggcaagaatcatgcactgacagagctcgactccggagggcacaaggaagtagacaacaaagcaggaagaagcaggaggcgcacagatgggcgggatctgcactggaggcggtgcgaccttgcggggcggtgcaagctggcggaacccgcgtgagagaggttggtgttgcgaagggaactagtccggtgcaggacgtggggcttttgcagctcagctggttccggctggggaagatggcggtggctggggcggtgtccggggagccgctggtgcactggtgcacccagcagttgcggaagactttcggcctggatgtcagcgaggagatcattcagtgagaacagttcgggtccaagcggggaaggagctctgggatgtgcactgcctgagcgaaccgggaagcggggag

SEQ ID NO: 103 - DNA, 738 bp, Artificial
  control peak
cccaagctgtgctttccttggttccaggaaagcgacgagcttgaagctctttgcacatgcacatgccctgctgtctacctgcagtgctcttcccgcgcacggccacaggcctgggaggccgtccggttaagtacccctcctacggttccgattctaccctggcaggacaaccctagctcctcgtacctggcgtggcccgaccaatcgtctgccactcagttgacctctgacctgttagtacccgcccctggagcgccgcatccggtagcgagagttcaacttcctgactaggagccaatcagcggggccgcctcgctgtctacgacagagggtggggcagacgctccgtttccggtggcagggtctggggaagcggcggcaggcgccatgtccggccgcgaaggtaagtgttccggaaccgtgaggactgcggggacggcggggtggggaccgggcggcaggggcaggccgaacgtgctcgtgtcgcccacaggtggcaagaagaagccactgaaacagcccaagaagcaggccaaggagatggacgaggtgagggcgggcgcggaggcactggcgggtgcgggggcgctgggagacaggcctgagttgaacacgctctgcctctccccaggaagataaggctttcaagcagaaacaaaaagaggagcagaagaaactcgaggagctaaaagcgaaggccgcggggaaggggcccttgggtaagtgggggccgaatgg

SEQ ID NO: 104 - DNA, 677 bp, Artificial
  control peak
tgtggacaacacagcgagaccctgtctcacaaaataaagtaagcccggactgagtgcggaaaggcgggcctggcgggtctggtctccccatgcgggccaccagaggccctgcagccttcagtcgcttgaaggggtaatggcgcttccactcacaaacatggcggacagagcgtgtgaacgagatgaacagcccctcaaaaatatggccgccgaggctggacggccgtgccccagcagcaccgcctccgcgccccacgtgatctctcgccgggcacagcgctgaccgcggaggtccaaccggaagaatgtccggattggacattcggaagagggcccgccttccctggggaatctctgcgcacgcgcagaacgcttcgaccaatgaaaacacaggaagccgtccgcgcaaccgcgttgcgtcacttctgccgcccctgtttcaagggataagaaaccctgcgacaaaacctcctccttttccaagcggctgccgaagatggcggaggtgcaggtatgggctccgcgcgggccggggcggcaaggggccgggtgggatccaggccggaatggggtcgcaccctctctgtcttgcatgttttgcggagcttaacatccataatgaagcaaaatggaagtcttttgggataaggggaatctgtaggaaatgccgcggctct

SEQ ID NO: 105 - DNA, 687 bp, Artificial
  control peak
aagcgaccctcctgcctcggcctctcaaagtgctggagttacaggcgtgagccactgcgcccggcctagaagtcacctttcaacttagtcctctatacatgggaaaagtgaggtccagaaagggtatgagttgtaccaggtcgctgaccctgaacagaagctagatgtcttaattcccaaataggactcgctgctaactctttgggcataccttcgcgattcaaggctcttgggtttcccgctttggccgcctcgccaaacccaagtcttacactcttagtgaccccctcctttcctccttcccccttaggtcagcagggtttactcattccaattgcaatgctccatgttgccggagtagctcctcgctagaagagagattagttatgtcaccgtcggctcctcccattggtccgggcgtgtggagcgcggcgccatgacgtcagacgcccaaatgagtggggcggtgaggggaaggaggagggaagtaggacttcaacatggcggctgcggcactggcggtggctacggtgacggcctggcccggagcgggcagagttggaggtggtggcgttcgctctccctaggggctgtcgggagctcagcggggaccgagcctgggaggccggccggtgccagcacctttcggcttctgagacggcggcagcagcggcattcaggtgaggg

SEQ ID NO: 106 - DNA, 599 bp, Artificial
  control peak
ccagcagccgctccaccacctgctcgtggcgccccagcagccgtcgctgctgcgcctccgcgcggttggcgcccagcaggctgagcagcccctggctcacctcctctatgtcgcggaaggcggccatgactacgccaggctccaacccgcgcaaatttgactgtccgcggggcccgccctcttccagaagaggttgctttccattggattttttgatacgtcagtcataagcaatggccaataggcatggggaacaggatcttagactgggctaagccgcctttggtctggcctggtgcgcctgcgaccactcctccgtcagtcatcggtgatgaccaatcggcgcaggaagggggaggctccgttatccttcccgatgcgcctgcgcgggagagaaagcggctgtttatttctccataggtcgagcgcttgctctgtgcagggtggggttctaacggcaaagcagcgagcagcttctcctggaacttacatcctacagtgtgtgcgtgggatacacaataagcatagagaataagtagattctatgagaggctgcatagcaaaatggcgtctgcagccagatttactgggttcgaatc

SEQ ID NO: 107 - DNA, 1011 bp, Artificial
  control peak
tcagcgcagcgcctgacacacagcaggcagctgacagacaggtgttgagcaaggagcaaaggcgcatcttcattgctctgtccttgcttctaggaggcgaattgggaaatccagagggaaaggaaaagcgaggaaagtggctcgcttttggcgctggggaagaggtgtacagtgagcagtcacgctcagagctggcttgggggacactctcacgctcaggagagggacagagcgacagaggcgctcgcagcagcgcgctgtacaggtgcaacagcttaggcatttctatccctatttttacagcgagggacactgggcctcagaaagggaagtgccttcccaagctccaactgctcataagcagtcaaccttgtctaagtccaggtctgaagtcctggagcgattctccacccaccacgaccactcacctactcgcctgcgcttcacctcacgtgaggattttccaggttcctcccagtctctgggtaggcggggagcgcttagcaggtatcacctataagaaaatgagaatgggttgggggccggtgcaagacaagaatatcctgactgtgattggttgaattggctgccattcccaaaacgagctttggcgcccggtctcattcgttcccagcaggccctgcgcgcggcaacatggcggggtccaggtggaggtcttgaggctatcagatcggtatggcattggcgtccgggcccgcaaggcgggcgctagctggctccgggcagctcggccttgggggcttcggggccccgagacgcggggcgtatgagtggggcgtgcgctccacgcggaagtcggagcctcctcccctggatagggtgtacgagatccctggactggagcccatcacctttgcggggaagatgcacttcgtgccctggctggcgcggccgatctttccgccctgggaccgcggctacaaggacccaaggttctaccgctcgccccctcttcacgagcatccgctgtacaaagaccaggcctgctat

SEQ ID NO: 108 - DNA, 563 bp, Artificial
  control peak
aagactaaggttgccaaaatgtttgatcagctctgcgtcagccttgacattacattacattacagatcccctgatcgccaccgtcgcagaagctctgaattaaaagcgtgcatgtagctgcacatccattgctggcacttcttccgttagcctccgctggaactggttagtgaggacccctccatttcacaagacagcttcggttttgcttcgttttgacttccgcaccccgaagctgtagcaccagtttgttttctcacggcacaggggcggcttccggggcgcggcgtgctgggggttgtagtccgcgctctgcctcctacttccattacagaaggcgatgggccgagagctgaggaactacaactcctagagtactccgcgccgccgggacgccgcgcggctgcggggctgggcgagcgcaaagatgtccgcgcccgctgccgggaggcgaggtgagtctttgatcgtaaccaggagcccggagctgaggcagttcctgcacgtgtcgcggggccggagaagctagggccaggtattccagggatgcaagaatcctgcaa

SEQ ID NO: 109 - DNA, 984 bp, Artificial
  control peak
agaattgcgggggttggagttaggtagagggatttaaggtgctttctcccagaattgggggtgaaaacgggtggattggacggtggggagaacgagaagtcagtttcctagaatcctctccaatagctacccaggattctctagaggaaaacaataatggccagttacccacaattggggcgggggcaggggaaggtgacggaaacggctagttacccagaattctctgggggaaccagaaaaatcggttatctagaattctcccacggcagcggtgaggagggggatagggcaagggtcagatgtgttcctgggatgcttacggggaatgggtcattaccaagatttctccatggcggatattttggagcgcgtggaattagaacgagtagggggagcgcaagcgctgtcagctccgcgggaaattccagtttccccagttctcccccacgcctagggctcaacctaaccactacggcgccaaggaccggggtccgaggcgcttcttccaatcaagaagggctcggggcatattttagccaatcagaagggctccgggcgcatctaagccaatcagaaggagccaggtacggcgaccccaggagagagcgtgtgacgcacgcgccggaaaaagaggttacggggcgccggtgggacaaacgcagccagacgccggacttgttgggtggcgttagaaagtaactctgtttttagtcttcttgaacaaaattacgtctaggtttgaaaaccaaacttttatgctttctataactttaggaactctaaaataagggacacacaaggcaaagcactaggagtcgctgttgggtattcccagcggggcttagtctacctcctcttcctgcggtgccctagtgagccactccgccacctctgaccctgttgcccccaaaactcgctggcatttcctattatttccagatatgggagagaaaaaaaaggaagcgggagccggactcctga

SEQ ID NO: 110 - DNA, 524 bp, Artificial
  control peak
tctcccacgccagtaccccagagcatcacttgggccccctgtccctttcccgggactctactacctttacccagagcagagggtgaaggcctcctgagcgcaggggcccagttatctgagaaaccccacagcctgtcccccgtccaggaagtctcagcgagctcacgccgcgcagtcgcagttttaatttatctgtaattcccgcgcttttccgttgccacggaaaccaaggggctaccgctaagcagcagcctctcagaatacgaaatcaaggtacaatcagaggatgggagggacagaaagagccaagcgtctctcggggctctggattggccacccagtctgcccccggatgacgtaaaaggaaagagacggaagaggaagaattctacctgagtttgccataaagtgcctgccctctagcctctactcttccagttgcggcttattgcatcacagtaattgctgtacgaaggtcagaatcgctacctattgtccaaagcagtcgtaagaagaggtcccaa

SEQ ID NO: 111 - DNA, 574 bp, Artificial
  control peak
tgcaacctctgcctcccgggttcaagcaattcttttgcctcagcctcccaagtagctgggactacaggggcccgccaccacaccctcgctgaccggctgcagcgcgcaggacagcacgcctgggagggcggtgccaggaggcgggcctcggattgggcctcacgtagtgggctggacagaggatggagcctcttggcgtaagcgcggttgggcggggcattcctggattggcccactccggcgtcgcagaacgcttggcgtatcgtccttcagccaatcagcgtggagctgctgcctcgccccgcctcacgccggcgtcctgggggcgcggagagggcgcccaggttcaccgcgctctcagcgcgcacttccgggcacggtcagggcgtgacgtcgggaggagggcaggtaagtgccggcgggcggaggagcggggagggccgggggtccggcagccgcctcaccctggcctcggagctgcccggcgcgcccttccgcgtcgggggacgcggagctgaggacccgatttagatgcccggagccggcccaggtcccagccaggaccaaagtccct

SEQ ID NO: 112 - DNA, 627 bp, Artificial
  control peak
cggtgagcccggcctgaggggacgcaatagggggcggctgcaaggcccgccacggcgctagcctggagcggacgaaacccttgcactctgcggtgctccccccaagggcttgtggcccctagtcctcccgccgcccgttccccggccgccagccggtccccaacggcgcctcccctaccttcgttcgccaggtccgccattttacttccttaagccctcccacaaggccccgcgccggcccaggctcgcctgctttctgccagaaactcccgcgcgtgcgcacttaacaagccgactccgtgtgaggcggccagctcgcgcctgcgcgctgaatgccgcgcggaggcgagccccggatcgcctgccctgcagagcagcctgccctgacgagccgcgctcctagcacgcacctgcgcacgagccatctcccgcctccttttcccgccgactgagagggcggggaaagggccgggaagggcgcatgcgcagtggcagcgactaaggtttagtgaggcggcaccgcgcggcccgaggcgggtcttgaagcactcgttttcccactcgttttccggggttcccccgcggtgggacagtgagcagttcagggcgccgcagccgaggccttcc

SEQ ID NO: 113 - DNA, 823 bp, Artificial
  control peak
aagtgaaagcaaattattgaaagtggtactgtttacaaaatgtctagcgcatggcaggtgctcgaagtagtatctcttagtattattactactgtcttatctcgatcagattcagctctttaatgagaggctctaaatcttatgttcgattcacctttgtaacccacaccgggcctggaatagagcactgttcataattaatgctgtgcccgtgtaataagacggcggcgagcaaagggcatcatttcagaggaagagtgctgtaatggtgggatgggagaaattcctagaacctaggtgggagccagaatcttgcagagaagccccttatccgtctgcaccgaaagcaggtagagtggatgccgtaagattcctaaggacgcaggtagagcgcctcctctgtgccgcgatgaggtaggtgcatccgtcacttcactcctccctctccacttgatccggaatgggaggaacgcaatgcgtcccgcgcggcgatctgggatagctgcgcaggcgcagcttccggctctgtgtctcgggaattatttgacgccaccggttcagccccgtagaaagctctgattaggcgcaccgcgcacgcgcagtggcccacttggctggggagttatttgacgccgccgcccctggcagtcggaagttgcctgagcagatcccagccggctggctcgagtggccttcgtcgtcccttggcgccctgggagagtcgctgacgggtggactgacggaccgcctgaggacggccggccagggcggtgaaagcgccagccctatggcgcgggtcgcgtgaggcggaaggccgaggacg

SEQ ID NO: 114 - DNA, 646 bp, Artificial
  control peak
tgggagagccaaggctgcccaaggcatagccgcggctcctccggggtcccttgccctgcatctggcagggaccagccacgcactcacctcggcggctccaccaccggctctgtgccgccgccgccgccgccgccgccgaccacggtgacagctccctgagcgcccgcacttccggggtcaaactgcgattccgcaggagcagacaggaagtctcggcctggccccgcccccagagccgagcaaactaccgccgggaagccggacccgggttgcgcctctcttcttaagactccgccccacactcggcgtctgtagccacgcccccttcccctcctatctcccttcctgcttaagcagagaataggtcagctgagggcgggctgtgtgattacacctctcgggaaatccaagtatggcacctgagaggacgtctcggaatcccaggcttccaccttctgcccgacctccttagtcaccgttttacatatttaatccagaagagaaattttaacaagtcaaaagcacgaacataatgttaagcagatgtacaagtacagactgcgatttcatttacgggaaattctagaacagacagaactcatgagtagtgatggtgttaaaattggtggtttcctctggggctgtg

SEQ ID NO: 115 - DNA, 971 bp, Artificial
  control peak
gagatgaagggaaggaaaaaaaatgatgaagggtagaaacagataatcaaaataaactgatgcgatccagagacattatttcccgttccatttccccttttcttacaaggttaagtcggctgaagtcggggtcgcgttagaaaaagcacacacacaccttgcgccctggaaaggtgaggttcgctccgcagcgctccgtggggctcctggctccgccccgcccgccttgggcagctgagcccagctcacccccgccccgggaggatggaagagagggggcttctagcggtctaggcagggggagggggaggcggctgctccgggtgccaggctcacggagcgcaggactccgcggtgcgccgacaccccctcccccagccggctgccgcttgctcccagccgccgccggaagccagccccgccccctcacctccaccctggctcttcgcgagccctctcctgtctgcacagccaatggcgagccagggagccagcagagcgactcgccaaacagccaactggggaggcccacagggaggtcagccaccgggataggcccgctacccgcggacccggcccgactcggctcctctttgttagcgctgcccccatctctcccggcccctaccccaaagccaggagccaagcatcccgtagctgggccaggtgaagctgcgcccctttgcaccccttctccctctgtccccttgtcccctcccactttctgcctgggtactaggagacccccccccacccacgcgatacctcccagagtgcctcagacttcaacagcaattgccacctggagacactcgacagtaaaaagatgctccccaagcctctctgcctctagtctaatcaactgcactgcactgtaacagtggacggatttaggattgcatattaaatcaactgcacacatcaatcctccctgtcggaaactcgtgcttcccgtgacgcagaaattaagc

SEQ ID NO: 116 - DNA, 521 bp, Artificial
  control peak
agaaaaaaaaaatttaaagaaaagaaatacctgacacagaaaaagcgttcaataaatatttcttgcatgaataaaagtactttcgctgggacatttttctccacacctaccctccagccagccccgggatctataacactctctccgagtgaagacacagaggtgatgagatcgggtaacccacgggcgcccgagccccagccgcgcaagctcacgattgggccagaagtgaggatgaacaagcatttagcccaatagaaagtcgtagttctcttgctgggcgtggcttgaatgacttcagtggcctcctcctgggagggagctgaagccgctcgcaagactcccgtagtccccacctctctcagcttccggctggtagtagttccgcttcctgtccgactgtggtgtctttgctgagggtcacattgagctgcaggttgaatccggggtgcctttaggtgagtgtggagggttctgtaacctgggaccccagtctagcgggctgaggagccggaccccag

SEQ ID NO: 117 - DNA, 850 bp, Artificial
  control peak
ttcagtccttgaaatgaagaaaataacgagtggggtaggggtctggtgccctctgacaatttagtgcacccgaaatctctggggaagaacccacccttcacggcggggcttcacggacacgaaggccatcggtgtttttcacctactggggagaagggatatgtgctctctgactccttcaaatgttttctacagcagagatggcagtcgagattaatacacacgcctcgtctcactccagaacacaatgactcaaaaccgcacgacgtgtacttttggaaacagctccgcgggagagcacccgggacaatccccaaaccccgttgcccgcggggtggggccgcggacgtgcgccacgtcgtgtgcgtcccgggcgtgcgtgcggcctccagtgcgcgtgcgcgtaccctcctggggcgtgctcgcggctataaggggcggaggctgggcggcgttgctctgcgctctgcggctgacggcgcttttgtctccggtgagttttgtggcgggaagcttctgcgctggtgcttagtaaccgactttcctccggactcctgcacgacctgctcctacagccggcgatccactcccggctgttcccccggaggtgagcggccctgtgggcttccccggctccccacccccatcgctagagtgctcgccgcgctagcagcctcgcgttccagggctgggggtgcgccgccgggatcctctctgatcgcccacgccccatctggggtccccattcagccgtcagccctgttttgtcctaaggtggtcctaactaggatccacgtggcccatcccacgcccacaggtgctgagttaggcatccctgccatgccctccc

SEQ ID NO: 118 - DNA, 633 bp, Artificial
  control peak
caaaagcgaaactccgtctcaaaaaaaaaaaaaaaaaaaaaccaaaacaaaccctcaaatatccctcgggctgggtgcagggaggaaactcggcctgagaggcgcagtggctagaaccagtcaacactgctacgcacagccacgtttccatggcttcctacgtcactcaggccttagggggcggggtatgtcaccctatccaatcaggggcgctggggcggggccctgccaactgtccattcagatgggcccgggagaggagggcagggcctgcgcctccctacggagcctttgtttctggcgaccgtccccacccgggctcgcgtctccgtttctccgagaggcccaaggtgtctccgccgcagcctctgtcgcgccgtgacctgtacaggtcgcgggagtcgtagggaggacgccgggacacctggaagccgagaaatggtgcgtgtgaggggtcaggcgtccccagacctgggggaggggctggttggaaccggccggaaccggctgtggagggacctaggcctccctgcggcgactccggggtctgggacccgagttccctcggccgcaaggtggggctgggccaggagctgggaccccggtcgtcctgtcccgtccctgcgcgacgcc

SEQ ID NO: 119 - DNA, 890 bp, Artificial
  control peak
gtctccccaactcgcagcccatccctgggagctcgggccacgcgcctcctcacgatccctcccctcgccgcgcgccccctccctccaccctacccactctgcctcacgcgcctcagtaaggcgcgcgccccctcctcagtctttcttcctcctctccgccccctccctcccgccgccgccgccgccgccggcgcggccgggaaccctcaccggcctagaaagagggcgctaagcgcaactagcagcaagaccaacagcgcggagaagaacagaggcatttaggtccgggtccggcccggcttggccccggcccccgccatgccgggccccgacgccggatccgcgcagcaccgccgcgggccgaccagctgcgcgcgcaggagcaacgctgagcgaggacgcgctggtgcgcacacgagccccgccttccgcagcccgccagccaaccaacggcctcccaccccagtcccgccctgtcctacgacgctgcagggccgacgggctgcgtgcgcgaggagcgggcgtggttccgggaggagggcgagggggagcggaccaagagctgaaacacgccccgggcgcacaccacggtgcccggaaccgcggggctcggggacccgcgggggtcggagtccgggcccactacctgggctggcggtggacctggctgactcatttcggtccggggagctcactttgaggggagttggagtccgcctagccacttaggcctggactcagggttctgcggaagttacacccctggtcagacgtggtagcactcccatcttattcaagatcctgtttgaaaattactgaggcagaaactaggaggtaattaaaaggcctcaaaaatctcgcctcattccatccacagaggaaactaatga

SEQ ID NO: 120 - DNA, 1144 bp, Artificial
  control peak
aatttaggattaagactccagttaatactgccaagcagaaggcgtccttggagaaagagaagtctcctgaacatacccactggggtacctagaatagattttgcagcgacacacagatcctgtgttgtctggagtctctgctgagtaatgtagattctgggacagcaaaaggggagctggtgactttgaaccaaattttacctttcaactgaatccaggggaataatttgcaacagagtggcagacgatacaaagtgctatgttgaacactagattgtgctttattttttccgcccaacttcacgtttccatgcaccaaaatgttgatgctataaatattggatgccacgcttacctgacaggcttcttccatcccacctgggcgcgggggctcagagcgagcgcgcgagccgcggctggagcccgcctctcgcggctggagaggactcagccggccgcgggttctgctgcttgcccgggtgccctagccgcttcccagccagggctcccgcagtcagccccgcgcgcgcacgcgcgctcccctcgggcggcctcgacgcctcagggcttcggcagggctgcgactggccggctccagcgggcggggcggcgagcgagtgctcgcggccacgtgaccgacgccaacatggcggcgcccagtggcgtccacctgctcgtccgcagaggtaagcgcgtggaggagagccccgtgagggttcgcacggttgctcactaggtacgcacccgggtccaaagggatgcgcacccgcgctcagacttctcggcgcacacgactgtactttcagtgtttacgaacacacgcaggcacacacgatgacacattcacacaccacaggatcacacatacaaatttgttcactgatcacccgcctagaacacgtcaccacgtggcatcttttcgcaattactccttgagatttgcgggccattgtctgaagtttttttccccaacttcggttgtatcattagtgtaaccaacatttattctgtatgccgagcaaggaaacctatggtaattctgaaatgcatctggacacccggttcccttccctagagcttttatgcaggcatgctttttggggacttgcaaaatgtgatggttttcttccagtgacttaaagtt

SEQ ID NO: 121 - DNA, 675 bp, Artificial
  control peak
agtaaagcgtttagaataatgtggacggtagcaagcacggcgaacgtgttaacgattattaagaatgtattttagccacatttcccagccccttccccggcctagcataccccattttgtggatgggaagtctgcctctcagcaaggtggcctcttggaggacgctgtgcccgcgtcaggcgatcctgtcgactgccgccaagacccggcttccggctttgaggggccacgccccgctcgcactccctggcggaacctttcagtgctgcgctcgcgttgccaacccggcgcgcctcacgcagttgcgcccgctgttgggactacttccggcttccccgccccgccccgtccccgggcgtctccattttggtctcaggtgtggactcggcaagaaccagcgcaagagggaagcagagttatagctaccccggccgcggagccggctcactgcactacccccgcccccttctttcctccagacgccgaagtcgcgggcgctcatggcgggcctggaggtactgttcgcatcggcagcgccggccatcacctgcaggcaggacgcgctcgtctgcttcttgcattgggaagtggtgacacacggttacttcggcttgggtgtcggtgaccaggtacgccacggagccggccagggtggaggagggaactgtcttctgc

SEQ ID NO: 122 - DNA, 912 bp, Artificial
  control peak
aatccagaaagcactgcttcaacgcccagtgcgtgcaaggttccactctcgggccagcggtcctctcccaaagcccgttcttcctaagtgtgcggccgccatccaggacacgccacgctcgtaaaaggtcacctgaagcatctaccctccacgctcaagaggtaggcggaggagcgcgcccttttctccaaaatggctgagcagggtcacgtgggctcgggggcggggccggtgcgctcgccggagcctggaagtgggtcacgtggctcagacgggggcggtagctgtgggggcgtggccgtcacacggcgcgatccgtctgggcagcggctcttcgggggccacgccctccgggccctgggggcctagcgcgcgacggtggggtggagctagcctcgtgaccttttaccccaacatggctgcgcccgtgggaccggtgaagttctggcgacccggtaaggcccttggtggaacgctgggcagatgcggcggcctgacttcggtcttggcgccgcggccggcgccctggggccagcagacctgctcgcaggcctgacctcggcgagctgggcgctgccgcctctgtgcggggggagctctggctcaggctccccagtggtcccgagggatttgggggtgctcccctagcgtgagcagaaactctttgtgaccttgttaaagtggtccagcctcctcaggcctcgtcttcctcgttactgatgcttgtcctgcccaccccgcgagatggggaatcctaaagctccctctcgtgtctcgttagcgccctggggttgcgctgtggcccgacagacaatgctgctcgctgctggaggcttgcaagcccaagttatggagtcagactgtccgggttcgaattttcgtctctgccttgtagtagggtgaccttgggcg

SEQ ID NO: 123 - DNA, 646 bp, Artificial
  control peak
acggcaccgcaacaaagagccggagtccacatgccgacccaccgcaggaggaaatactcacccgggcagaactcagtgctgggatgcccacactgcccaccacgcggaagcctggcccggaagtgactccgagactaccactcccataaggtcttggggctttcttgcctagaactcccagaagcccatgcgacacttccgctggatgcgcctacggctccggggtcccgcttggcctacaactcccagaaacacttgcattgctctttctgccgctcccaaaatacactgctccccctccccccgcgtttactttctgcgcctgcgcgcggccgcagggagcggcggagctgcgtgtactgttgtcatggcagcgcgctgtcacccggcaggttgctggatgggcgaagtagttgtattctgattaactgattttgtgggattattataggattttaaggggattacagggcctatgatttctgccctttgaggcggggttgcgggtgggaattgaaggtggaactgcaggggttggtcgctagtgtaaaaggcgcagatagcgcatgcgtagagcagacttcgaggattggagtggaactccgcgcgcacgcgggggctgtcgtgatgcgcgtgctggagaccc

SEQ ID NO: 124 - DNA, 616 bp, Artificial
  control peak
cggccgcccgagggccgatcccgagaaaggacgaggcggcaccctccgccccgcacccctaggcccattaccggccccgccgcagctgcctcctcccgtgatggtctctgccgcccccgctgccgccgccgccctagcccggtgggaaggagggaggagcgagcgagtcccgggccccgcggcgacggccggggagctcccagggcggcagggcggggagggagggcggtgggcagcccagccggcaggcagagagggccgggcgtctccggcaactcgggcgcccccggcagcagacgcccagaaccgccccggctccggctaacaataggatccggccgcagcgcactggctcttataggcgccggcacgtcctgaccgagacccggggctggctgggaaatggagtctacgggccggcccacacccagcagcatgccgggagttgtaggcggtaactgctgcaaagccgaagccggcgcgcaaaccggctcccgaggcggcagctctgctgcaaccttttaatgggggaagagaggagactcagctacagatcttaaataaacgaattctcctccaaagctccctttttctttcgtgtcctgaccacacgcca

SEQ ID NO: 125 - DNA, 656 bp, Artificial
  control peak
aacctgcacgttgtgcacatgtaccctaaaacttaaagtataataataaaaaaaatgcttactttaaaaaaagaaagaaaagaaaaaaaaaaaaaagaagggttcaggccgctgtgggaggcccagcaccacccagcacaagacagtacggtatcccgccgcgcgcccgactccacctggcgagcagggaggggccaagcttacgcgaggcgtggcatcccgcccctcgctgcattgtacttccgggctcacatttctgcatccggagtatgagaaccctcaaccaggctgaccggatccggaagtggattgcgagccaggaggaggaagccggcggtggccccgtcagcagccggctgctgagaggccggtaggcggcggcggtcccgaggggcggcggccgcgctgctccctgagaacgggtcccgcagctgggcaggcgggcggcctgagggcgcggagccatgaagctgtacagcctcagcgtcctctacaaaggcgaggccaaggtggtgctgctcaaagccgcatacgatgtgtcttccttcagctttttccagagatccaggtgagcggcacaggctggtgggccgtggcggtcgggcggagaggactggggtgggggtccgagtcggagtgggcctggggtcggcgga

SEQ ID NO: 126 - DNA, 738 bp, Artificial
  control peak
ccaggccaacacaacttaaaccccaacacaccagaagaggaataaaggccaaccaacttccgcccacccactacaaacgtcgagagaaattcgtagatgaggccttgtcaaaattcagaggcgtcctacgtctgctttcccaatgagacagttcctttttgtttgtctcagagactggggtttttttttttttaaaagactgtaggcagaaaaaacaccccaaccggctgttgtgacaatcaaacgtgtctaagaaaattcagccaatgagagtgcgagagtgcatcttgtgttggccaatgagaacagcgacccgtgcgcagggccgcccaatggggcgcaagcgacgcggtatttgaatcctggaacaaggctacagcgtcgaagatccccagcgctgcgggctcggagagcagtcctaacggcgcctcgtacgctagtgtcctcccttttcagtccgcgtccctccctgggccgggctggcactcttgccttccccgtccctcatggcgctgctccgacgcccgacggtgagtgtgcccggggaccgctctcagaggcgagtccgtcggccccacagctcccctgtcgcttctctcatctgctccgagcttctccgtcccaaccggggctgcttttctcttctccggagctcactgcttcgcccgcgtccctggccctgcggccggtcctctccggcccgcctttccccgtctggattccgccctcccctaaccc

SEQ ID NO: 127 - DNA, 999 bp, Artificial
  control peak
accgccgcaatggccttctctaagctcaagtcaccaacctttctccccgattctgccctatcggacaggtagttccctctctcctttcatctccgcctgacgcactcctccctcccttcaactccatttggagtccatttactacagcgcccagaaccccggcactacggctctttttagtctgaggactagctcctttttgggcccaatcacaactctgaagaatcctgcgcctatccgctgcatgtctcctcactaaactatttcttgaggggacagcctgactctatcattctcgtgctgcattcccaggggctggcctcgttgccgcttgagaaataccaaccgcagccgttgtcccaaagacactactaaaactgcccggaccctcccgctcccgagcctgcccgttagagcgcaaggaaaactcaccatccggcccccagtgcgtctcggaagagctcccgctccgaaacaaagctcacaacttccggaagcgatggccaatccccgtcttcttcgagccagtttccggataggccggctcggggtggccatctgtttccgggtcctggtaggaggggtgcttccgcccatggtcccgcccattcttccgcctccccaacctgggtcccgtcaacggacgcgaaggagaacaggggctgtatatcacttccggcgaaggaaatggaagaatctatgggctgggaccggaagctggggtctggttttgagtctcggctttgtcttaacctgtgttgggcgttgcaccgggcgacctcagtttcttcctgtacaaggaaaagtactgaccaaaatgagttctacatacatttccgctgctggagatttctttggtttttgagacagagtcttgctctgttactcaggctggagtgctgtggtgcaatctcggctcactgcaacctccgcgtcccggttcaagccattctcctgcctcagcctcccgagcagctgggactacaggcacccgccacc

SEQ ID NO: 128 - DNA, 506 bp, Artificial
  control peak
ggaagaagtctttcccaaataacctgggagactccaatgatcagagatcccgtttcccagtagcctgaaaaaatctccctcaaagctcctcgggcactagggccataggacgcgagctttacctgccgcctcagccgggttcctcggcgtttccgtcctattgtttctctcgcaccaaaatggctgcggccaggccgagccccgccccctgcacctcgctcccggtgcccctcactattggctcgtcctaggagctgcgcacgcggactggacagcctgaaaggcgctgttgctagggtgccaggattcgctgcagcagcttccggaaaaggcggtgactgggggcggggccagagagggcggagcattgtcctccgtctgggagggtgggccggaggtctgacgggcggttgggtggatagactagctgcgggccgatggacagcaggatacctgggaaacagatggattgaccaacagtcttggtttgttcagtagtgagcgat

SEQ ID NO: 129 - DNA, 595 bp, Artificial
  control peak
ctcctcggccagaaacggcgggcggtcctgcaacgtgcaggcgccaggtccaagcccggactcccctcttcacccacctgccaaagccgcaaattccgcagctggtgtccttcaacgaatgtaaccacctctcggcccggccggaagaagccacctcccccgcacgcgtttagccggcccccgcgattcggcctaatgcgacgattggctatggctccaaatcccgcccttccgattcctgtctctaggtcacctccgcaacacagcaacggaagtagggtgttgagcccgcccacacttccggaagaattgcactggactgtgggtatcctggtctccgcgtgtgtgagtaccgcggcgcgaggttagtggtagctgggtgcagacgccgtggcgctggtgagtaaaggcagaggcgaaaaaggaggaaacctcatggctgtcaggtctgtcccaggcctggttccctgccaggcacacgctagatgagtgtggttcggccaccttagccccgcttcgaagccggctctgttgtagcagcccctgtaaccccaggctttgtgttcggaaagtagcctaagtatcttctgtgccc

SEQ ID NO: 130 - DNA, 592 bp, Artificial
  control peak
gtcccccgaggtcccaccaccgctgcctgtggccccggcgggggttaacagggttactcgcttctccttaggcctgtgctccttctcttccttcatggctacgtcccccgctgaagcggcttggccggaacggggttaccccgggcacaaacggctcccgggaagctcccgtcaaagctccgcccccaacaaatgccccttgtgataagtcgggccccacgttccattgggtcctgccaagctctcgcgagacttgggcctttcccagtgcaaaggttttaacacctggtttttacttcccgcccgccaagttcctgcgccacccaatcccctcgcggcccagactccctcggggcccgccccttcgcgcgtcattagcaattccgccatcttgaagtgactctgtcggggactacaaatcccggcatacaaagcagctacccgcttttgttgggcgttcaggggccagagctatctcagggtccctgtttgtgagtaaatcgaatcctttcatcttcagagttttccaggtcgaaagataggttcttcacgcttagtttccatcagataaactcaatgccgtccaaatttt

SEQ ID NO: 131 - DNA, 859 bp, Artificial
  control peak
taccaagggcaatttctatggaatgagatgagccatattgtagaggttcgaagagtggatagtggtcaacacggatacatcgtttcctcccctcacggcatctgtctctcccactagacttttcctttaaaggcagtgacttatgaatattattccactgttttcaatcaattccatccaccagtgagatatattgcatcaggttacctttcattccaccgcatttgtgccttgcacttagtaggcgatcgataaaataactgatgataaaggggcttctgcagactccccaattcatttggtaaagaccgctcagcatcatggtcgggccctgattgctttcacaaccgcttcgaggagtgtcattcccatcacggccacaaggtggagcccacctccctgctctgggacctcaggtcgcgaccccggaagcacatccttcatcctctaccttccttctagcagaaatggcggctgcggcggctcgagtggtgttgtcatccgcggcgcggcggcggctctggggtttcagcgagagtcttctaatccgaggcgctgcgggacgggtgagcttccctccaggccggtcctgtcctcgagatctcgccagacccggcgcagcgcacctgagagtcggcgccacagatccgaacagtgggtcgggcagggcctcctcgacccggacccgccgggcgccaggcggcctggggctgctgaactggccgggggtgactggcttgtggccacccgccaggagacctgctagtgacgtgtgggatgaccctggttttcggctcctcccgccagctactgaggagtgcatgtttgaccactaaaaacgtagtgcttgcaaattggg

SEQ ID NO: 132 - DNA, 415 bp, Artificial
  control peak
caggcttgcgcttgctcctgacaccacttcctttacctccacggctccatctttgttctgcgcgagtgcgcacgcgcaggctccgaaagcgggccgtcgcacagagggaccacaactcccagagtgctccgcgtccttgctttcgcctctacttgtgctccagggcgcacgcgcagccctgggagcgggttctcgcgcatagggaccacaactcccagggtgctccgcgtcctcgccgctgtcgccgccgcggagacaaagatggctgcgagtaagtgcaggttccggtggcgcacggggctcgggtagttctgggaacctctgggcggtcctgggactgaggtgcggcagggcaggggtggaagcgatggggtccgtgctggaggggaacgcagaagtcacgagggggctcctc

SEQ ID NO: 133 - DNA, 767 bp, Artificial
  control peak
agggtccgaggagacacattccaaacccggaaagggaaatttggaatttaggtttagggtaaggagagattaaatagagctgggataccgggtttctaccctgggagatgaatacgctcaagctggctggacaagacaccttatgacaatacagtgaacgcacgtcactccttacctgttgcctaagtccacggccacagctcgagaccctgcgctactccatagtgctcgcacttgcaactgggtgagtgcccgatcccgccgtggcagaaggaaaggaccatcgctcaggggccggagccggaggcacgagcagcagtacctgcaggccgccatgacacccggcacgcggtcaacgcaggctgtgccctgattggctcactccgcagaccgtggggcggagcctgatggagtttcgtttccgcggtgcccgagggacgccggggtaacggaaaccttggccaccttctacgaagactagagagggtcaaacctatcagaacgcgattatgttgcaaacgatctttgtctttatgtgccaacgaaaggcaattagataaataaggtttttgtgtgtgtactatcgtaatttaaaggagaagtcctagaataatgtaaatgtttaatatgcagagtaaatttatatttacttttaatactagaagtcatagaacaacacattattattatttggatctgtgaactaagaattagaatttgggtggtgggccttggaaagagatctccttctccccttctccgccctg

SEQ ID NO: 134 - DNA, 510 bp, Artificial
  control peak
gtcaatgtttcctcaactatcaaatgggtaaaatggcagtgaaggagcttcatattattaagcatttcctggtgggtggaatacgccatgaactaggtgcggcctcaagctggttactcgccctgcctactccctctcgtgtcctcccagtacccccttagacccaccagctctgagggaatcttcgcgacctgcgcaggcggtcgctctgataaccgcatcccggtttccgggacgacagcggaagtcacttctcacacgcctttcttattggataccggcttttcatggggccgagcgccgctgggtaggcggaagtagccgcaggcatggcggcggctatgccgcttgctctgctcgtcctgttgctcctggggcccggcggctggtgccttgcagaacccccacgcgacagcctgcgggaggaacttgtcatcaccccgctgccttccggggacgtagccgccacattccagttccgcacgcgctgggattcggagcttcagcggg

SEQ ID NO: 135 - DNA, 588 bp, Artificial
  control peak
tataaaggagaaaatgaagaaggtgggtaatgcaacgaggtgaaaagaaacgttcctactggcgagaaaatgaataatgatattggtctccttcgccctgtaaggagccaaccaacctacctacagtacctcctttcctcactcgcgccttcccttcttctgggcagcgaacacgtttctatcgcgtgagctacctcttccggggttcctgggtgacaagttccggatcaagaatcacaaagcgagcagtcattcttcctcttgccacacccacagggcccgcactaagagcctaactgaaatcccgcgaggatcaaccgagctcgccgaaaggagggaggaacgtatcccttctggaggctgtctcagggggcagagggaccggaccggaagtgacgtgagcgggttccggttgtctggagcccagcggcgggtgtgagagtccgtaaggagcagcttccaggatcctgagatccggagcagccggggtcggagcgggtaggtaaactaactcagggaattggggaccgaagcctgaggctgtcctcagagcttggcgtcttccggagctctcccacttaggcattg

SEQ ID NO: 136 - DNA, 500 bp, Artificial
  control peak
tgagtacttgcccccttggaggttgtgtaggttggctatacagattctacaacttcccagacgagtggccctgagacttggggctgaacgcgcgagcacggaaagaaatcccaccgtaggacggaacccagtctcttcagctccatcctaaacttcgcaagttttctctgagtctacgcgcttccttaggactacaactcccagcatgccacgccagcggtcaaagcttattggtggtcctgccaggtcgcttgcgttgtggcagtcatggtagccaatggaaataaagaattatcccaagaaaccaatcctggagtagtagggcggatccttcctgggttccggctccggctccagctccttccctcgcctacagaggcggcgctgtccctgatcgagcggcgggagcgaggtgggtccgggttgggcccgaggtcgcggggggaggcggacacgacttcgcgggaaactctgtacacggccacttccctctcaccgag

SEQ ID NO: 137 - DNA, 699 bp, Artificial
  control peak
cagaactcggtcatcgaggcttaggcggcagcgggggtcgtcagacaccagttcgcagagcaggtacctgcggcaggcgagaggaaggtggacactagcggggccgcgaggacccagcaaggcactgggagggtctggaagggaatgcttacctgtgcttgaaccgcaccatggctgcctccgcgctctccggtccggcgtgcaaaccggatgtgaattctgtccgctgccaatgggaagcttgcttccgcagcctttcagccattcagagaaggagacaggatattagggcggagctagaaagttgagaggccgaagtctgtcagctctctcaagccccgcccaactgcgtttgtaagcgcgcatgcgtcctgcggagggtcactgactagtttgagggctgaaccccgggatggagcgccttgcagctgtaatggatggttctgttgcacactgaagtttacaatagtgtgtaggatgtagccgctacccttggggccctgggaaccttgcaccacagttatgtatgcgagtatattctctccccaaggtgaacggtggagcttttgagggcaagaccctgcacctcacagcccaggcattgacaaacgggaaatcggacttttattttcaggtttgagctcagtttaattcttccatacagctaaatatgctattgctgtttgggcctttcagcaa

SEQ ID NO: 138 - DNA, 692 bp, Artificial
  control peak
cgctgcctcctttttccctctccggagcccccagctccgcctccaccttctctccgcatccaagccccagtcccgcttccccaacccccgctccgagtcctcagagccccttcccttcccgtcctacgtcccctcccagcctcactccgacacgcaggcctcccctcccggcggaccggcgggggccgcaccttctctatcagccgctgcatgcgcctctggaagcggcggcgactggccctgagcttctgcagcagctggtcgtcttccacgtcctcgccctccatggcgcgcagcgtacccagcatcggacccagccagtacccaagcgccaacccggactgcagggcctcgcgcgccacaaactcaaataggagactccgccctccctgccttcctctcggttttctattggctagagcggagaagccgggcactgattgggcctttcaaacaaatttgcgtcccaccttccggccaatccactcccgagtttccagccacgccccaatgtccggcggaagggagggagggtctccggccgccacggccggtggtagttgtacaaacagctgtgtgcttattcatcagcggtgctgtgcgagatccggtgggatgcgtgtcatctcccgcgctgtgcaccggagataagtgcggcctggagatttctgcgtgacttgctctgggtgtcg

SEQ ID NO: 139 - DNA, 442 bp, Artificial
  control peak
ccttggggagggttaccactctgggacctcagtcacactatctacaaaatgggacacttctctcagccttccttcttccgggccgctttcaagcctgggcttctggaaatacgcaggcgcgagatgaattgtttgattggctattcattgtgatgcacccacgagctgaagctccgtctaagccccgccctttcctgtcgtgacttaacgcacgcaagcggctccagggtacgtccccgccacgcgcgctcgcaggatcggtgcgtggtgacgtttcgccggcgcgggcgccatcccggaagcgcgagcaaggccgccagatgtgcaggtgccgccgctaccgacgccggggccgagtttggggtggggctggggactccagggccgcggggaaccggtccgggtcgggcgcggcccccgggctgcggtggggtggggtgcg

SEQ ID NO: 140 - DNA, 861 bp, Artificial
  control peak
caagaatccagctccgggtgaggacttgaaacagcgcgaggcgggaggacgcgtcagaaagggttttgctaaggaaagtttcgcgacaggaggtgtctgcacggccccactgcagaaagaccggggacgggaccagcctcagggcaactttaaactcaaaaggaagcgactcacggactctcacccggacgtttcaatttgctctgggttgagaaaagaggggccggaacctgcaggtctgtggggaccccacgtcccctatatagcagcaccagacaactaggaaacgcacacgccgccgtgcaactttcagtccacgccatccgcttccgggtctgtgccaatcggcgcaggacagaagcgggacctgggccagatccccgagaggtgggcggggcctgggcgaggccggggcggggtgcaaaggacaaggggcccggcgcggaagagaagagtcttgggctgggaggagtccgcgggggcgggaaaggggcggggcctgagcttttctccatctcgctctccgcgcctctgttttcaaggttttagaggtgaaacccgccagtgaggctggagcagctcagcagtcagggaaatagtacctccctgagaagtcgccgtgtggcggtaattggatccgggaaagttccttgctattgaaatttattttagcaatttaattccaaagcctggaagttgtctacggcaggaatgcaaactagaatgcgaaatgcaaagccttgcctgagcgaaaggtacaatttcatgctgacggtccacagaacagaatagaaatcctctccctttcaattctccattcactggagtgggagagtccacactgtgctcagctcctgagac

SEQ ID NO: 141 - DNA, 714 bp, Artificial
  control peak
cgcctctactaaaaatacaaaaattagccgagcgtggtggcacgtgcctgtagtcccagctgcatgggaggctgaggccggagaatcacttgaaccctggaggcagaggttgcagcgagccaagaccgtgccactgcacttcagcccaggtgacagagggagacgccctctcacaaaaaaaaaaaaaaaaaaaaaaaagtgcagtctagcccgcatctgcggcctcttcatctcgatgatagtcttttcctgtgtcttagcgttgcatgatgggagaaatagtctactttccggtagcggtgccagggcagtggcctaatacggaactccatttcccggcgtgcctcgcggaggccgctgaactcagaagcgggaggccggttccggttgcatcagcgagggattcacggcgaaatgagactgttcgtgagtgatggcgtcccgggttgcttgccggtgctggccgccgccgggagagcccggggcagagcagaggtgctcatcagcactgtaggcccggaaggtactcgtgctggtgctggtgggcggtggatgggggggcgggaccgaaacacgccagattctctgcagctgtctttgccaaacccctagccctcgccacacaccccaatcgaccacttctcctattatccttgatcactccacgccttcttccctcccagtcacatctttcacttcctttaattaccctca

SEQ ID NO: 142 - DNA, 472 bp, Artificial
  control peak
cggctgcccccgtcccaccccttcaccctcccctcgcccccttggggacccaggcgcgcccaccgatgaccccatcttcaagaccccccgcgcccgctagcgagcgcgacccccagcccccttctcacttcatggcgccggccggactgtgcagcggggtcgacccgcctccctcatgaatattcagcgagaggccgggtcgtggacatcctcgagggctcgctccacctatattacgagaccattggctaacctgcccgtcaatccgctagggcagagccaatcgggatactgcgcgtgcgcacggaaaagcgagggcggctgactctcgggtgaggcggtgcgggaggcgtcactgaggatcgtcgagggccaatcaaaaagaaaacatggaagggaaagagccgagagactcgatctcattcactagaatttggtcctcctgcgcctgccaagattgtctgagtattga

SEQ ID NO: 143 - DNA, 451 bp, Artificial
  control peak
accccagctagagtaatacaggtagaggaaccaataggtacaaaggcttggaggtgcatctgagccatgaacatttcagaacctggtaaccatgttgtgtcatggaaagtggggaactacattacccagaaagctctgctttaaacgacagccggtcagagccagtgagcactcggaaagaaggcatttccacgtgtgcacgtaacgtaaggccgagacttccggggtctctagtagcggctgtgtatcggcgatgcgggtgtttccccagtttgtggcccctgagtgctgggtgggaccgcggtgactgaacctagaaggtggagaggaatcgtcctcggtgcccagaggcggctctgcagccccgtgacggcgaccactgctcccgggccgtgcttccccaagtagtccgatggcagcggctgtgccgaggcgcccaactcaggtaatt

SEQ ID NO: 144 - DNA, 503 bp, Artificial
  control peak
cagccactgcactccatccccggcaacacaacgagaccatgcgtcaggaaaaaaaaaaaaaaaccaagaaaataaaaaagaaaccacctaaaggcgtcctccgcgcgcccgcccctccgccatctttgcgtccgccccgtcctgcgccgccacttccttcccagccagaagtttcgcgccaaattgttccgcacacaaaatggagcccggaaggaggcgaggtcatgcactttgcagccaatcatcttcggcagacagtccgggcggcggggctggagggctcagaacggtccaatcgctggccgcgaggctccggcgaaccaatgggagcgcagcggctgcggcgtccgaacaccgcctcttgtttttcccgcgaaactcggcggctgagcgtggaggttcttgtctcccctggtttgtgaagtgcggaaaaccagaggtgaggctagtgggagtgggactgggactgggagccagcaggcatctggatttccgggtgtagt

SEQ ID NO: 145 - DNA, 530 bp, Artificial
  control peak
aggttggggggacccctgaacggcgctgctgtagcctagcagccctggcctgtcttgatttacccttgtcgtccatggcgtcctcctgctgcggagccccaggggacccgagccgcccgcgctgtctcgaacgggtccgccggccgcgccgcctgcgagccagtctgacagcgcgctgcacacctccgcaccccacttccggcgcacttccgtacccctcttccggcgcactccccgcactccacttccgcagattagggggctggcacgagtcacagcacctcgtgcgtcacttggcggcgccggacgttgggctgcgcaggatttgagacgctcccctgaccaccacttgctctgcgctgaggtgctgggacagccatggtttcagacggtgaggaccctgcagggcgggacttcgactccggggctcggctgtctcggtccggctacgactggccagatggaatgtgtcttttgggctggatagaatccgtttggtctttagaggagtagccaaggcattcagtcat

SEQ ID NO: 146 - DNA, 476 bp, Artificial
  control peak
agagggaactcctgcaattcggagctgcggtattgcagccggttatacaacgtggggaggcagcctggctccccaaagacagcgcagcctcgttcccggagggcggcctgcctgggacctgccgggcactccgccaccctacggtgatgcagcaagagccgcgcggtccctttaagaaacccggctaggcgaggcccttctgtgatcccgtctcctcccttggcccgcgcagctccgacggagcaggccagtgagtgacgggcaggtcgcccaatagcagcgtgcagaggcaggggcgtgccccggcgctgctacctgcgcgggcaagctcagcgcacttggcttaaggggcggcgcgctccctgcctgctgctgggcggagggaaggcggcaagagctgcggagcccctggaaggtgagaaggactcggagagggaagaaggcccgagactcgagaatgcggggttggggccggg

SEQ ID NO: 147 - DNA, 544 bp, Artificial
  control peak
ccggatctccctcacgagcctcgggccgcggcctcctccgcctacaacgtgctgggatcggcaggggctctggcccgctccggccgacctgccagcccaccccagcaggacgctgcagggcgccgtccccagcgagcctgggtagatgccgggctcggcgaggcccacgtgcctcccctggagccgaggcctcacgcggagccatactaaccacaggagccatggcggcagcggagttagaaagggaggtgagcgaactacgcagacgcaaagagcccgcagcgcgcaaggcacgcagggtccaggccgcactaatcactttgccacgcccctcgtccgccaccttttctcttggttatgtacgataggggagcgattggtttttcctgaatgcaaaacgcgtcctctcccaggcctggagggtttctggggaaggaaggaggcccggcgggctgtcccactgcgaacgatcggaggggggaagccagactggacgagcccaaagataaataaaggggttggctttactttttgaaagtcttgg

SEQ ID NO: 148 - DNA, 766 bp, Artificial
  control peak
gtcactgtttcttccacacaaacttagctgctctttggaattttctggggtgtttttataaatactaaggcttgtgcctcatttccccagtgtctgatttcaccgacttggggtgtactctaggcactgagacttgtaatagctgcccctggtgactctaaggtgcagcaaatgtcaaagaccaggacttggtgacgccaggtccctggctgggctgcggtcaagactgcatgacctgctgaaatctccccagactggttagcgcggaaagagcacctccgggttaccctttcgtccggaagccccgccttcgccgtcgccccgcctttccccggctcggccacccctccttcccgcccagctgtggccccggatgacgcaaaccgggaacagcggctgagcctgcgcttcggaagcatggatgtgcgcctgcgctgcgctagggcgcggcgggcggtttgaattttgcttacagagtcccgtctcaccatcctgggcttccaacggagactgcggtatccgcagctggagacccagcggcgagtagccttttgctcccggacggacttgaggttggagtctgtgtgttggggaccctgcctttggaagcgcccgaggtagctgcttggcagctgggggccgcggaccgcgccgggcttgggggaggacggggctggaggctgggttggatgctttagaccctccccttctctctcaccggcggagtgggtgggaaactggttctttcccatttctcggcctcctt

SEQ ID NO: 149 - DNA, 720 bp, Artificial
  control peak
ctgaatgaaatgggagccatcagggcatttcgacagggtagtgacatcttactgaagcttaataagcatcaccaggacatcgcgggagcccagcaaacgcgcttccctgccactgggttagggaaggcttgccggaaggacgatttcggaggacagcaggtgttagccaggcagcggagcgagcgccaacaggcgcactggggcggagagacgccgtgagcaaggtgaggcagagggcacgcgcggcgccacccgccacaggctaggacgcacggctgccagagaggacgccttaggggcccgcgagcaggctcgggaataaaacagggctggcagggcgaggtggagagactcgagaaggaccggccagatgtcgaagttcctagagaggagggcgcccaagcctccgccgagacgcgtgactacaactaccacaatccccagcgcctgcggttccagcgtcgccttgctctagacgggcggcggcgctgcgcggggaaccggcgtggggtcacgtggtaccggcgcatcacgtgggcgagttaggtgacgctgcggggcgggcggacagactgcgggacggacggtggacgctgggacgcgtttgtagctccggccccgccgttccgacccccgccgccgtcgccgccatgacggggctagcactgctctactccggggtcttcgtggccttctgggcctgcgcgctggccgtgggtg

SEQ ID NO: 150 - DNA, 551 bp, Artificial
  control peak
taagctgccttcgacggccgagaagcgcctcgattcttgcccctggctcatttccctggcgcgttttccagacgcctccgctcccaacgctgggctcaaccctgtcttccccagctggtctcccagcacgtggcctggagcccagcctcccgccccgctcggtcacgtgagcctggctctcacccgcgccggcttccacaccccgcgggactgagaaccgccctcccactagctcgcaggcgcaggcgcagggcagggtcccctcagtttgggtgacggaaggggcgtgtcgcagcgcccaaagatgacgtgttctgccagcgccacgtcatgaggaagtgccggcccactgaagccgtggggacgcgcccagcggagctaatcaggtgccactgaactgaaggggtgaactagaggagaagagggtgactgggcggggagcagctgcgggagaagcaaagggacgactgagggaataatcaggagaccactgaggcgtgaagtactaggcgtgcgataactgaaggagttagtaactgggagcacagagt

SEQ ID NO: 151 - DNA, 628 bp, Artificial
  control peak
gggaagagcagccggcagagtcgcgatggcaaacctgtcacgacagagctcgcaggtcccgaagtgggggtgagggtgggggttgaggatgggaagacctggggcaggggaacagcaggcaggagaccgaggtggggcctcggcggcgggcgaggcctcttccgccctgcgttgcctcccgccctccctccgggccggccctccccgcctggctgcccgccgcgccccggggcctcacctgctcctgcgggggccggagccggggccgcagccagcgaacccccgccagggttgtcaaggagcgaaacaagagtgtcttagcaaccagcaaggaagtcctgccccctcaccggacgttgacgagaatttcacttattattggtggataaaagtatcaatcagagtgaaaggcccttataagtcaggcgtttgcaaggacaggggcgtggtttagaggccagggaggcgggacttcacttatagttgtcttaagcgtgcccgaggagcaggggacacattgaagttctttttctagggaaacaaacttcgaaaagcagtactgccttgggaggttcttttagtcacttgaaataacaaatgcaacttgttggtgtcgtgacacttagcc

SEQ ID NO: 152 - DNA, 684 bp, Artificial
  control peak
gcgggacagggccgggaatggggcacgccgagaccaagaagccgaccgagagcaagaaggcccagtcatgcgcacccggcgcctacatcctcctgcggtcccccaggcctctccccataggcatatgagctctgctccactcctcgcttgccggtggacgcaggcgccgcggccgccagctctgtgaacgcagcggggtctcacctccaggctacccccaacctcccgctcggtcgggggcccctccacggccgctatcactgggtcgcggctcctagcgccaccatcttggaccccgcgcctgggccaggcgagaacttccggtgcgctccccgggactgcggtcccccagtccgcgccccgcccagccgtcccccgtcggctccagccaatccgcgcgcgctcccctggccgtctcccaccggatcaggtgtctcctctgcgcagactcctccacgacgccagtcggcgctggcgtagctcggctcagcccggcggagtctgacttccggagggccagcggcgcgctggctggttaccaaggttaccgccgagaggccgcgtcctcagctggtcctagacatgcgtggcgtcggggctgtggcacggtcgggtccggggtgagtgaccccttcaagtaactacgtttttgttctgcccttttgttttgtttttgagatggag

SEQ ID NO: 153 - DNA, 736 bp, Artificial
  control peak
gacaccagatgacaacggtagaagtgggggccggactgggagggacgtggccatgtttgagttgagctcccctcacaggaacggtgggtgctggagacaggggcctgccttcttctaagcggcgtgggctgggagagtccggccggagacaaaagaccgtgattttgagtgtggggacacggccccgtgtgaatgggactgggtgggtcactgggcccccctgtccccgggcccctagcccaccaccgccgcctcacctgcctccgcagccctttctgcttccagtagcccgcgccactggccaacctattcagttgccaggcgacatcgttgccaggcgactccagcagcgtccgcggcgccgcggtcgacataatcagcacccactgcgcaggcgcaaacagcccaaggctgcggccggctgcaaagcgggaacaatcatttacagggttgctgtggagaccaaaggcagctcagctttgttcaaagtacagaagggaaaggaagtgacttatttacgcccttctctgtactgggtactttgcatgcctctgatcctcagagtaaattcataaggtggtaacatctagtttagagacaagaacctcagagaagttcagtcgtttctcccaatttttttttttgacggagtttcgctctttcacgcaggctggagtgcagtggcgcgatcttgactcactgcaacctccgctccactccgcgccaccgcacccaa

SEQ ID NO: 154 - DNA, 737 bp, Artificial
  control peak
aatgagttcccacaaaacagaagagaaaactgaggctcagggtacagcactgcccacagtcgccctggaagtcagaaaagggctccagaagccacgaaagggctccggattcagcatccggaggcgagcagggtcccgcctccgtaatccagcgggggccagggagccgcagggggttccggagaggacacgggcgctgttcccatcctgctgaggctcacgcggcaccttcgggggcttccggcctgggatcagcgggaagcaggcagcaggaggcgcggcccggccccggcgaaggcccccacgccactggttctcacgcctccaccatgctgccctgcacggggtcccgccgcccgcacttaccgggccgcacggccgcttcctgccgcgctccgccatgttccgcacacaccgtcggtcccgcctctgccaatgagactcgcctcttccggccacacctccattaagctgtccaatcggagagccaatatcgcccaggggcgtggcctgggcgcccgacgggctgcaggggtacgcgggctggagtaggttgggcagagtaggtgtccaaataggcggcatcgcgggtactgtgctgaattttggatggctcggagcccccctctgagttgggacaaatacaacactcagacctctgagtagttcacttgtttaaaaataattttttttgagacagggtctcgctatgttgccgaggctggagtgcagtggag

SEQ ID NO: 155 - DNA, 673 bp, Artificial
  control peak
gtgtgcgttacagttaggagtatggggggagcggggaggcaaaaggaatctgatgttaatccctccaccgccccaggctgtccccagggccacaagtacctcggagattgccctcgtcgctgaaggtagtggtaaggaccccctcggtgaccacgcagccgcagtcggagcacaccagctggctctgcgaatagtgcgagtcttccaccagctccgtggagccgcagtccgggcagcggcctctgcctggcatctcacaaccggccccaaagccgcggaagccttcagagactcctgggtctgcaacagcaaccgtgaggcagcaagaagtaggaggggacacttcaaagggttgcacccccgtcttccggccagcgcgacggaggcataggcggttccctccgctgggaggctgcgcgccacttccggtctgtgcctccgcgggctcgggtagaaacgtgagctaggagaggaggttcctgggctgaactgtagctgtctggtttccctgcactctgtagttagtttttgggacctagactggggttgaatttgggaaagtttttgcctacatggacacgtataacagacagaagattgttgtcacccctgaacaaccctccttttaaagtcctaagggaatcgcaggttggtaatgaatgtttaattggcc

SEQ ID NO: 156 - DNA, 548 bp, Artificial
  control peak
cggaggcggatctccggcatgtcaacgtcgtgggcttcgcctggccagaaggatgaggtagagtatggggtccgaggggcagggagagtgggagggggcggggtgcgccctgcgctggctaaagtgcgcaagcgcgcgaggctcgggcctttcaaaccccggcgcgccggcgccggcgtcgacactgcgcaagcccagtcgcgcctctccagagcgggaagagcgctgcgttccttagcaacgagcgtttcctccagccccgcctccctccgccacacacaaccccgctctagcctcgcctcccaatgacgtccgaaccctgctttctgggagcctagcaacagaagcgacctccgggattggtgtctggaagaaattcgcctttctcattgcacgatggagttaccgcccacactgaagccgagaaagcaaatctttttgctgtattattatttattttattttttaaaaattttttgttgagacagggtcttgctctctcacccaggatggagtgcagtggcacgatctcggctttctgcaacccc

SEQ ID NO: 157 - DNA, 744 bp, Artificial
  control peak
cagacaacggagaggaggtgggtgggtgcgccgtcctatggggaagaatgaaggggagagggtgtactgggggcacagggctttgagaaactgggagcaacaaagacgtgaatggacttgggtaggtctgtaaggggttcttagaggctgtgagtggatgggggtacggatagtgaaagaagtggggagcccggagaagtggtcgggattgtaagcagaaagtggggacctcagaacaccgagctcaggttggaatgatctaaggacacaaaagcatggggcctcggggctggggcggtgcgactaccggaggcgcaagttgcgcacggggtcccgggagcgatacactgcctccccggtgtcagtgctccagacggtctccgccatgacagctgcgacgcgccgcgactgcgccggaaagcgcgccgctctgttttcctggcaacggagacgctggatcgggaggcgacggaggtgctgcgcgcgcaacccgccgttcccgtcacggcgccacggtcgcagttggccacacagtgcaagtgcagcccaagccgaacgcccaagagggctccggggtcctaccgatcaccgcttaccatgtatgcgataaaagctcaccgaaagcgggacctcgtgcgctgtgcaaatgaggaaactggcacagccatcagcccacttcatatagacagatagatgcagaaatgatgacggggaccaggtggcctgagtcttcgccctctacct

SEQ ID NO: 158 - DNA, 530 bp, Artificial
  control peak
aaaaaaatgaatgaatgaaagaattatgcagtgatactgcccagaggatagagtgcacgtgaaatgcttaacagtgaagtcggtgttcctgatggccgcgggtcggctgaaccggactcccagcgcccgagaggaggccaggggaactacaatgcccacaaggctccgcgccttcctcgatgtacttccggctggcctgggactcaggctgaacattggttctctgtgcggggcggtggcggggattggagggcgggagctgtgctgacttaggattggcaaaatcgctatcagccacgcctctaaaacccggaagtaacctccccggtagtcccacgtgtagcggagaaacagtagttaggatggctgaaggggatactcaccggctgaaggccgactgtgattccccctacccccacaaggcgattttgaccccctgagggctgctctagaggactcaggccccgaagctgtcccagggaggtccccgctgcatcccaccacccaagctgtgcctcatggagtcgatg

SEQ ID NO: 159 - DNA, 796 bp, Artificial
  control peak
tggtgggaggatcacatgaggccaggagttcaagaccagcccgggcaacatagtgggacccccgtctccataaaatttttaaaaaattagcctggcgtggtgggcgcacgcctgtagtcccagctactcgggacggccagaatgaacggcattatcgtctgtctcttcattctgattctggccagcaaggggtggcctccccctgcggtccttgctgttaggtccctgcgccccctcagctttgcgctgaaggtcgctttaccggaccccacttcgccctaccaggcgcaggatgagcgtctctgttcttttaatgtggcgtcccccacaggccaccgtaccaggccgccggtccggggttagggcacttgacgccggcgccgccatgttgtctccgggccacttccgcgtttctggcgcgtggtcttgcatttcctacttggtcctgttcgtggcgccgcgcctccgggtgttggggagtccgggatgatggggaatccggggctcgccctagtcgcggggacaccgccttccaggagctgtccccaggcaaactcacagacatcttcaaagagccttcttggacattctgctttttgctcctcataacaggcttcggtaagagaacaggcaaacatcactgcgggcgcctctagggctcgtttccctgtcccagtaccgggaatcggggaggggtccaagcagagttcacttagttaacaaatcagtgaagcagttggaaaatctcagttatattttaaatgagatcgagggcggtgactcacgcctgtaatct

SEQ ID NO: 160 - DNA, 691 bp, Artificial
  control peak
cgggctccagcccactccccgggactgccgccgcggcttcgtaagacccaaccaagcccctcacctgcttttgcacgtcagcatcgctgagagccatggcgagagcaatgctaggccggtgaacagtaggctcgagtttaggtttgaaaggtgaggtgagagaaatcggcaaagggaacccctgcgcagatctcgggttcctttactttataaccgcgggttccggttcctgccaggtgactgcacagttcatcctcatgacccttctcagccaatgggaagagagcgacgcccaggaagtcccgccctgtcccggcctgtgggcgcgtcctcgggtccttctacgtcgctgactcgtgacctgaccggtatttttttcctaaactgggatcttgggtaggaggaagaaaagataaggagttcctctatctgaaattagtcgggctgttttgaggagtactggttaggtatattggagaatgtgcctttattggaatttttcgcataattacacagggttatgggcttaggggtacatgattgttggccgggcgtggtggctcacgcctgcaatccggagactttgggagaccaaggcgggtgtatcgcttgacctcaagttcgagaccaggctggacaacatagaggaaccccgtctctaccaaaagaaaaaaaaaagggggatacatga

SEQ ID NO: 161 - DNA, 604 bp, Artificial
  control peak
gcactttgcgttccccgcgcgggcgacgaaacacgctctcgaggggagagagccgtgcgtcctctgggccgtgaagccaggggaagagggttcctctagacagctcgatgtgcccgggagagacacccacgctgaacctgcaagctggggtgctccaccagcgcgaacctctcgccccgcactcgccgcagcactgctctcgtggtaggcggggtgggcgggacggcaaatccgcggctcctgcgtctattgggctgctcggggcagcgcagctccgcattggccaggccctcgctccgcccggcttccgcgagcccattggtcgcaggcgcccgaccgggtcgcggccgcgcgctccgcccgccgctgcgtccccactatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtaagggcctgcccagtgcgcttcggggcgcgatggctgaccgcgaataacggtcgccgctccgccccgcccctcccccgc

SEQ ID NO: 162 - DNA, 989 bp, Artificial
  control peak
gggacgcgggggtagagggctgcaaggcacagggagcctaaggcgccaggccctcggacttggaggttgcctggaatgggtcagtcacctgggagccggttcctctgcctcgggcttcggacgcacacggtccgcaccaagggcaccagcttctgcttcaaagcacccgccgccaggcctccacaccggaacatttcggcgaccgctattcgggacctcctccttccctttcctctcacgcacgaccgacgggtcaaacaccgtgagccccggccagctcccccagtctcttcacacgcacattacgcagactcctcctcccagctccgggtgggcattggacagagaagccggcctgctagagccggggaaggggaacggcgaccggaggcctactgcgcaggcgtagcactcgctgccgcattgcgattggtccgcacggcgagaggatttgcgcatgcactagctggccgcaagctcgcaaagacgctggctctaggtagccggttttcgcgagtttgtggcacagtgttggactgaacgagttaaggaacctgttcaaggagggactcgtggggccgctttgattggcggttgtgcctggtgctgccgctgtgttggtgacgagaactgataacagcatctcacaacagcgtagccccggcatcgcactttgttttttggttttaggacaccagttacttgataccgaagccttcctgattcttttgcagtgatccagatccagtttttccatgagtccgaggcgcggaacctggcttcccagatccggcgtgtacttccatcttttaccagttgccggctctttcccacccaacccgtttcctggacaagtaattctgcgctcttcttcccgcctgtcacctttatttattagttcagttgtgtgtcctagatgctgtataccaggtgctacagatacacacagtctcctgctgtggtttctgtgcttgggagcaggatgttag

SEQ ID NO: 163 - DNA, 821 bp, Artificial
  control peak
cgtggcctaataagtggcaaggaggaacacttcccccaaatttcttcttcttagtgctttgccagatcagatctgggagatttccccctcccgccggcttgctggatgacagcgatccggcctggactctgccctggggaaggcaggaagaaggtagtgaaaatgattaagtcctcttattgcaggcgccaaaatggggcagaaaccctgcaggaaggccaggcctctgagcccagttccacccccgttcccctcctcggatctgctccggtcctccccatccggaccccatcccttcggacgacctggccagctcctttcctttccccatccccacccggggagaccccgcgcccagcagctcaccgcgctcgggcactgctccctctgccacgcctgtcaaggctcggcaaacatccggctccgcggtggaacgggaaccgccccctcccgctctcttctatccccaaccatttccgccacacctccaacccacttccggccccaaacatggcctttccaggagccccgcccacttcctgcacatgcccgcctaagttcctgcctcggggaggtaacagcataactgtccgactcgtcgctcccaaagagatggctggaacaattagttccgctttcccagctagtcgaatcactggtgcacctccggaatccgaggttcgcattgctctcggacagagttctgcctccccagtactcccaaactcccagtcctgtgcaccaatgaggtccagctctgaggcttctttcctgagaaaaaaatttggaagtccgtgactgtttcctggaggagctgagaa

SEQ ID NO: 164 - DNA, 547 bp, Artificial
  control peak
tgccgacccagcctcgatcccctgcggcgtccaggtcccaatgccccaacgcaggccacccccggctcctctgtggactcacgaagacaaggtccggccgctcgggccgcgagagtcgcgccatcaccaccatttttctggatgcccaggtacaggagagagtcgcgcgcttatgacgtctaccactgacggcccgcccctccgagccagacccgttttctattggttaaatcatttttgacggacaggcccacgttattgacttcttttcgacgacgccagcgaccggaagcagaatagaggcgccagaagatgcgccatcaggatacacattggccaatcagcttcagcaatggagcgtgcaaaacaccagtgagcttctgtcttgctggagggtcggctttgggcggaactggctttgttgaccgggagaaacgagatgggggtgaagctggagatatttcgggtcagtggacacaggagtgggttgggaggctgggcaggggatcttcctggatctgaagatgaggttggaaatggggggact

SEQ ID NO: 165 - DNA, 991 bp, Artificial
  control peak
tatgtgcaaactgctatggaactgtcacgtactagatgctatgaagttaataaaagaattgagtaaaggtccctgtctttgataacttatattctatttggagaaacatggggggtaaatcttggctaataactagcaaatataacatgttaggccaaggagtagagataattaagagaactagtcgagaaggcattggctattaaaaaatggacaggatttatgcaaactctcatgaaaggtgggacttcatgatcctacagtcaatctagctgttctgggctttgaaccccagcgaccccatccagaagaaaccaggagaagcacaaactggcgctaggttcgctaccatccacctgacccttgagcccggggaaacccacgggtgcagcaagccgggaaagctgcagacgaggcaggcggaagaggcgggacttcgcgggtgacgtcatcggggcgccggaggcccggggcgcctgggaatttgaagcaaacaggcagcgcgcgacaatggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggtgctggcctaggaagggaaaggacagggcttggtggcgggaaggaggacaaggaaatggaagagggtgacaggagttgggccgtgagtgagagcaaaagagccaagcaggcagaccgaccctactcttggtttggggagtaagcatgagaagcacagaggccaggggttgggctcctcgagagtcgtctgttctttcacccacacgcttagacacgtgttttgcaaaagtcgggaaacccagtttggaacgctttctggcactgaagcacacttgaatgaattctgtatatttaactctttaagaaaattagacttgcggccgg

SEQ ID NO: 166 - DNA, 791 bp, Artificial
  control peak
ttgatgaataactcagcagagcccggtcgaggggtgacttccgatggagaaatgtgttcgctactggaaccaggtgggacgaagggtgcttgacatgcaagaacctttagcctcaaagcagaggccaacccaggacgggcaacacaggagacccacgttcctcaaatctgttctccacgtgggctcctgggaccccgcactcaccaaagtcgtaaaggttctggagcagagcgtcaagaagtgtcggagaactgggaggcgtggaggaccccggcccttgctcctgcgacattgtggggtcagccgaggaatcaaccctcattctgccgcgctgcacccccgcttccgggttccgcgactgcgcgcatgcgctgcagggccctgcgcggcttccgtcctggctgagatggcggcgcccgggatcctgtgtagcggctgcagagggtgccgccgccctaggcgaagtagggccgtcctgagcgaaagaaccgcccccagcaggagcaccaccacggcttagcaaagaatcccagaccccgcccgggaaggcagccgcaccatggagtcttccagttcatctaactcttatttctccgttggcccaaccagtcccagcgctgtcgtgctcctctactcggtaggcgcccagggaaaagaggcccagagcggagccgcgcgaaatagtacccctttctcccagtccctattcctccggcccaccacccttgcccaaaagagctggcccctaggctcccgaggaggggttagttggttggattggtttccaggac

SEQ ID NO: 167 - DNA, 700 bp, Artificial
  control peak
acattatacgtttatataacttgcttaataaataatagtagttattaatatccaatggcaagccgccgggaaacgacggagctgggatttgaggattggctccgcgaaccccacaacacccggggactccagcgaaccacacggggtggatccaacagccgcaggggcatcgtggtcctgcggagacgccgcccgcccggaggcggcgctgtgacccaggttggagagtcaggagcccagcgccgcgctgcggggcctgctgggacttgtagttcgcaggttcggggtgcgcaggcgcagtgccgagtttacctctgcgcgcgtcggggttggtggcggcggctgcggcgagttcctgaggggcgcctgcggggggcgtccgctccgagcgccgaggagccgagcggaggaggcgtcccagggacacgcaggggaggccggccggcctgcacggggcgctcccgcctcgggggccctgtctggcgcctcagcgcgcgccccgatggtcacctgcgcccacctgggccggcgcgcgcggctcccggcagctcagccgtaagtgccgccgcctcccgcgccgcgacctccgtgccgggcctgccggggctctggtggggctgggtctggcggggccgggtggccggggcatgaccctgtcgcgatagggtccccacgggagggtcgggagccggcagctcgc

SEQ ID NO: 168 - DNA, 635 bp, Artificial
  control peak
agctcctcagtaaagctaaactcagtctccgactccggcccacgtgaagaaattcccaccggaagacgagttaggagagctttatatgagcaggcgctaaacctggtttaggtatttaggcagcgcgcggcccggctcgtaactcctcctgctgcagcaggcgtatcgttggcctcgcctcgaccccggaagtgacttctggaagtgacccaggaagtgacagtgagctgcctctctgcgattggttttgcccgagttttatttttgtctgtctcagctagaaagtgggagactttcatttaaaatggaaattggggcgaggggttggcggctgggcgaagggagcattcaaaagcggagaatgtcactttacccgagaaattcactacgatgggccattggtttctgctcggctcgcaggcgcactgcacgagtggaggtgtggctagtggctgtgatgagataaatccatgcatagccttttcattgcgagcttgaaagttcttttctattacagttttagctttaggtttaattggttcgactgccttctccacaatttgggcgagaatttcctttgccttctcagcaaaagttgttctgcggacccgtctgggtcaactttcatgagggac

SEQ ID NO: 169 - DNA, 691 bp, Artificial
  control peak
gccagggcacaaatggaagccagtattgactgcccagcagtgtggatgggggccgggagtctgactaggcacctgtctggggcgaccgcctgagcagttggatacggcactagtccacctgggagccccggcctgcttggggttaaaatgagatgacgggctgactgacggtgataacagggagtatgacaggactgaggaaccactgcgactggctgataacgtgattggctagcagtgtgacagacagaccagcgcacgggcagggcctctggcaactttagctaccgccgctgtcgctcccgctgccgccgccgcccagacgcactctgggaaagcgcggcgagcacccgcctccgggaccgagccgggcggcagggaggggttcggtcacggctctccggttggtccgcgcgggtgctgcgagtcgggagccgcggccaataggctgcgcgttcccggccacgcccgcgcgagcgctcttctcgcgaggccggctaggcccgaatgtcgttagccgtggggaaagatggcggaaaatttaaaaggtgaagcagtggaggcagaggcgcgggcccgctggccgggccgggtgggctggaggggtggcgttgccggctcggggccccggcgtccgaggccccggcgccgggagccggaagctcggcgcggaaaggatgaggctcgggga

SEQ ID NO: 170 - DNA, 715 bp, Artificial
  control peak
tgaaactccgtctcaaaaacaaacaaaaaaggaccaactaaaaaaagaatataaatacaataaataacaggacttacgggggcaggctgacccctaaagtaaggggtcgggctggaaaggatctcaaggggtcagagtttgagccgccgaggcgcgcgagccccagcactcctaggcagctgcatccgccccggaagatcccctggggccctgcgctggctttccacagcgccatgttggacggtcctcccacttcccaccttcccagtcctcccgctactctgaccgttctacctgtgattctcgctggtggagtcaacctggaatggattcccgccgaaccacgcctccctttcctggttcgcccgcagtttcggcctatcaggttgagtcgggtaaaccaggacgggccaagcggcactctcattggatatcactggtgggggcgggaagaaggcggagcacgtgcccgggcggaagtagccgtcgcatcgcggattacgaaacgccgcggagctgaggcagttccgctggctagtgtgtacgcggcgagcttctcccggcgccgcccgctcggctcccatagcgcccgcgacagcggtccggacgccgcccgaacatggactccgccggccaaggtacctgccgggcccggccccttcgccgcagatgggcccaggctgcccgttgttctccgtctcccggggtcgcgtct

SEQ ID NO: 171 - DNA, 563 bp, Artificial
  control peak
cgtcagcaagacggctggtccctgcgttaagagcactttcaataaggacagatggagcctctcgtataaccttcaccggccccctgcaagagaagcgcgataatggcgcccccatctacccagttacccaaaccaaaatcccggcgtcgcccgcagtcacctccctggcggcctcgggggcccagctccgagccggagtccccgtcagacctgggaggcaggagtgggcggggcttgaggcttgcccgcaaggagggagagaaagcttcgggggcggcccctgagccccggttcaggattgggcagcccggccgggaaggggcggccttccctcccggaaggggcgtggctcttcagaggcggcaaattgcaaggagacgccgccgccttcatgctgccggcgggctgctcgcgccggtgaggcctgcgcggcaggagggggtgggaggatgcgggcgggccggtagccaggcgcggggcccgaggcccgacgctggccgaggtgctgagccgccggtgcgtcccccaggctggtggccgagctgcagggcgccctggacgcc

SEQ ID NO: 172 - DNA, 463 bp, Artificial
  control peak
gacccatttgtttgcaccctcccgccggaaacccctagataaatcaggctctttgctccccacctctgggttcgggcagacactcacttgtgccgacactcagatgaacaagcagatcgtagccggcctgtggctaagctccgaaactagaaatttttcaaatgctagaggcagaaaatcacccaatcaccgactggatctttaaattctccagccactcagaggtaggggcgtaacaatgggcgtgtctttctttgcgtctctactccaggagcttgggctgtaccaattgcctgctagaggaaggaggtttatggtggtggctgagcataccaaatccagtggacggctgggcgtggtgggaggaggccgccgctgcggcagccacccggcggcgcgtggtgtcgggtctgaaaaccctacgcgactccgaagatttattttcagaaccatccttccagca

SEQ ID NO: 173 - DNA, 627 bp, Artificial
  control peak
agagccagaagcccggaattccaccccatccatcctacctctctacccgggggacgaatggagaggccgggcgagggcaggacgtaggtttttcaccgcccttccagattccactgccgcatctcccggccgaatgctaaccgggcgtgcaggctggggttcgcaggaggctcgcgatccgccgctcggagctccatggcccccgggccgggccgactgggctggaagctgatccgcgcggccagaggcagggggcggggccggggccggggccggcgaggaggggcggagactcgcgcgccgggggcggggcctgcgcgccgcaggcggatcctacgagcgcggcctgcgcgacctggggagggctctgcgcggccgggacggggcgggacgtgaatggctggcggcgaggcctccagctaggggcggggcctcagggccgggggcggggcttcagagccggggcggagtctgagtaccagcatctcccttttcggtcctgccgaatttccagcaggctcctgcctgggcgaggctgtcgggccgcaggggaaagagacctagagggtgtcgggggccaccaggcagtggcggcctggctgtccggacttgagtgtctccatttct

SEQ ID NO: 174 - DNA, 406 bp, Artificial
  control peak
gagggacacacggccttcagcgtgcccccgaatccctcagagcctccaatccccaagaagaaaccccaatccattccctcagtgtctccctggaccccttcggctggacctcggccctgcagaaccccccacttcgcgggacgctcgctcaccatgacgcgtgcgagaacgcggtgcactcacccaccggcctagggctccgggcactctgcggaaactcgaccaattagagcccgactctggtctgacgagcatcccgtccgaccaatcagtgtgtcttgagccggcaaatcaccagggacgaggaggccgctcgcgttgcatgccggaaattgtagttcagatacgctggttcggatcgatcaaagtgaagattgtgtctgggaggcctgcgtggccgacctct

SEQ ID NO: 175 - DNA, 731 bp, Artificial
  control peak
cgcgcataatcaacagaaagcgctatagaaacgtaagggaatgctttgcccttcataaactgactcctatgaacgcatctaaagcagaatcagtttggaaaagaaactcagttccctgtcccttctcttcggggagggtaaagaatgatacggctccctgaagtttgggaaggtgccctcgatcgcaggaaaccccagccctatcctcgaccgccccgcccctacctttttcagtcaccacgaagcactgcttcaccggccccacctgactgaacagttcctccagctgctcactgcgggccgagggcgggaggcggcccacaaataaggtcaggccggccatgagaccgggaaacccaaagcgcgtgaggacgcgagcaaactaggccggcgcacgcgagccgaaacgctggctttggtaggacaaccaagctcacacgccgagagattccggaagtgctcgttgcccaaaagagaccggaggaaaacatggtcgggagggggataaaaggttccggaatgaaaaaataatggcgccccggggcgtagcttctggagcggggctccgccccctgccctagcatcgccctcgccacttttgtgtaaacttggcatcaataatttctaccctagtgttacagcttttttagaatttgcctagcaggctttctggtttttgccggaaggcctccaaaaagtaattttttttcttaatatacaaaataaaaata

SEQ ID NO: 176 - DNA, 511 bp, Artificial
  control peak
aacgcggcgggcagcaagccgggctctaaggatactcccgggagggccaggccactgggcgccgcgccagcgtccctccatcactccctcccttgtttcctcactccgccccggccttactcccctcgcttaccctgcccagccgggccagtcagcgctcccggctcagcaaccggctgtctcctgggcgtccgtggaaacgccgtccggacggaacaacttccggcccgacggcgccgtcattggctgttcgcgcgggggcggggcagaggccgccactgattggctggcggccgcggcgtcgcgtcggaacgtgactggatggtagtgtgctggccggtctgcctccgggagaaccgagcgcttccggtgcgtgtggtgagcggcgggccccgggctggaggggccgggactgggcagcgcctaaagcgccccgtgccgcttaacgcggtctctcatagccgctcgccttcctttcacttcctgttccttcagaaagcgctgtgccttg

SEQ ID NO: 177 - DNA, 489 bp, Artificial
  control peak
agccctgcctgttcgctcaataaacgttggtcgctccttactccttaccactgagtctcagggcgaaagagtggcgcctgtctctttaaattatgcaaatagactgcgggtggcgtaagggggggaacataaaaaggagcctagtcttcggctggaccaatcagcgcgctgagctggcggagctgggcgcctaaatggcccaatggggggccgcgcgggatcagagcccgccctgcctcctggcgccgtcgcgggggtggggactttctaggccacgccccgggggcggagcccgccggcagccgcgccgcagccgcggagggaggggcggggcgggctccggtgtcagcgaccaagccgcaggtggcggcgagcgcggggctcggcccgagctcaggaggtgagtgttgccccccggaaccccctgggataccctggcccgcgcagcctcccacgcgcaccgtgccctgcagccccggcggctgcgga

SEQ ID NO: 178 - DNA, 660 bp, Artificial
  control peak
gattcctgggtcctccccgcagagttgctgatttagtaggtttaagggcccaagaatttgcatttctaaaatgtttccaggtgatgctgaagcttctggccggaaagacatcctttgagaacatatttcctccccagccctcggcccttaagccccagaacgctgccccgccctgggtggaacgaagctgagctccaccctagaggggcggggccctttagctccgccccactcaggagaaccccacccctccctccccgttctcattggctgggactgcagggatcccctgcgccacagtgccgtgcgcgcgtcagcaaaaacgccagggacggggtctccgcgcctgcgcagtgaagctgggcgccttcggggcttgagcttctgagggtcgggtccagcgcgtgggctgctggatggcggaaccccaggcggagtcggagcccctgctgggcggggcccgcggcggtggcggcgactggccggcggggctgaccacttaccgcagcatccaagtcggccctggtgagccgcccggacctggggaggggactggcccgggagaccagggtcgcgcgtgggggtcccgctgtcccagcaccccgctttctgccgggcgcgcccccaccaggttcgagtccgagcggggcccgggccgcg

SEQ ID NO: 179 - DNA, 529 bp, Artificial
  control peak
tggtccccgctcccggcgggcggcaggctgcttttaccgtccggtcgaaactcaaactccaggaactcgtggccgaacttgcccttgtgccccacgtagtaacgcagataaaagtcactctccatggctcccaaaagacaaccgagcctgaacttccaagagcaagccgcactgccgccgtctgcgcccgacactgacgtttgcggcggcgcgcggaagtgacgtcaggtcccgtcgtcgctgcgtggaggggttccttctcgctaacctcttcgcccggaagtggcaacaagactgcaacatcccctcacgcctagtctgtaaagaactttgtttaacccggaggcggggccagccagctccgcccctccagctggacgcgggagcgaggttgaggttcatacccctgggttctctccaggcctgaggcggagagccagccgcctgcctacctctgggctttgaaccccgggtctggtgacttcgcttaaagacctcgggccgaaggcccgccaggctcaacctcggt

SEQ ID NO: 180 - DNA, 881 bp, Artificial
  control peak
gcggcgcggagcgcggccagaagccagtagagagccctgaaggaaagaagagggtgagcagagatcccatctggccagacacgccgcccctcggtcctgaaggcatctccataaaccacgaccctcaggatcctcggagaatcacatggggaccatgacctctgaccccagtcctctccaaaagttggaatttcgctcttttccagcggagaacccaggtaatctctgtcctgaccctctggaaaggttgcctcgctctcaccctcagcaaaagggccccagtgccccgcgccgcgcgcgccagcatttcccaccgccgcagcgacctccggaaccaacgagacgagcggagcggtcgctcccagaagcctacccgagcctttcctcttgcagttccgctcccccggatgtacttccggcagctccctcccccgcttcaacactcacccaatcagcgttacttggtcgaacggcgcgtcaagttgttccctgggaaacgcagaccttctagagaagggcgaaggctgtggttgttctgggtttagctgttcacgccgaagatccccggcctgatggccaaaatggaggctatttttcgcgggcattatcactcagacaccacgcactgaattattcgtcctcacccgagactcagtttgcccaccagagcgtagtatcgcccccatctgtcaccatagcgactagtggattggagctgactcgttgccatggttgccatagtgatattctctggtagtcccgctgtgttgtgcccaggactcccactggggactgaccccaatcccgttgccatggaaactgggcctaatattgcctggcgattgggccgagattttcccctcccctctttcctcaaagac

SEQ ID NO: 181 - DNA, 676 bp, Artificial
  control peak
ggccggaagccagctgtggtcctcagtccagccttctagggccgcgccgcggtcgcctcctcccggcctctgcagcccgtcggtggctcccgcggcacccagaacaggctcctcctggactggccgccgcctccccgcgccctccccgcgccctccccgcctgcctgcccgcttggcgctccttcctggtccgacactgccccacctgtttgctcgacccggggaagccaccgaggaagcgccgaggagctgcgcagtcgcaccgagcccggaacggaccctcccacagaaccgggtccagagccggggagggggcgggctaggatgaagagggcgggccgggccgagctgcgcacacgcaggcttccccgccccctgcggggcctcagtacgcctccgagaacgccgggaaccggaagccagccgcggttccgcccggattcttccggggccgctctgggatgactggaggcctgcgccgtctatggtcggggagggggagagcccacggcagctgagcggagggtctggcgcggtgacgctgcgcatccgggccgcgccgcgctcgccccgaccctaaaccctcacctgaaccctaaccccgctgggctcccggctcccccgtcggtcaccatacatcgtgtgtcccgattcaccataaccaccagaaagtcgc

SEQ ID NO: 182 - DNA, 538 bp, Artificial
  control peak
tcgcccccgaagaagctgcagaacgacatggcgccaccggaaccgcagcgcgcttgccgctgccaactgaccaaaggctgccgacccgacgaccgccggtacccgcgtccacctccccgcccctcccgaggaccaatcgctccccggagggcgaatcccggcttccgcctccgggaggccccgcccctcctcagggctggtccaatacgatcttcccagctggccccgcccacgttgactccttgggaccactcctgggccacttccccaaggccccgccccccggtgccagtccaatcgcatctccccagcaggccccgcccatggaaactcccagggaccacccctaggccgcttccccaaggccccgcccctgccgcccctcgcgccctggtggctaggggcgcacatggggcctggccaggtgggcgacggcgcgaatccgtagggtcgtgtccccccagtccagtccctggtactaagcccggagctgccggaggtcatgcagcctttccgcgcccaggtgaaaccgcctttg

SEQ ID NO: 183 - DNA, 595 bp, Artificial
  control peak
catgcaagataagcagctacgtggctcacggtcacgtcgcaaaaaacctgggggctctcctccgcctgcttgtggtcccgtacaaggcgcttcccaccctgggtctctgagcccagtcggtgaaagacggaaggagaccgattcacagccccaacagcgccggtgatagtccctctgccacccctttccctccgctgccctcccctcccctcccctcccctcgatgacttcctccaacagctgacgaagctggatccctgggcaccgcctcttccgtccgcggcacccggagacgcccggaaatttgcgggcgcgccggaagttgaggggagtttcctgcgagctcggcttcctcaacatggctgcgcccttgtcagtggaggtggagttcgggtgagtcacagagctggggcgccgtggggatggattgaagtcgtcgggcccagaattcctttccttctgccgtggggcctgacacggccgaatcactgggtgtcccagtgcccgctgtgagctacgctacccgcctaggagagtctgagaggccagtctctcttggcagtgaggccgtctgtccggaaatagaggcgcacat

SEQ ID NO: 184 - DNA, 447 bp, Artificial
  control peak
taggggtacaaaggccagatcccccgccccaaccatgcgaggtccccgagggcgcccccttttgacgtcacggtacccaccatggtgctggcgccgcgggcagcgggccggaccgggaagacagcagggtgctgcgagcaggtctggggaaaccgaagcgcgagcccgcgcgtggggcgaggcgggaccgcgcaggcgcagcgggaagcgacgcagaaagctccaagcgctgacgggcaaagcgcggccgacttgcggctggggagcgcaagctgggtagagtagaggggaggaggaagccgggaaaggggcggggtttccttcattccgacttcctccctggccggccggctcccattgcgcaggcgcggaccctagcctgggctgccagacgggtggcgggactcagcgcctgagctcaaaggattttgttcttttccagaatcc

SEQ ID NO: 185 - DNA, 443 bp, Artificial
  control peak
atgagaaaactgagactcagagctaacaggtggagaggatgaggattaaaacacaagccttttatctatggtgcccgtaaatgtttactcgcatggattccttggctcttgtaggaaacttcttgcaagtaaaattgacaataataattcccgtgccgagctcatcgcaggaagaggttaggcgggacagcccgaggcatgctggttgttgtagtctagcgttggcgcagtgcctgatgggagttgtagtacgtcccgcgcacacgctccctcgaagagtttctgccccattcctggacgtcacagccggtccccagagcaggattccttccggcgcctgcgcctgatcaccgctctgcgcttgagctgataaactcagctgatgggataagagtcttgttttatcggattttggggaagattaaaatggttaatagaactag

SEQ ID NO: 186 - DNA, 430 bp, Artificial
  control peak
gggaggccccagcagcacggcccggatgcctttaggatactcgggttctgccgctggcacgctgggagccatgtccgccgaagtctctcactgccaccagttcgcacgcctcacaggtccagtgcttcccaggtcaacgcacgcacgccacgcacgtcccactcgccgctcacactggcccatagccgcgtccgtcagcccgttccgcccgcccctgagcacgacggactcttcaccttaccaactgcacggcgatactgaggaactttgcttttgattggacagaattggtgcgggataaggaaggggcgggccagcatgtactaagggtggcgggtgtgcggagtgggcgggaaaacgcgaaggaggacaggaggtcaggccggaaggtatacttcggcatttgacagttgacgacaccagcaacaac

SEQ ID NO: 187 - DNA, 634 bp, Artificial
  control peak
gcttccctctgttctgggccggctccgaaacgcgctcaagagaaatcggaatcagacacggggcagttcagctttggaaatgaggagctcgaggctcctgggagtccgttgcgggcaggtgggcgccgctgttgtgtcgggagagccaggagcgacagactcaaactcagaaacccaaccacctccgctccctccgcgatgcgtgtaacgtgcgttgcgcggcgtcgccctttaataacgacgggcgctcggagtgctgcgtcactcgccaccagggacttaggcgggacttccgggcgcgcgtaggcacaacttccggaaggaggcggaagagcttctcggctctaggctctggagtcccgggagcagtgaggggccacccggggcacaggaaagggccgctaggggagggccgggtgcactcggggtgtctgggccgcgggtctgagggatgaggaggggccatggccagcgacggggccaggaagcaattctggaagcgcagcaacagcaagctcccgggcaggtgggtgtgccgcggagggccaggtcggggtcaggggtcagaggtcaggtggccgcgttggcctcctgggctgcgggtggagtcgggggtctggagagggcaacttgg

SEQ ID NO: 188 - DNA, 489 bp, Artificial
  control peak
atccgtggttgtgctcaggcgagatgcgatgagcgtccgaattggatggtgcttgaatttgggttccagggcgctgtcgggagagccctgggtccaagtccagccgcggatccgcggaggaaaacaatgcgaccgcccggggctctgggctcggccccagtggccccgcccgcctcccagccgggcccacctcctgcccgcctttgggtcgccgcgactaatcccgccctcggagggcggctagacaccgccttctcgtcacctctccgccaatagtgctgcccggggaggggcgccaggagcaccgtccacccagagggccaggccgggaggctgaactggagaccagagacagacgcgagatggagagacccacggacagtcgccgacaccgggcagaggaagaaatacatacagaagcagagacaggctggcacaggagaggctctggggagaaatttcccaagagaggaagaggagctgggagga

SEQ ID NO: 189 - DNA, 433 bp, Artificial
  control peak
caatgccgggaaggtcggcgtatttagggtccgccatggcggcggcgagacgggctgggggacccgggcctcggtggagccggggccggtgttcgggtaggggagaggctgggttcgggtcccgggctaaggcggcggcaaagggagcggcagatgagcaggaagtctcgcgacagcagtagcacagcagaagggtaaagcaaaagtcgcgagaacagccctgctgccacacccctggagaggccgggattggggagaacgaggccagggaatgaggtaatggccaggggacgccgcctaggattgtgggatgtgtagttttggtgtactggagcttgggcggggagcgacaataacgaaccttggggagctatttcatacagccttttctctcacttttacagcaagtcagtgaaagaacatttataattcc

SEQ ID NO: 190 - DNA, 501 bp, Artificial
  control peak
agggcttattttaaaaagaaagggaaaaaaggtggggcgcgccgtctatctttttagaataattctgtaggaaaacgaatcggaggtaaagccatgccctctggtcagggtttctggactagaaactgcctgagctttacgcacaggaaaggacagtggctcttcactctcccggccgcatcactcggcacctaattccgccaggctcccagaggggcggttgcagctacagcctagcacgccgcgtagccaataagaagccagaattccccctattctcaaatcctgaccaatcggtggcctgaactttagctctctggccggcgcctctgttgcccggaagggaggcaagcccccttagagttgccagtaacggacatggctgcggcccccggaggaggggacgtgaagtgaggagggggttgggaggggagaggacgcgggcgaggaagaccagccccggggccccggtaagtacgagaaggcccgtggcccgaaagt

SEQ ID NO: 191 - DNA, 628 bp, Artificial
  control peak
aagctgcctagcagaccttcaagcatcctcaaggtatcttccacctcaagtttatgccagaaggaggtcgttttatataccatgtctcacttaatcctcacaacggttccctaaggcaggcattagccacattttaaagatgagggaagcaagactcagtaaaatcactggcccaaggtcacagatcgctagtaatggggcaggaacactatccggtggcgacctcaccgctccagtgggaccagctacttggctgacacacatcgagcccgcgctgcccagggccccagcgccctcacccgtcccgcggaaggaacccggccgcactgccgccggtgtaaacactcactctggccctctcgccggaaaccggaaattccgcgctgcattctgggagttgcagttcccgctgtggccccccgcccccttataaacagccagtctacaagaggcgctagttgcaaagcttccggttctgggggaaggcgtggcttccgcgctctttgaaatgtccggcgccggaatgtctgggtgctttggttggagagggtcccatttccagtcattcttcagcctggtgattcccgcgctcccctgaggagcatatgaagtacgtcggttctgccag

SEQ ID NO: 192 - DNA, 571 bp, Artificial
  control peak
cagtgaggatacagcatttagatccctcggctccaaacaggcggttccgggggaccggtagctggggaattgggtttagcactttccaacgcttggaactggacatccacggctccgagtcctttgaggtcttgctcgggactacgtttcccagaaggctaggcgataattgacagctctatcagcctatggacagagaaaaattctagccatggaaactgaaaagccaatagcaagaggatgggggcggtactttccggccgctggctgtcgaagccggagtcccacctgtgtccccacagccctgtcacgaatcccggtcgggttctgggaggcacagcctcggggttgcgggccgggtgcggctcggcggtggaggactcacttcctgctccatccccggctgggccctggggcggtgagtgattcaaggagggagaccgcggcggcagcggcgcgctgctgaggggcgtggagggggcttgcccgccacccagacgttttctcagcggggttcccgcgctggcctttgggagcccccgggtcttgctgggctgtgggagaggtagct

SEQ ID NO: 193 - DNA, 545 bp, Artificial
  control peak
cagtcttacttaaagcgctgagtaaactcacctgcaagaccagcaagccctgcgcttctctttctgcgcggttccccgcagcacatggcgtgtccacccgggacccacctgcggccacctgaagactgggaggactggctccgcccccgcgcttcgtccccgcccactcatgggcggagcctccacagggcgcctcggccccgggctgtcactcgggactccgccccttcatggacggagcctccgctcggctctctgagcgcatgcgcagccggtcccacccgcggctccgacacgccggagcccacgtccttccgggagcgcgcgcgccggtgcgggtgcgtgcgtgagcgtgctcgtctccgctgccgctgccgctgccgctgccggtgccgcacccgcgccctctgccgccgccgccgccgccgcccggggtctccaggctgaggagtgcgtcggccgctgcccagcagcgccgcgaatcggggagcggagccgcggagccggcgggagggcgggcgggcgggcggacgggcaggtgagct

SEQ ID NO: 194 - DNA, 709 bp, Artificial
  control peak
ctccagtttgaaagtgaggatttccaccactttcccatactttaaaagactggcatctgccaagtaatcaaaacacacaaagaggccttttaaccaagtttagcttggaatttccaactgctaaccacaagcaggtctggcaagctaggaggctggttggctgcgcagctactccagagtgtgcggtagccgagggctgcgtccgcgtaaagcccagagctgtaatgatgtcaacttgttatttactttccaaatctgaatgactgagcaaggcgaaaggagaatcaatcaaccacaatgtgaccctacaggcggaaaaaacgcagcactcccaccagcaggcggtgggggaagggaagcacgtgtagccagggcagggttgcatcatgagctcgttcgggttttctattgcacccttcgtttttctattgcatgtaatacctcagcatcagtctgcacaagcccagggagcccaggagcaacgaagggcaggctcaagggaaggccccaccatgtccttctggggggccttgaatggcaactgatacataataagacacaatctattctgctgattcacaattatggagactgtgtaagtgtgtatttttttattgatgtcctctaatgatggtgtagcaacagtcgttttgcatcattgacgttttacagcaatattttgcagggtaattttgaggtccgccgtttt

SEQ ID NO: 195 - DNA, 495 bp, Artificial
  control peak
acaggccccgccatgaattcttccctcagaattctcgtatcacaccttggcccagctcccacccgagcaccgcactcttctccctccctgaaccaagccccaagcaaggcggcacctgcggcagttatcaaaagagaagcctccaactggtggagcatacaccgacacagccgccatcttcccaagaaagcagttccgggtcggaggcgggtgcaaacaaaaagagccgagcacttccggcgtcgctggcaacgggggctgaaggggcggggcaaacgccgcactagaacgattgatcttagagattctggttcctagagaggccagagatgtttgaagtatatcgccccaagtggcacggaggagaaagtgacacccggaaccgggaaaggacttgtccaaggtgaacgcaccagcgaccaggtctcctgccgctcagactggacttgtttctaccgttaatttggttctgcggtgcttcagagacagacagac

SEQ ID NO: 196 - DNA, 732 bp, Artificial
  control peak
tcccagggctcagcggaatgcccttgatgacctttaacaatcacccttttatcatggggacaagagcggcctagaaggaggtttttggagtcaccagacccccatggcccctcaccaggccccttcaagctctcccctcccccacgcccgcgcagcgcaggtgcgaaaaccttaggggccacccgggcgcgacagccccaaagcgggggggatgggaccctgggaggggcgggggtgggggtgggggccttcccggtgttgcttggtgaccacgctctccgcccagaccgttccatttcttcagcagggggcgccccgggccgggccggcactgaccagcctcgcagagccaccctcttccggcctgcccccctctgccgggcgatggtgtgcgcccgtgacgtcacagaaggcggggccagcgccgctgccgggtgctggaggcgccattggagccggcttggctggcgagcccggctgaggagcctcttgggccgcacttaccgccgcgtccgctcccggtccctggcccctcagcggcatggcgtgcggggcgacgctgaagcggcccatggagttcgaggcggcgctgctgagccccggctccccgaagcggcggcgctgcgcccctctgcccggccccactccgggcctcaggcccccggacgccgagccgccgccgccgtttcagacgcagaccccaccgcagagtctgcagcagcccgccccgcc

SEQ ID NO: 197 - DNA, 658 bp, Artificial
  control peak
cgtctcaaaaaaaaaaaaaaaaagtttccgagcaattcaccttttcgcacattggggcagggtatgccatttcctgggaggcacagtagtttgaggttgggaatagggaagtctcattgtgacccctagaaagacaagccttcaggctaacccggtaacacccatcgtcaggaatccttcacggccgccagagggcgcaaggagctgcggctggaagtactcggagggccggcggaggaggcgcgcggagcctgtaactcgcagcgcgcgctggaggtgggcgcggggcggtgcggtgcggtgcgcgcggggcggtgccgcggcggcggagggagccgcgatggagggcgctccgactgtccgtcaggtaagccgcgggcctcgctggggcaggttcccatctgtggctttagcctactttttctcacggtgattgcttgaaccttctcagattacagttaatccctcccttcccttttcttcgtgctctttggagtaatacggaaagttgttaccgaggaaacggtttcctgtgcctctgcgagccctttccctacgggcggtggtggggacatttgcttccctgtgggcgttgagggcttgaatttggtcccgaaatgtcctggtgcggatgtctcagccccaggcccccttgggt

SEQ ID NO: 198 - DNA, 483 bp, Artificial
  control peak
gatcctcctggctttcatcctgaacgcgagttaacttaagttcagaagcggggcaggcagtggcctgggaactacattacccaaaagacacagcggcggacacaagcagcgagtgtagccaatgaaggcctagcagagcggcgtctacgggggttcgcaatgcgtgtgggcgggacttcctgcaacgcctcctggggttgtcaatatggctgcgttgggatctgttcaccttcaggctgagtcgagactgaggtgaaaaagcggaaaaacgcgagaaaaggtttccccgttgtacagaggctagagtgaggctcggttgaatcggttgcaggcgttggtgcctctgtcagcgtccaggtcactgccgctcccgccccgctcttccctggctgtgctggcggaggctgcgccgatgaacctgactgaggtgggtgccgcgtcccagggcgccccgcccgatccctcctccgagtgccgaagccc

SEQ ID NO: 199 - DNA, 637 bp, Artificial
  control peak
tgttgaggggactattcctagtttatgaggtggttaaggatatcggtggggtgggctggagcggtgtcgggttaggtctgagagaaggcctcgcacaaaacactgtacaaacccgaaaggaagtctgagagacgaaccgccttcctccctgaagcttctagaactggagcagaaagaaggtgtggcccagggccagccccgcctcctccccgggcggaagctgtgtcagttgccggaagtcggcgtgaggtggggcttatgcggcggcgtggtgaaatagatatggcgaccgagggggatgtggagctggagttggagactgagaccagtggaccagagcggcctccggagaagccacggaaacatgacagcggtgcggcggacttggagcgggtcaccgactatgcagaggagaaggagatccagagttccaatctggagacggtaaggttggccaagagcatgtcggggcgggctgagagcagaggggggctctgaggctgtagctggaagccagcgctccaagacggggttctgatcccgttggcaggaggaggcaatagggtagagccgagggaaacagcggtcaccggagcgcgccgcttgcctaaaggagcttgatagtgggcttaagggg

SEQ ID NO: 200 - DNA, 493 bp, Artificial
  control peak
tcctgctgcctctgagccgccgcctccggccgccgaggcgaagggccacaggccccgagctttggacccgctggcgcccatgtcagggccgccggcgcatggcgggccccgtggccgctctgctcacaccgcagtccgcactaccataacctgccccagccgctgatcgcaggtgccgccgccgccgccttccgcaggctcggctgtctccacggaaacctcacttccgcttccgcacgctgccacctctgattggctgttgctcaagtcgtgggggcgggaccaatgggcgcccttagatgctgggcatcccagcatgcgttgcgagatcccaggtcccagcaagcgcgatctgaccagctgggacagctcccaccgaggaaagtggtggccgtgagtcctcccggtcttttgggtgtatttggcggctctgacgtcccccaacacctgagttcaggcctagcaggacttcgcaagcaccaatcctcaaacc

SEQ ID NO: 201 - DNA, 432 bp, Artificial
  control peak
ggggcgacgggatttgggggtgtcctagccccggccgatggaggggaggtgggagtgggaggttgggcccatagcggtaggaatggtggggggctgtccccccagcaccctccctccctccctttctgctgtctctctgagggctggggctgctgccgccgctattcccccgccacccctccccaacgcctgctggtttccggggccggccaggaagtggagggcggtgatgggcagcctgttttgccaatcgtctcccagaaactctggcatctcctccccacatctaccagtgtcctcttgcgagccccgccccagggctctccctcggtctttgcccccatctctggctccagctgcatcttttttttctctaactccctttcagctctggatcccctggtgctgtattcctccttccgcacattcctt

SEQ ID NO: 202 - DNA, 424 bp, Artificial
  control peak
ccgagcacccagcgcccagtcaggcccccagccgccctcagcctggcccggccgcgccgctcaccctgctgcccggaggaatctgccatggctgggaatcccggacccgcagctgcggctacagacacttcctagaatctgatgcaaccgccgccccggaagtcgcgccccacgccgggcccgtcttccgggacacgcccggtcgctaggcaactaccaggggctccggatgtcgccggcccggagcatgctgggagctgtagttcaacccaatggcgacccgcgctccggagcatcgcgcggccaagcgttcagtggccgtgctgggagtagtcagcccagccgcggggagacaagcactaaagactgagcgtcctgtaataggtgtttaagtcatgattgcgctaagcgccgtggggaccag

SEQ ID NO: 203 - DNA, 490 bp, Artificial
  control peak
cctcgaaccgggcccgacgctcgaagtgcagcggcccaaagcgcgcgaccccgctggactcgggcccagggcccccgtcctccagggacagctcgaaggcatcatcaatgctgaactggggccgggcggcgctcatggccccggcccgctcaggcgccgtgcgccgccgccatctgcgcccacacacccagcgtcgccgctgccccgcccccgcatgccccgcccctccgaccctggctccgccccacgcgccggtcccgcccccgtccgccctaagcaggccagagttgccgagccctgagcgggctgatgggtgggagcctcttccagacggagcgcgcccgccccgtgacgccactcagtggccgccatggcaagcgacagcccgtgccgggctcggacacatcccccaggcgtgtatgtgcgggggcttggaggggcttccacgccccaacctccaggtccctgacagggccttttgcaccgcagc

SEQ ID NO: 204 - DNA, 667 bp, Artificial
  control peak
agaaagacttcctccacctccgcccggtcctcccacctcggaggactccaggctgcccaggggcggacccctggggcccaggtgagaggtggcagagggtgccagcatcgcgggcgtctaggccgagaggccggtcccagcccggccaggccgcgccgccccaagcctcccaatcccccgggttccctcggctcccctcgcccaccggcggccttgttcccatctccctcactcccaacccggcggctctcacctcggtcccagactcagccctatggaaggcactacacatcgccccgaaggctcggcgcaggagagactcaactttcccgccaccaacgccattcaccaactgcgcagactctacggccagtcaaacccgcccacccgcgcgcgtgcgcactgcgccaggccgcggccggggaggcgggagcatgcgcaatagggtgtgcgggcctccccgccccgcccccagttcctgtgacgtcagacttccgcgtcccccgcggcgctctcccgcgtccccttggagatcgctccggggtggcggggacagctggtgcttttcttgtctgttgtaaactttggttcgttattattatattaggtaaagctggtcttatgtattaatttgtttaaccattaaaaataattgtattgcacaagc

SEQ ID NO: 205 - DNA, 520 bp, Artificial
  control peak
cgagcgccgcacaggcacgatttccagctcgccctcctccacctccatgggctccggctcgccacggtcaacaccggactccgcctcaggggacgggagccccagggccggccccgccggggcctccgccttcaccgacacccgcaggccccgtacggccgccatcgctgcctgccggccgcaccactgagagcccgcccctcacggccgccggctagagcgccgctgccactggccacagaggagccgccgcagctacctagagcgtcgccggcttcggcccagaaggccgcccttagcgccgccgccccctggtggcgggagaaaaccgggccggctgcgcgggcgggacctggggaggagaaggcgggactgagcggggacgggggcggggcctgcgggaactacggagcttggaggagaccggggtagtcaagggttacaatgtagctacgccttgaggctaaagctgtactaaaagccaactttcctgagtgatcactatgggccagacacttcc

SEQ ID NO: 206 - DNA, 559 bp, Artificial
  control peak
tctaaccccgcagagaactcctcgcaccccagtcctcaaaatcccgccccggacagcggaggcccgggcagtagcttccagacccaattgcccgcgcaagggggcgtggcagcatcgttgggcggggccgcctgtaagagaacgcggctaaaggtggggccaagcgtgcggtttgctgcgcctgcgtgaggaaaaagaggggggcgtggctccaggccggaagagggagtctgtaggggcgggccggctggcgtcccctttccggccggtccccatggaggcgctggggaagctgaagcagttcgatgcctaccccaagactttggaggacttccgggtcaagacctgcgggggcgccaccggtaggccgcagcggggccggggtcgcgtggaggggggcgtcctagagcttagcccgggctcctggactctgactggcctgccggtgtctgagggagcggattcgaggccattctgaccctcgccccttgtcctgcagtgaccattgtcagtggccttctcatgctgctactgttcctgtccgagctgcagtattacc

SEQ ID NO: 207 - DNA, 518 bp, Artificial
  control peak
ttccaagctccaggtggctgcccttccaagtcccacactcttgtcctgatggcctccgaccccgggcctcgagcccaaccaaaggcaccgaaggagagaaaagccaactcactagggtgccctctccagcccccactgcacttccatcagcccccgctgccactctctcgccggctccactcccgcagtcacttcctgcttccctcccgcacacgtggccctcccgcctccgggccccgcccccttggccgcaactggcaactcccgcctgaagaatagattctctggttcacagccgtctgcaggctcaggaacagatctgggcgggcagaacagagccctgaggccaagagcccagcagtctgcgagtgactgcagccctaagggaagctacaaacacccaaggaaagagccctttgtaacacccctgatgttacttatgcttctacacctttggcagccaggtccatgccactgggtgcgcagcagagcaggatgtgctgtgtgtgtgttgcggg

SEQ ID NO: 208 - DNA, 604 bp, Artificial
  control peak
tgttgaggaagaaaagcgcggcctggcggcgccgcgagtccccgcgcctccgcagcgcctgcggcggggcccgagcggccgaggtcggcgcggcgggtggcgggggcccggcggggccgggggccgggcccggggctccaccggccgcggccgcggccatcctcagactgcgcccgccgccgcgaagcgccccaccccctgcccgcgcgcccgcgcgcccgccgcccattggccgcggcgcgcccacatgcaaatgtatcgccgtcgcgctctcccattggccagcgaggtgccgcgctcagggcgctcccctgggtcctagaggcggtcggacgcagcagcagggagtcgggagctgcgcgcctccccgccggttcgagtccccgtcttgggacctgcggccgcggggctctcgctttctgttccctttgctgtatggcggccttcgcggatggcctccatcgagcgcacattttaaggctccggacccagggcggcgtgggtgaggccccagcacacttccctgtccggaggtgtgtctgggcggcgcttcagcgggcgcagggcggcccctggaggagccccaccgcttcccggaagggcc

SEQ ID NO: 209 - DNA, 385 bp, Artificial
  control peak
cctaagactggcctaagactacatttccattcaagaccgaacaccatcgtcaaatccgtggggaatatgtgtctctagacgcactaccctccgtcctacgtgccgaactgtaaacgaagccacacttccggtctatcggtaaacacaggactggagtatgccacacttccgagtaccccagtgttcattcacaagctggtatcgcgttatcgacaagccacgcccctcgcatcaatcaaaaataaaccccaaggcggatccctgagtgccacacttcctgtatctatgaaagccacacttccggctgggcgctgtggctcctaccggtagtcccagcactttggaaggccagggcaagaggatcgcttgaggccaggagttcgaa

SEQ ID NO: 210 - DNA, 507 bp, Artificial
  control peak
ccctccgccgtgaccttcccacggccctgaccccgcgtcctccccacctcgacctgccactgctaacagcccacacctgtccgcttcccgaagccctgtcccgaagccccgcgctctcgccaccacctcaccgagcggcgggcgcgcaatagcacttgtgccccggcggtagcggcccgacagaccacggacgccgccatcttccagctgcagtcggccctgttgcgccgcgcaagcgtagactgggcgcgttggcggtcgcggcgcaggcgcagtaccgtacagttggtgctcggccgcctcctggcgggaaccgggacagtctattgtgctggcctggctttggctgactggctctgaccgcgctgccgccagtccctgcgcacatcagtccacctgccgtccatcagcccgagcaggtccgcccgccgtgcagtccatctccccattccacgcgtggaagtcagagtgcattatgtttctcaccagccgcgctgcgtactgagg

SEQ ID NO: 211 - DNA, 639 bp, Artificial
  control peak
agtccacccctccccctccggccgaagccgcagagcggtcagcctcggtggccgcagcaagccgtgggggtggggctatggaggggaccgtgggaggaggaggctaatgggacccgggggctctagagtgaggatggggaagatacacaagcgtgaagacgaagtgctgctcctgagcttaggctattcgagggtcccctgatcacctgccctctgcacggcgtagtgctgcggccacacgtatttgccagtgacttacattatacagtcaccgggtccctgccccgcgaggccgcgcagtgtcacggcctgtgtcacacgccctcgcagtgccacacgctccctatcacactcacaccaaatcacacacggtgtcacacgcagtgtctcacacactccaatactcggtttctcccattcagctctggctctctggcgccatctagaggtctctgagtagcttgggaactccctctcctgcgcttgcggtttggtaacccaggctaccaagtgtcccagttcaacaggacctcctgccttcttcgcctgcaggcccaggcagagtgttctaggtcaaaagaatgaggaaggttatccctagccaggcctagtgctgggtgaggaaaggctcttgttatg

SEQ ID NO: 212 - DNA, 389 bp, Artificial
  control peak
ccctatcgagagtcccagcaatacaaagaacagggccagggtttgctccaggatttccccagtggactacaactcccggaaggcagagaagtggactacagctcccagaaggctgcgcgacatacagccagcccggtgctctcgtgaggcgtgccagtcagattccgcgggagagcggcagagataccgcgatatttgggagcggccccgagacgcgcctggcgcgggtgagcagtggaagggggctgggaagctgggttctcgcggcggcgcgggaggggaggttcggggagctgtgtccgcgagcgtgggtccacggctgtctgtgtgtgtccttattttcccctgtgtgcccatgaagctctgtttgcgactgtgcgtgggtaggc

SEQ ID NO: 213 - DNA, 403 bp, Artificial
  control peak
ctctgatccttaactttccctatctgtaaatgagggcgactagatgggtgcggactgtcatccgtgttgagacttccggcccccgcagaatcaaccttaccggcgggagcacctcgaggggacgccgctcgcactagggggcgccagggccgctgcagagcccgagccagagccagggggcggggccgcgtctcgggagcgaggcctgagagcggcggggcggggcgtcggcgggcagtcccctgtgccaggtgtggtttggtcctcccagggcgccgtaggcggtgcatcccgttcgcgcctggggctgtggtcttcccgcgcctgaggcggcggcggcaggagctgaggggagttgtagggaactgaggggagctgctgtgtcccccgcctcctcctcccc

SEQ ID NO: 214 - DNA, 507 bp, Artificial
  control peak
attagagacaaggtgagggaaattgagggagttgcctgaggatgattctcctcccagtcaagtaggcacctgctgagagaaaggataagccgagatgagggagatgatgatggtttggggcgcagaaagcgggaatgaaagacaggagctaaagccgaagttttttctctctcgctacttaaaagctgtaagataacgaattgatcagcaggtggcagtgtccgcggaggctcgcatcccgtcggccctggctctcaaggcggaaacacgcaccatggtgactttccggcctgcatcgcctctgctcccctatcccggctgacgttggacgacaaagatggcggcagggaccagcagttactgggaaggtgagggcgagaaggcctccgggtgcgtcctacgagggtgagaggggctgagtgaggacagatcttgggagaagcagcacaggaaagaaccagactcccggagcggccgagctgagcagcggggcttgcctaagtggga

SEQ ID NO: 215 - DNA, 664 bp, Artificial
  control peak
ctctaacgcgagcgaacaccggcttcccgaggccgtcgctcggcagaggaggggccaggcgcccgatcccagatcccctttccccgccccgccgcgccccgcctgctctggcacagccgcactcccgcaaatccctcccgcggaacccccactcctgcctgcgcgggcgctggaaggcgggcagggggcggggcgccgcggcgcgtccgttgctaaggccgcgcagccctagtgaccgaacgccctgcggcggccgcgcaggcgctgtggtgggctccggtgcactgtgggatggaaaccggagcggcgcgtcctggcaggaccgggcggcggcggcggcggggccggcggcggccagggacccgggcttaggctcggccaggccggctgaggggcgcgggcggccagcgggcggcggacgccgtcatgaacgcggcggtggtgaggcggacgcaggaggcgctggggaaagtgattcggaggccgccgctgaccgagaagctgctgagcaagcccccgttccgctacctgcacgacatcatcacggaggtgggcgccggggaccgggcccggccaggtgcgggtcgggattccggtgggcgccgaggtcagggcccgggccgggtggcgccgggtgcgagccgggctcgggctcaggtgcggg

SEQ ID NO: 216 - DNA, 577 bp, Artificial
  control peak
tgacgaggcaggcctgagcaaactggatgtgccttgatacaggccacggcgagaatcagagatgggggtccgacgggccccggggggggtaccgtgggggaacacatcggacgggtaccctgggtcagcgctctccgcgactctcctagccctgggccgggggcaggaggggaaatcacctgctccgtcgggcgccgccggcgacttgtcccgcgcggtgcagctctcatcccccagccccaccaacgagatcaaaggcggggcccagagcggttcccttccagaggcgccgtggcggccatgtttgagaagggcaagttgcccacaacagggcggagctcctgctctaattctctaacgtcccagggctatggacgggctcccttggcggccagttttgttgcgcgtagggaggcgttaggcgtgaaggtcttgagagggggtggagtcgaagtgggtaggactgcatccccgtcttgttccttctcatccgtcagagacccgaaacctttctctttctttctctctctctttttctcgtctctctctctctctctctctcacttttttttccttt

SEQ ID NO: 217 - DNA, 569 bp, Artificial
  control peak
acctggatgttttcttcgaaccctccccattccgggccagctccatttcgggcgccgcccgccggcgccgctgtagttgccatgtcccttgagggctcccgtcgctgaaacccgcgctagccccgcgcgcggagtgggcaagatgtgggcctccggaaggtagacgtccagggtcgaggagaggtgggatcggccggaacacaaaaaggagaagcccggatgtttggacaaaagcaacttccggaagctggggacgtggacgggggcggggaaaaatctcgcgatatttaagattccaggaggcggtgcgttgccacggagacggagcagggctactaggacgcacgcgcgagatagaacctctagtctcgtggagagattgaagatggcggcttctcaggcggtggaggaaatgcggagccgcgtggttctgggggagtttggggttcgcaatgtaagccttgtggccttgagctcgggcgggaggaatgagagcggaacagggatgggtcttgggattggcgtggggatagctgtgggctcactgtcccttcgtggacaaattcgtc

SEQ ID NO: 218 - DNA, 541 bp, Artificial
  control peak
cactcatcccacagaactccccatcactcagcccacgacccctagcacttgccccctcaacctcactctctctcgataacagattttctgctcgctgctcaggagaccgttacaaagcctggccccaacaagcacttccgcttcgggaccgactctttggcaccgcagcctaaagggctgcaggggtcggttctgcgcacgctcagtggcgcctttgagaccccgtccgccaatagtcaacatggggccgcagcgcaggcgcagagggaaggtagccttgccggtgacgtcactgctccaggccagatggcgtgcccaccgtgtccttttgctgctggggctgcgtcgcaaagcggttgtttacttagttatgcagaggcaggcactcctaagacttcagacaccaattttcttacggcagggcggggaggggaagtgcccaactaaggaaaagattctagcaggcggcaatttcgcactttgaacttggagggcagcaacagagttgcaggtgtaaaataacgggaaggcgggatgcgtg

SEQ ID NO: 219 - DNA, 784 bp, Artificial
  control peak
ctcctcctcgggggcagcatgagggagggtcgctgtcagggcctcgaggactaagggttcagggtgcccgccgctcgtcccctcccccctcccccccaggtcgtggaggccggtccccagcatctctccagcgtccccactgtcgtggctccggggcgggcttctcccttttcaggcatttctgaaatcgccgcctcacccgggctctcttcctccggctcgccctccccagccccaggtgccagagagaactttccccaaaacgcgaaactgatcctttcagacgcctcgcgttcgcccaacagcacctctagcctcggtcgttgtcacgtgacgcggccccaaggcccaggccacgcccccggagctcgccacgcccaccgggtccgcccccacgcggccgcctgcacagggggttccagccacccgcgctctcccaggtttgccgaccttacgggcccttctccgccagccctggctgggtcacccccacgacccgggactcccccgccgttccctcccgggctgcctgcagtgaggtccgctcctgaggcctcagcctcgccgggggtagggccctctcccgccgtcgccgcagctcctttccgtcccggggcctggggggcaccgtcccacgggtgagggccgcctccagcgcctggcccagccatgcgaacgcctggagcccgcgagctgcagctgcggccctgaaggtgccgcatatcctgccgagtgggtgaagcgtggcactttggaaaacaaatcgggttagtgctcagcagcc

SEQ ID NO: 220 - DNA, 453 bp, Artificial
  control peak
tcccaggggctgagtgggagcccagtttccaggaatgggcgtcagtgcattcccggcatctggatgtagcctttacttgtccctgtgcagcccgagggacccgcgagactccagtgatcatagaggtgccagcggcacccggctccaatcagctccctctagaggaagttgctctgtggccaccggatgcagacgcacaggccgacagcctgtagggtgatctgggtgggccaggcccaggagcatgtcccgcccactttccccaccccacctcccaggcaggcagggcggctgtcagtggcccactgcttttcccatccctgattctcaatccttgtctttgatcttctcccccacctctacatcaatcaataccaataaaaaacccagggtgagaagtttcttaagcactgctgcatgcctggcactttagcaactatttgtctcattgga

SEQ ID NO: 221 - DNA, 466 bp, Artificial
  control peak
tcccatcaccccacagcctagccgggccccctttccactcttccaccacgttccctgcagcctctctgagacttcaaggctctcgcaccggcgcctctgaaacccccggcccgcagagggcgccgcgtcaaggacagcgcgggcagactcgggagggggcggggccgcagccacggtcgctttttatgaatgggggagcgggcggtgcgaggccttattactatgccgcgcaacgcgcgctgcgccccagcgcgccgcgcccattggctgatcggaccgctgacgtcaccgaaccggtggccgggggcggggcggggtgactcacgccgcttctcccgcggccgcgggggcttctcggggtccacgcacgccctgcgccgccaggacccgagcggagcctccccgcggcccggccgcgcctggtcctgagcggtgagtagcgggccccgccgcgggcaccaggggt

SEQ ID NO: 222 - DNA, 438 bp, Artificial
  control peak
atgtaacatctttttaaaaaaaaaaaaaattagaaggaccgtataaaaagttaaagaagacttactgctcacctgaccacccttctcatagacccgaaagttagattttactttctgatttttcacaatttctcctggatgctgcggtcccgcccctcgcagcattctggatgttgtagtccctcgctccagggcatccacggtagggtagttgcgcgccccgtggcagtctgggtagtgtagttccgtgcccaggggcagtccggctcgcgtacttccgcgttccggcatcgtcgcctggtgcgtggtgagtgtcctttcacagttctggtgagctagcccggaggtggtccccgctttcgcgggccttccactggaaggaaggtcgggaaagctggcatgggcagtccatgtcgtggccacatgttccttggtgtt

SEQ ID NO: 223 - DNA, 564 bp, Artificial
  control peak
agcataccgccgattttactttccagtatttaaaaatgctgtgtatccttttaaatgaaaacgacttaaaaagcgtggctctcaggctagcaatagagataccaacccgggaatctccgcaccaccccccaagaggaagcagcgtctgacgtcttccggaaacgagctccactggccttatgggaaatgtggtctccgacggatggcctacacccgggaggagtccaggaactctaggtggacagttacttccgcacacgcgtagtaggacggtagccggtattcaatcttcaaatcagcgccgcgggaagtgcgggcgggtgttgctcccctgtctctggacgacgctgtgactgatcccaggcttggagccggagctgcgagcgaggcagtgaaagggtggcaggtatggctttgctggactcggaggacgtaagagaaaggcggggactggcttctaggtttaccctaccgaagataaaactttagtctccaagcgcagtgacttcaggtttggatatgtgggtcctgttcaactcgagtagctgagagaaagcaagagtt

SEQ ID NO: 224 - DNA, 377 bp, Artificial
  control peak
cagcgccgacagcggcagcaggtagcggctcatgccgggccggggacaggcgtcaggcgtcaggggtcggcgcggagcttgctgcacaccagccgcctgggtagctccgaggaagagcgcgcgacgcagccacaggcgagcggaggcgcaggcgcggctgggcccgcgtccggaactgggctgcgaggggcggggcgcgggcggagggggcggggatcctagggacgggacctatgagcatcggtcctgagcgctgtcacagctgggattggtggtttcaggagcctgtgggcgtggctagtccgggggcggggcctatggtttgttcgaatgacgtcacacttgccgcagcgtataaggcgctacgcagttctgga

SEQ ID NO: 225 - DNA, 584 bp, Artificial
  control peak
agtaagactctagaatgtaccatctgtgcaacttgaggcaagttatttcacctgtctgggtctcggttttgccatgaatatgaaagctagtcgtgtcactctaggagaagcggtttgtgttggtggcaggtgcgccccctcccaataggtgacaagaacacgcactctcccgttcctgcgttacctcccccaggccgcgcggtggcgctgcgtctctgcagcggcccgggtctctctagggcgctcgggcgcgctggcccctggggacgccgagggcggctgcgacgcgccgagaggccgcggtgagtgcagcagcactgggggggtggtcgtcccgacccccgggctcccgggtgggaaggagcagctgggctgcgcggagcaggcccccgtgagccccgggaaggagcgcagcgcagggtgtggagccaccctcagcatccgaccgcaggcggggacgagagccttgggcaggagccaggagaccccttccctgacccctgacttgccgggggtccctggcatcacttccccgccaccgagccttggaccagagggtttctaaggccctttcggtgccggcg

SEQ ID NO: 226 - DNA, 667 bp, Artificial
  control peak
aactcgtcaacgtttcgctagatcacagccttgggttgtccacaaaaaccactcaaaaaagcttgagacttcacacaaaggaacctaaaccttgaaaccgcgagctataacatcccaaactggtccacagacaatcaagagacaaaatatagcaatacagtatacctcggatatccaccgccgccataattccgagagcgtttacaggttctctttccggcgcaaagtaaacgtaagcctaggccgccggaagcagcgggttgaagagagtgcgcatgggtgaggagggacgtagagcggaagagggggcggggcgggcaggagagaggcagggggtggaggtgcgggtgggaggggaggagacgcgagggggcggaaagcgggagaggcgcgggaggcgggaaacgtggggtggggtggggtggggtggggtaggggaatgggaggggacgggacgcagaaagattggtgcaggggagcagggaaagactagaagagggatgggccaaagaaggagaacgggagggcggattactgggaaaaaaattagaagacttagaggacagaaagaccgagtggagtcccgccccacgcccaccactgatggaagaattctgccttgatcgtctgtgatcgtcaagaaaacaaattctgatgctgtagagtt

SEQ ID NO: 227 - DNA, 457 bp, Artificial
  control peak
ttcaaaaaaaaaaaacaacaactccgtcttaaaaaaaaaaaagaaaaaagaaaaaaaatcttatgtacactataaatatatacacctactaagtgcccacagaaattgaaaacaaagaaaaaaatccatgattaaaagcttttatattaacgggggaacctctgaccagaaagagccgacagggggcgttcctggatgatctggggctgtcaattgcgagacgcggaggattgtgggacttgtggtttttctcgcatcatttaagctttctggactacatgtcccaaaatgcaaatgcaatcagacggtcccactgtggggtgtgaagtgtccgtagagctgtgagaggtaagtgtgttctgtgtgtgctgcggatttctagaagttgtggatttcttgtgagggattctatttctcctctttgccctcggtgggacctggaagagagtagttgttc

SEQ ID NO: 228 - DNA, 494 bp, Artificial
  control peak
tcagataactcgcttcactccaaagtttctgattctgcaggtttggggtagggcccgagaatttgtcattttaacagtatctccggtgttgctgatgccgctggtcccagaaccacacactgcgaacaactggcctagaacgttgtcattcctttctcctacagattttgaggcagccggtgggctccgaaacaaaacagaggtcactggatccgattcccagtaccatagaagtgactcaaaccagcgattcaaccgacaagcaggatccgggccacccaaagccgcgcgagagtttacgtgacgacatcggcgtcagcgtcacggaggcgtcggccacgttcagcggacacgggagcaagatggcgattccgggcaggcagtgagtgatccgggagttagggtcaggctgggggatgagaaactacggcgactgtatctttggccgaggagttttaaatgcgctggaagtgaagggactgtgtcgtcaaggg

SEQ ID NO: 229 - DNA, 536 bp, Artificial
  control peak
cgtccaagcgccgtctcttctctgacatacagtcctgtctcccctgcccgaggctgccggtcgccttcctgaaaaagcggaaaaacttaaaagtccagacagccaaagcgtgagaaccctccagggactgcaccacgcgcccacagtctctctgcgcatgctcacacgcgacctccccacaaagctactccgcgggcctgcctggctactgagtcgcgaaaatatatcgtagtgccgcaatgacttctgggtactgtagtccaacatgtggactggccactcggcttgaagcttccttgggattggatgttgggaccgtccaaacacggcgacttctcatttgtcagcctgcggcagtttaaattgggtggcggtggtggcatgtcctgttagctgtggagtcttttcctcttatcttgcgcctccggttgtgccttaatgacacttttgacattgtgtatcacaaccaactggcaaaaactagtaaatcacgggctcctgacgtggcagctctggctaaaatgcaggtggacgtc

SEQ ID NO: 230 - DNA, 484 bp, Artificial
  control peak
cgtttcctcttcccaccctacagagcacggctccggcaattccacagctcaggcttacattttcccatattctggacgctggtgcccaccacgtgcaccaggcactggccagacagaactaccacccccagcatgccgcgcgcatttgggccgtaccacaacctgtagggtcatctgggtctgaaacgggcaccagcgacgcgggagcagggactgacgcccctctagcaaagcatctgtggtacatcccagacccggggctctccaaggccccgcgcttccgagctccgcgcaaactctggcttctcttgtacgacagaggtggtttgctcttccgttgccccgtggcttcagctcatctttggcaggaaggcgaggcttccgcccggcacagggggtgagctggagcaccgtgcccccaagcctctggacagcatttggaggagggtgcttccctaatcccaacttcggggaaacccctcat

SEQ ID NO: 231 - DNA, 388 bp, Artificial
  control peak
gattccgagtgcttcctttcagaaagacaattctggatcaactccctagagcaggaagcctgtggtttgtaattggtgttctagctctgactccctcccctgggactgcaatcacgctctctacctgctgacaaagtgagggtggagctgctagaggcaaccactccccaccctgcccccctcctcctggtgctcacacacaacacttacttaaccagacccgttctttcgctctcccccctccctcagctcctctactccctctactgagccacagctaaactgcatccaattcgatcccgagtacctttctctgggtaagcaagctaccacatgcaaatcagggccactcaaagatatctaagcagactctgcacccaacagagca

SEQ ID NO: 232 - DNA, 569 bp, Artificial
  contol peak
cgatgtaagcaccaccaggtgcccagcgcggaggtggatgtgggcgaaccctggacgccccacaaaggacagctgcggcctgggaagggaaggagatggagcggggagagggggaagaggttgctccgcgttttccgcccgcccgcctgctttccgcgaggcactgaggcctgaaaaccgcgaggccgcccacgcgcgcctgacccgcccccccctcccgcccgccccaggccccgccctcccgcgcccgcccctgcgccggccccttttgttccctcccggagccgggccgctggctccgctggctccgctggctccgcgggcgctcagctcggctcggccgcggggcgcgcaggcggctgctgggcggcctcggtgcgcgcctcccgccttcccagagacgtggcgcgaggcccgggccctgagcacctatcgcggggatccccggcgccaggagggggtgcagccggtgggcagcgccgcgcagggaggggccgcagcatcctcgccccccagcgcgcccgggcccgagaggaggaggccggggctctccgggcctcccgccgctt

SEQ ID NO: 233 - DNA, 608 bp, Artificial
  differential peak M1
tgtgttggaattataatggtaaaataaggtctgtgccagccactcattaactcatctgattctcacaagaaccctctgatcttggtggcacaatcatacccatttaccaaagaggaagcataggcactttaagtgttctgtactttgcctgaagactcagctagtaagaacagagttgcaagctggacatgtgcagtcagactccaagagctctaaaatcatactgagttcctcttgaatacaaacaaaaggagttaattatatggttcagatctttgatagcatcagactatgatttcctgttgcagccatttccacaaccagactcatgtttgtctcatgagtcaacaagcagctaaccttggctgggagccatgtggctcctttcaagggactatctactgtggtcctttaagtgtagactttgtggtttaactgtcataaggcaatcttaatttgctttattttcagattcctaatggtgggagcacacatcactttttacgctctagttctttatgttttctttttccccctttctgtttatacattctggtgttgtgcttgaatgttaatatcacagcattgggtatttgtgtagacttgtc

SEQ ID NO: 234 - DNA, 628 bp, Artificial
  differential peak M2
atgaaattgattctgggtttaaaaactcagaaattcagagagatttttaattctcactttaagacgtcattacatatagtatgtgttgaattaatgcataaatgaatacacttctgtacatactcatgtaaataccttgtaacaaacttaaccgtatttaccactgaaatgaaacctctggacgaggaatacaatcctcaatgggaagctgtctatattctcaaatattcataaaactccacttcaaaacaaaaatagaagtggtaactatttatatgggtttgtgacctaatactaaaacctcaaaaaaaaaaaaaaaacagaaattgtgtgagatccctatctgttacagcaagtggatattctttttaaaggtcacaaagatatgatgagtcataaccccccagtaagctttgccaaatagctggctgaaaaccaactcaaagaacacagacatgccatgggaagtaggcatgtcataccaagttacttttaattttttttcattgaaatactaccctgcaacaggttacttttaattttcagtggcaaattgatggatagaaatgcacaaaaaggagatatggcaaaaatatccgaaaactgttaaaatactggtcaataatat

SEQ ID NO: 235 - DNA, 574 bp, Artificial
  differential peak M3
aagtggaaagactataatgtttccctgtcttacttctatccattcaactaacaagtatttgtttggccagacctgttactacaatgcagacagctctagtcactttggtctcccttttggctcagttttgcctcaatccaaggggttaatattgactttgggcaggaagaggacaaggagctcaccttcaggtcaaattccagttctccaactttggcctgctccatgtccccaaactctcctggttctgaatttctgctttatatcaaccattctctgtaatggacttcctgtactagacatcagtctagtctggcagtatttcctgccaaagacatgtgggcaaggggctgagtcacggcattctttgaactatgccagctgatcatcagttctatgcctgctctaacagccatcctgcatggtggatttgttttgggcaccttgtgaacagtctgcatattccaacactcctcttggcttccactgttcctcctggttatgattgcccctgaagttcatcactttcattttacattgaaacttctggccaaagacttgttcaacattccca

SEQ ID NO: 236 - DNA, 689 bp, Artificial
  differential peak M4
ccaggatttgccttttcttcccctgtttcttgctgttagaatgttaatctggctaaatccattcagcttcactcgtttggaaatcagtcatctaatgcttgtagcgaaaaaaggagacaagctaatacatctcaacagagcagatacgtgggagccactcccacagatcaagatccttgcagaaacccaagccattgttcagccaatagtaaataagaatccgagtcattctaaaactgagtagaaagctgagtcatccttccctctatcaagagctttattccttttgcatcctgtggatcttggttggttaacacattactgaatggtgacgtgggtaggtctgtctgcctgcatcaaatcctgccctaaccacattctcaagatagttttcaccacatttactaaacttccttgaacctcagtttcctcctctgcaggaggagggtatgaatgctttattagttttctactgctgtgtaataaattagcacagatctagagggtaagagctactcccactcatcatttcacagtgctgtaggccagaggccccagatggcgtaaccaggttttctgctaagagccttgaaacgctgaaatcaagatgttggttgtccgtgtcctcacctagagctcagggtcttcctccaagctcatccctgttgtcagcagaatgtaggttcctg

SEQ ID NO: 237 - DNA, 822 bp, Artificial
  differential peak M5
aagcttcccctatcaaacgacctggtggcttttccaggatggactgcttagactcttaggaggggcccagatagcaggcccagcctggggttgcgagtagtgtttcttctaacaaagctatggcaccctgagaaaggaggctcagtattcccctgtgaggatctttgtgggtctttcagttgccctaggatggtgaatatgacaataacctgagctcccaaatcatctgaagcaggacctcttaatcactcaccgctgcgctctgagccgctgggaaccaaggctggttgctaaggacgctgtgggagtcgctctgagacagacgttgctcttttacggtctcgtgaccagggcttagccaatcaggacttataagtgaatttccacctgccctgtcccgtctccaccacatttgtgttcaatttctaaagaataaatgcaatgttgtgtgaaagacctttattcgtttttaaagaacatagtaatgatttgtttcgaatattgtacatacttgcatatcctgcaagaagccacggcaagaagcggcttcacagtgctttaatgtctcctcagtgtcttaatgctgcttccccaaaacaaaccagcacgcctatcataccaaaagtagctatgtctttaaattaaattgccgttcgtaagttccttttacatagaaaatttggagcagtcagtgtcctcagcacccgtcagtgagtaaacaaagcacctagaatgttctgctatgtactaaacaccagagggcgaatttaaacacaaaagaacgcctctttttaaaggtagtatgcaatagaattatctaat

SEQ ID NO: 238 - DNA, 604 bp, Artificial
  differential peak M6
tactttttagtctacaacctatttaactacaaaagtgtgccaatcactggtattaggattggggcagatgtatactctgaaaataaaagtgcaaaataaatatcccatctggaaataaaagtgcaaaataaagagggaggaaaaatcagcccttccatttcttttcctttgtaatgccagttgaattggtttagtgagtagttttgcaagacttgtttcactttgtttttggacatagctgagccatgtacttcaaacagaaggcagccaattactaacttctggttgctaggtgtggcttcctttaaaatcctataaaatcagaagcccaagtctccactgccagtgtgaaatcttcagagaagaatttctctttagttctttgcaagaaggtagagataaaggtaagtcttggttttccttttagttagttttggttgttttgaaaacttttctcatcactatgttaaatgttactgcttctacaggatttatggttagttgcatttaacatgataagtcattcataaatctttctgtagaattacattggatatttatattttaggaatcacaaattacaatatttttgttctacttagtt

SEQ ID NO: 239 - DNA, 412 bp, Artificial
  differential peak M7
tcaggttcaggaggaagagacagggatggcaaagttggaaagagaaaatgttacttcattctaaaatctacacagtttgtgaattcaagggaagccattagtaggagttcctattgctgtgcagctgatttgttttgaggtagtcaccctttgttgaagcctggctggcttccttgaaaacaatcagcaagtgactaaccaggcatagtaagactggtgctgacacaggaaaccaccaaatgatgagagacctggggatgcccaaaggaagtcaataaaagcatactctcaaattcttccagattttggcttgcaaagcctcccaagacttctggaaatccttcctaaatgctactaggaatgttccagccaaagggctctgagaaatgattctttaaaatcttggatgctt

SEQ ID NO: 240 - DNA, 875 bp, Artificial
  differential peak M8
ctcactggctcacaggtgagaagccctgcagagcggtacctgtatgtgtcctccggtgagccttcaggtgagaactttttgtgtacactttgttgcatccctcaaaatcacatctgtggatacgccgttttctggagtctggggattcagaccgtctctggcgtcttgtgctctcaatactaaatggtgaaattgaactggaaaaagaaaaagtggagttacacggtgagatgcacaccgccccattttggttctggtggcatgggaacattgtctcagacactacagcttcacagactcaaggccaccatgaccacagttcttgctaccactatcttgggcagatctcacttttcgaaacctgaaaggcaggcagcatgagttatttccggcaaaacactccaagggagagaaagacatgcttcaaacaacaataacaaaaactgaaagtaaatgaaagcagtggtaggtgagtatttcctcattatcagaagaaacaatctggtaggggtaaattaccatctcacagttttgaatttctgaatatcctgatagagaatattttaagaaggcttgctatagacaaatgacaaaatgatacatttaataagtaacaagagggtctgcactgcatgtgacaacatagaaactcctgtaaaatcacaaggtacatttctaaacaagggagtcaggctgaaagaaatgacatcagtaacatgaatcatgacaagctatttctgtttggtgaaacgcacctgacatttaacttttctagttggagtttctttgtggtgaggaataacgtgcattgtctttaaagattagctgtaccaccattataaaaaaaagttttaactaagaaatttggccctaac

SEQ ID NO: 241 - DNA, 517 bp, Artificial
  differential peak M9
gctacattgggggttaggggttcaacatatgcattttgggggacacaaacattcagtccacaatggtaggtaatattgtactccctatttctcaggttaggaaactgaggccacactgattgagtaatttgcccaatgtcacacaaataataagtgtcaatactgggacttgaacgccagcatccatacttttaaccactatgctgtgctctctctcatcatgagtcatgacgatgatcacgttagattgtgttagatgatgttagatcatgagtcatgatctaacatctagcaacccatcaacatcatcaacatctaacaaatccaatcctttgaacctattatagaatcatcagagctttaccacgctttttagaaaaaatttccctacaagaatacagtctctcactattattattatattagagatagatttctatattatcaaatttagagcaaataacttatatttgctatcaactagctcttaaaatgattatatcttgattactctagt

SEQ ID NO: 242 - DNA, 812 bp, Artificial
  differential peak M10
ttaatggtcctcccttaaaaggctttacaaaagattttttagctagatgaggtgggaatatatatgctagcaccagattccacttatcagtcatccagcagttacagcaagccagcagctctgcctgcgcctgagttggccatgtatagacaagtgaggactgcagaggcaggaaatatggggttacgttgatttccctctattagcagcagcttaaaccggcatgctgggcagcactgtctcctattggctacagctgctgagagcagcaggatgagggttgacctatatctgtcaggtgtaggtcaaaccctgaatttttgttgcaaacttcagtcttctagaacgccaagtttcacttgtgctcaaccacatacaatatttttatcaggatctgtgatatactccacctgcaaagagcttgcatctgtgggtttccctctctggattcttttgctttctttcttgctgcctctccctcctgtcaccctcttaccatttttgtcccctgcttccgtgtcatcctctggctgagaagggttgagatggtcacagctgtgctccttctagtaatcacagtaacaccagcccttctctgcctgccccagtttccatctgaccaagctgtgcccgttcctttcagttgcagttattgccccaggttggcacagcctgtgcacatatgtgctcctctgggccaccatggtttcggaatgtgtcagttgctaattacaactgcagctgtgcacagaaatacagcacacattcaaaatgtgggtcctaaagttcctacacagtaaatatttcttgat

SEQ ID NO: 243 - DNA, 667 bp, Artificial
  differential peak M11
ccaggcttttggggtatatttcaaagagtccaattactcttgagtcagaactcagtgaagctctacaagataccaaaatgttctccaaggggtacgcagcattcgcaaatcagagtcatcagatgcaaagaaagggaatacaaatgggcttatgaaatcattggaaaaaatacacaatttttcaggaaatattaagaactcagttcctcctccaattcagggcatagcagagcctcagggcctctatgtgaagaaaatccctcctgtgtttggcatctgagtgactcacccagaaaaccatccactgcgtgcagcaaagaggaaatgaatcatggaagcttctgacctttgcacttgtcatctctgcacagggaaaccacccacagacagtctcagcagaactggagtcagggcagtcaaacaaagcacacacagaaatgatgcaaattcactggcagggaagagcaagagaatcagggcaagagaacgcagctcaggggttgcaaatacaggataaacattggttttgtgatacagaaaacttagcagttgaaacggcattgaggatgatttggcagtgcccactgacctgttgttttaagcaggaatctgaacccttaccctgctggctgggggtgggagtggagtggaatggcatggaagaaaa

SEQ ID NO: 244 - DNA, 521 bp, Artificial
  differential peak M12
actaaaagaagttctttatctcccacttgctccatctctacattgccttatttcttttctccatggtacataatcaatgctttaagtttcactatatttacttatggcatgaatgaataagccctgaaaatgtagttggaaacgtacagcaatagagcaaaggtagcctcattcaataacggaagggagcaatgatgccctattttccgcttgaacatggctggtcgtgaatgtgctggctaccacagagttctcactgcctttgatgtggtgtctctgagagagcatgataatgcttttcatccttctcatggctacctcttttggcattaagtcattcagttactctttagtgactgtaccaggaagaggatagaaatagcaagaggagaaatcaaaagagccagtgtcgatactctatgagaaagagttggggcaaaaatttggcaaaaaatttgattattaaattatccaattgtagatgtaatatttcccaaggtgttcctatttactcgctaata

SEQ ID NO: 245 - DNA, 588 bp, Artificial
  differential peak M13
attttaaagtgagtgaaacttcccttgtcatggaattgtctttgataatacaaaagcactttactctgcagaaaagggaaagaaaaggctctacttgggtgtttggctgattatttctaagcctacttttctctccagtactagtgtgttaagggttaaagcaagttgggcagggtggttagtacttctctttttcccctcttgcttagatgtaccttcaaccggaaacctatgagctcacactttctaagcatgttcaataatgaagtggtagacctccacagaatgtttcaggactcaggaagtggttgagggttattcaatagttggtgttctccctttatgtctgcaccaatcctggtctgcactacagggttgggaaatgttgtttgtgttggaagtggtgtctttcagaagagatgtaaaaaccaaggtctgggttgcttgtggtcactaaatataccgtaatgttttcggtgagcatagggttgttcttgagtttcctttctaaatttacattcttttattcatccacatttttttcaagtaatttgaaggctgtatttctcttttcttaaacaaaactag

SEQ ID NO: 246 - DNA, 549 bp, Artificial
  differential peak M14
tggttctgagcaaaagtgtaagttacactattatggaaaagatctatgcacattttgaaactcgaaggttttccagaattattgagtttatacaaattctgtttttaaggctgaattagcagctatcagaatccattacaatttttttgttttctttttctttttctttttttctataatagaataggcttgctgttgaggaaatgtgtgaaaataccagtgggggagcagatttttctaactgattaaaatatgtggagctgctgcaatgatttcctctgtgtttcctttggctacagataataccacaaagctaactcagtgacgcacatggccagctgataacagctgacacatgtgaatcgaaagccaagtgtgtattatcagaaagtgagttaaaaacccataaaactccccactgtctgacttgaaaacattcctcttgaaattttaatgacatggtaggtaatatttttaattctgcgtgctaaaacacttccagtgaaatctgagagtaaaagcatgtccctttaatcaatatgagctaga

SEQ ID NO: 247 - DNA, 538 bp, Artificial
  differential peak M15
ttttaaaaatcaaagacaaactaaaattgtcagtaggggtcctacaggcaaaaaagacatcatctaaaaaagaagtcttaaggagggagtgaaaaagtaaagatggacgagaacagtgtaagtaaatgctgtatatcttcctttaggttttgagcaccttttggattaatgccagaagaaatccttcaaaagtaagtgggtgtgtatggtcaggcctggcttatgggcttccctgcctttatagaaatcatgtcaattagtttcagcttcatgaaacattagtcataaaggtgtgaaagcagtggtctaattgaaccacaggtagcttaatgactcattttaaacactttactctcactttctcaagaaaataggaggcacaatgaaataaattgctaggctaaagccaagtatcaagaatcctgatattcaacatagttaacccatagggtggatgtagaatctgcagcaaaggcccagatcaggagctaagactcagataggactggatagattagagaaatcaaccatctgggcc

SEQ ID NO: 248 - DNA, 745 bp, Artificial
  differential peak M16
ctgtatgtctgttaactgttaacaactgcttcattttacacagtaagaaaccatatcatagaacttgcatgacttggcaaaggttacacattaaccataagctacataagggcaagggacatgcctgtcttgctcacccatgggatgccaggaccaatgcaagtgtcagataatagtaatcagccagggataattgtcattgaacagatagatcctctaaatagtgacttgtgtaaaaggctagtgttaacactacagtagtcacatctggcttaactgtacagaactataagcttaggaaaggtttagggtttaaaagtgagttacataaaaacaaaacttttcacagaagtatttggtttgtaccactgtatcattctctctttaatctgattagttgtctctccagttaccagggcaagagaaaaaaggcagtcagtgatagacaactttactcaagggtgtggatgaatcatctctgcgatttttttaaggctgacgctacatttattccaaggaactggcagcatactctatctgatgtgccagattattaaatcttctacataaaagaaaaaaaaagtatttttacgttgccatgttaaagctgttaaagcagtcatcctagacatgattactggtagaaagttcctgcttttgtttgttttgagacttgttcctcattattgttaagcaagttacaaatttacaaggcagaattctgcaaggattatatcccttgaga

SEQ ID NO: 249 - DNA, 757 bp, Artificial
  differential peak M17
attggaggaaggggaacagagaataagaatatgtcagcacttcatgaaaattttttggtagattggacagtaaagatgctaacctaattactttccctgtagctagttccagaaattgacacgatttaaaaagacattaatcattgacttaatgtacagtgtgtacatcagttgctagtaacttgtatgccagtgaaatctgctgttcttactttagaaaacaaaaatccatatatcctgtttttctatctgaaaaggtagcgttctcaatttctctaaaacacattttttttagaaaaggtatctatctgccatttgatcatttttctatgtgtgaaatatgacataccatctcggttatgctgaacagcaggtgcgcagagcttcaaaggcctaactcacaggtttgcatttccttcactccacatggcttctgtggcatcctgtgtcatgttttgtgttctttcctgtggaaactctcatacccacattacttcagtctagtttagcaatcagaaaacatggtgtctgcccagtagattttgcaaacattttaaggaaactgtggtagtcatgcttgattgaactgttctttctttcacttataaaggaactactgctcatgccttcattcagccatgaatgcttggctttttaaaaacagtttctaggacactaaaaaaaggagatttttgtttttcattcatctccaaaaaaaaaaaaaaaaacaaaacgaaacagcataggaaatactcct

SEQ ID NO: 250 - DNA, 748 bp, Artificial
  differential peak M18
attttcttaaaggctaaggacacttatagagctctccgatttcctatctgataaaaaaagcccaaacaaagctgcggaagctggcagagccagcagatattttgtaaggcagcttcacctctgctggcattttgtggggcaggagagggggagggcaaaggtattaaatttaggcttaaaagctcctcagaaattctttacacacaagagaccaagttggagaaaagaagatgacttgtgccaaggggaagaaagagagagactaatgcctgtggttttcaaaggttaacaacaacataaaaaagccaaggtatttgcacagggatgaatgctcattggctcttggccaacaattatacaagggcttgtgcccacatcctgtagctgtggtttagggacgctgctcatgagtgacttcttatgcttagtggcaggaaactttagtcactgatgtgtaaactcacctttggtctgcacctcccacaacgtgctccgaaagcagttaggggactaggcctcttgatttccttgtagataccagttccccagaagctaaggaaaaactctgaggccttggagttactgagagtggaccaatacttgtcttattctacctgattttggcaactgcttagggttcttctgtatccacaaaaaggaattatgatatcctctgctaaaccctctctttagcctcaaagcccaagaccaaaaaaaatgagccatatttcattgcaggcctgctgcc

SEQ ID NO: 251 - DNA, 722 bp, Artificial
  differential peak M19
aaaatgcctggaaattgccaaaattatacagtcaaaaattatagagttggaccacaagaccttttatacttgagtggaataccaaaaagaaaatattgatgcaaagctttaagattttactccgggttacttcttaaggcactagctcaaataacactcaaaagttttcaagatgcatgtaaactctacgtaagctccagtcatctatgtgctgactacaggatgctgtggttagtcaggttgaacttccatcagccaccttcatgttcagagtaaacaagtaacaattctgggacttgcaaagaaaccatgggaaaccctgaatgggactttaactttgaattaaaggaaaccacaggccaccacactgccacccatgaacatgtttacattttgtctgagtgcgggtgagtgcaaatggagtctcagtgacttacagagcactgttttaactatgatgtggttaaaacagtctaataatataactcctccagcaggctgctgagtagtatatgctggggcctcagcaatacagacagaggtggggaagaagattggagaggttaggatcagagcatccagctgacattaaactacagggtctgaaggtccacggcaaacttgggtttgtgcaggatgggtattactgactgttgggtgtgtgtgaggagggaaggaacatttatacaagcaggaggcatcattcaaaatgacataccatc

SEQ ID NO: 252 - DNA, 754 bp, Artificial
  differential peak M20
aaccttttgatccctggacagtctttgttgagacgatcaccaggaggcactcattcctccttaacgtggtcaccgaatctaagtttcattaagttttcttttaggaagtccagagacttgaggaataaatcatagtggaagtgcaaggtgtctcttgcaaggcatgggtttactgtgcttttgtggatttctgcccttagttttggtttagctcctttctgatgaaaaactttatgagaaagttactttatgagacaccaagtcaccccattcttgtgagaaccctgagaagtttaccaaatagttaagtagtcagacataggaaaaccaatgttgtggtaggatatggcggctcctcctctcaagggtgtgaaccatacagggctttttgatgtgtgtatttttacatgcgtgacttgtggaggcaggcagagtttctgggttgctcccctacctgtctgtcttcagctagaatgcttctgtaacagcccacaggactaagctttgcaaggcctgtggttagtgtctccagtacccaaaagatacatatttttattcagttaaaattggccattttaaccatttaaaaatgttcacttattgacttccagtacatttacattgttgtgcaattaacagcaaccacaatttaattgcagaacattttatcacgccagaaagaaacggagtgcccattaagccaggcttgtccaccctgcagcccatgggccatatgcgacccaagacagctttg

SEQ ID NO: 253 - DNA, 857 bp, Artificial
  differential peak M21
gtccttaagctttaccaaatacagggcctgccttgatctagagtgagctttcaatggttctggaactcagtttcagtcagcaaatgtacacagcaaatgcactgtgctgggcctgccctactccactattcttaggcacactggaacaactttgctggaaccagggagctagctgaatggcacgactgtcttctggtagcatctcacgcttgggtacaaaggcctgtctactggcttgctcaggcatgttgagagtctgagtctgctgttggtagacctgaataggtccacctgttccctttttctgagacagtgtccattggctacatgggtcacatctctgggggtggcataaataactacagacagggtggcaggaagggcagcctaaggaagcttgagcaagctctgagggtgactcagaagcacccacttcctgtgtcagttcagcctagtgggtttctttatatgaagcccaccttattaaaggaagtggaggtgggggagcaactagagacaactagtacttgtcacctcaagcaagatgggttatctacctacattcaacctccactctctgcaatgtttactcctgccagtgacttagtcggctattttcagtgacaaacactccagcttctaggccaggctctctgattttggcttcctctgggtgcacagagaatgaaggcagctgatgctccactagcggctgcctcggtgccagcaacctggctgtgctgctgttggtgccttgggtctcttttcctgtctgcccttctcagctcccttggttgctgactcctcgccatctgaccatctgcctcgctctgccccttccacccaccacacatttc

SEQ ID NO: 254 - DNA, 545 bp, Artificial
  differential peak M22
acccctccagtccatttaaaatattaattaccaagtttcctgccttggatattggagttcttaatactaacatcaaaccaaatgttttatcctcatttctaaagctctactccattgcagacaccgtgacatctaacacttagcagatagaaaaaaagtatgccattgtaaatctgaaaacttctgggcttgcatgtcagagactggaagtggtttgtttgaaaggagcttatactgaggcagcatagcataacacagtggttaaaagcaagtctgtgaaagtgagaaagactggggtccaaatcttggctctaacacttctaagctacgtgactctgggcaagttgcttaaattccctgagcaccagctttctcatctgtatgatgtaaaaaaaagagcagcactgtttagtaaacatggtcttggctttataccacttcctaatatttctacaactgtcaagtcaatcaacatgttaccttagaaactgtggaaaaggtataacctttggaaaatgtaatcaggggggaacaacttttcctag

SEQ ID NO: 255 - DNA, 883 bp, Artificial
  differential peak M23
tgaagtcagaaatgatcccactgatgattttaagacttgacctgaaaaatggaggcagagtttacatcaagaaatcttcaaatcgcaaagtttagagacataaccactattcatttagttgctaagacgatctgagagttctccctcaaaaatccctccgattcatcccccctttctcatctccatagccagcaccccaatcaaaaccaatgtaatcttgtgtagacagctgcaacaacccagggaaatgaggtggtgtagggagtttgaggacacctgtagaaagggtggaagtctggtaccgcttgtaaaagtgaaacagtacaaacctaagtcttcagattttgtctcaggaagcaacgcaactcttgtgcgagcaatccatgagcaggaagtggtgaaatactttaaaggtttattgaattgctcattctagttatctgatcatcttgataaatgtagggcaagcaacaatgatgccttggtttttctggtttacttttctgtgagcatacactacacagctgctcccaacctgatcccaccatgcacatacatatgtgtaacccacagatatctctcgttagaagtaaaatgtgttagataagtgtaactttctttcttactctgcatacagcttcacctagatgaactgtcacacacacacatacctatcagctcttattttgtaagctatgttctgactttacgagccttgacaagctcaagtagcatgatatgaactgccctctacagttggtaaaagctgaaaacttaaccatgatagtttaaccatttacaatttgatcagaaaatgaatgaataattatagataatgacaaaactaaatttcattgccttttaaaaatactgggtgtctaag

SEQ ID NO: 256 - DNA, 542 bp, Artificial
  differential peak M24
tcgcagaattcattcaggataaaatttcatgttgtagacaccagaaacagttggtgagggagaccgacagacttccacccacttttcgttggactatgcaactaagaaccttaatatagatttttttttagatggtacaagtgaacaatttagtctagattacctattataacaaaggatgatcaagtggccattcagaattttcaagaacacacagcagtgtgttcctcacagacgagccttctccacagtagggttgtaaagtcctaactgtggtttcttctatacaggaacttacacctagaaaccacagagtaaattctgcatgctgtaataagtcaggcaggaagggactgaaagtatgcaaatttttctacccacagccaattatatgagaaagacaaaacaaatttagaggacaaaagaatattcattattagaatgtgtgagttttccagatcactgacattttaaactagattgatttctcaagtcagctttcttgactttgtaaatgctttaaaaagcaataaaacaggtgg

SEQ ID NO: 257 - DNA, 653 bp, Artificial
  differential peak M25
cagtatcattcaccttgatttctatccagggtaatctgaaaaacatcgattctaaccttctgtaatttgtgttctagaatctacttacatcaatctttaaattttttgaccactctacctcagtcccacaggagaccaaaaggtaggaaaaagattccagggccccatttgtgcttcaagtcgatcagctagtcccacggtgaagtcaggtgccctgatacagcttttcaggtcttttcctgtgtgaatcaccacctagaaaagtcaggggcctgcctttggtttctaacaggaaatggtgccactgaagtgcaaattcacacggtggggaagctgcagggctggagcaacagcagagcagaaagagccagcaagttagacagtgtttctcttaaaaatagagtagaataaagaagcagaatagaaaagaacaatgggaaagagatgcagaattagtttgacctaaaatacgaatcaactgcttatcaataagccttaaaaaacaacctaccttacagcttgcctgagaatttgtgaagaaatacgaaggcaatgagaactcaaagtgtaaagctacttctttcatatctccatgtcttcttctctaactgtcttcttttctctgtagttctcacacttccccctctcccttt

SEQ ID NO: 258 - DNA, 852 bp, Artificial
  differential peak M26
cttatgttctctacttaatcaatagctggttaacttaaatctgttatgccaggataaaaacaaatttgaagatggttctgctcagtccacagtcacttctgcctagttgctatcattatagaatgtgaaataaactgcagctgtggccacaaagtcccttttgctgaggctgggagggcccaggttgcagcagatgcggaggcctctgccaggcacctgccctggtcttgctgctgacctccttgcagggtttcggtggtggggctcctcctgtggtcaggtcactgggatttcgaatacgatctgtcctgtcatcactttcactttgattagcttcattctgaagaactttcgacagcattcgcaaacatgggtcggcatgagaaaaccagttcctctgtataggactgatttaatctctttgtatagaggctgatcttgggaaaaaatacactcctgtttacgtcaaacatacaggacgcgtcagcccacagccactctcaatgcttcctgggttttcttggtgggggcccctctgcatcgctgggacagtgagtgaagagattggccctgatgtgtcagtccctgctgggcttggtgctgttgtctctgggtgtaatcaaacaatctgtatcttagtgggacatcctgcttgctctgtgtgactcagagctctcaggcagctggttgaagctttgtttatagtagtgccacccactcactccacacccccgactccctataaactactccacaggtggccagacttttggtgcaaataaaagtttgtcaactgttgcaccctttctcctgtctttttttttttttaacttatctaaacaaaattaagct

SEQ ID NO: 259 - DNA, 621 bp, Artificial
  differential peak M27
caatgacagagtttgtcccttctttcaagataagaatctctcatcctgggtgggcaactgtcttgaaggactttttgtcttgtgaattttcagatgaataatcacaaacattcattcactcacccttttggctagcaggcatcacatccatccgttcatggctgacagcaaatcagagaagggatcgaagccctcaccattgccgaaatgttttgttcggaaactggaattgtcaacaaaagcttctgggacactgcaacacaaaaatattttttacacataactaagatgtaacttctaaaaccaccacttccttacacttgaaaacagcaagacatagattcttgaggtaggggaaaacaccgttcaagccagggtatgtcaatacatcaagtgtgatgctgttaaatatgaaccggaaatactgttttgaaagatacatttactaatcacttcaacacgtacactgtaaagttgttatttattatgggctttgtaccaaatattttaaatattttagcatatgaaaataccacttcccattatcaagtcttcaggtaacaataaactgttttaactaaaatattacacagataaatgtaccattccctattaagttat

SEQ ID NO: 260 - DNA, 799 bp, Artificial
  differential peak M28
gttgtgggactactattaatttctccacctctcagatttctttatttctccatctcttgatttattttttctgcaaacactcattcaataaacagtcactaagcacctgtcaggccctgtgctggagatgatgaagaaataaataacaaatagtccctgccttcgaagaactcctagtgtagagcaagagctgaatatgtaagcaaataactctttatgatggtatgtaaaagttataccgagcatgattagctttaaagtggcatttgctttacttacaacccgccacattgccacaaaagagctcagaagcttaaaaaaaaaaaagtgcagctgtcagtagtgattaacacaacggctagacactgtgtatgagaacggatataggtggttttataaggatcatgtggttaagtgggacacgcatgttgaacagagtctgcagacattttgcccaaaacttccttttgcagtgtgcacatgtaaatgagccttgcgtggaagtttttgcttccggaataagattgactagccagtgaagctccttattactcatttcagcaatcatttgcttttcttgatgcaatatataaaacgtgtacaaaatacagcaaaggaacttaggtcacccacgttaacttaggacgtttctgttgactaactttgggcgttctatttattaaaatttgaaagtcataaatgctgggcttaaaagaaaagaatctctcagaacctgaaatgaaaatttagcattatatgacagttttgagaaaaatgattcagagttctttaaaaatta

SEQ ID NO: 261 - DNA, 707 bp, Artificial
  differential peak M29
gagatataaatcaaaacagcattaggatgataatatctttcccgccaagcaaaatgaagccggcgttttccaagaacacacgcactgtgcacatgaagcacagtctattgttcctgggtcaccaggactcggagacggaaggcaaacccagtactgcagaggcccacgtggaaacaagccaggaagagcatttcactgccttctcccaacctccacacagccgaaattaatctccttgcgactcacacacactcctatgccgacttgaaataaaggcctgccacctgggtatataactcaaatggaagagaaattcaaaacaattagagagcaaaattaagcgcacactcaccagcccaccaaaacggagagggaaagaaagtggagagacaggaagtcgccttgggaagtgtcttcagaacctgattggctaatgagttcaagaggattccctcctcctttaccttaaaaggtgctgaaatgacgtaacccactccacccctctttatggaccagtgatgcttcttgcactatttttggagtgaacttcttaaaccgagtttccttttgagttaatgtacttaggcaaaaacaggctaatttaagctatttatacacgttgttgtttgcgtgggcctactgcattctattctaaagaattttcatcccttaagggtaatttcttatcctttgtgacctcaacatgc

SEQ ID NO: 262 - DNA, 744 bp, Artificial
  differential peak M30
catagactcttacctagaaaactctaaatagactctttttctagaaaatagggcatttgagaatcttaaaaatatcctcccacagcagcctcatatgtagactgaagtagaaatttgggagtcctcagtaggtagataatacataaagtcacaaaactggatgagatagacaaggaaaaattggtagagtcagtgaacaagagtcaatataactaaagagttatagcaattgagacgatataataagcaggagaagagcagtgttagcttgccattgagatttcagaggtgctactctagttattggtgatgacaataaaagtgtgtggcaaaggagggggtaggggaaaagatgggtaggcgtgagaaaccacagtgcttgatgacttctttgtggttgttgacatcaccaagaatgatagcagctttagtagaggagggaaatcccaagctctgctgtcttcatcctttgttaaatcaagattcaggtgtttttgtttttgtctttatccctttcaagtgttttagattctgtcaaaaaatagatttttcaaaaagcaagttcctaagctatggcaaactataatggattattttgtgagaaaataagcctaaaggagaaaatgatcagagaaatctcagttaaggaagtcagtgtggtggatctctcacttcacatatccccttggaataagagctcttctggagaagaacaagtctgaaataaaatgttggaaaaaaatc

SEQ ID NO: 263 - DNA, 727 bp, Artificial
  differential peak M31
gtctacaggaagagagaggggaagaaagtgaagttctgcaaaaggacgactcctattctaagctgggtcaggaggggagggagatgggcaagggatggaggaggaccagctctgagtacctgctgtaacattccaggtactcattatggtgggtaccacactaagcatctttcctcaaattctacaacacagattcatcatagggcatattgttagagggtgaaaacaatgttggattgctggctcaatccttcgctcctctttgactgggctgagcgatatatcctcttcaagtttcaaaaccagagattagaactttgaagggatctatcaacctctctttgtcaccatcaatgatgactcagtcagaaaaggcaggaagtggcaaaggctttcctgtctgtaattctagagaatcacatggttactgacagcctcttgggaacagcatttcagaggtgccaggatgctttatagcaggttgttggagacatgctggagtagactagggggagctgaccaggattttgtgtgaggagaaaggaactttgggacattgtgtcctggttcctgatgtccacagtgatcagacacaccagctgccaaccatatttaccaatgtgaaaaaggccagaaaattaatagctcactgcatttttttggtgtctaggtaatcctgtgcattacctagtcatcagcatcaaaccatcagattcaatctattcat

SEQ ID NO: 264 - DNA, 593 bp, Artificial
  differential peak M32
tctaattgaaaaaataaagaccaaacttttgctagaaccatattctgtattatctgaaatgctggtagaacatgctggcagattagcaatctgaatgatacacattagcaatctctcaatattgatgaaaagaggcatcacagaagatgtaataattcatatgtcccttaggtggcagcttaattttttgctttagatattttctctcatatttcacaaaggttattcaaactgtgttagaccatacctattaaattcaggctctctttgaaagcactagtgagttagcaagcatgagaaacagaaggccaatcagactgtcagaatgtacaaacattcccctaaaaggaaaaccacagagttcacttgttcctccaactctagtaccccaccactagggctgtctttcctggctgtatgtaacggtaagaagagacagtatctttatattttaagatgaactgatcaaaaagcagatccactgatatggatacagcagtaccatttaaaagtcaccacaaattacctaaaatacactggggtttcataaaacaccatctatccatcacaaataatgtttttaggaagcatgt

SEQ ID NO: 265 - DNA, 820 bp, Artificial
  differential peak M33
tattatccaattcacatttctccatctcatttatatggcaccatgacagagcctttgccaacatccacacacatgaactccatgaattcccccgatctgctaatctagtagtaatccagaaaaagaaaaaatgtggttagtttggcatcacctattctaaattctactcatgactaaggctgaaaaactgactgctgaatgataggtactaatctttaaccaggaagtaacactaacttgttcataattcttagactccacatttccctctttaaaaaaaaaaaaaatctttacatcagaccctttgggtgtttgtcgtgtctctctcattgccctaattgttcagccatcaccccgcaacagctcccagccccctgcaatatgactcatctgagctcgaagttctgtctgctctatctcacgcactcaggatgttaaagctgtaggctcctccctctctgggtttcatagctctctaagcttatttttattatacgcttcctggtagaaagatcattttccgtgatacagaaacaaaagcgttattgagcagttctgagtagttctgcattcttcttggcagatatttacactacagtctcccttaggtctacgagttgtcttccttgttccctggctctgatagatcaataaagccctctgggttggccccagcatttgtctcaagccctgatctctacagtgtttagaacttcctcccgtggttcttaaaggtccaggcccttcttttgagaatgtctttggatggctggctacaggaccatttacctgtgggaactatcttggaataatttctggg

SEQ ID NO: 266 - DNA, 515 bp, Artificial
  differential peak M34
gtgtcctttttcttccctttttagttttttttggcctggaaaccagcagggtgcctccatttgctctctcatcctctggttcttacttgagtgtggaagtcgctccactggtggaccttgcagtggcactgagctgggtcacttcagccgtgaaaggccaactgtactactgtcaaggcttgggacagttaagcgtccgtgaaggtactggatacagcgacagcaggaagtgcagcagtgagaactgaaactaatttcacagactgtggtgttgcctgtgatgtagtctccctccctggctcttctatctcaaaaaaggaaaatagccacccttgaaacatctgaaggctgcgtgccaagaattcacacagtactcctgaaacaggatattactgaagcatttttctcaaacagaaacagatgtagttccctgttactgtcgccagtgttagtagatccttaaaattacctgacagcagtgtgcccctaaatgtactccccgctcctacacacat

SEQ ID NO: 267 - DNA, 751 bp, Artificial
  differential peak M35
tcagcatatattagtgtgtttaactaccttgtggtgaattgtttgttgctactggtcgtctgtaaaatagcatcttgattcctcctctgataaacacaattatgttattatgaaactttcctctctcgttttacctccttagagcttgggaattaggccaattgtagagctaagtcttctgatagtcttgctgttttaaatattattgtctatcagccattcattactacctttttccatgcaaagggaacaccagtgttgttcaagaaatcataccttagtgaaaatgcatggccctctctccagctcaggaataaaagcctattagtttaattcagtcatgtgacttgtccctctggcctgccttctccctcctcccctaactgattgattcagggctgtctaggaacttctctctggcctctgaaatgtgaggagtttgctctgtggcttctggggctgactttcattttcatttctggatgtgatattgggactgcagcacctgtcatgtgggccacacgggaagttagcccagatgtagtttgaaactgctgctatactgtgagaaacagaaagacaaaatgggcagaatttgagtctttgggaatgagtccttggttgagccgctgaattaataatagacctcctcaccccgccctaaaccccaaccctcctgcccataatccaacatacatttctgcacttatatgagaaaatcaaggtccttatgtttaaagtcaattcagta

SEQ ID NO: 268 - DNA, 575 bp, Artificial
  differential peak M36
aatatagcatcctatttattgttaatgttgggaagttatttctatctggataaatcaagatttatatgtctcaagtgtgtgttagagatgaaataagagagactgtggtgagggaaagttcctgagtaacgtgctggaataagtccacacaactttattgctcatatttttgcctttacaggcataattgcagctgaacagtcattaggaactgtgtggtggtttatcttcttcaaccgcattactcaccttcaggtcccgatggaccacgcccatctgatggcagtgtagcacagcctccaggatctgctgaatgcaatgactgcatgcaaacaccagggggcgtgtgttagttccaagcttacactcactgtctgtataccctcagagagactgggttaaagtgcagagctagcagacaactggggttgtattgtaccaccttcagtcatgtaaacatattgctcaagtagagtgactactacctcagaacaatcagatcttaccagatgtgagccagaatgtgggtccatttgttctagaaaaatccattttgaaaacactttatgtcattc

SEQ ID NO: 269 - DNA, 623 bp, Artificial
  differential peak M37
cctgtctcaaaaaaaaaaaaaaaaaaaaggagggtcatatttgtcttcaactttaaaaatgtctgtcctttgagcgggagctgcaagttaaaattttaagaaatgtttagagtaaaatggaacaattctccctggtgagagaattcacttcttcattgctggtgctatttctttacagagttttgcaatagggattaggaggaagggtgaatcagagcccccgcttgtggtttcactgtcttatttatctcctcgacttgctgagacagaaggggaaatccggggaggggttgggtggaggtgagaagaggagaaacagataaagaaacacagaaaaagagagcagataaatggagagggaacacgcatattataagggagtaaaaaatgagaaccgaaagaggcataagttaggcattaaaacttttatgtcatcttgtatttacttgctagaatataagcatagagattagatggtttaatgagtgtgtgtgtttgtgcacgtgcacatggatgcatgctattttcagcagggatagtataagtccttggagcacttagcagcaggctaaacaaattgtctgaactgccttgaatgctctgagcaaacttgccatttttat

SEQ ID NO: 270 - DNA, 632 bp, Artificial
  differential peak M38
ccagattctcatttgcttgggaagcccgtctgcatgtcagaatcagaaatggctcattgttaacctattggtcttaacaaagtttgtacgtcaatgaaggacacggtcttccaagtactagtgtcagattcaaggaactgggactagaatccagtgatgggggatgttcttgtcatctttcctgagtggccataacagaatgtgttgagggaagatggctctcagttttaggagataaggttcagttaagtgctttggggacaggaaactggttcgtaaccttcaaagttaaaacctgtgatacttatctgatgctcctctgagttagccaatcacacttgaagctgctcaatactgggtaagttgctgccagcacaccctgaacagcttgtggtttctttacacactgagctttttgcatttccatttgacctgagatttctactttctcataatgcgttttctaatgagaaggacaaatgtactgtgtgcttctcccaacaactttttaaatgactcctatagcttgaagaaagagcagaacttttctaggtgtttattcactgatacctggaagtagtcgcttactcatttctctttttttttttttttaaacttgtagaagtacaaag

SEQ ID NO: 271 - DNA, 776 bp, Artificial
  differential peak M39
ccaagaagttaattcagcttacccaagattacatatttgcttagggccagagctaagacagggacagactctgtttacttcatttctcactaaactgtaccactgcagtttcgacttctttctaccactgaaggcacagaatgcactgaaggcattctgtgccttcaggaggaattttcagccccttcactttcctgatgcccgggaattctttttaagttatgactaacaaagtataaaacatgagtcaaaagttagtccctaataaagtcattgtgactttaatgacttcggcagttgtttcattctgcaaaaacaggaagatcccacacaataaggcgcctgtctcacagctgggctcctgcgtagacggccacccacaagagtaactaactgctggggctgccttcctctctttgacacctttttattctctgttcttacagttttctcctactgacctcatcctgaggttatccatgagaggaaggtacctgctttagctagtctttgatagggtgaagaatagttttaatcacttaggagtcactcctaagtgattttcaggtcaccctcaacaggcctaaattagtcttagaagaagctctttcatgggctttatactagcttcctgtgttatcactattctggactgttttattctcaaaaacataaaatccccaaattttagatttgagaagagcctgcgatcattccagttcactcctcctatgtgattagtttaactaagaactcattaccaagatagtgcctat

SEQ ID NO: 272 - DNA, 864 bp, Artificial
  differential peak M40
tatacagctgggcaaactgcctaaagtttcctcaagcctcagctttactattttcttgaatggctaaaactcacaaaacacatctagcgtcaaaaactttgcgaccccggaagtaaaaggttaaggaagggggtcacaggagaaggctatttccgtttccgtacggaagcaaaggagccaagaccatggcgaaagccggggataagagcagcagcagcgggaagaaaagtctaaaacggaaagccgctgccgaagaacttcaggaggctgcaggcgctggggatggggcgacggaaaacggggtccaacccccgaaagcggctgcctttccgccaggctttagcatttcggagattaaaaacaaacagcggcgacacttaatgttcacgcggtggaaacagcagcagcggaaggtacgcgagaggcgggggctgccgggcgcttgcgcgttgttcctgacgcttagggcggtcgcggggcgcacatctgtggttgtctgctggtctcaaaagtgttctctgtggctccgtgggaagggtggcgtcgcggcccacgtgttctggtcccctcgccctagtttggagactctaggttcagccttttgcctgagcgtcaaggtggaagattatcttgataggctctttcctgggacgctagattcacaaggaaagctgtaaggccagttggcttagtagtgaaaactacgccctcctggcccagaaggcaaaagtctcagccctttggtccgtctaggtagcgttggttaatttgcagaatctgaaatggaggcaatacgtaataaatccaaaatactgcttctctgattcctgcaggagctccttaccgaaattagtactgtaaagt

SEQ ID NO: 273 - DNA, 876 bp, Artificial
  differential peak M41
tgtggcaatagggatattcctcggtgagcccaagcttgtgtctgactccccacattctcctctagccagtcactcactgcttttcctgaaaatactcatcttcacctacatctctcaacttttgactaagctgctttctcctcctagagagccttgccacccccttcacctatggtgacttcactcattcactcttcctggaaacacagcagggttgatcaccacccatttccgttccccttcttcctgcccctccctccttgaagtcgggcatgtcctccaccgtcgatggatgatgagtggaagtgacctgtgtcactgttgggtgggaacttttgtaaccactgtgtgactcagctgctcttctgactcttgccgctgtaactagccaaacaccaaatggtggaggctccattccctggggcttggaggaaggaaacacaatgcagagcctgcaactgacctaaaatggacatgtaatgtggcagttgctaaggcaggaatgtgtgtgtccccactgccccacagattcacatattgaaagccaaccctcaatgtgatggtgttagcaggtggggcctctggaaggatatcaggtcatgagcgtgaagccctcctggatgggaccagtgctcttatcaaagaaaccccagagagatgcttttacccttctgccttgtgagattatagtcaaaagatgactgtctacgaggaacaggccctcaccaggcaccagggccttgatctgggactttccagccttcagaaccatgagaaataaatttctgctgtttataagctactctggttataatattttgtaacagtagacaggaaagaaagaaaacctgtttccatctgctgctaagattatga

SEQ ID NO: 274 - DNA, 722 bp, Artificial
  differential peak M42
aaacagttaacaaccactcaaatagaatagcctttttaagatagtaatattgtttagataggaatttttcttcttctcttttgatgctttcagaacacctcagtatgacatcttcaaatggtgtacacttcagttttttctttgctaaagacagtgtgtatatttccactatgactagcaacaatggtgcttgtattgattgttctggcagctatttaaataggcttgttctctccctatctcaatggcctcacaatgctgatactacaatgctcaggaacttctgagcaccctagccagatgccccttactgcttgtggacaggcattccccagcctcttggctgggtgacagcaaccacagcagcccacaagacgggtggagaaccaatgagtaaaatgataaacatttgtgatgaaacaggatgttggagacaggggggtgaggagacaaaatatctcaacgctgaactactaacgctactttggaactcctaacagtgttcccttgttgtaaaactaggagcagtaaaccgatatttttctctggtgttttctcctttattataattaatttcagaattactgctaaacaacacaccattacccaggcacagaacttaagactgagctaactgaaccacaagcactatacccccataaatattgtaaatgttcattggagacacttaaacaattatgtgaaaataaaatgttttctgg

SEQ ID NO: 275 - DNA, 579 bp, Artificial
  differential peak M43
gcgtaggagcaaaatctcggggatgtaaataggacttcactgtgaacttatatatgactggggaaatctgttgtatcttttttcagttccggtatttctgttggcctgtcggcttgcttagtgggagggagagagagctggtacaatgggattgttttaaccattggtgccccgctgctaccttaggcacatacttattatcagcatccatttgttcagctacaattctatgacgtccattgcagtgagtgagtcagaagccacaagctaacaggtctattccttcccaccgtcaccttggaaaccaatgcatcatccacaggaaatgcaggccccgatggggagtgtgcccagatcgagttataaagctggcaatcagtttttcaaaggaagtcaaataataatttgtagaatcttagtctagggtgagaggtggatccagcctcttgggtatggattgagggctgtcaggcagaaatatcacgagaaattaatcagttaaaatgtaagtagtaagtaaaatgtaagcagtaagatgtctgcttgataagaatttgaccaaaggcaagaaatatagag

SEQ ID NO: 276 - DNA, 663 bp, Artificial
  differential peak M44
tcataaaatgatgtgggcatacctggaaagtaacgtggaggtgtaaccacggcgggggggagttctgctaagaaaggaaaggtgagaaaacacttgggagaaaatggcttttgaaaatgtgaattgaaaagagaaggagctcaaagccaagaaactgacactgacagcgcgagatgattgaagtgcagattaggattgagttggggcgttgaaaaataaagtacagatctgaaaaatcttggagagaaaaaaataacaaagcttagtaagagctccttgagcaggcctggggagagagatgtcagtgatgactcagatgtcaggagtggcaaggcaggccagctgcaggactgccagtgacaggaggaagtaggagaggaaatgatgcatgcgatagctaagaaacactataaatggctacatatctgtggtttctccagcaaaaatgcatgtgcaaaacgttctgtattcagtaccctagttaaaatataactgtggggagagcaacagattaactacacaaactgttgctttaagatagcatgcctaggaatgtgcatgggaaaaaaatactgaggcaaaataaaattcaattgtggttagctctagagtagtattatattaataatataagtgacttttgcctcctcttttgtgctgcta

SEQ ID NO: 277 - DNA, 745 bp, Artificial
  differential peak M45
agtctttgcctttgactttttccctcccctccaggctgcatgatttccctttttctttttatgactttgcttatatcaaaagttaagtcttatgtcaaccttgtcatgagaacaaaagaagctttctagcttttaactaaaaggatgtcagttaacaacggaaatatatgtcaaacagaaacagggtctcagctttgtggtcttgggaaccctctcttcctattactactgcttattatccaggacctggcgtcttccctttgtcacccagggcccggtgtcttcccctcaccacccggcacctagtagtcttccctggagctagtgtcttccctgagaggcaagacagaccagcccagccagagtgctgctggtaacatcacagagacggcaggccccatctttggcagctccccagactctctgtggtttggttttgtagcctgacactcgaggtggctcacaacttccttctggagtgaacatttaacatcctgcctgcccgtcacccctcccttcttcagggaaccgtactcgctttcttctgagcatcactctgcgcctcttactgcccctctctgatatgtgagccaggccatgctgctgtaataaacaccaaactatcagtggctcaagacaatgcccatgtcacagcccaatgtgggtgcccctggttggacgctgtctatgtggtcattcactaacccaggtccttctcatctggtagctctgctctccttaaaat

SEQ ID NO: 278 - DNA, 563 bp, Artificial
  differential peak M46
taattttctctgtttcatagataacaaaactgagctcaaatgaaattgccaaagataacacaaatggtaaaataacgaagaactgggattcaacctggaatctgtctgactcttattttccattgcctcaggctgtacctgattctcctctttacacatgacaactatgcccagcatcttgctgttcataaactctgttttcaccatatcacacttctctttaaaaaaaaaaaccaaaacaggaaaccatcagtaacacccctactgctcttttcatcagtcgagactcgtctgtatgactttcagggatggtgacagcccaccccaccctgcctagccaaccttccttcccttgttccccagattcattcttcactatcctgtaaacatattattctgccatttctgtactgtcaaggggtactcccgtactattaaggacatttgttgaaaatatattaaggtgctcttcttttttgccttttcaaatacaggatctttgccctggttgactcaggacatctctgcctggagggtgggtggtgcagtatagagttgggtgg

SEQ ID NO: 279 - DNA, 1042 bp, Artificial
  differential peak M47
aatctttcaaataaaaataaaaatgattttatagaaaacagatattgaagtaatacaacctttaagcttccacttgcatcccagattggaccccacctagaagaaatttggaatagagtgaattgaatttagtggttgactcataatctgggcaagagacttttagtctttcccttcctgtagtttgctgatttccctagaacacttactgcccttcagaacaagcaggcttctatggtaaccagtgaagtaaatggaccccgttgccaggaaaggtacatgaaacaaaaccaaaacaaggcagacaggaagacaggggcttccctagcggttgaattaagagttgtcaggggaaacatagaaatacaattatgaaacagctcagtgcgatgtgttaatattgattgtgataatgatagcagtagcatatggattaatatgtggtaaaagctgcggcctttttctttccttttagaactttgcctttgattacatatgcacagatttctatgacactgttcaataactaacatctattgagaaccaactatttgccaaaattttacatttagttttaaaaaccattatagccaccttaaaagtacattgtgatactaattttacagattcttccaaactcagtttatcagccatccctgataagccacttaggtctctcagtcaaatggtccagtggaatcctgtggagctcttagaacgtttgtaattatgcaattaatgatcataatacttaaagtttaatgttcatgttctctgccagtttttagagtgagacaagcacaatgccttgttcactggcccatctctagagcctggcatggtagctggcacacagtgaaagctcagtagacacctttggctgagatataaaaagaccaaggctttctgacttttgctcaattgtacacaagtattgaaacctcagccaggggcttttcactaaacactctgcctgagatgttgttttgtcttaagaattcagagtacatagacattctatcctcttgggtattaatcacat

SEQ ID NO: 280 - DNA, 712 bp, Artificial
  differential peak M48
ccagtttcataatcacatagtacactcaccctacaggattatgttctaggcaagttcgaatgttaagttgaatttcattctcttcaggaagtgacttttactcaagattgaatacagctgttagcacagatcactgttccaaaaacatttctatgagagctggaagaaaacaccattttaagaaatgcaaattcactttcattatttcaaatctgtgcaagcaaagctggaagggaatctctggtctctgtttgttagttaaattaggttattataaatatttaagagaaaacatgcaggatggctacgagatggctataagactttccatgtacttctggagcctacatattttaatcaccagtttgtttataacaaatacagtggcttggtttttgtttcaaaaatcaaagttatactcaatttctccttaaatttcactctctaatacaacttggttttctctctttatattcacagaaatgtagcctggcaaaaaacgtggggtggcctcagcagaggaaatgctgccagctgaaaacaccggatgtaatcgagaccacaagagacacagacttagttataaatgtcgaactaaaaagaggtattggaaagaaacaaagtctaactttttattggctggattaattattgtagtgaaatcttgctatttcatagtttagtcccaaacaaaacagcatgtcggggaatt

SEQ ID NO: 281 - DNA, 568 bp, Artificial
  differential peak M49
actttataaaaatggaaataaaggcattagtttgaacaatcctctatgctgctactgttgtaaagtttggagcctataactcactcaaattggtttatagccatgtgtttactaaatcttttttcatattccttttctccaggcatacaggaatttatatctcacagccttcttagagtcagtcaggttgagcctggtgactagttcaggacagtgaaacgtcagcagaagtgaaaaggccactcacttctggcagaggcaaaggagagcccttacaggattctccaatctagctcttctgccagcccggaagtgctgggagggcagagccactgacgcaagcagcagggaagctgagtcaccatggggacgtgagactgacttacagatgacgagtgcagcctcacagcctagtccatgttgagataaacacaatacagtcctgcatactttatagtcataaacgagggagaatagaaatggaaaatgaccctctaggcctctaagaccaaattataatgaaaaaatttatgtcttcagttttgaaaattaaattttttgaccctga

SEQ ID NO: 282 - DNA, 574 bp, Artificial
  differential peak M50
aaaaaatcacaaagacatacaaaatgctttagcaagaagatctaaaactttccagaatttcagtgctttgtttccccctagcaattaagttcaatcaggcatgtgactgaccacacacattttatcagagggttaaagaacacattcagccatgttcttcaacaggtaagaaccaccctctgtttatcagctcaagaaaaagccagaagcaaacatacaaaattgtaacaagttaccttcaagtattctttctctatcgtcttttgagcttgaaaataaaaagcaatccgacaagttttagctgttaaaatggctcctgtagattggggaagtggtgtctgtgcttcagcttatgagtcatacatgaaaccaagtgtttctaagggaggcacgcccatgttgctgtggacccaattattctgctaggtcaaacatgcaaatctgcaggtaaaaacggtataggaaagtggggatgaagaggcaaggagcaagatctaccttgattagaccattactaagctcatattaattgccataatttaaaactaacaataaaatagagtaagaacctaca

SEQ ID NO: 283 - DNA, 612 bp, Artificial
  differential peak M51
aattcggcatgtgaccgtccctgccaagaccccagaaattaatttattatttaaccaaagccagtgattattgcataatttcatccttccaatcaagaatgctgctctttatttaatgactgagacctacttacttagcttccaaatatggtagtaattacttgatatacagattaatgcattcccaggctagtgccttgcacagaactatcaaatataattggggtttctaaccttgtgacacagccctacaaattgccttctttatttccttttcttaaaaagtacgttcagtctactctggactttcccatctgtcgtcaggtgacttcctgctctgttagctgaccgtgagtcacgtcgttgctgcacagcacgttcagtgaggctgcctctgcgtgtgaggcgaccaggagaggaaaggacagacttgagctatcagacctctctcctggtgggatattgtttgtttcccctaccctgttcatttaaaacctctcagttataagctttgctcctcagtttatgcctctgtgatgatgcttcaagagacagggaaaccagtttcctacattccaaaataaaaatgacattttaaaaagtctatccttt

SEQ ID NO: 284 - DNA, 672 bp, Artificial
  differential peak M52
ttgcccaatgatcacaggtgaataagatttaaggatgagtagctgctgatgaacccaatttgggatctggcaagaggaaatcagagcgtcaaatcaaggaggggtggcagcaaagaggaagcaaaagagaagacaagaggaaagagagtttatggagagaggaagccaatttgggaagtggggagaagggcaggctggccaggggatgcgacgcgacagggcagagggagtgggaagaagcgggcaggaggcggcctccacgcagctggcagggcaggcagcgctggagagggaggaggagagggaggctgcaggcaccgcgcagcagccgaggatgagagaggtaaccggagggaacgggcgcacacaactgcttccgcaggacttcctgcgataacacgcccacagcccgggcgccgagcacgcgtggtggcggccgcaggcccggggctcctgcgagagcgaacgccagcgacacacacgcagctcctgttgtgtcgccgctgccgcctaggcagggccgcagacagcagcctccttcccagactgagccaactcactctcaccgtagctgctccagaatccgtgaaaagaaaatggaatgctatttctgttcctgatctgcagttctgattcttttttcttcttaaaaacaaaaggaaaaattttttt

SEQ ID NO: 285 - DNA, 778 bp, Artificial
  differential peak M53
ctggggagtaggatgagggcaggcggagggtcttgacccaaatacacatattagcaaccatcagagtaactggatttgctgtaaaaaagttttcagagtaggactagaaaacttaagctaggatcttcaaaaacacagatataaggggtgaaaggaagaagttaagcataagctaggcaagtggaaaaaaaaagttaagacagtggtatcaaagaaactaaggaaagagtgttttaataatgaaggcactgtcaggagtcttagactactaacaggtcaagctagatgagaaatggaaagtacatctcatttagacaagcttactgacagccctaagaagagcagtttgagggaaggaagtggcagggccacagatagcaagggtgaaatggttacatctttaagtgggactttcccagctgcacttctgatcccagggcctgacgatttgtactgtcccccaacacaccttccccatcttccttagctgagtgaagtgttaaaaagggtttagaaaatggaaatagatagtacagacaattgttttaaaagtttccttattaaaatgaaatcaaagaatcaaacacaggcagaaggtcaagcctcatacagaaagacaggcatctcttaatgctaactggaaagcaagctaagcaggggcaggaacataagaagttaacagatttcttatatgcaaattcaggggtttctcagttgaaggcttctgatttttctgttaagcaagagaggtaatctgctgacaataaagatggaaaca

SEQ ID NO: 286 - DNA, 801 bp, Artificial
  differential peak M54
agaagctgatgccagagcaggtattttttctatagcaatgtcaaaacagtctaatacagagctcaaacagaattagctatttggtactttacattttattttccctttaaacgtaacaagaaacttaggagaaagatacctctatgatctttatttagaagaggagaaaattaaaataggctaaattgaaggactaagtggctaagttggctggacttccctaaggggactttcccctaagccaaaatgagtcatagctgcaagctaagggattgaaacttcaaccaatcatatagggagtttaagctctagctgcagtctgatgtttttaatcaatcaggccccccaacccacaagcagatagaaaataagctaatcctatagggcagaaaaaggaaaaggggaggagccataaggggatataagcataagacacccaagccagaaacagcaactcttctgggtccccttccaccgcgtggaagctttacttttgcttttgctttactttcactttcacttaagtaaattttgctgcctacactctttgggtctgcatgtttctctaatcgagttgtgacactcaccgccgcggtccacggcttcattccttgaagcctatgagaccacaaacccttcgattgagaaaaacctttgattggaagaagacttctcatctcaaaataactctttcaaaattaattaataaattttgagaggtgggattaatactgagaaacttcaggacatattactgcatttaaactctatagttcatgtgatttctatagattctcatga

SEQ ID NO: 287 - DNA, 553 bp, Artificial
  differential peak M55
gtgattgctgaaaggctatcgtacatctgtaaatgggcatgccattcctggtaccttctaactttgtgatcactgtccccatgcataaaatatggagtcctgcagagcagctagagaaagggtaagtagctcagagtctgtatcaatggggagctgtttaaaaattgatgttatcaagtaggttcacacattaaacacccagaaaatgttgggcctcctcctatgtggttttcctgaattcttgatgcaacctagaagaactaacgttaaaaagtaaagagcacagtacagacagtaagagttctgtttgtgaagactaatacaggaagtgatgctatgtctgattctagaatatggttagagtttcacagacaagaaacaagatctgtaacttgataattatgtaaatacagacctatgtgcaatcagtctagaagctccctgaaggcagggatgggttacccatattttgtctaacacttctttacattcttctaaggtgcgtcttggacacaataatcattgtgaacatacttgataacatcaagtatta

SEQ ID NO: 288 - DNA, 620 bp, Artificial
  differential peak M56
cccagctacttgggaggcttagataggaggattacttgagctcggaaggccaaggcttcagtgagccaagatcgcaccactgtactccagcctggacaacagggcaagaccttgtctccaaagaaaaaaataaaaaagagtatgtgaatatggctttccttaaaaaaaatttggtttcatgtccagaatgaacttttatggcccaaatatgtgttgtgaaattctgataggtccattaaaaatgcttgcacttaattccactatttggactgctgaatataagccttaaaagttgacatttatattggcaagcccaacagggcacaaaatctataaatggcagtgatggaggagacagaaatattttttcaagtacttcagcagtgacgctagggtgcagcattgtcccttttctctggcaaggtaatgcattcagaggaaacagaacataattttgatgacacaatgccagaaaccacaaagtaaaatctaaaggcacaaaccatagaactctacagcttctttgctgttggcaacctacgattgtcaggactttgccaaaagcagaactggcttttctttggaagcaggtcaaacatgccggtcaaatgttccttctg

SEQ ID NO: 289 - DNA, 717 bp, Artificial
  differential peak M57
ggtccatatttatatagaagttctgttagtcctgactacagcaaatgaagtcaaagatttgagtgaggaaaggcaataaaaaggaaagaaaggcttaactgtgcaaagacaaaaggtatctacagcaggcagagggaaaaaaacagagtacggttggaaagaccgtgaagttcatgtagttcaatcaagcatcaggtgcttcaattctatcatgttactgtcttattggtgccaagcttggcttggatgcttccagttataggaactcacttcctcctctacttcattatattgcttaactctgacttctagaatattctcctttatacttggctgaaacagaaagatgtgtggcataaattttactttctcttttacttctgctactcaaaggaagaggaagctgtgggtgcagatgttgctaagataaactacaacttcctccatgaccagtttctgcagtttaggaagggcagagccgaacacgtggctgagttggggagttaactacagacacttaagcaaatcagaatcctttgaaacaaaatcatcaaaaggccaagaagcatttgtaagggcttttgactacaacctaacattcagagagagttgatcagagagagttgttgacacactaaaaattcattacttttatttacattgaaatttatttactttattaaaaaatatttttgttggttaaggttgtttgtacaa

SEQ ID NO: 290 - DNA, 705 bp, Artificial
  differential peak M58
ggtataaatacctgagatgagaagtcttctccctatcaaaggtcttttctcaattgaagggatcgtggcctcacaggcttcaaggaatgaatccgtggaccgcggtggtgagtgttacagcttgactggagaaacgcgtgtacccaacgagtgtgcggtggcaagattaaagtgaaagcgaaagtaaagcgaaagcgaaagtgaaagtaaaagtaaagtttccacttggaacgggacctagaagggttgctgtttctggcttgggtgtcttatgcttatatccccttatgacctttccttttttcctttttctgtcctatagaattagcttattttcagtccgcttgtgggtttgcgggcctgattagttaaaaacgtcaggctgcagctagagcttaaactccctgtatgattggttgaagtttcaatccacagcttgcatctgtgactcattttggcttaggggaaagtcctttttgattggttgaagtttcaatcccttagcttgcagctatgacttattttggcttatgggaaagccccttcggtaagtccatattgacccaggaagtccagccaacttagccacttagtccctcatacccttaaaactaaccaaccgtaagtttttttacttaagtaaatacctgaaattcacagctacaaaaatggttagtagaaaaataactttaaatattggctatc

SEQ ID NO: 291 - DNA, 510 bp, Artificial
  differential peak M59
acaagctgaagtgaaggcccttgttataactgggaaattagcctccagcctctctaaaattggaagtgcagagagagaagggaaagtgaacaagtatctgtttcttcttccctctcccgctccagcccctcccccttagttctccaacccgggcctcactataggtcaaaatgaatgcagctgcccaacactgtgatgcgagctcgttcacactgacggacacttttctcatgttcacgtggtaggtcacagagcagtaggggaaagggaagtggtgatgcagtatctactacctacgctaggggtaggggaccagaaagttgctaggggaaaaaaaaatattcgaacagtgaaatggagcgtgtaacaaagtggaggagagtgactctttgaaccgctatggtggcaggaagagcaaatcaggtgctgtgttacaaaagaacagggaaaaaatgtacaagtgagatggtatatatccaagttatttgaagaccctgaaatccttcaaat

SEQ ID NO: 292 - DNA, 1005 bp, Artificial
  differential peak M60
cccattatcagggtccactcttgtatatcatgactacctattcccacctcaccctattcctcattccgtcagtgactttcagccacccgcaggcttacaaggttctgcgtcgccctaggcattttcttaatactcatccttttacaaaagacgtaatccgcaacagcttcgctttggggaccctttagcaaaagcaatgaattaggaacacctgggtggcaccatatcggccagccgttcctgtgggcccagccaaaatcttatcagcgattattcaggtactcaactacacagtacaggctccaggtggttttataagtccacgaggaacgaaagaaaaaaacatcgcaactcaacgtacacaaaccccaggcttcggttcccacataacatcaccttggttctccggcttcggcttctggttacgaaattcactttttccagcgcctttttctggccgctccagcgagggagacgccatttgcaagctgcttccggtggctccggaaatgaaactgttcttaaaccctaccggctaaaacgcctacactcaaaatgagttccgcggaactcagacgaggagatctgcgcagtctccgtgcgtacgtgcgttaagcccgcgcttttacacaaaccacagcttctcctacgtggccaatgaagagccggagactttctcccagtatgccctgaaaccatggagtttgcgtactgggacttgtagtttagcaggcggtataggcaaattggatactgttttatccgtagtttcctcgtggcctcacggcctaacctggaccatcggacagcggagtgctaccaacctgtcattgatgtcattcattctttcgataaatatttatatgttggccaaatatgcgctagatgttgggaggaagtacaaatatgagaatacaatgtatattgggcctccactgcaacaagtgacaggaatagctactatttattgtgagactactttgtttcagtactttatttacatta

SEQ ID NO: 293 - DNA, 1151 bp, Artificial
  differential peak M61
cgaggtgctattacaatcacatttctcatcagcccttttcttttttgcaaaagtgtttatcctagtgctgcggacttacgacaaacgtcaacagttaataagataaccaaaaactgtgaaaatcaaacgttcctggaatgcatcggagcttcacactttagtccttcgcttgagactcaaaggccacatggccagataggtctttcacctctgcaaaccgttaaacatccttccagtaagacataatcgttaagcccacggtgttaaagaaacccagtgagctgttccccgcaggtcaccgcctgcctccaccctcctcccgcgcactgaaggcaaaacacttagaccggaacgagcgaggccagaggagacaacaactcccagcaggcgtcgcggcagcgggaagtcacgcacgcgcaacccaggccacaggagtaaaatctctctgcccgtcttctgggaagggagaatggcggcgcccgggctgcggctgggagcgggaagactctttgaaatgcctgcggtgctagagcgactgagccgctataatagcacgtcccaagcttttgctgaggtgctgcggctgccgaagcagcagctgaggaagctgctgtacccgctgcaggaagtagagcggttcctcgccccctacgggaggcaagaccttcacctgcgtatctttgacccaagcccggaggacatagccagggcggacaacatcttcacggccactgaacggaaccgcatcgactacgtcagctccgccgtccgtatcgaccacgccccggaccttccgcggccagaggtgagaggcgattctcacggttcacctgcgcgccggcgtcacagcgctaaccgcttccctggccgtccggctcgcgggtgattttgcggttgtattttctagctttatgttggcgcatatattattttaactcaataggcatatgtcgatgctccctgtacgtggggatacccgagaaacccattgttaagtacaaaatgcagtcaatacccataaacccatggtaaagtcaaaaatcgttaagtcgacccactgtaagcagagactgcctgtatacagcttaaattattttggaatgaatatagaagacttcaaaatgctttaaaagcttttcaaacccaaatta

SEQ ID NO: 294 - DNA, 606 bp, Artificial
  differential peak M62
tgagggctgagaccatctggctttctaaagttcactttctggtggaggagggacccctatctggggacataggctcactagttatagatactgaggccctgggggtacagatgctggactggaggaagaggctgttagccctggactcttccagtctccactgctatttacccatgaccacacactccctgtcctctggtcagtctttgcagcctgtgcttgtcaaatcaaactatgtgttctgggaaatatgagcattttaaggctccgccttatctgataaacccttgaactgaagaaggcaggaagccacaggctcctgggtgggcagtacctacctggggttacccccgacctcagaggggaaggcagctgttctgagtcatcaagtaagcctaacaactaacacccacttacacgttcacatcatcagagagggaggcgaaggccaagttgaatgcctggaacgggggcactgagaagagcaaaggctttgggatgacagggcccaggttctagttctggctcttccacctcctagccatatctattagataacctctctcagtctctgaaagcccaataacaccatggtgcaatgcgatc

SEQ ID NO: 295 - DNA, 944 bp, Artificial
  differential peak M63
acaaaaaaagaaaggaacacacaaaagagctcatcattagttactccacaagcagcagctcatcattagttactccacaagaacaatgacctaaagcacgtattcataaatactagtacatttgactttcatatgatgaaatcagacaaaattctttcatcactgaatcacttgtagggagtagaagacaatggtgccacataacttttatgaagtactttctcttttcatcatgtgatgacagcaagttcttatccatacacacccaatcttaaagtctttcgctttcaacttaagttgaatgcaggtgtgtcaaaatgcttcatgtttgacacatctgacaatactacaggtgaactgaaccaggcagtaaacagttttttactgaaacagtgactcgatgttttggatactctacatagatttttaaatgacaattttttttaatacgtaataaaaagaagaagcaaagataaaagtttttttctaaagaaaagatactgagatgtgttagagatgtggcttcctcctggtatgaaagataccatcaaaaatacctgaaaagaataggcattatctatgtcatttcaatgaagtgtgtgttctgcagtcaagagaagcatttgacagtttgcatggggagaagtctgtcacacagatgtactcacctacagcacctgaaatcacctaaaaggaaggtttgtggattaggttgtgtggctgcgctgcctcactgctcccctctaaaaaaaaaaaaaaaaaaaaaaaaccacaagggaagtcacactgtgtatcccctagaaaaggatgtacacgtagagcagcctagaaatttaacagaacaaagaatctcttttaaaagaacagcttcacaaatttgtgtgcctttcttgtgtagggtcacgttaatcttgtcttgtaacctgggtataagagcagaataa

SEQ ID NO: 296 - DNA, 758 bp, Artificial
  differential peak M64
gaggctctttgctcagaatcctgaaaaggcttttgttactttattttttcttttttttcatcttctgggagcacagaataggcttctgctcctttaaataactttaacaggcaccaaaggaacactgctagctctttcttaattctgtaggtccacatttaggaaaaagaaattgtcagcctctgacttatttccagcttacaaaaaggctgtgatgttggcagcccggggaaagcagttcccgtgagtcatgctttcacctttcagccagtgaagaacaggaaatagtcacatcactattgccaacaagcacagcgaatcgcttccaccaccgggctttcccagatgacgtcagtgagccaagtgcagggcatcacccttgccagatggccccggaagagtctcagctgccctgtcaagtttagcttctcaagctccccagaaccagcatggcaaggatcacccctccagaaaaggaaatgatttctctgatcatgatcaaaccatgtcataattttagctgtaggtggtgagtatcagtgactgtattatagatggttttggttcacagctattttttttctcggtatatttactctcaagggcaaaggggtggcatttatcacaatgctatgattcactctactagactggctgggtttcatttcatctttgagttctagacctaaggccaaaaggatgtcagaatcgccacccagagctagatgcatgctcaatgcaacctggccatctctctc

SEQ ID NO: 297 - DNA, 636 bp, Artificial
  differential peak M65
taggataccgtaagaatccttttttcttgggctactttataaaacatacataccacagagtttcaggctctttaaaaacccatctttacactgatctaactgctcagattaattttgaaacaggcaacgtgcagaaaaaaacaacatctaaaaattaaacctcccctctttccgttctaaagtttcatttcaaaagtattaggaaatcttactttaagcagcgagctgatggcgttctccagaactcggtttgagcaacaaacttgagacaacctttgtctggaagcaagtcaagcgcttaataatgcatgctgggatttgtagtttccctgctttcagagtttagtggctccatccatacactttataaatgtatgtgctgccatctagtggaattcaggtagaactatgaattccaagtggaaggtgaaaagtggaaaagcaccttagagcttacttaggatgcggaaatcatttccttcacttcagtctctccacttctctgttttgtttatgcctactatatgcggtgccagggtagacgagattggctgagctggaaactgcgtgtggaagatagcagagcaacaaaatagagggaacccaggtcactgaaaattctggagccttcctgtc

SEQ ID NO: 298 - DNA, 900 bp, Artificial
  differential peak M66
ggtcccctctaatcacaccatcccccctcttttaaaaaaattttcttctcagataaccaagttaaatctagctcaaatgaaccttttagtttttgaggagggcaataatagtagtgaacaccaaaacgaaaggggatctgggcttcaaaatactaacagttctatttcctagccagtcgtctcccgggctggatgtgccgccgtcccagatctcagcggcggccgcagagcgcagaggtagggagagaaaaaggaggcgtcggcggggcgcgagcgaccagggcgctaagtgccgcctcacagtcctgccagagacaggagacgccgggccgcccgcgccgtctgtcccaagagctcctcctggacatccgcttgactctccgtcgtctccaattggccgtcggggaacggaagccgaagcaaactagtgaacccggaagtgcttcgcggcggaggcccgggcaactcttttgaatggaatcgggctgattcatcgccggtttgcagacagagccgcgtcgggtgtgcgccgctgctgctgttgcctctgtcttcgcgtcaccacagaggcaagacaagggtccatatcgcggcatccggctcccgcccgtcttcaggtaagacccagaaggcaggcctgggaggtctggggaggcagccgtgctaggccgccgccagccccgtgctttcctgagtcacgttgcccagctgagttgccacaggaggcctgggccgcgagggaggagcccggcctccttcattctcttcgcctgctctcagggctctctggcagggtcaggtctggatgtcgcctcctttctccattcttttctgtttggccgaattcacatcatgggaacctcttccatctatatagagagaatccttgtttatggcctg

SEQ ID NO: 299 - DNA, 1017 bp, Artificial
  differential peak M67
aaataggacatttcaagaaaaaggcatcctggtaggagagagcagaagttttaaacctcatttgaccttcatgaagttgtcaaaatcaccgtggctccgtaagaatgtgagtgcatgttcttattgcaagcctgttttaccaggccacaccagccataagcatggtctggagcacgagtgacattgctgtggtctctgacactactcttgcttctgggtttccaagtcaggttggttccaattcccaggggtgctgaggatgctcctggccttccatggaggctatttcagctttgcctgcctttgccacgatggctgttcttgcatagtctggccatcatttatttctaccactgttgcctcacctgcttcatcccctgaagagcctcgaggtggtgccagagttcacttttgctgctattcttgcttgtggttatttgcatcctgttgctctcaaagcttcctctgctggcctgttacgctggtgctgctcaccgcaactgggtaagctttttttacccctcttcctgtcgctcttcaaggtttccaaaagaccatctttcagcagttgcctcaattaaggccttggaaccaggtataccggggactgggcatgagggtaactgagggaccccctttcctggaggtactttaaagtacttctccctaaggatggtcaacccagtggcacaatccaagtgacaagtcttttgacttcccctgtctcagggtgacttactgcctttataggccaccccttcttcccgcagaagtcggagcaacttttgacttccgtctaggttcatgagcatttctcctaaaatcctaaggccacccttcattttgtttcctatttcagcctagggggttcctcatgcaaattcagacatctgatggcctgctctcatataccagagttttggagatgggcatgcagattaatatccgctccctgctctccatgaccctttgctgcagttaagactgatttgcagctctagaaatgaaggccatgtg